SEQUENCE LISTING (4 sequences)

SEQ ID NO: 1 - DNA, 718 bp, Homo sapiens
acaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctc

SEQ ID NO: 2 - protein, 206 aa, Homo sapiens
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 3 - DNA, 34 bp, Artificial Sequence
  Description of Artificial Sequence  Synthetic Primer
ttttttgtcgacatggcccagggccaagaattcc

SEQ ID NO: 4 - DNA, 33 bp, Artificial Sequence
  Description of Artificial Sequence  Synthetic Primer
ttttttgcggccgcgagcttgtagaatttctgc

(19) United States
(12) Patent Application Publication
(10) Pub. No.: US 2004/0009939 A1
(43) Pub. Date: Jan. 15, 2004

(54) Methods of enhancing immune induction involving MDA-7

(75) Inventors: Sunil Chada, Missouri City, TX (US); Abujiang Pataer, Houston, TX (US); Abner Mhashilkar, Houston, TX (US); Rajagopal Ramesh, Sugarland, TX (US); Jack Roth, Houston, TX (US); Steve Swisher, Fresno, TX (US)

Correspondence Address: Gina N. Shishima, Fulbright & Jaworski L.L.P., Suite 2400, 600 Congress Avenue, Austin, TX, 78701, US

(73) Assignees: Board of Regent, The University of Texas System; Introgen Therapeutics, Inc.

(21) Appl. No.: 10/378,590

(22) Filed: Mar. 3, 2003

Related U.S. Application Data
(60) Provisional application No. 60/404,932, filed on Aug. 21, 2002.
(60) Provisional application No. 60/370,335, filed on Apr. 5, 2002.
(60) Provisional application No. 60/361,755, filed on Mar. 5, 2002.

(51) Int. Cl. A61K 48/00; A61K 38/17

(52) U.S. Cl. 514/44; 514/12; 424/93.2

(57) ABSTRACT

The present invention relates to compositions and methods for the enhancing or inducing an immune response against an immunogenic molecule by indirectly activating PKR. More specifically, immunotherapy is improved by co-administering a MDA-7 polypeptide with an immunogenic molecule against which an immune response is desired. Such immunotherapies include cancer vaccines, and compositions thereof are described.

This application claims the priority of U.S. Provisional Patent Application Serial No. 60/404,932, filed Aug. 21, 2002, U.S. Provisional Patent Application Serial No. 60/370,335, filed Apr. 5, 2002, and U.S. Provisional Patent Application Serial No. 60/361,755 filed Mar. 5, 2002, the entire disclosures of which are specifically incorporated herein by reference.

The United States Government may own rights in the invention pursuant to grant number CA 86587 from the National Cancer Institute.

### BACKGROUND OF THE INVENTION

A. Field of the Invention

The present invention relates generally to the fields of immunology and molecular biology. More specifically, the present invention is directed to diagnostic, prognostic, and therapeutic treatment compositions and methods for enhancing or inducing an immune response to an immunogenic molecule by providing an effective amount of an MDA-7 polypeptide. In one embodiment, the invention relates to enhancing the immunogenicity of a vaccine, such as a cancer vaccine, by administering an effective amount of a MDA-7 polypeptide.

B. Description of Related Art

Immunotherapy is a rapidly evolving area in cancer research and exploits the body's natural ability to protect from foreign invasion at sub-cellular, cellular, molecular and macromolecular levels. Immunotherapy, also known as biological therapy, biotherapy, biological response modifier therapy or immune therapy, provides a treatment option for certain types of cancers either directly or indirectly by fighting cancer cells or lessening the side effects of alternative cancer treatments (i.e., chemotherapy).

For example, the immune system may recognize tumor cells as a foreign substance and thus tumor cells may be targeted for destruction by the immune system. Unfortunately, the response typically is insufficient to prevent tumor growth. However, recent research in the area of immunotherapy has focused on the development of methods that augment or supplement the natural defense mechanism of the immune system. Examples of immunotherapies currently under investigation or in use are immune adjuvants (e.g., Mycobacterium bovis, Plasmodium falciparum, dinitrochlorobenzene and aromatic compounds) (U.S. Pat. No. 5,801,005; U.S. Pat. No. 5,739,169; Hui and Hashimoto, 1998; Christodoulides et al., 1998), cytokine therapy (e.g., interferons), and (IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Edward and Villaseca, 1998; U.S. Pat. No. 5,830,880 and U.S. Pat. No. 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311).

One area in which immunotherapy may be employed is treatment for cancer. Normal tissue homeostasis is a highly regulated process of cell proliferation and cell death. An imbalance of either cell proliferation or cell death can develop into a cancerous state (Solyanik et al., 1995; Stokke et al., 1997; Mumby and Walter, 1991; Natoli et al., 1998; Magi-Galluzzi et al., 1998). For example, cervical, kidney, lung, pancreatic, colorectal and brain cancer are just a few examples of the many cancers that can result (Erlandsson, 1998; Kolmel, 1998; Mangray and King, 1998; Gertig and Hunter, 1997; Mougin et al., 1998). In fact, the occurrence of cancer is so high that over 500,000 deaths per year are attributed to cancer in the United States alone.

Currently, there are few effective options for the treatment of many common cancer types. The course of treatment for a given individual depends on the diagnosis, the stage to which the disease has developed and factors such as age, sex and general health of the patient. The most conventional options of cancer treatment are surgery, radiation therapy and chemotherapy. Surgery plays a central role in the diagnosis and treatment of cancer. Radiation therapy, chemotherapy and immunotherapy are alternatives to surgical treatment of cancer (Mayer, 1998; Ohara, 1998; Ho et al., 1998). Chemotherapeutic strategies are often limited by the difficulty of achieving drug delivery throughout solid tumors (el-Kareh and Secomb, 1997).

The cDNA encoding the MDA-7 protein, referred to as MDA-7 herein, has been described by Jiang et al., 1995 (WO 95/11986, incorporated herein by reference). The protein encoded by the mda-7 cDNA was recognized as a potential regulator of melanoma progression. Jiang et al. used a subtractive hybridization technique (Jiang et al., 1995, incorporated herein by reference) to identify genes involved in the regulation of growth and differentiation in human melanoma cells.

A cDNA library prepared by subtraction hybridization of cDNAs prepared from actively proliferating human HO-1 melanoma cells against cDNAs prepared from interferon-beta (IFN-β) and mezerin-differentiated human HO-1 melanoma cells was used to identify several melanoma differentiation associated (mda) cDNAs, including mda-7. The expression of mda-7 mRNA is inversely correlated with melanoma progression as demonstrated by increased mRNA levels in normal melanocytes as compared to primary and metastatic melanomas as well as decreased mda-7 mRNA expression in early vertical growth phase melanoma cells selected for enhanced tumor formation in nude mice. It is not clear how apoptosis is achieved by MDA-7 nor does it appear that MDA-7 has been implicated in mechanisms involving immune response.

Gene therapy is another emerging field in biomedical research with a focus on the treatment of disease by the introduction of therapeutic recombinant nucleic acids into somatic cells of patients. Various clinical trials using gene therapies have been initiated and include the treatment of various cancers, AIDS, cystic fibrosis, adenosine deaminase deficiency, cardiovascular disease, Gaucher's disease, rheumatoid arthritis, and others. Currently, adenovirus is typically the vehicle for the delivery of gene therapy agents. Advantages in using adenovirus as a gene therapy agent are high transduction efficiency, infection of non-dividing cells, easy manipulation of its genome, and low probability of non-homologous recombination with the host genome. The primary modality for the treatment of cancer using gene therapy is the induction of apoptosis. This can be accomplished by either sensitizing the cancer cells to other agents or inducing apoptosis directly by stimulating intracellular pathways. Other cancer therapies take advantage of the need for the tumor to induce angiogenesis to supply the growning tumor with neccessary nutrients. Endostatin and angiostatin are examples of two such therapies (WO 00/05356 and WO 00/26368).

Genetic immunizations employing naked DNA or non-viral vectors as carriers of immunogenic molecules have demonstrated considerable success in animal models of cancer and infectious disease. However, these studies have not correlated with results from human clinical trials, where, in general, limited immune induction/ augmentation has been observed. Also, the ability to enhance an immune response has prognostic implications. A patient may be tested using the methods of the present invention for immune induction to determine if a patient is a good candidate for immune therapy. Therefore, it would be of great benefit to improve the reliability of genetic immunizations and of identification of immunogenic molecules. Thus, there exists a continued need for improvements in the area of immune responses, both from a treatment perspective (immunotherapy), as well as from a prophylactic and possibly diagnostic perspective.

### SUMMARY OF THE INVENTION

The present invention is based in part on the observation that MDA-7 induces and/or activates ds-RNA dependent protein kinase (PKR), which leads to the phosphorylation of eIF-2α. PKR has been implicated in methods of enhancing or promoting an immune response. Other observations on which the inventions are based can be found in the Examples section. Thus, the invention relates to methods and compositions for enhancing and/or promoting an immune response involving MDA-7 peptides, polypeptides, or nucleic aids encoding an MDA-7 peptide or polypeptide, and any compound against which an immune response can be induced or is desired.

Compositions of the invention include an immunogenic composition, wherein the term “immunogenic composition” refers to a composition against which an immune response (cellular or humoral) can be detected or induced. Immunogenic compositions, in some embodiments of the invention, comprise a molecule against which the immune response is desired or can be detected (in the presence or absence of MDA-7 compositions of the invention) and all or part of a recombinant MDA-7 polypeptide or a nucleic acid encoding such a polypeptide.

It is contemplated that the MDA-7 peptide or polypeptide may comprise at least 10, 20, 30, 40, 50, 60, 70, 80, 90, 100, 110, 120, 130, 140, 150, 156, 157, 160, 170, 180, 190, 200 or 206 contiguous amino acids of SEQ ID NO:2 or comprise all of SEQ ID NO:2. The recombinant MDA-7 polypeptide may be modified, or it may be truncated at either end. In some embodiments of the invention, the MDA-7 polypeptide comprises amino acids 49 to 206, 75 to 206, or 100 to 206 of SEQ ID NO:2. The secreted form of MDA-7 has amino acids 49 to 206 of SEQ ID NO:2, but the first 48 amino acids are absent, and it is specifically contemplated that the secreted form qualifies as “the MDA-7 polypeptide” and may be used in any composition or method of the invention. Alternatively, an MDA-7 amino acid sequence may include a heterologous amino acid sequence, such as a secretory signal. In some embodiments, the secretory signal is a positively charged N-terminal region that has a hydrophobic core. In other embodiments, the secretory signal targets MDA-7, or a truncation thereof, to the endoplasmic reticulum or mitochondria.

It is contemplated that in any embodiments of the invention involving a polypeptide, that a nucleic acid encoding that polypeptide may be utilized. Thus, in some aspects of the invention, an MDA-7-encoding nucleic acid and/or a nucleic acid encoding an immunogenic peptide or polypeptide is utilized. The nucleic acid may be contained in an expression vector or construct. The vector may be viral or nonviral. In some embodiments, the construct is a viral vector, such as an adenovirus, adeno-associated virus, herpesvirus, retrovirus, vaccinia virus, polyoma virus, rhabdovirus, or alphavirus. Compositions may include about 10³ to 10¹⁵ viral particles, about 10⁵ to 10¹³ viral particles, about 10⁷ to 10¹¹ viral particles, or about 10¹⁰ viral particles. Nucleic acids may further include a promoter operably linked to the nucleic acid sequence. It it contemplated that a single nucleic acid may encode multiple polypeptides, such as 1, 2, 3, 4, 5 or more polypeptides, including both an MDA-7 polypeptide and one or more immunogenic polypeptides.

The MDA-7-encoding nucleic acid may encode any of the MDA-7 polypeptides described above or may comprise or be at least 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, or 618 contiguous nucleotides of SEQ ID NO:1.

Compositions of the invention contain an “immunogenic molecule,” which refers to a molecule that is capable of eliciting an immune response, alone or in combination with compositions of the invention. It is contemplated that the immunogenic molecule may not be able to induce or provoke an immune response without MDA-7 and/or an adjuvant; alternatively, the immunogenic molecule may be able to induce or provoke an immune response in the absence of MDA-7 and/or an adjuvant, but the MDA-7 or adjuvant may lead to an enhanced immune response against the immunogenic molecule. In some embodiments the immunogenic molecule comprises one or more polypeptides.

In other embodiments of the invention, an immune response to an immunogen may be invoked by administration of MDA-7 or a nucleic acid encoding MDA-7 in combination with a cytokine, chemokine, or analog thereof, including, but not limited to interleukins and interferons, in particular IFNα, IFNβ, IFNγ and lambda IFNs. The cytokine or chemokine may be provided as a polypeptide or as a nucleic acid encoding the polypeptide. A therapeutic benefit, such as an immune response to a pathogen, a cancer cell, a tumor cell, a hyperproliferative cell, or other disease conditions may be invoked by the administration of composition(s) comprising MDA-7 and a cytokine or chemokine. It is further contemplated that the cyotokine, chemokine, or analog thereof may be in a pharmaceutically or pharmacologically acceptable formulation.

Compositions may comprise MDA-7 and a cytokine or chemokine, or two different compositions comprising MDA-7 or a cytokine or chemokine may be used in combination. As separate compositions, they may be administered simulataneously or one before the other. One may be administered 1, 2, 3, 4, 5, 6, 7, 8, 9, 10, 11, 12 hours or 1, 2, 3, 4, 5, 6, 7 days or more before or after the other.

Cytokines, chemokines, or analogs thereof may be of mammalian origin, and it is specifically contemplated that they may be human versions of the polypeptide.

Immunogenic molecules of the invention include antigens or epitopes against which an immune response can be observed or is desired. An “antigen” refers to a substance or portion of a substance that is specifically recognized by an antibody or T cell receptor. It is used synonymously with the term “immunogen.” Antigens of the invention include one or more epitopes, which are antigenic determinants. An antigenic determinant refers to the structure of an antigen molecule that interacts with the combining site of an antibody or T cell receptor as a result of molecular complementarity. The immune response against immunological molecules of the invention may be cellular or humoral. It is contemplated that compositions of the invention may include nucleic acids encoding antigens and epitopes of the invention. Such nucleic acids may be comprised in expression vectors discussed above with respect to MDA-7 encoding nucleic acids. Furthermore, such nucleic acids may be operably linked to promoters. Nucleic acids encoding immunogenic polypeptides include immunogenes. In some embodiments, immunogenes encode Mycobaterium tuberculosis soluble factor (Mtb), phenol soluble modulin (PSM), CMV-G, CMV-M, EBV capsid-EB nuclear antigen (EBNA), gp120, gp41, tat, rev, gag, toxa antigen, rubella antigen, mumps antigen, alpha-fetoprotein (AFP), adenocarcinoma antigen (ART-4), BAGE, CAMEL, CAP-I, CASP-8, CDC27m, CDK4/m, CEA, CT, Cyp-B, DAM, ELF2M, ETV6-AMLI, ETS G250, GnT-V, HAGE, HER2/neu, HLA-A*0201-R1701, HPV-E7, HSP 70-2M, HST-2, hTERT, ICE, KIAA 0205, LAGE, LDLR/FUT, MAGE, MART, MC1R, MUCI, MUM-1, MUM-2, MUM-3, NA88-A, NY-ESO-I, p15, Pm1/RARalpha, PRAME, PSA, PSM, RAGE, RU1, RU2, SAGE, SART-1, SART-3, TEL/AML1, TPI/m, TRP-1, TRP-2, or WT1. Any proteinaceous immunogenic molecule can be provided in a composition as an immunogene.

In some embodiments of the invention, an antigen is a tumor antigen, a microbial antigen, a viral antigen, a fungal antigen, or other disease/condition associated antigen. A disease/condition associated antigen is one that arises as a result of a particular condition or disease or is an indicator of a particular condition or disease. A tumor antigen is an antigen is a disease/condition associated antigen in which the disease is cancer. Tumor antigens include, but are not limited to, PSA, CEA, MAGE1, MAGE3, gp100, AFP, her2, tert, muc1, NY-ESO, bcr-ab1, trp1, trp2, MART, BAGE, GAGE, or PMSA. Specifically included for use with the invention are human tumor antigens, some of which are disclosed above. In some cases, it may be advantageous to use xenoantigens, i.e., those derived from rodent species as, in some cases, xenoantigens appear to activate the immune system better than the syngeneic antigen.

Microbial, viral, and fungal antigens are antigens derived from microbes, viruses, or fungi. Microbes include, but are not limited to, any gram negative and gram positive bacterium, as well as others discussed herein. Viral antigens specifically include, but are not limited to, antigens from viruses discussed herein. Thus, it is specifically contemplated that methods and compositions of the invention can be used to induce or elicit an immune response against viruses, microbial organisms, or fungi.

The immunogenic molecule may be a small molecule, a nucleic acid, a peptide or a polypeptide. In specific embodiments, the immunogenic molecule is a T-cell activation molecule.

Compositions of the invention may constitute or comprise vaccines. A vaccine is a preparation of an isolated antigen, in some cases from viral, bacterial, or other pathogenic agent, that can be administered to a subject prophylactically to induce immunity.

In further embodiments of the invention, compositions also include a colloidal carrier. The colloidal carrier includes, but is not limited to, a proteinoid, an emulsion, or a liposome. Compositions may also include adjuvants other than an MDA-7 polypeptide.

It is specifically contemplated that compositions of the invention are included in pharmaceutically or pharmacologically acceptable formulations, diluents, or solutions.

The present invention also includes methods involving compositions of the invention. Methods of the invention generally involve promoting an immune response in a patient comprising administering an effective amount of an MDA-7 polypeptide or an MDA-7-encoding nucleic acid under the control of a promoter to the patient. In alternative embodiments, methods may comprise the promotion of an immune response in a patient by administering an effective amount of MDA-7 polypeptide or an MDA-7 encoding nucleic acid in combination with a cytokine, chemokine or analog thereof, which includes, but is not limited to, IFN α, IFN β, IFN γ, lambda IFNs, IL-1, IL-2, IL-4, IL-6, IL-8, L-10, or IL-12. An “effective amount” refers to an amount that achieves a desired result. Alternatively, an “effective amount” refers to an amount that yields a therapeutic benefit to the patient. Thus, in certain further embodiments, it is contemplated that a subject is in need of promoting or enhancing an immune response. In some embodiments of the invention, “effective amount” refers to an amount that results in the achievement of a particular goal, such as enhancing, increasing, inducing, improving, or promoting an immune response, which can be detected, directly or indirectly, by a variety of methods known to those of ordinary skill in the art.

In further embodiments, a molecule that is ultimately immunogenic is also provided to the subject. In such cases, the molecule and the MDA-7 polypeptide or MDA-7-encoding nucleic acid may be provided in the same composition or one may be provided before the other. The method may be performed in vivo, in vitro, or ex vivo.

In some embodiments of the invention, methods concern therapeutic or prophylactic purposes to induce, promote or enhance an immune response in a subject. When performed in vivo, the immunogenic molecule composition and MDA-7 composition (refers to a composition comprising either an MDA-7 peptide or polypeptide or a nucleic acid sequence encoding an MDA-7 peptide or polypeptide) are administered to a subject. In various embodiments, compositions comprising at least one cytokine, chemokine, or analog thereof may be included in or administered with MDA-7 compositions of the invention. Any of the compositions discussed herein may be employed in methods of the invention. In some embodiments, the subject is a human or other mammal. It is contemplated that these methods of the invention may constitute a vaccine regimen against a particular immunogenic molecule.

Alternatively, methods of the invention may be used for diagnostic or prognostic purposes. In these cases, it is contemplated that an observation of an immune response against a particular molecule is indicative of a disease/condition or its prognosis. All methods and compositions of the invention may be employed for in vitro, in vivo, or ex vivo use.

An immune response can be detected in a variety of ways including, but not limited to, measuring an increase in cytokine concentration or production, an increase in T cell proliferation, increase in B cell proliferation, increase in T cell activity, increase in NK cell activity, increase in macrophage activity, or increase in antibody production. In some embodiments, the cytokine concentration of an interferon (e.g., IFN-α, IFN-β, IFN-γ) or an interleukin (e.g., IL-1β, IL-2, IL-4, IL-6, IL-8, IL-1 0, or IL-12) is indicative of an immune response.

In some cases, compositions are administered to a subject more than one time, and at least or at most 1, 2, 3, 4, 5, 6, 7, 8, 9, 10 or more times. Compositions may be administered hourly, daily, weekly, biweekly, monthly, or annually or they may be administered every 1, 2, 3, 4, 5, 6, or 7 or more days, 1, 2, 3, 4, or 5 or more weeks, or 1, 2, 3 ,4, 5, 6, 7, 8, 9, 10, 11, or 12 or more months. Compositions may be administered to cells or a subject orally, intravenously, intraperitoneally, intramuscularly, subcutaneously, by continuous infusion, by direct injection, regionally, intratracheally, intralesionally, or intraarterially. Systemic administration or systemic therapy is specifically contemplated as part of the invention.

Administrations of compositions may be in combination with other therapies. In some embodiments, anti-cancer, anti-microbial, or anti-viral treatments are provided in addition to compositions of the invention. In some embodiments, an anti-cancer therapy is chemotherapy, surgery, radiotherapy, hormone therapy, or gene therapy. Gene therapy may also be employed as an anti-microbial or anti-viral treatment. Additionally, cytokine or chemokine therapies may also be employed, such as IFN-α, IFN-β, IFN-γ, IL-1β, IL-2, IL-4, IL-6, IL-8, IL-10, and/or IL-12.

It is specifically contemplated that aspects or features discussed with respect to one embodiment of the invention may be implemented or employed with respect to any other embodiment of the invention discussed herein. Another embodiment of the present invention is a method of enhancing an immune response against an immunogen comprising providing to a patient a nucleic acid sequence encoding the immunogen; and administering to the patient an effective amount of an MDA-7 polypeptide, wherein the MDA-7 polypeptide enhances the immune response against the immunogen. The immunogen can also be as provided as the product, peptide, or polypeptide. In other embodiments, MDA-7 may be administered in combination with at least one cytokine or chemokine, such as IFNα, IFNβ, or IFNγ.

The immunogen can include Mycobaterium tuberculosis soluble factor (Mtb), phenol soluble modulin (PSM), CMV-G, CMV-M, EBV capsid-EB nuclear antigen (EBNA), gp120, gp41, tat, rev, gag, toxa antigen, rubella antigen, mumps antigen, alpha-fetoprotein (AFP), adenocarcinoma antigen (ART-4), BAGE, CAMEL, CAP-I, CASP-8, CDC27m, CDK4/m, CEA, CT, Cyp-B, DAM, ELF2M, ETV6-AMLI, ETS G250, GnT-V, HAGE, HER2/neu, HLA-A*0201-R1701, HPV-E7, HSP 70-2M, HST-2, hTERT, ICE, KIAA 0205, LAGE, LDLR/FUT, MAGE, MART, MC1R, MUCI, MUM-1, MUM-2, MUM-3, NA88-A, NY-ESO-I, p15, Pm1/RARalpha, PRAME, PSA, PSM, RAGE, RU1, RU2, SAGE, SART-1, SART-3, TEL/AMLI, TPI/m, TRP-1, TRP-2, or WT1.

The invention further provides a method of treating cancer in a patient comprising providing to the patient a tumor antigen; and administering an effective amount of a MDA-7 polypeptide, wherein the MDA-7 enhances the induced immune response and provides a therapeutic benefit to the patient. A method of treating cancer may further comprise administering to the patient an effective amount of a cytokine or chemokine wherein the MDA-7 and a cytokine or chemokine enhance the induced immune response and provides a therapeutic benefit to the patient. The term “therapeutic benefit” used herein refers to anything that promotes or enhances the wellbeing of the subject with respect to the medical treatment of his condition, which includes treatment of pre-cancer, cancer, and hyperproliferative diseases. A list of nonexhaustive examples of this includes extension of the subject's life by any period of time, decrease or delay in the neoplastic development of the disease, decrease in hyperproliferation, reduction in tumor growth, delay of metastases, reduction in cancer cell or tumor cell proliferation rate, and a decrease in pain to the subject that can be attributed to the subject's condition.

In certain embodiments, the present invention is directed to a method of treating a tumor in a patient comprising (a) providing to the patient an immunogenic molecule to induce an immune response against the immunogenic molecule; and (b) administering to the patient an effective amount of a MDA-7 polypeptide, wherein the MDA-7 enhances the induced immune response and decreases the tumor as compared to treatment with the immunogenic molecule alone. A method of treating a tumor may further comprise administering to the patient an effective amount of at least one cytokine or chemokine. The resulting decrease of the tumor refers to a decrease in tumor size or a decrease in tumor growth rate. In some embodiments, the immunogenic molecule is a tumor antigen and can include PSA, CEA, MAGE1, MAGE3, gp100, AFP, her2, tert, muc1, NY-ESO, bcr-ab1, trp1, trp2, MART, BAGE, GAGE, or PMSA.

In certain embodiments, the present invention is directed to a method of releasing cytochrome c from mitochondria of a cell comprising contacting the cell with an amount of an MDA-7 polypeptide or a nucleic acid encoding an MDA-7 polypeptide effective to cause or promote the release of cytochrome c from the mitochondria. In some embodiments, the cell may refer to a tumor cell. The tumor cell can include, but is not limited to a lung, head and neck, pancreatic, prostate, renal, bone, testicular, breast, cervical, gastrointestinal, lymphoma, brain, ovarian, leukemia, myeloma, colorectal, esophageal, skin, thyroid, liver, or bladder tumor cell. The nucleic acid encoding the MDA-7 polypeptide may be contained within a vector. A vector as used herein referes to an expression vector or a delivery vector. An expression vector contains nucleic acid sequence necessary for the transcription of an mda-7 encoding polynucleotide. A delivery vector is a means of transferring the expression vector into a cell. In various embodiments, the vector is an expression vector. The expression vector may be delivered by a viral vector or a non-viral vector. The viral vector may include an adenoviral vector, a retroviral vector, a vaccinia viral vector, an adeno-associated viral vector, a polyoma viral vector, an alphaviral vector, a rhabdoviral vector, or a herpes viral vector. In still further embodiments, the MDA-7 polypeptide or a nucleic acid encoding the MDA-7 polypeptide is administered to a patient or subject. The MDA-7 polypeptide or the nucleic acid encoding the MDA-7 polypeptide may be administered by intratumoral injection, intratracheal injection, intravenous injection, intrapericardial injection, intramuscular injection, subcutaneous injection, topical application, mucosal exposure, orally, lavage, subcutaneously, or as a direct injection to an immunocompromised site. The MDA-7 polypeptide or the nucleic acid encoding the MDA-7 polypeptide may be administered in an amount between 10³ and 10¹⁵ viral particles. In still other embodiments, the MDA-7 polypeptide or the nucleic acid encoding the MDA-7 polypeptide may be administered more than one time. The MDA-7 polypeptide or the nucleic acid encoding the MDA-7 polypeptide may be administered to a tumor bed prior to or after resection of the tumor, or both prior to and after resection of the tumor. In some embodiments, the MDA-7 polypeptide or the nucleic acid encoding the MDA-7 polypeptide may be administered to a patient before, during, or after chemotherapy, surgery, immunotherapy, hormonal therapy, or radiotherapy. The MDA-7 polypeptide or the nucleic acid encoding the MDA-7 polypeptide may be administered 72 hours prior to, 24 hours prior to, 72 hours after, or 24 hours after chemotherapy, surgery, immunotherapy, hormonal therapy, or radiotherapy.

In some embodiments, the present invention is directed towards a method of promoting or increasing the expression of tumor suppressor proteins, E-cadherin or PTEN, in a tumor cell comprising contacting the cell with an amount of an MDA-7 polypeptide or a nucleic acid encoding an MDA-7 polypeptide that is effective to promote or increase the expression of one or both said tumor suppressor proteins.

In various embodiments, the present invention is directed towards a method of decreasing protein expression of proto-oncogene, PI3K, in a tumor cell comprising contacting the cell with an amount of an MDA-7 polypeptide or a nucleic acid encoding an MDA-7 polypeptide that is effective to decrease the expression of PI3K. In some embodiments, the proto-oncogene may modulate cell-cell adhesion and/or intracellular signaling.

In still other embodiments, the present invention is directed towards a method of inducing G2 cell cycle arrest in a tumor cell comprising contacting the tumor cell with an amount of an MDA-7 polypeptide or a nucleic acid encoding an MDA-7 polypeptide that is effective to induce G2 cell cycle arrest in a tumor cell. G2 cell cycle arrest may be induced by Cdc25c pathway inhibition.

In yet still further embodiments, the present invention is directed towards a method of inducing anti-angiogenesis in a tumor comprising contacting a tumor cell or an endothelial cell adjacent to the tumor cell with an effective amount of an MDA-7 polypeptide or a nucleic acid encoding an MDA-7 polypeptide, wherein the MDA-7 polypeptide binds to an IL-22 receptor that is effective to induce anti-angiogenesis. In some embodiments, anti-angiogenesis results from inhibition of migration of endothelial cells toward growth factors. Growth factors include, but are not limited to VEGF and/or bFGF. Anti-angiogenesis may result from inhibition of endothelial cell differentiation.

In some embodiments, the present invention is directed towards a method of delivering MDA-7 to a cell, comprising obtaining an MDA-7 targeting construct, wherein the MDA-7 targeting construct includes a DNA molecule encoding an MDA-7 polypeptide or the nucleic acid encoding the MDA-7 polypeptide and a targeting sequence under the control of a promoter, and contacting the cell with an amount of the targeting construct that is effective to deliver the MDA-7 targeting construct to the cell. In some embodiments, the targeting construct comprises DNA encoding MDA-7 without a functional MDA-7 signal peptide, with a nuclear localization signal peptide, with an endoplasmic reticulum signal peptide, or with a mitochondrial signal peptide.

Other embodiments of the invention concern the use of MDA-7 in combination with inhibitors of particular molecules and/or their activities.

In some embodiments, methods of the invention involve inducing or enhancing cell death in a tumor cell by administering MDA-7 protein an MDA-7 encoding nucleic acid with an inhibitor of NF-κB. Inhibitors of NF-κB include IκB and Sulindac, a non-steroidal anti-inflammatory drug. In other embodiments, inhibitors of COX-2 protein or activity are part of the invention. Also included in the invention are inhibitors of Hsp90, such as geldinamycin or analogs thereof. It is also contemplated that inhibitors of protein kinases or their activity are also part of the invention. Moreover, other anti-inflammatory agents, in addition to Sulindac, may be implemented as part of the invention, such as naproxen.

As MDA-7 binds to the IL-22 receptor, the IL-22 receptor acts to inhibit angiogenesis. Other IL-22 agonists can be used as anti-angiogenic agents alone or in combination with MDA-7 in aspects of the invention.

Furthermore, it is clear from the interactions and activities of MDA-7 that it is involved in certain pathways, which may be taken advantage of as part of the invention. MDA-7 affects the β-catenin and P13 kinase (PI3K) signalling pathway. Also, MDA-7 promotes the secretion of IL-6, IFN-γ, IL-12, TNF-α, and GM-CSF. Therefore, in some embodiments of the invention, there is a method for promoting secretion of 1L-6, IFN-γ, IL-12, TNF-α, and/or GM-CSF in a peripheral mononuclear blood cell (PMBC) involving administering an effective amount of MDA-7 to the cell. Also MDA-7 activates STAT3 expression and can be used in methods and compositions of the invention to achieve such activation.

It is contemplated that embodiments discussed herein with respect to one aspect of the invention may be implemented with respect to other aspects of the invention. Furthermore, it is contemplated that any compositions of the invention may be used in any methods of the invention.

Another method of the invention concerns the use of MDA-7 protein to induce anti-angiogenesis of a tumor. Tumors become vascularized and angiogenesis is induced around the tumor. The present invention uses MDA-7 polypeptide to inhibit or reverse that process by inducing anti-angiogenesis. The phrase “inducing anti-angiogenesis” refers to a reversal or inhibition of vascularization or to inhibition of angiogenesis that has already begun. In some embodiments, a patient with a tumor is administered an effective amount of an MDA-7 polypeptide to bind the IL-22 receptor on IL-22-receptor positive cells and induce anti-angiogenesis of the tumor. IL-22 receptor-positive cells are cells that express IL-22 receptor, which binds MDA-7, on their surface. Thus, in some embodiments, the IL-22 receptor-positive cells of the patient are given an effective amount of MDA-7. In further embodiments, the IL-22 receptor-positive cells are endothelial cells. Therefore, it is contemplated that endothelial cells in the patient may be given MDA-7 polypeptides. Futhermore, these cells do not need to be adjacent (“abutting” or “next to”) to the tumor or to tumor cells. It is contemplated that they may be remote (not adjacent) with respect to the tumor. Moreover, in some embodiments, the MDA-7 polypeptide is the secreted form MDA-7 and is glycosylated.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1A. Cell death in A549 (wild-type p53) and H1299 (null p53) cells after treatment with Ad-mda7 (2500 viral particles). FIG. 1B. Dose dependent PKR expression in A549 and H1299 cells after treatment with Ad-mda7. Expression of actin was used as a control. FIG. 1C. Immunofluorescent confocal microscopy of A549 and H1299 cells after treatment with Ad-mda7.

FIG. 2A. Expression of PKR, phospho-PKR, eIF-2alpha and phospho-eIF-2alpha in A549 cell lysates after treatment with PBS, Ad-Luc or Ad-mda7. Expression of actin was used as a control. FIG. 2B. Protein fractions of A549 cells treated with Ad-Luc or Ad-mda7 for 48 hours after immunoprecipitation with anti-human Tyk2, Stat1, Stat3 or p38 and staining with anit-human phospho-Tyk2, Stat1, Stat3 or p38 antibodies. FIG. 2C. Expression levels of Bid, PARP, caspase-3, caspase-8 and caspase-9 in A549 treated with PBS, Ad-Luc, or Ad-mda7. Expression of actin was used as a control.

FIGS. 3A-3C. Lung cancer cells treated with 2-AP after Ad-mda7 transduction. FIG. 3A. Cell death in A549 cells treated with Ad-mda7 in combination with 2-AP. FIG. 3B. Immuno-stained protein fractions from A549 cells after treatment with Ad-mda7 and 10 mM 2-AP. Experiment performed in triplicate. FIG. 3C. Protein synthesis in cells after treatment with Ad-mda7 or Ad-Luc in combination with 10 mM 2-AP.

FIGS. 4A-4C. PKR-dependent induction of Ad-mda7-induced apoptosis in MEFs from PKR+/+ and PKR+/− cells. FIG. 4A. Expression of PKR, MDA-7 and actin after 48 hour treatment with Ad-mda7. FIG. 4B. Cell death following 48 hour treatment with PBS, Ad-Bak, or Ad-mda7. FIG. 4C. Morphology indicates apoptosis solely in PKR+/+cells after treatment with Ad-mda7.

FIGS. 5A-5B. Adenoviral mediated overexpression of MDA-7 suppressed proliferation and induced cell death in lung cancer cells. FIG. 5A. Cell viability was determined with the XTT assay. FIG. 5B. Percentage of cell death in A549 cells following treatment with PBS, Ad-Luc (2500 vp) or Ad-mda-7 (2500 vp). The cells were analyzed by flow cytometry after transduction. Triplicate experiments were performed for each cell line, and data are represented as mean±S.D.

FIGS. 6A-6B. Ad-mda-7 effect on mitochondrial membrane potential changes and apoptosis. Release of cytochrome c from the mitochondria was measured by immunoblotting in both H11299 cells (FIG. 6A) and 1549 cells (FIG. 6B).

FIGS. 7A-7B. Effect of Ad-mda-7 on mitochondrial membrane potential. Measurement of mitochondrial membrane potential after transduced with Ad-mda-7, Ad-p53 and Staurosporine (1 μM). Where indicated the cells were pretreated with CsA (10 μM). H1299 (FIG. 7A) and A549 (FIG. 7B) cells stained with the potential sensitive dye, tetramethylrhodamine, ethylester, perchlorate (TMRE), and fluorescence was assessed by flow cytometry. Results are the mean i SD of three separate experiments.

FIGS. 8A-8B. Cyclosporine A (CsA) does not prevent loss of the mitochondrial membrane potential. H1299 (FIG. 8A) and A545 (FIG. 8B) cells were treated with Ad-mda-7, Ad-p53 and Staurosporine (1 μM). Where indicated the cells were pretreated with CsA (10 μM). The cells were then lysed, and the mitochondrial membrane potential was determined with the fluorescent dye TMRE.

FIG. 9. Ad-mda-7 up-regulates the extrinsic pathway. Ad-mda-7-treated A549 cells were assessed by immunoblot assay for changes in BAK, BAX, Bc1-2, TNF-α, TNF-R1, TRADD, FasL, Fas and FADD expression.

FIG. 10. Schematic demonstrating the effects of several pro-apoptotic genes that induce mitochondrial membrane potential changes, which open MMP-dependent pores and allow the release of cytochrome c and the formation of the apoptosome with APAF-1 and caspase 8.

FIGS. 11A-11C. Ad-mda-7 does not significantly alter steady state levels of β-catenin. FIG. 11A. MDA-MB-435 breast cancer cells were either untreated (Lane 1) or transduced by Ad-Luc (Lane 2) or Ad-mda-7 (Lane 3) at 2000 vp/cell. Cells were harvested and lysed 48 hr post-treatment and analyzed for expression for MDA-7 protein, β-catenin and β-actin by Western blot using specific monoclonal antibodies. FIG. 11B. H1299 cells or HUVEC cells were transduced by either Ad-Luc or Ad-mda-7 (MOI 1000 vp/cell) and 48 hr post-treatment, stained with anti-MDA-7 polyclonal antibody and observed for subcellular expression of MDA-7 protein by immunofluorescence. A representative field from two independent experiments is shown. FIG. 11C. Apoptosis in Ad-mda-7 transduced H1299 and HUVEC cells. H1299 and HUVEC cells were transduced with Ad-Luc or Ad-mda-7 and 48 hrs post-transfection, the cells were analyzed by apoptosis by Annexin V staining. Results shown are representative of >3 experiments.

FIGS. 12A-12C. FIG. 12A. Regulation of β-catenin by MDA-7. Breast cancer cells were treated with either Ad-Luc or Ad-mda-7 at 2000 vp/cell for 48 hr. The cells were fixed and analyzed by immunofluorescence microscopy for β-catenin localization. The result shown is representative of 3 independent experiments. FIG. 12B. MDA-MB-453 breast cancer cells or HUVEC cells were treated with Ad-Luc, Ad-p53 or Ad-mda-7 (at MOI of 1000 vp/cell) and 48 hr later analyzed for β-catenin staining by immunofluorescence. FIG. 12C. MDA-7 regulates -catenin transactivation. H1299 cells were transfected by lipofectamine with either LEF/TCF promoter-based TopFlash plasmid or LEF/TCF-promoter-based FopFlash. Three hours after transfection the cells were transduced with either Ad-GFP or Ad-mda-7 virus at MOI of 1000 (H1299). After 48 hrs cells were analyzed for luciferase activity. Data are shown as mean±S.D. of triplicate samples. This study was performed twice with identical results.

FIGS. 13A-13C. Ad-mda-7 up-regulates E-cadherin and inhibits cancer cell migration. FIG. 13A. NSCLC cancer cells (H1299, A549) were treated with PBS, Ad-mda-7 or Ad-Luc (MOI of 2000 vp/cell) and 48 hr post-infection, cells were trypsinized, washed with PBS and incubated with primary antibody against E-cadherin. Ad-mda-7 increases surface E-cadherin in both lung cancer lines as seen by FACS analysis. Data was plotted as mean±S.D. of triplicate samples. The study was performed more than 3 times with identical results. FIG. 13B. H1299 cells transduced with either Ad-mda-7 or Ad-Luc were evaluated for cell migration. Ad-mda-7 treated cells migrated less than Ad-Luc treated cells. FIG. 13C. HI 299 cells transduced with either Ad-mda-7 or Ad-Luc were evaluated for cell-cell adhesion. Ad-mda-7 treated cells showed greater homotypic adhesion than Ad-Luc or PBS treated cells. These studies were all performed at least twice with comparable results.

FIGS. 14A-14C. Ad-mda-7 regulates molecules in the β-catenin and PI3K pathways. FIG. 14A. Ad-mda-7 up-regulates (i) APC and (ii) GSK-3β. Lysates from MDA-MB-453 cells were probed for steady state levels of GSK-3β and APC proteins. Lane 1, untreated cells; Lane 2, Ad-Luc treated cells; Lane 3, Ad-mda-7 treated cells. Cells were treated with 2000 vp/cell for 48 hrs. FIG. 14B. Ad-mda-7 down-regulates PI3K, ILK-1, PLC-γ and FAK. Lysates obtained from Ad-mda-7 transduced MDA-MB-453 cells were probed for steady state levels of PI3K, FAK, PLC-γ and ILK-1. FIG. 14C. (i). Regulation of pFAK by Ad-mda-7 in H1299 cells. Lane 1, untreated; Lane 2, Ad-Luc treated; Lane 3, Ad-mda-7 treated; Lane 4, LY294002 at 20 μM final concentration. Cells were treated with 1000 vp/cell for 48 hrs. (ii). Ad-mda-7 upregulates PTEN in MDA-MB-453 cancer cells. Anti-PTEN monoclonal antibody was used to probe cell lysates via Western blotting in untreated (Lane 1), Ad-Luc-treated (Lane 2) and Ad-mda-7 treated (Lane 3) MDA-MB-453 cells. These studies were all performed at least twice with comparable results.

FIG. 15. Schematic illustrating Ad-mda-7 induced modulation of β-catenin and PI3K pathways. MDA-7 upregulates tumor suppressor proteins and down-regulates proto-oncogene expression.

FIG. 16. MDA-7 expression in DU145, LNCaP and PC-3 prostate cancer cells. The cells were infected with Ad-mda-7, Ad-Luc or treated with PBS as a mock control. Cells were harvested 24 hr, 48 hr and 72 hr after infection and lysed with SDS sample buffer. The proteins were then blotted onto nitrocellulose membranes and probed with an anti-MDA-7 antibody. The corresponding β-actin levels are shown as loading controls.

FIG. 17. Cell viability assay of DU145, LNCaP and PC-3 prostate cancer cells and PrEC epithelial cells. The cells were infected with Ad-mda-7, Ad-Luc or treated with PBS. Cells were harvested and stained with 0.4% trypan blue daily on days 1-4 after infection to reveal dead cells. Viable cells were then counted using a hemocytometer. Data were generated in triplicate; the average percentages of cell viability rate compared with PBS treatment are shown. Bars, standard error (SE).

FIGS. 18A-18B. Induction of apoptosis caused by MDA-7. FIG. 18A. DU145, LNCaP and PC-3 prostate cancer cells and PrEC epithelial cells were infected with Ad-mda-7, Ad-Luc or treated with PBS. Cells were harvested 72 hr after treatment, and cells in sub-G0/G1 phase were analyzed as apoptotic cells using flow cytometry. 20,000 events were captured for each treatment; the data are shown as histograms. Data were generated in duplicate; the average values are shown. Bars, standard error (SE). FIG. 18B. 72 hr after infection, attached cells were analyzed using Hoechst 33258 staining. The arrows indicate cells undergoing apoptotic cell death. The degree of magnification was ×20 for all cell lines.

FIG. 19. Cell cycle analysis of DU145, LNCaP and PC-3 prostate cancer cells and PrEC epithelial cells infected with Ad-mda-7. Cells harvested 72 hr after treatment, and cell cycle analysis was performed using flow cytometry. 20,000 events were captured for each treatment; the data are shown as histograms. The cell cycle phase is represented on the X axis. Data were generated in duplicate; the average values are shown. Bars, SE.

FIGS. 20A-20B. Target proteins of negative regulation by MDA-7 in DU145 and LNCaP cells. Cells were infected with Ad-mda-7, Ad-Luc or treated with PBS. Cells were then harvested 72 hr after infection or treatment and lysed with SDS sample buffer. The proteins were blotted onto nitrocellulose membranes and then probed with various antibodies associated with the target pathways of MDA-7. Activation of apoptotic caspase cascade (caspase 9, −3 and PARP) and target proteins of negative regulation by MDA-7 in Du145 cells (FIG. 20A) and LNCaP cells (FIG. 20B) were analyzed using Western blotting. The corresponding β-actin levels are shown.

FIGS. 21A-21B. Downregulation of proteins, associated with G2 cell cycle arrest, by MDA-7. DU145 and LNCaP cells were infected with Ad-mda-7, Ad-Luc or PBS. Cells were harvested 72 hrs after treatment and lysed with SDS sample buffer. The proteins were blotted onto nitrocellulose membranes and then probed with antibodies which detect the proteins regulating the cell cycle. FIG. 21A. DU145 cells; FIG. 21B. LNCaP cells. The corresponding -actin levels are shown as loading controls.

FIGS. 22A-22C. FIG. 22A. Endothelial cells (HUVEC and HMVEC) were mixed with various concentrations of sMDA-7 (b,c,e,f) or endostatin (h,i,k,l) protein and plated in 96-well plates. Cells that were not treated served as controls (a,d,g,j). 24 hrs later the ability of endothelial cells to form capillary-tube like structures were examined under bright-field microscopy. sMDA-7 inhibited tube formation in both, HUVEC (b,c) and HMVEC (e,f) cells and was dose dependent. However, the inhibitory effect by endostatin was not observed in both, HUVEC (h,i) and HMVEC (k,l) cells at these concentrations. FIG. 22B. Immunodepletion of sMDA-7 protein resulted in restoration of tube formation ability by HUVEC cells (b,c) that was similar to the untreated control (a). (×4 magnification). FIG. 22C. The ability of sMDA-7 to inhibit HUVEC cell migration induced by 100 ng/ml VEGF. A significant inhibition of HUVEC cell migration by sMDA-7 was observed when compared to control cells that did not contain sMDA-7 protein.

FIGS. 23A-23B. HUVEC and HMVEC treated with sMDA-7 were analyzed for pSTAT-3 protein expression by Western blot analysis and by immunofluorescence. FIG. 23A. Induction of pSTAT-3 expression in both HUVEC (a) and HMVEC (b) cells was observed at 4 hr and 24 hr by Western blot analysis. FIG. 23B. Immunofluorescence analysis demonstrated nuclear localization of pSTAT-3 in HUVEC cells treated with sMDA-7 compared to control cells which demonstrated cytoplasmic localization.

FIG. 24. HUVECs were treated with IL-22R1 blocking antibody (1 ng/ml and 5 ng/ml). 24 hrs later, cells were harvested, mixed with Matrigel containing sMDA-7 (5 ng/ml) and observed for tube formation. Untreated cells (a); treated with 5 ng sMDA-7 (b); treated with IL-22R1 antibody (1 ng) (c); treated with IL-22R1 (1 ng) and sMDA-7 (5 ng) (d); treated with IL-22R1 antibody (5 ng) (e); treated with 1L-22R1 antibody (5 ng) and sMDA-7 (5 ng) (f); treated with anti-IL10R antibody (5 ng) (g); treated with anti-IL10R antibody (5 ng) and sMDA-7 (5 ng) (h); (4× magnification). Semi-quantitative analysis of number of tubes formed by HUVECs treated with sMDA-7 demonstrated significantly less number of tubes than those that were not treated or treated with IL-22R1 antibody. The inhibitory effect of sMDA-7 correlated with increased pSTAT-3 expression while in the presence of IL-22R1 antibody, pSTAT-3 activation by sMDA-7 was inhibited. Error bars denote standard error.

FIGS. 25A-25B. sMDA-7 and endostatin (12.5 ng) were encapsulated in Matrigel containing bFGF (60 ng) and implanted subcutaneously into athymic nude mice. Matrigel that contained bFGF served as a positive control while Matrigel alone served as a negative control. On day 10, Matrigel was harvested and examined for neovascularization (FIG. 25A) and hemoglobin content (FIG. 25B). A significant reduction in hemoglobin content was observed in Matrigel containing sMDA-7 compared to controls.

FIGS. 26A-26D. FIG. 26A. An equal mixture (1:1) of A549 tumor cells and 293-mda-7 cells were combined in Matrigel (1×10⁶) and implanted subcutaneously in nude mice (n=10).

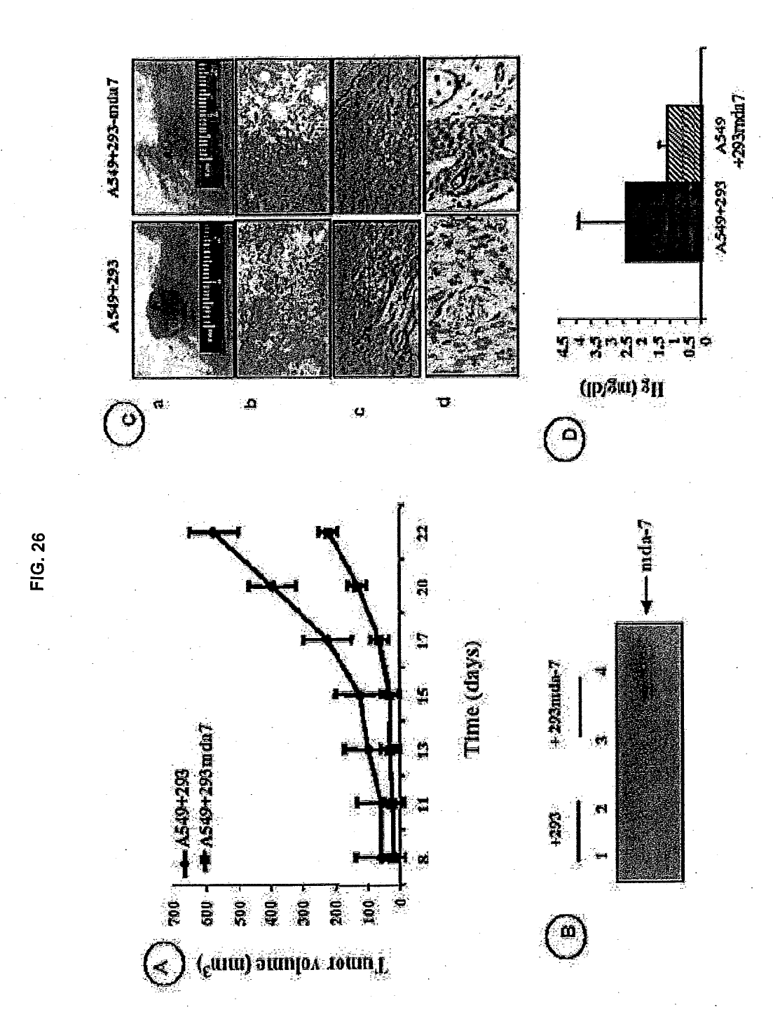

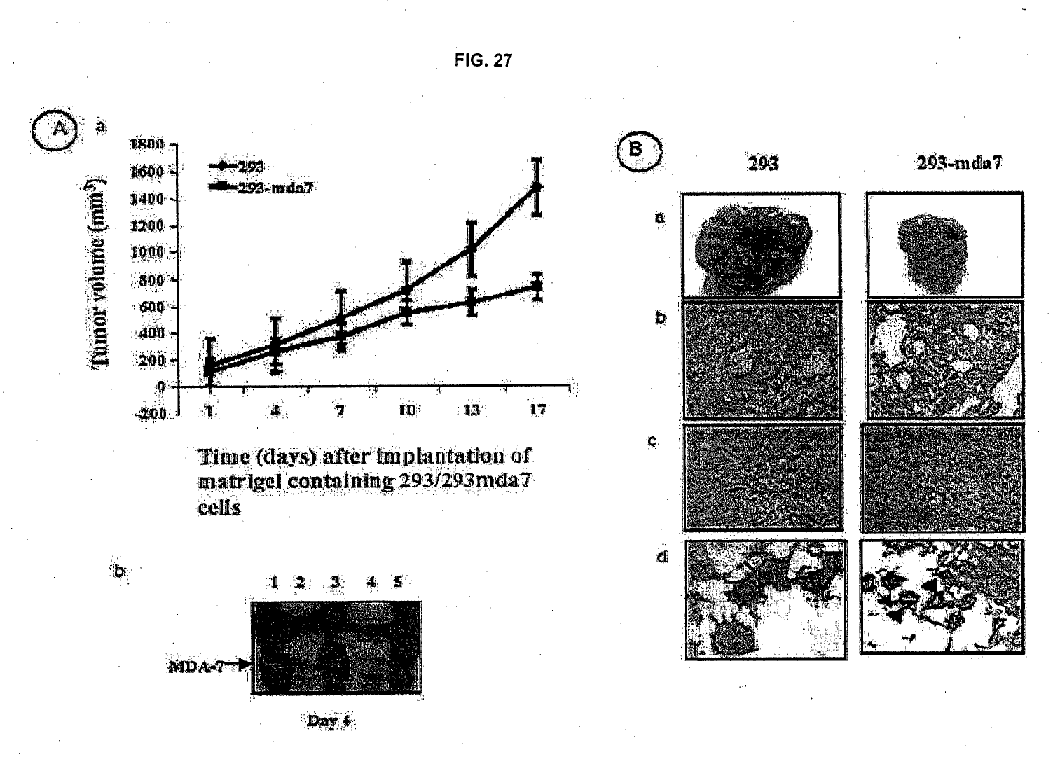

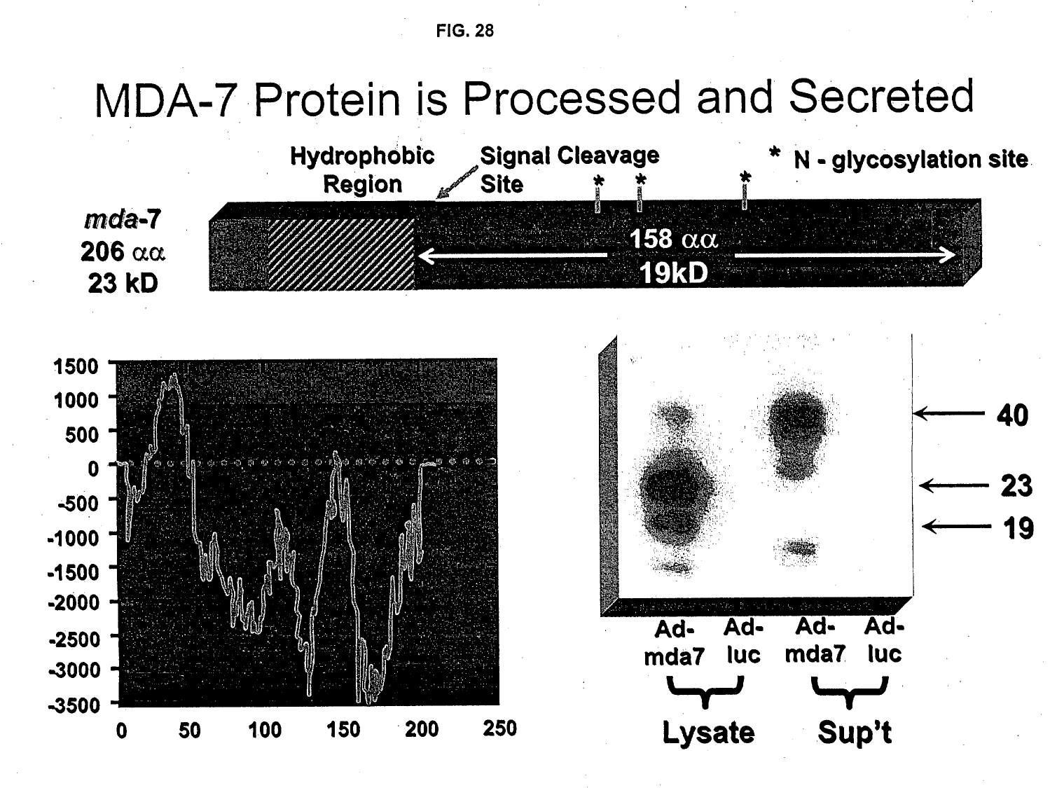

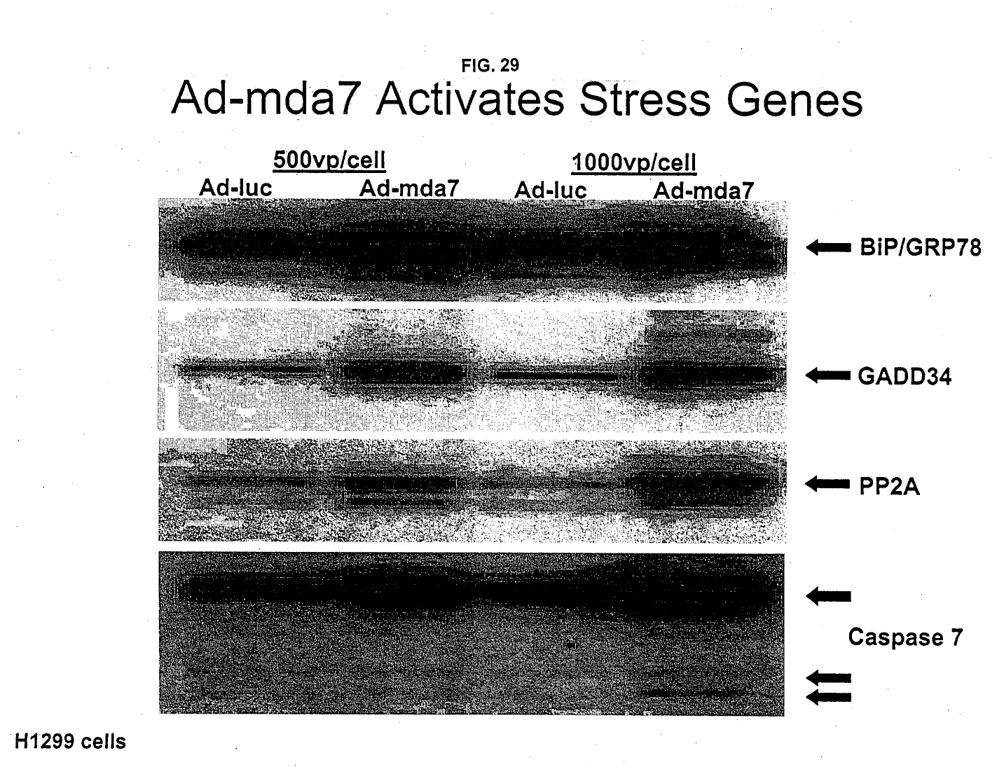

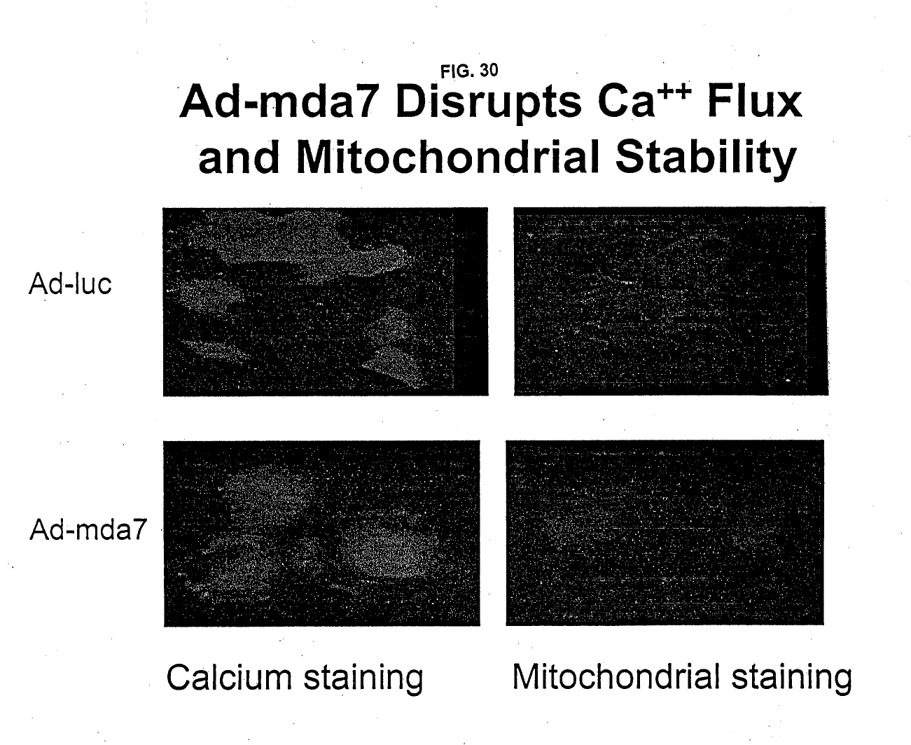

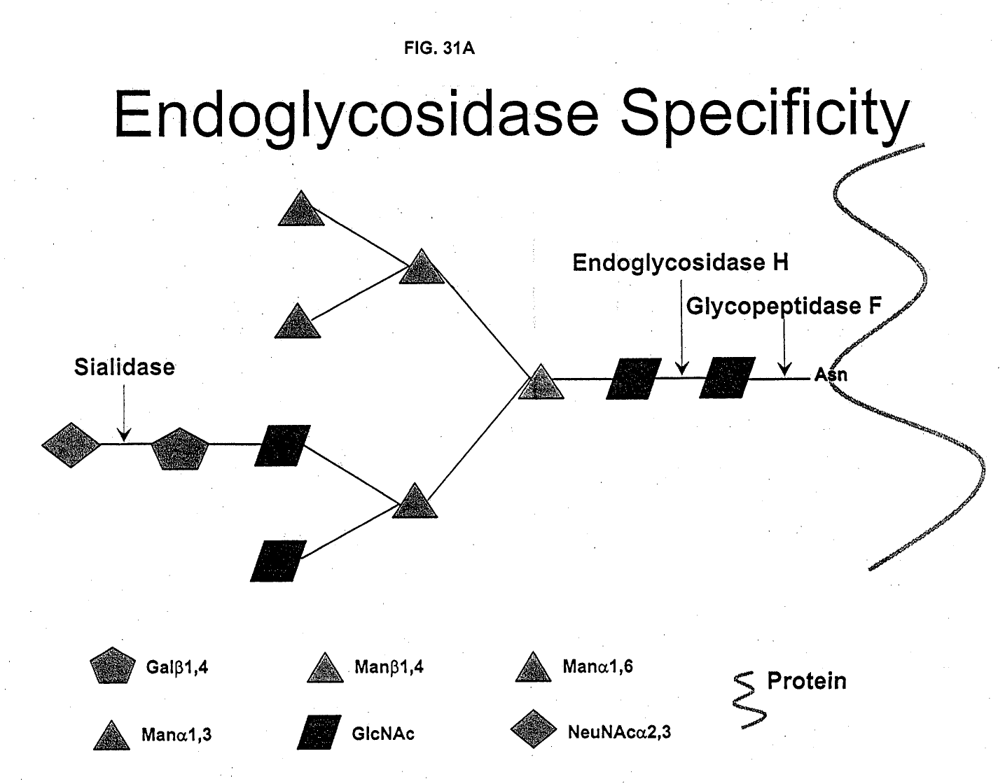

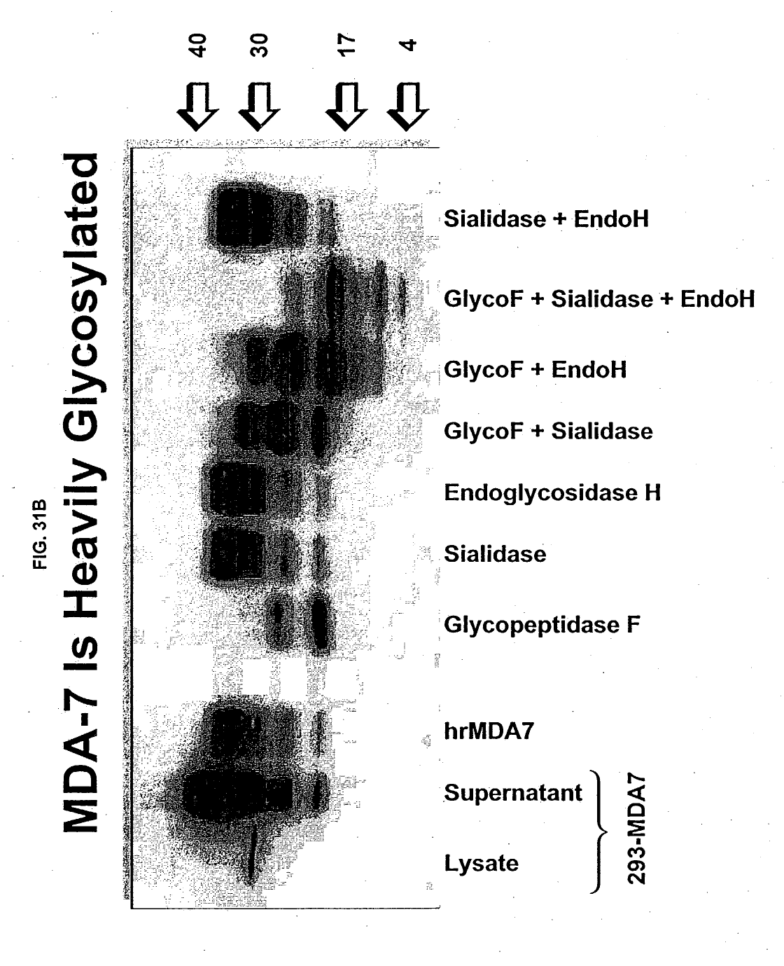

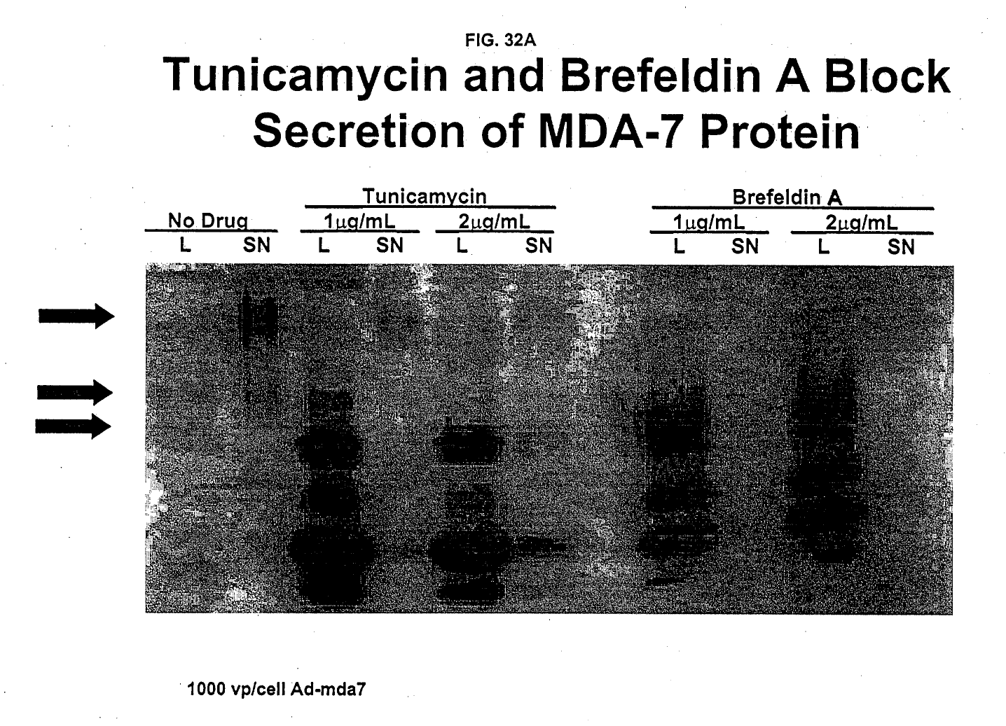

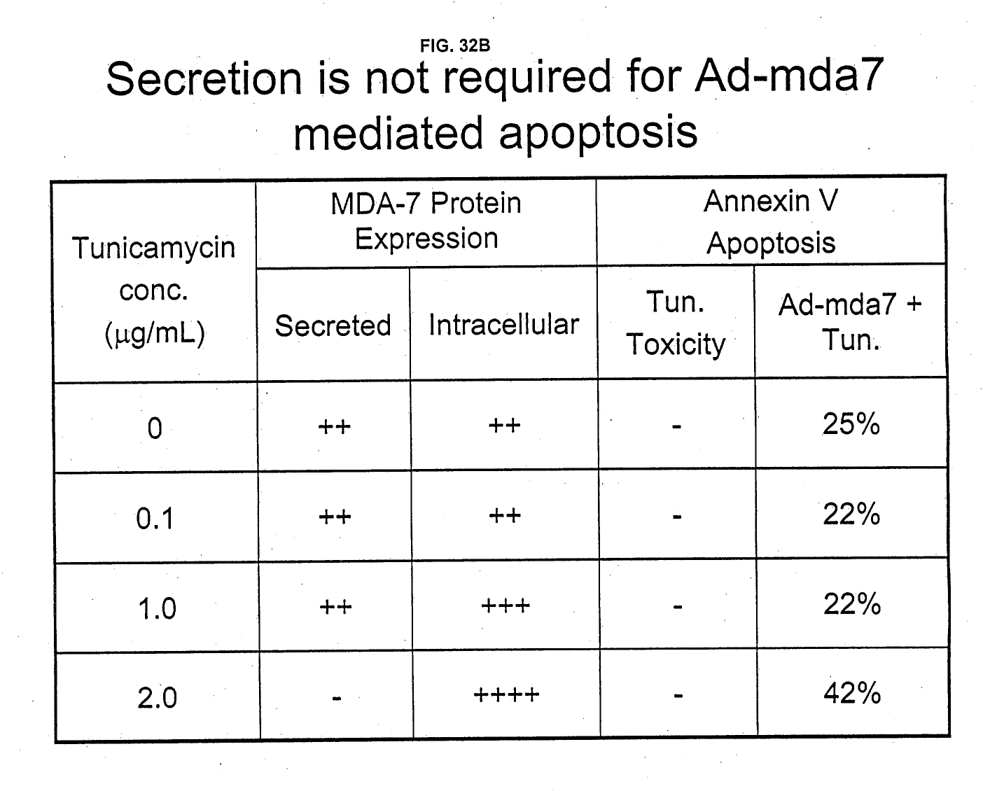

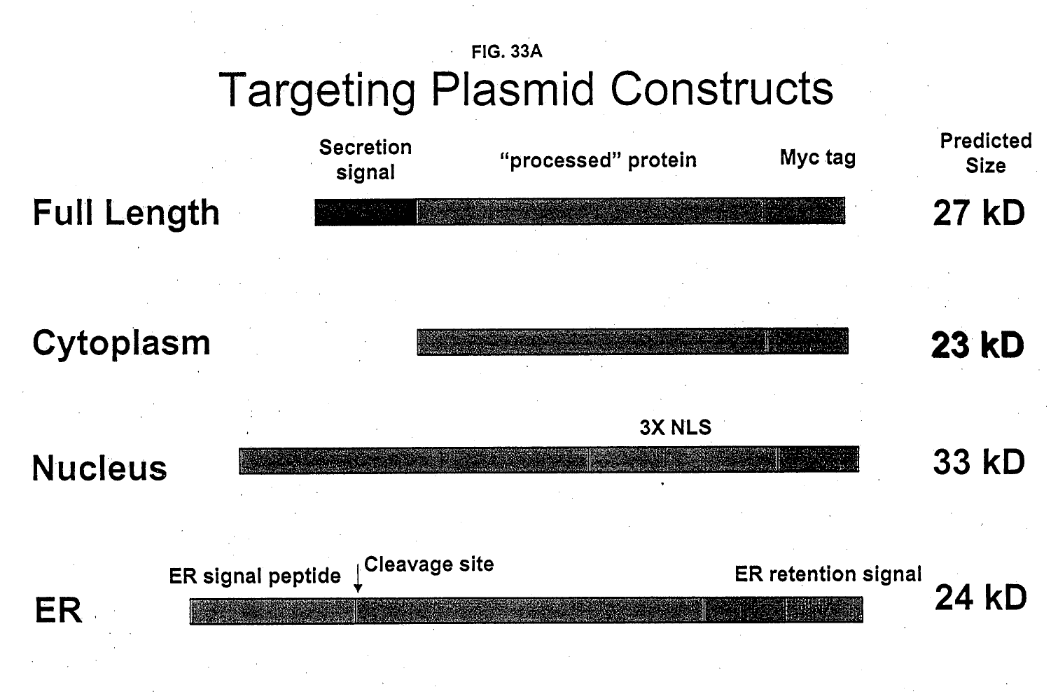

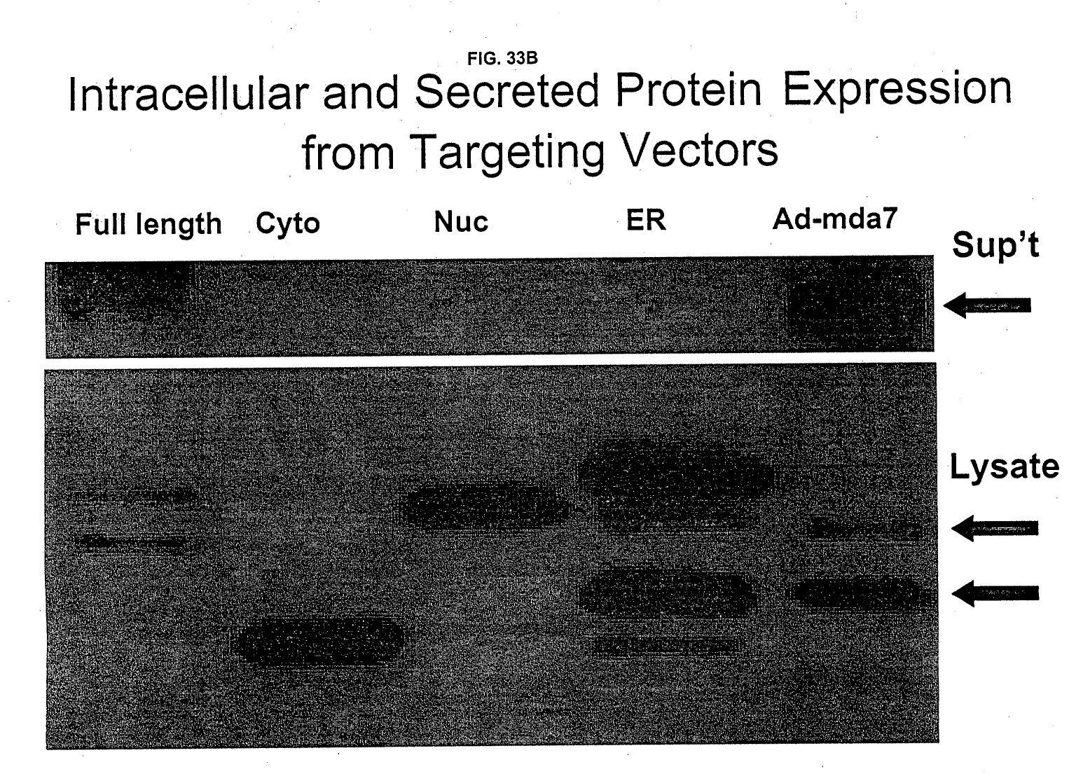

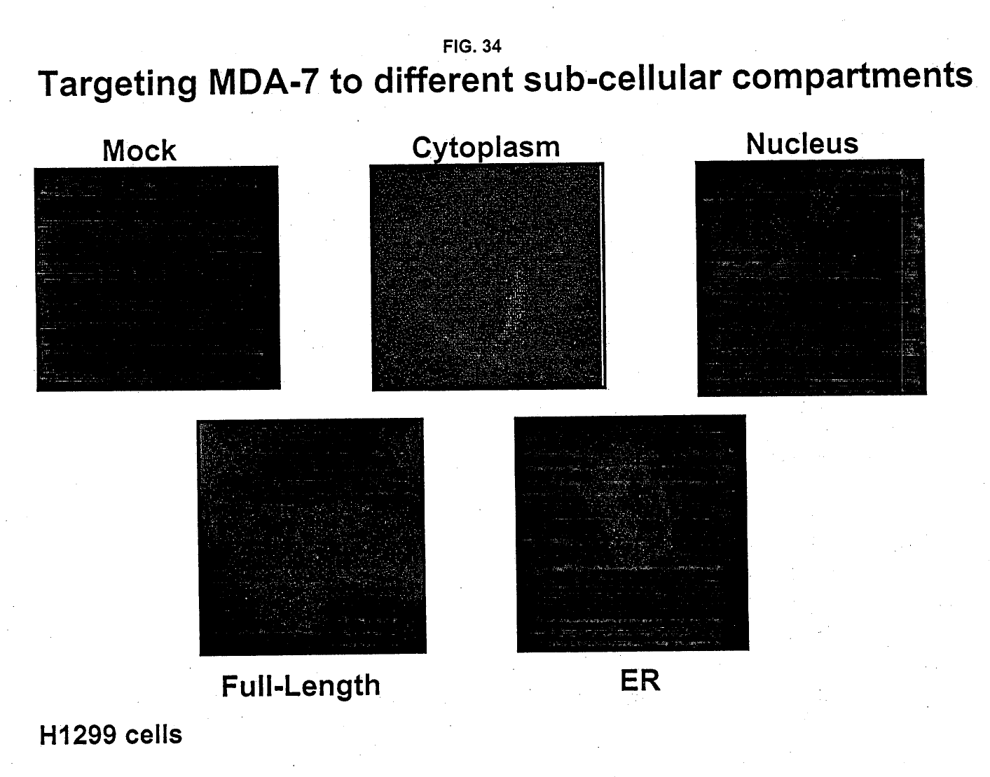

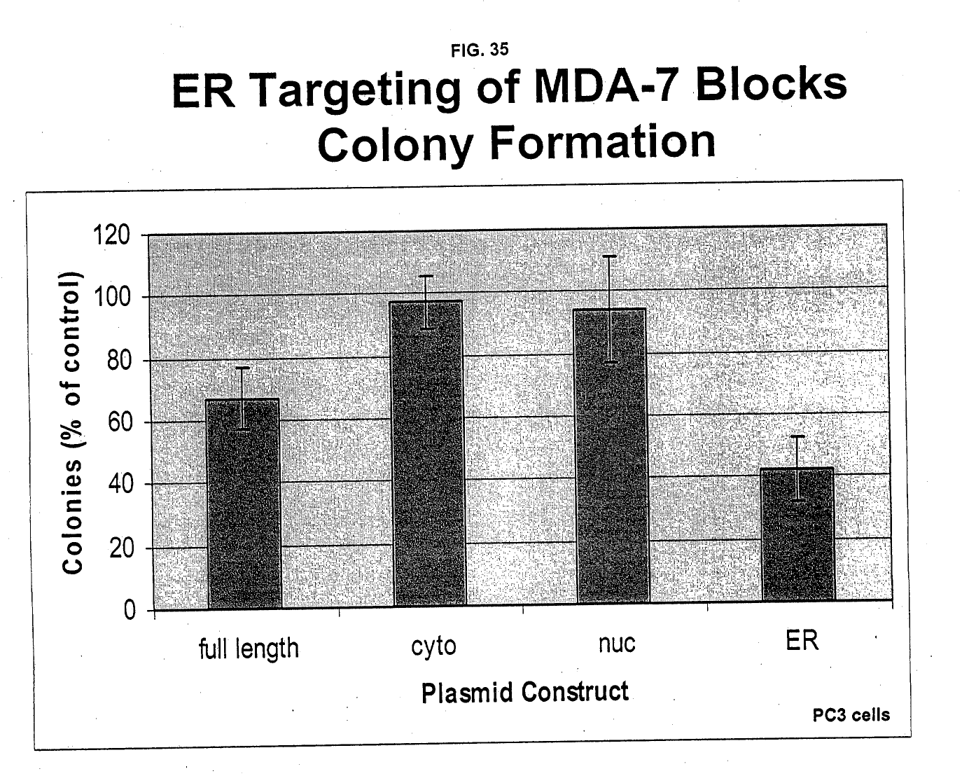

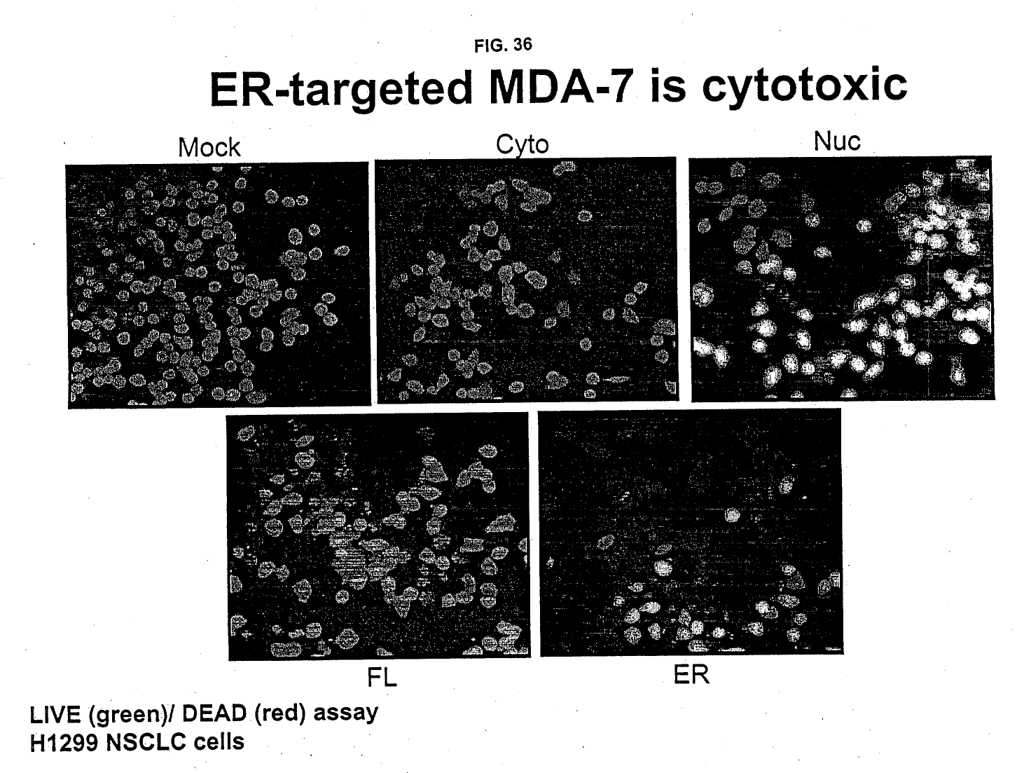

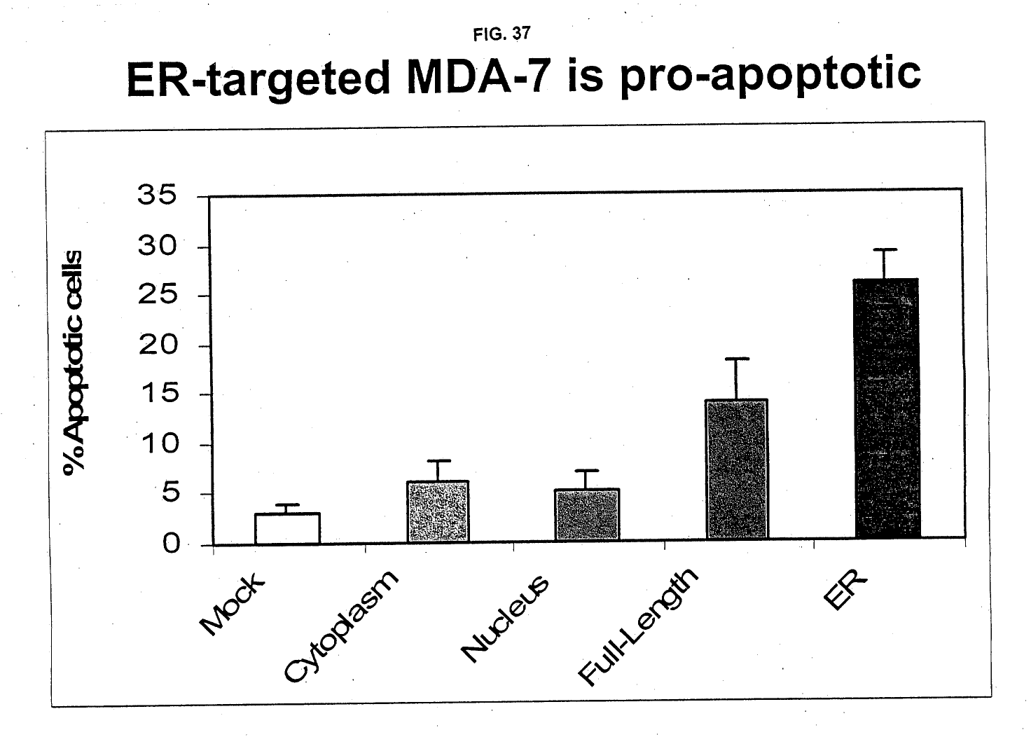

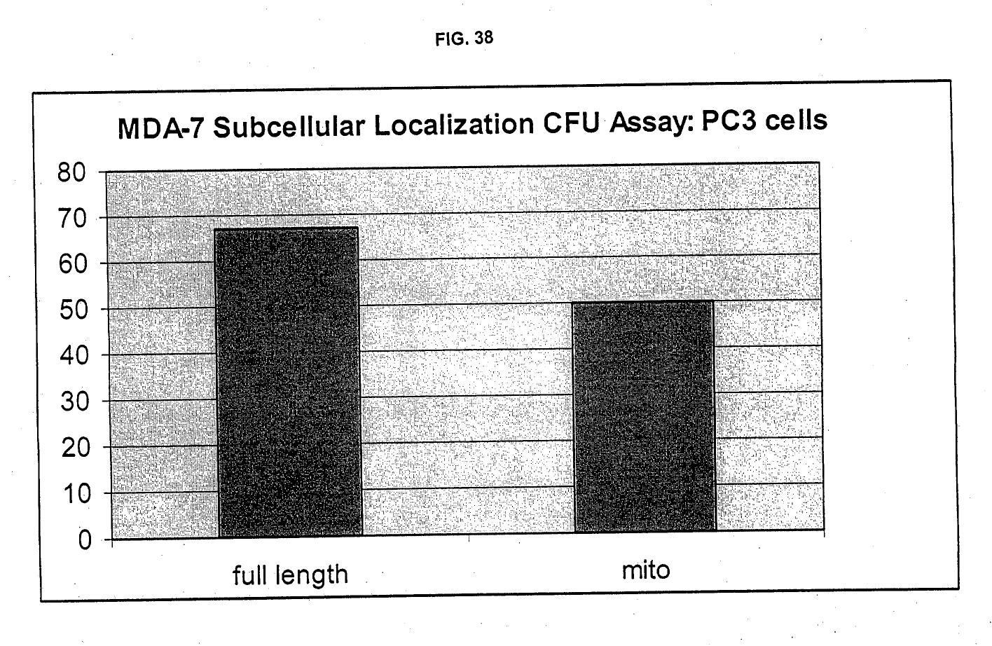

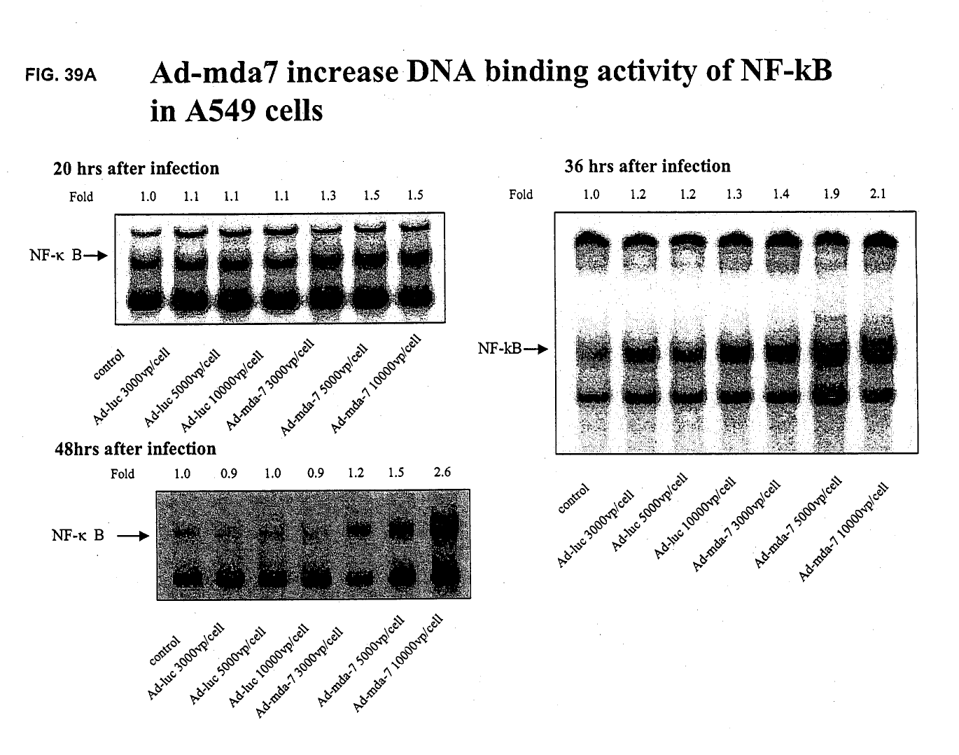

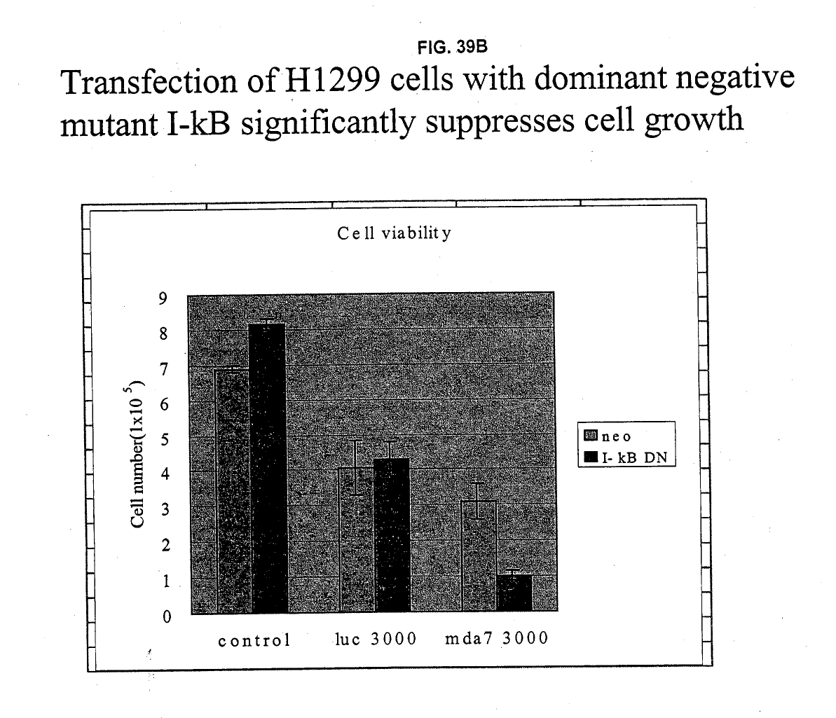

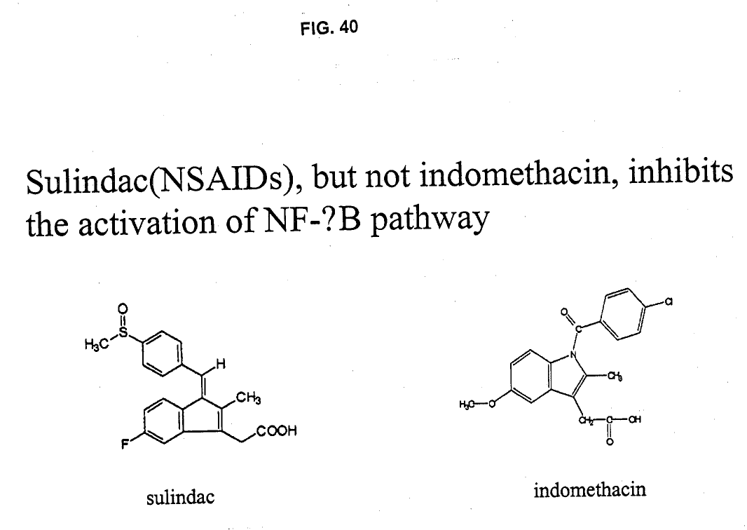

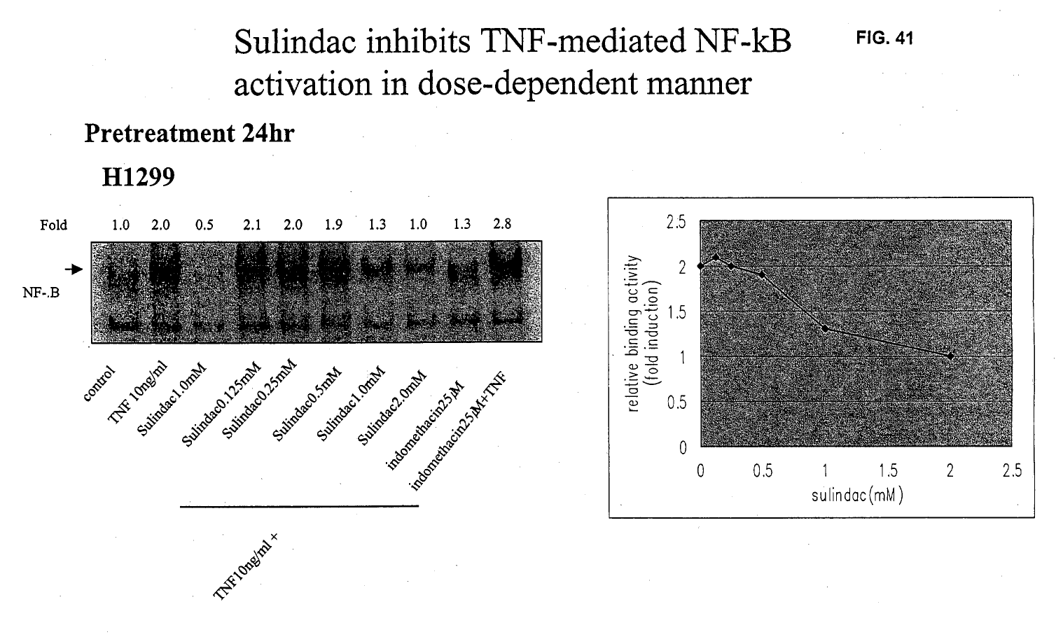

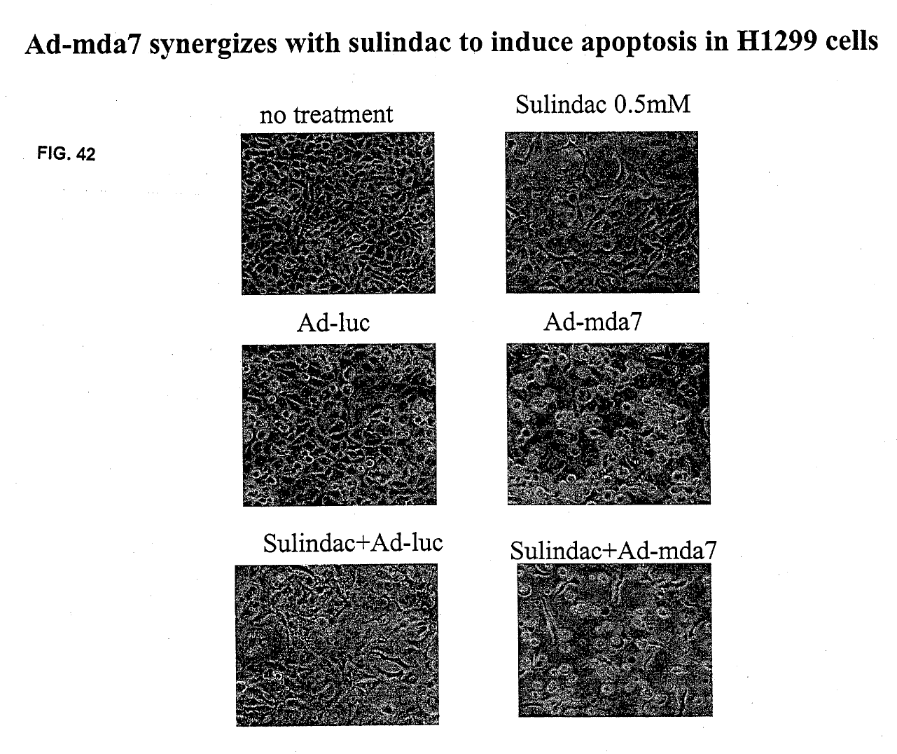

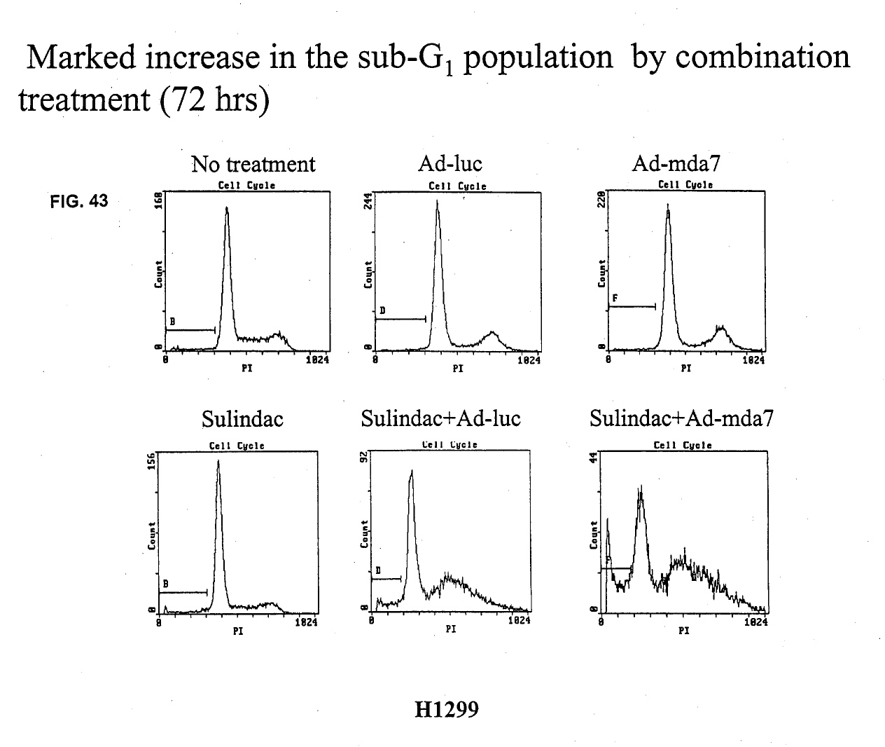

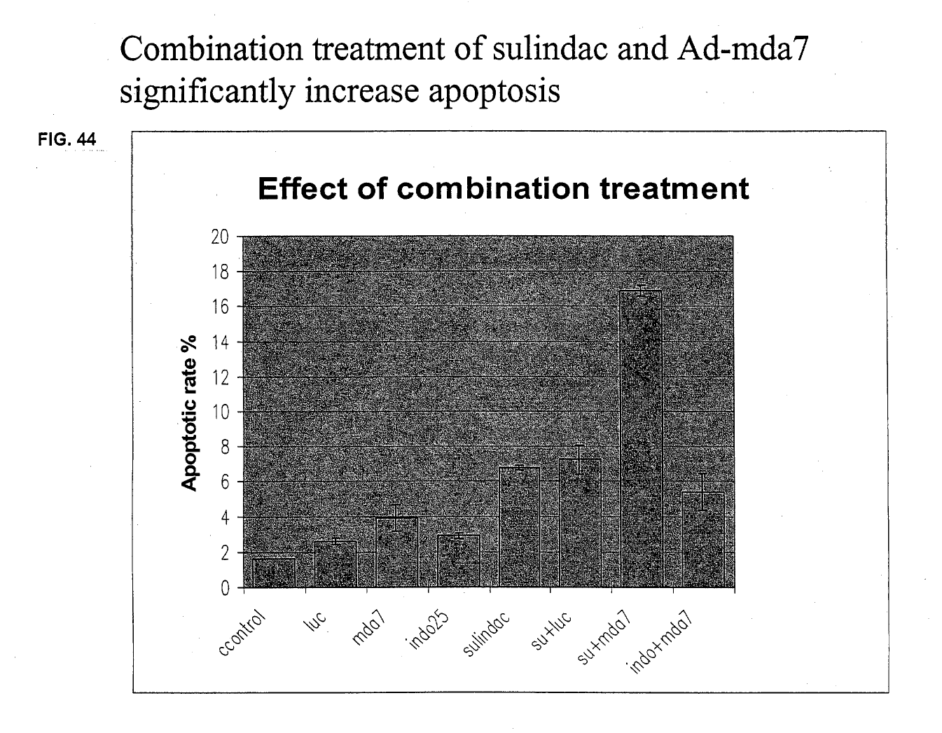

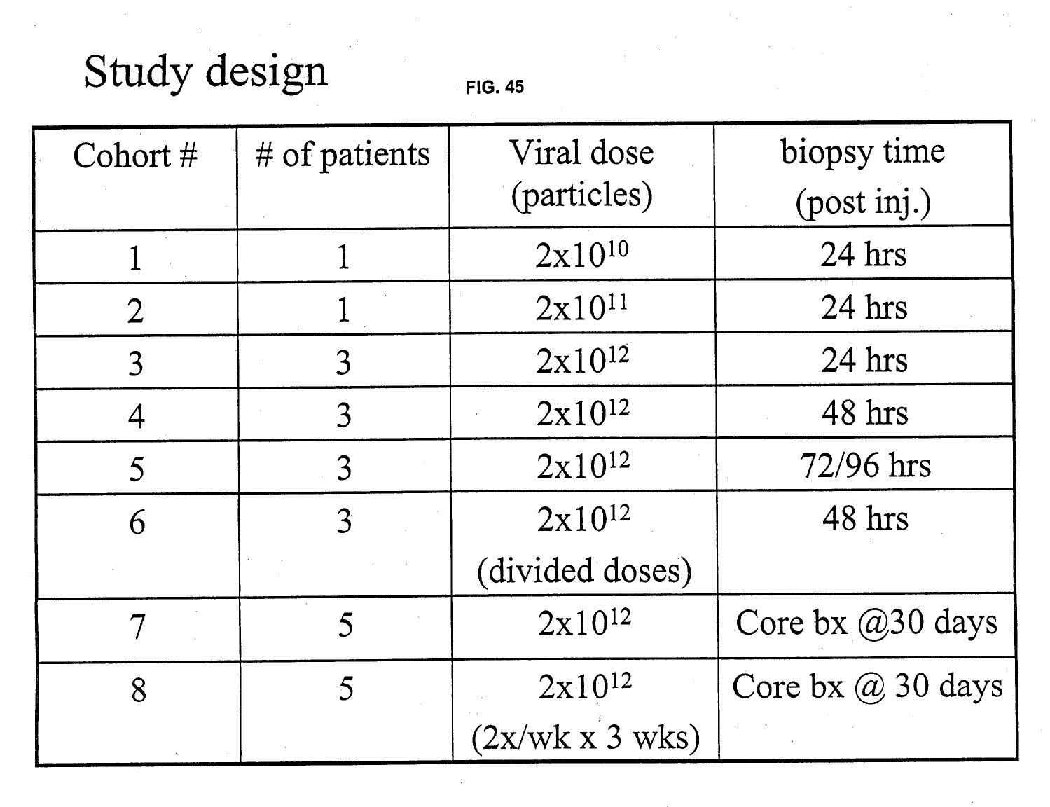

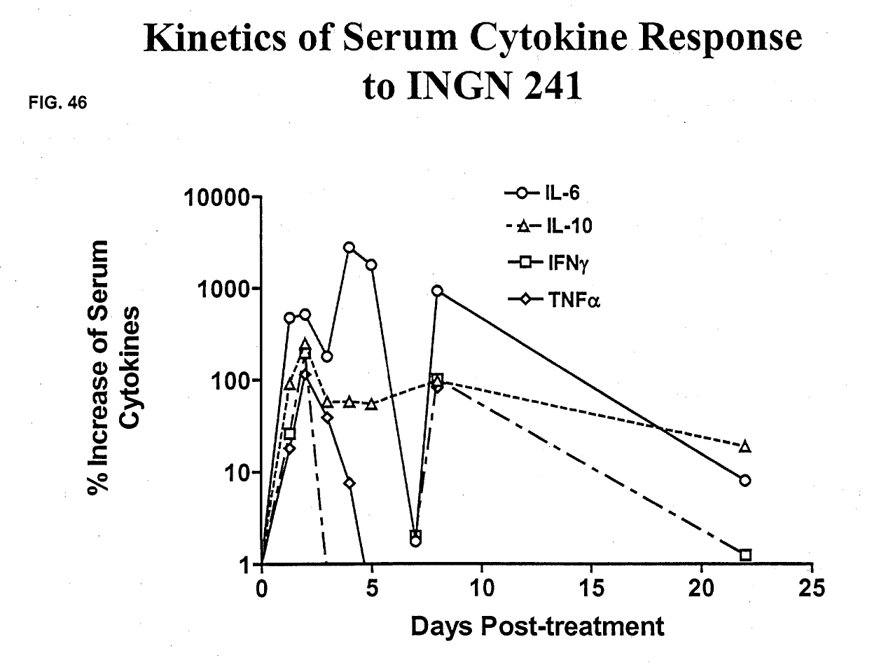

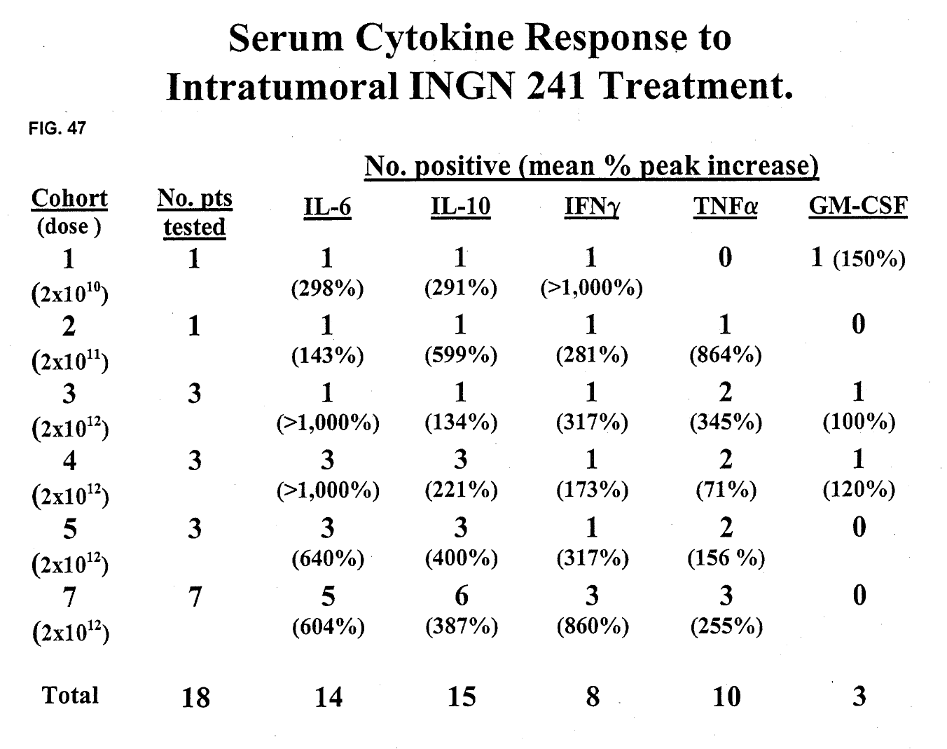

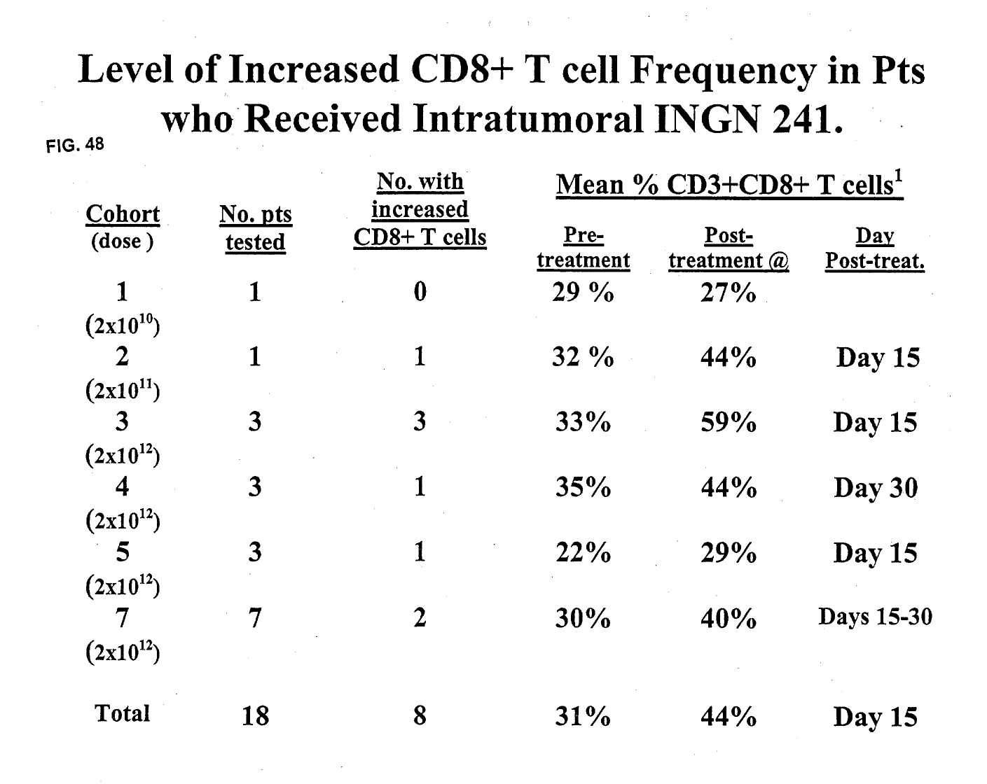

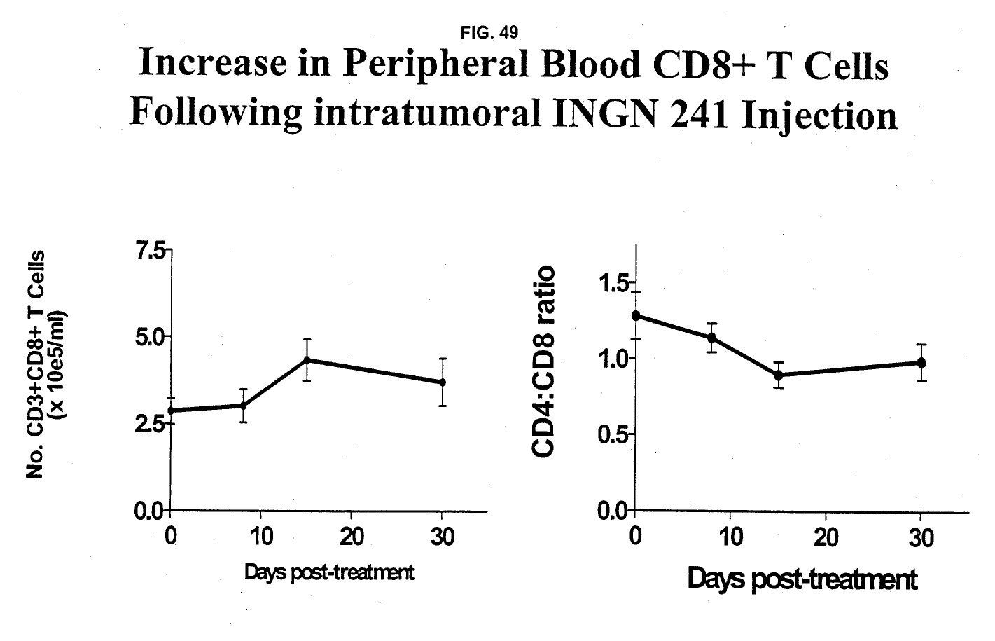

Following implantation, tumors were measured using calipers, and the statistical significance of tumor volume changes were calculated using the student's t-test. Animals that received an equal mixture of tumor cells and parental 293 cells served as controls (n=10). Tumors containing 293-mda-7 cells showed significant growth inhibition (p=0.001) compared to tumors treated with parental 293 cells. Each time point represents the mean tumor volume for each group. Bars represent standard error. FIG. 26B. Detection of MDA-7 protein in tumor tissues containing 293-mda-7 cells (lames 3 and 4) compared to tumors that contained parental 293 cells (lanes 1 and 2). FIG. 26C. At the end of the experiment, tumors were harvested and analyzed. (a), gross tumor appearance and size from animals receiving parental 293 cells and 293-mda-7 cells; (b), Hematoxylin and eosin staining of tissue sections; (c), Immunohistochemical staining for CD31 showed reduced vascularization in 293-mda-7 treated tumors; (d), TUNEL staining showed endothelial cells and surrounding tumor cells undergoing apoptosis. FIG. 26D. Analysis of hemoglobin content in tumor samples demonstrated reduction in hemoglobin in animals receiving 293-mda-7 cells compared to animals receiving parental 293 cells.

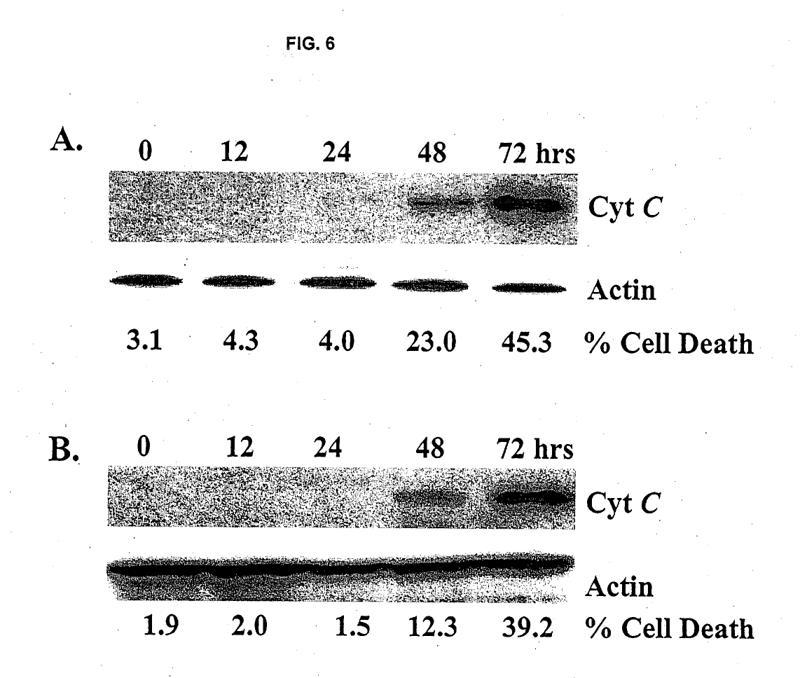

FIGS. 27A-6B. FIG. 27A. (a), Subcutaneous A549 tumors were established in the lower right flank of mice by injecting 5×10⁶ cells. When the tumors were palpable, animals were divided into two groups (8 animals/group) and treated as follows: control group was implanted with matrigel encapsulated 293 cells on the upper right flank of mice while experimental animals were implanted with Matrigel encapsulated 293-mda-7 cells. Tumors were measured using calipers, and the statistical significance of tumor volume changes were calculated using the student's t-test. Tumors treated with 293-mda-7 showed significant growth inhibition (p=0.001) compared to tumors treated with 293 cells. Each time point represents standard error; (b), Western blot analysis for sMDA-7 in the serum demonstrated production of sMDA-7 in the experimental animals as indicated by the intense banding (Lanes 1, 3 and 5) compared to controls (Lanes 2 and 4). FIG. 27B. At the end of the experiment tumors were harvested and analyzed. (a), gross tumor in appearance and size from animals receiving parental 293 cells and 293-mda-7 cells; (b), Hematoxylin and eosin staining of tissue sections; (c), Immunohistochemical staining for CD31 showed reduced vascularization in 293-mda-7 treated cells; (d), immunohistochemical staining showed Matrigel encapsulated cells staining positive for MDA-7.

FIG. 28. MDA-7 is processed and secreted. The top panel is a schematic representation of the primary amino acid sequence of MDA-7. The lower panel (left) is the hydropathic index of MDA-7 protein. The lower panel (right) is the Western blot analysis of both endogenous and secreted MDA-7.

FIGS. 29A-29B. Ad-mda7 activates the Unfolded Protein Response Pathway (UPR) proteins. H1299 cells were treated with Ad-luc or Ad-mda7 and 48 h later, cell lysates were analyzed by western blot for stress protein expression. Cell lysates were analyzed for expression of BiP, GADD34 and PP2A (FIG. 29A). Cell lysates were analyzed for expression of caspase 7, caspase 12, and XBP-1 (FIG. 29B).

FIG. 30. Ad-mda7 disrupts calcium flux and mitochondrial instability. Analytical studies were carried out on Ad-mda7 transduced H1299 cancer cells. Calcium flux and mitochondrial integrity were analyzed via Confocal microscopy.

FIGS. 31A-31B. MDA-7 protein is heavily glycosylated. Secreted MDA-7 protein stably expressed via 293-mda7 cells was treated with differed deglycosidases including glycopeptidase F (GlycoF), sialidase and endoglycosidase H (EndoH) (FIG. 31A). It was demonstrated by Western blot analysis that MDA-7 is heavily glycosylated (FIG. 31B).

FIGS. 32A-26B. FIG. 32A. Tunicamycin and brefeldin A block secretion of MDA-7 protein. Both Tunicamycin and brefeldin A (used at 1 and 2 μg/mL) inhibited the secretion of MDA-7 protein and caused increased concentrations of endogenous MDA-7 protein in Ad-mda7 transduced H1299 cells. FIG. 32B. Secretion is not required for Ad-mda7 mediated apoptosis. Secreted MDA-7 protein is unable to induce killing in cancer cells and is not required for Ad-MDA7 mediated apoptosis and eventual killing of cancer cells.

FIGS. 33A-33B. FIG. 33A. Targeting plasmid constructs. Different mda-7 constructs were made to target MDA-7 protein to various sub-cellular compartments. This included a full-length version with signal peptide in the N-terminus (for secretion), an mda-7 construct devoid of the signal peptide (for cytoplasmic expression), an mda-7 construct with a nuclear localization signal (NLS), and an mda-7 construct with an ER signal peptide. The constructs were cloned in the pShooter vector (Invitrogen). FIG. 33B. Intracellular expression and secreted protein expression from targeting vectors. Western analysis was used to look at the expression of MDA-7 protein in lysates and supernatants of transfected H1299 cells using different constructs of mda-7 as described in FIG. 27A.

FIG. 34. Targeting MDA-7 to different sub-cellular compartments. Expression of MDA-7 protein in transfected H1299 cells using different constructs of re-targeted MDA-7 (as mentioned in FIG. 27A) by immunofluorescence.

FIG. 35. ER-targeting of MDA-7 blocks colony formation. MDA-7 protein targeted to the endoplasmic reticulum (ER) inhibits cancer cell proliferation as seen by colony forming unit (CFU) assay. When MDA-7 is expressed in cytoplasm or nucleus, no anti-tumor activity is seen.

FIG. 36. ER-targeted MDA-7 is cytotoxic. H1299 cells were transfected with MDA-7 targeting plasmids and evaluated in the live/dead assay. MDA-7 protein targeted to the ER inhibits cancer cell proliferation as seen by increased dead cells (red, Ethidium bromide staining). Mock, cytoplasm and nuclearly targeted MDA-7 show minimal killing.

FIG. 37. ER-targeted MDA-7 is pro-apoptotic. MDA-7 protein targeted to the ER induces apoptosis as seen by Hoescht assay.

FIG. 38. MDA-7 subcellular localization CFU assay in PC3 cells. PC3 prostate tumor cells were transduced with plasmids encoding GFP control, full-length MDA-7 or mitochondrially targeted MDA-7 and evaluated in colony formation assays. Full-length MDA-7 resulted in a 35% decrease in colony formation compared to control, whereas mitochondrially targeted MDA-7 further reduced colony formation and viability of PC3 cells.

FIGS. 39A-39B. Relationship between Ad-mda7 and NF-κB pathway. FIG. 39A. Ad-mda7 increases DNA binding activity of NF-κB in A549 cells, as shown in the electromobility shift assay. FIG. 39B. Transfection of H1299 cells with dominant negative mutant I-kB significantly suppresses cell growth.

FIG. 40. Sulindac, but not indomethacin, inhibited the activation of the NF-κB pathway.

FIG. 41. Sulindac inhibited TNF-mediated NF-κB activation in a dose-dependent -manner.

FIG. 42. Ad-mda7 synergized with sulindac to induce apoptosis in H1299 cells.

FIG. 43. Marked increase in the sub-G₁ population by combination treatment (72 hrs).

FIG. 44. Combination treatment of sulindac and Ad-mda7 significantly increased apoptosis

FIG. 45. Study design of Phase I dose-escalating clinical trial wherein mda-7 was administered via intratumoral injection to patients with advanced carcinoma using a non-replicating adenoviral construct (Ad-mda 7). Study design demonstrates number of patients, viral dose, and biopsy time per cohort.

FIG. 46. Graphic representation of kinetics of serum cytokine response to Ad-mda 7, demonstrating % increase of serum cytokines vs. days post treatment. Results demonstrate a transient increase in serum cytokines following intratumoral injection of Ad-mda7.

FIG. 47. Serum cytokine response to intratumoral Ad-mda7 treatment per cohort. A majority of patients demonstrated transient increase in systemic cytokines (IL-6, IL-10, IFNγ, TNFα, GM-CSF).

FIG. 48. Level of increased CD8+ T cell frequency in patients who received intratumoral Ad-mda7. CD3+ CD8+ T cells were increased by 30±13% at day 15 following mda7 treatment.

FIG. 49. Increase in peripheral blood CD8+ cells following intratumoral Ad-mda7 injection in subjects.

### DESCRIPTION OF ILLUSTRATIVE EMBODIMENTS

The present invention is directed to methods of enhancing an immune response in a patient. Enhancing or increasing an immune response bestows preventative and therapeutic benefits through the body's enhanced ability to prevent, inhibit, or reduce the incidence of infections, diseases, or conditions. Thus, in certain embodiments, the MDA-7 polypeptide functions as an adjuvant to therapy. In other embodiments, the MDA-7 polypeptide may be used in combination with cytokines, chemokines, or analogs thereof, such as interferons α, β, and/or γ to enhance or increase an immune response in a patient.

The invention is further directed to enhancing or increasing an immune response to improve detection and identification of a molecule having a previously unidentified immunogenicity. Therefore, in certain embodiments, the methods of the present invention are used as a diagnostic to identify an immunogenic molecule, in particularly an immunogenic molecule useful in immune therapy.

In other embodiments, the invention is directed to prognosing a candidate patient for immunotherapy. The methods and compositions of the present invention would be administered to the patient, and an induced immune response is measured. The detection of an immune response indicates if the patient is a candidate for immunotherapy.

A. MDA-7

The compositions and methods of the present invention employ an MDA-7 polypeptide to enhance an immune response. MDA-7 is another putative tumor suppressor that has been shown to suppress the growth of cancer cells that are p53-wild-type, p53-null and p53-mutant. Also, the observed upregulation of the apoptosis-related BAX gene in p53 null cells indicates that MDA-7 is capable of using p53-independent mechanisms to induce the destruction of cancer cells. Applicants' observed that adenoviral-mediated overexpression of MDA-7 led to the rapid induction and activation of double stranded RNA-activated serine threonine kinase (PKR) with subsequent phosphorylation of eIF-2α, other PKR target substrates and apoptosis induction. Specific inhibition of PKR by 2-aminopurine (2-AP) in lung cancer cells abrogates Ad-mda7 induced PKR activation, PKR substrate target phosphorylation and apoptosis induction. As evidenced by PKR null fibroblasts, Ad-mda7 apoptosis is dependent on a functional PKR pathway. These characteristics indicate that MDA-7 has broad therapeutic, prognostic and diagnostic potential as an inducer of PKR and, consequently, an enhancer of an induced immune response.

PKR exerts antiviral and anticellular functions, and is involved in regulating a number of physiologic processes that include cell growth and differentiation (U.S. Pat. No. 6,326,466; Feng et al., 1992; Petryshyn et al., 1988; Petryshyn et al., 1984; Judware et al., 1991), tumor suppression (Koromilas et al., 1992; Meurs et al., 1993), and modulation of signal transduction pathways (Leonardo et al., 1989; Kumar et al, 1994; Maran et al., 1994).

Upregulation of PKR leads to the induction of apoptosis in various cancer cell lines. Furthermore, in myelodysplasias, critical tumorigenic deletions of the IRF-1 gene on chromosome 5q (Beretta et al., 1996) appear associated with decreased PKR levels and immunohistochemical analyses of lung and colorectal cancers demonstrate an association with PKR expression and prolonged survival (Haines et al., 1992). PKR appears to mediate anti-tumorigenic activity through the activation of multiple transduction pathways culminating in growth inhibition and apoptosis induction. Activation of these pathways occurs after the latent, inactive homodimeric form is induced by activating signals to undergo conformational changes leading to auto-phosphorylation and activation (Vattem et al., 2001). Once activated, PKR is able to phophorylate various substrate targets, which are important in growth control and apoptosis induction (Saelens et al., 2001; Sudhakar et al., 2000). Stimulation of the immune system has been linked to apoptosis (Albert et al., 1998; Chen et al., 2001; Saif-Muthama et al, 2000; Restifo et al., 2001). Further, artifical induction of apoptosis has been demonstrated to enhance the immunogenicity of a vaccine due to the stimulatory effect of dendritic cells that became activated by transfection of the apoptotic cells (Sasaki et al., 2001; Chattergoon et al., 2000).

Mda-7 mRNA has been identified in human PBMC (Ekmekcioglu et al., 2001), and no cytokine function of human MDA-7 protein was reported. MDA-7 has been designated as IL-24 based on the gene and protein sequence characteristics (NCBI database accession XM_{—}001405). The murine MDA-7 protein homolog FISP (IL-4-Induced Secreted Protein) was reported as a Th2 specific cytokine (Schaefer et al., 2001). Transcription of FISP is induced by TCR and IL-4 receptor engagement and subsequent PKC and STAT6 activation as demonstrated by knockout studies. Expression of FISP was characterized but no function has been attributed yet to this putative cytokine¹⁷. The rat MDA-7 homolog C49a (Mob-5) is 78% homologous to the mda-7 gene and has been linked to wound healing (Soo et al. 1999; Zhang et al., 2000). Mob-5 was also shown to be a secreted protein and a putative cell surface receptor was identified on ras transformed cells (Zhang et al., 2000). Therefore, homologues of the mda-7 gene and the secreted MDA-7 protein are expressed and secreted in various species. However, no data has emerged to show MDA-7 has cytokine activity. Such activity has ramifications for the treatment of a wide variety of diseases and infections by enhancing immunogenicity of an antigen.

The mda-7 cDNA (SEQ ID NO:1) encodes a novel, evolutionarily conserved protein of 206 amino acids (SEQ ID NO:2) with a predicted size of 23.8 kDa. The deduced amino acid sequence contains a hydrophobic stretch from about amino acid 26 to 45, which has characteristics of a signal sequence. The protein sequence shows no significant homology to known proteins with the exception of a 42 amino acid stretch that is 54% identical to interleukin 10 (IL-10). Structural analysis has determined that MDA-7 (IL-BKW or IL-20) displays the structural characteristics of the cytokine family (WO 98/28425, incorporated herein by reference). The structural characteristics and limited identity across a small stretch of amino acids implies an extracellular function for MDA-7. The expression of MDA-7 is inversely correlated with melanoma progression as demonstrated by increased mRNA levels in normal melanocytes as compared to primary and metastatic melanomas as well as decreased MDA-7 expression in early vertical growth phase melanoma cells selected for enhanced tumor formation in nude mice. Additional information and data regarding MDA-7 can be found in patent application Ser. Nos. 09/615,154 and 10/017,472, which are herein incorporated by reference.

Additional studies have shown that elevated expression of MDA-7 suppressed cancer cell growth in vitro and selectively induced apoptosis in human breast cancer cells as well as inhibiting tumor growth in nude mice (Jiang et al., 1996 and Su et al., 1998). Jiang et al. (1996) report findings that mda-7 is a potent growth suppressing gene in cancer cells of diverse origins including breast, central nervous system, cervix, colon, prostate, and connective tissue. A colony inhibition assay was used to demonstrate that elevated expression of MDA-7 enhanced growth inhibition in human cervical carcinoma (HeLa), human breast carcinoma (MCF-7 and T47D), colon carcinoma (LS174T and SW480), nasopharyngeal carcinoma (HONE-1), prostate carcinoma (DU-145), melanoma (HO-1 and C8161), glioblastome multiforme (GBM-18 and T98G), and osteosarcoma (Saos-2). Mda-7 overexpression in normal cells (HMECS, HBL-100, and CREF-Trans6) showed limited growth inhibition indicating that mda-7 transgene effects are not manifest in normal cells. Taken together, the data indicates that growth inhibition by elevated expression of MDA-7 is more effective in vitro in cancer cells than in normal cells.

Su et al. (1998) reported investigations into the mechanism by which MDA-7 suppressed cancer cell growth. The studies reported that ectopic expression of MDA-7 in breast cancer cell lines MCF-7 and T47D induced apoptosis as detected by cell cycle analysis and TUNEL assay without an effect on the normal HBL-100 cells. Western blot analysis of cell lysates from cells infected with adenovirus mda-7 (“Ad-mda-7”) showed an upregulation of the apoptosis stimulating protein BAX. Ad-mda-7 infection elevated levels of BAX protein only in MCF-7 and T47D cells and not normal HBL-100 or HMEC cells. These data lead the investigators to evaluate the effect of ex vivo Ad-mda-7 transduction on xenograft tumor formation of MCF-7 tumor cells. Ex vivo transduction resulted in the inhibition of tumor formation and progression in the tumor xenograft model.

In certain embodiments of the present invention, the mda-7 is provided as a nucleic acid expressing the MDA-7 polypeptide. In specific embodiments, the nucleic acid is a viral vector, wherein the viral vector dose is or is at least 10³, 10⁴, 10⁵, 10⁶, 10⁷, 10⁸, 10⁹, 10¹⁰, 10¹¹, 10¹², 10¹³, 10¹⁴, 10¹⁵ or higher pfu or viral particles. In certain embodiments, the viral vector is an adenoviral vector, a retroviral vector, a vaccinia viral vector, an adeno-associated viral vector, a polyoma viral vector, an alphaviral vector, a rhabdoviral vector, or a herpesviral vector. Most preferably, the viral vector is an adenoviral vector. In other specific embodiments, the nucleic acid is a non-viral vector.

In certain embodiments, the nucleic acid expressing the polypeptide is operably linked to a promoter. Non-limiting examples of promoters suitable for the present invention include a CMV IE, dectin-1, dectin-2, human CD11c, F4/80, SM22 or MHC class II promoter, however, any other promoter that is useful to drive expression of the mda-7 gene or the immunogene of the present invention, such as those set forth herein, is believed to be applicable to the practice of the present invention.

Preferably, the nucleic acid of the present invention is administered by injection. Other embodiments include the administering of the nucleic acid by multiple injections. In certain embodiments, the injection is performed local, regional or distal to a disease or tumor site. In some embodiments, the administering of nucleic acid is via continuous infusion, intratumoral injection, intraperitoneal, or intravenous injection. In other embodiments, the nucleic acid is administered to the tumor bed prior to or after; or both prior to and after resection of the tumor. Alternatively, the nucleic acid is administered to the patient before, during, or after chemotherapy, biotherapy, immunotherapy, surgery or radiotherapy. Preferably the patient is a human. In other embodiments the patient is a cancer patient.

1. Nucleic Acids, Vectors and Regulatory Signals

The present invention concerns polynucleotides or nucleic acid molecules relating to the mda-7 gene and its gene product MDA-7. Additionally, the present invention is directed to polynucleotides or nucleic acid molecules relating to an immunogenic molecule. These polynucleotides or nucleic acid molecules are isolatable and purifiable from mammalian cells. It is contemplated that an isolated and purified MDA-7 nucleic acid molecule, either the secreted or full-length version, that is a nucleic acid molecule related to the mda-7 gene product, may take the form of RNA or DNA. Similarly, the nucleic acid molecule related to the immunogenic molecule may take the form of RNA or DNA. As used herein, the term “RNA transcript” refers to an RNA molecule that is the product of transcription from a DNA nucleic acid molecule. Such a transcript may encode for one or more polypeptides.

As used in this application, the term “polynucleotide” refers to a nucleic acid molecule, RNA or DNA, that has been isolated free of total genomic nucleic acid. Therefore, a “polynucleotide encoding MDA-7” refers to a nucleic acid segment that contains MDA-7 coding sequences, yet is isolated away from, or purified and free of, total genomic DNA and proteins. When the present application refers to the function or activity of a MDA-7-encoding polynucleotide or nucleic acid, it is meant that the polynucleotide encodes a molecule that has the ability to enhance an immune response. Further, a “polynucleotide encoding an immunogen” refers to a nucleic acid segment that contains an immunogenic coding sequences, yet is isolated away from, or purified and free of, total genomic DNA and proteins. When the present application refers to the function or activity of an immunogene encoding an immunogen, it is meant that the polynucleotide encodes an immunogenic molecule that has the ability to induce an immune response in the body of a human.

The term “cDNA” is intended to refer to DNA prepared using RNA as a template. The advantage of using a cDNA, as opposed to genomic DNA or an RNA transcript is stability and the ability to manipulate the sequence using recombinant DNA technology (See Sambrook, 1989; Ausubel, 1996). There may be times when the full or partial genomic sequence is some. Alternatively, cDNAs may be advantageous because it represents coding regions of a polypeptide and eliminates introns and other regulatory regions.

It also is contemplated that a given MDA-7-encoding nucleic acid or mda-7 gene from a given cell may be represented by natural variants or strains that have slightly different nucleic acid sequences but, nonetheless, encode a MDA-7 polypeptide; a human MDA-7 polypeptide is a specfic embodiment. Consequently, the present invention also encompasses derivatives of MDA-7 with minimal amino acid changes, but that possess the same activity.

The term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid molecule encoding MDA-7 or another therapeutic polypeptide such as the immunogen may comprise a contiguous nucleic acid sequence of the following lengths or at least the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52,53,54,55,56,57,58,59,60,61,62,63,64,65,66,67,68,69,70,71,72,73,74,75,76,77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135,136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000 or more nucleotides, nucleosides, or base pairs. Such sequences may be identical or complementary to SEQ ID NO: 1 (MDA-7 encoding sequence).

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid segment as originally isolated, and does not exclude genes or coding regions later added to the segment by human manipulation.

In particular embodiments, the invention concerns isolated DNA segments and recombinant vectors incorporating DNA sequences that encode a MDA-7 protein, polypeptide or peptide that includes within its amino acid sequence a contiguous amino acid sequence in accordance with, or essentially as set forth in, SEQ ID NO:2, corresponding to the MDA-7 designated “human MDA-7” or “MDA-7 polypeptide.”

The term “a sequence essentially as set forth in SEQ ID NO:2” means that the sequence substantially corresponds to a portion of SEQ ID NO:2 and has relatively few amino acids that are not identical to, or a biologically functional equivalent of, the amino acids of SEQ ID NO:2.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:2 will be sequences that are “essentially as set forth in SEQ ID NO:2” provided the biological activity of the protein is maintained. In particular embodiments, the biological activity of a MDA-7 protein, polypeptide or peptide, or a biologically functional equivalent, comprises enhancing an immune response. Further, in particular embodiments, the biological activity of an immunogen, an immunogenic molecule that is a protein, polypeptide or peptide, or a biologically functional equivalent, comprises immunogenecity, which refers to the molecule's ability to induce an immune response in the body of a human. In certain other embodiments, the invention concerns isolated DNA segments and recombinant vectors that include within their sequence a nucleic acid sequence essentially as set forth in SEQ ID NO:1. The term “essentially as set forth in SEQ ID NO:1” is used in the same sense as described above and means that the nucleic acid sequence substantially corresponds to a portion of SEQ ID NO: 1 and has relatively few codons that are not identical, or functionally equivalent, to the codons of SEQ ID NO: 1. Again, DNA segments that encode proteins, polypeptide or peptides exhibiting MDA-7 activity will be most some.

In particular embodiments, the invention concerns isolated nucleic acid segments and recombinant vectors incorporating DNA sequences that encode MDA-7 polypeptides or peptides that include within its amino acid sequence a contiguous amino acid sequence in accordance with, or essentially corresponding to MDA-7 polypeptides. In other embodiments, the invention relates to an isolated nucleic acid segment and recombinant vectors incorporating DNA sequences that encode an immunogen, protein, polypeptide or peptides that include within its amino acid sequence a contiguous amino acid sequence in accordance with, or essentially corresponding to the immunogen.

Vectors of the present invention are designed, primarily, to transform cells with a therapeutic mda-7 gene under the control of regulated eukaryotic promoters (i.e., inducible, repressable, tissue specific). Also, the vectors may contain a selectable marker if, for no other reason, to facilitate their manipulation in vitro. However, selectable markers may play an important role in producing recombinant cells.

Tables 1 and 2, below, list a variety of regulatory signals for use according to the present 15 invention.

TABLE 1
Inducible Elements

| Element | Inducer | References |
|---|---|---|
| MT II | Phorbol Ester (TPA) | Palmiter et al., 1982; Haslinger and |
| | Heavy metals | Karin, 1985; Searle et al., 1985; |
| | | Stuart et al., 1985; Imagawa et al., |
| | | 1987; Karin et al., 1987; Angel |
| | | et al., 1987b; McNeall et al., 1989 |
| MMTV (mouse | Glucocorticoids | Huang et al., 1981; Lee et al., 1981; |
| mammary tumor virus) | | Majors and Varmus, 1983; |
| | | Yamamoto et al., 1983; Lee et al., |
| | | 1984; Ponta et al., 1985; Si.e.,i |
| | | et al., 1986 |
| β-Interferon | poly(rI)X | Tavernier et al., 1983 |
| | poly(rc) | |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TFA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | Ela, SV40 Large | Taylor et al., 1989; Taylor and |
| | T Antigen | Kingston, 1990a,b |
| Proliferin | Phorbol Ester-TPA | Mordacq and Linzer, 1989 |
| Tumor Necrosis Factor | PMA | Hensel et al., 1989 |
| Thyroid Stimulating | Thyroid Hormone | Chatterjee et al., 1989 |
| Hormone α Gene | | |

TABLE 2
Other Promoter/Enhancer Elements

| Promoter/Enhancer | References |
|---|---|
| Immunoglobulin Heavy Chain | Banerji et al., 1983; Gillies et al., 1983; |
| | Groscehedl and Baltimore, 1985; |
| | Atchinson and Perry, 1986, 1987; Imler |
| | et al., 1987; Neuberger et al., 1988; |
| | Kiledjian et al., 1988; |
| Immunoglobulin Light Chain | Queen and Baltimore, 1983; Picard and |
| | Schaffner, 1985 |
| T-Cell Receptor | Luria et al., 1987, Winoto |
| | and Baltimore, 1989; Redondo |
| | et al., 1990 |
| HLA DQ α and DQ β | Sullivan and Peterlin, 1987 |
| β-Interferon | Goodbourn et al., 1986; Fujita |
| | et al., 1987; Goodbourn |
| | and Maniatis, 1985 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class H HLA-DRα | Sherman et al., 1989 |
| β-Actin | Kawamoto et al., 1988; Ng et al., 1989 |
| Muscle Creatine Kinase | Jaynes et al., 1988; Horlick and Benfield, |
| | 1989; Johnson et al., 1989a |
| Prealbumin (Transthyretin) | Costa et al., 1988 |
| Elastase I | Omitz et aL, 1987 |
| Metallothionein | Karin et al., 1987; Culotta and |
| | Hamer, 1989 |
| Collagenase | Pinkert et al., 1987; Angel et al., 1987 |
| Albumin Gene | Pinkert et al., 1987, Tronche et |
| | al., 1989, 1990 |
| α-Fetoprotein | Godbout et al., 1988; Campere and |
| | Tilghman, 1989 |
| γ-Globin | Bodine and Ley, 1987; Perez-Stable and |
| | Constantini, 1990 |
| β-Globin | Trudel and Constantini, 1987 |
| c-fos | Cohen et al., 1987 |
| c-HA-ras | Triesman, 1985; Deschamps et al., 1985 |
| Insulin | Edlund et al., 1985 |
| Neural Cell | Hirsch et al., 1990 |
| Adhesion Molecule | |
| (NCAM) | |
| a₁-Antitrypain | Latimer et al., 1990 |
| H2B (TH2B) Histone | Hwang et al., 1990 |
| Mouse or Type I Collagen | Rippe et al., 1989 |
| Glucose-Regulated | Chang et al., 1989 |
| Proteins | |
| (GRP94 and GRP78) | |
| Rat Growth Hormone | Larsen et al., 1986 |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor | |
| Duchenne Muscular | Klamut et al., 1990 |
| Dystrophy | |
| SV40 | Banerji et al., 1981; Moreau et al., 1981; |
| | Sleigh and Lockett, 1985; Firak and |
| | Subramanian, 1986; Herr and Clarke, |
| | 1986; Imbra and Karin, 1986; Kadesch |
| | and Berg, 1986; Wang and Calame, 1986; |
| | Ondek et al., 1987; Kuhl et al., 1987 |
| | Schaffner et al., 1988 |
| Polyoma | Swartzendruber and Lehman, 1975; |
| | Vasseur et al., 1980; Katinka et al., |
| | 1980, 1981; Tyndell et al., 1981; Dandolo |
| | et al., 1983; Hen et al., 1986; Si.e.,i |
| | et al., 1988; Campbell and Villarreal, 1988 |
| Retroviruses | Kriegler and Botchan, 1983; Kriegler et al, |
| | 1984a,b; Bosze et al., 1986; Miksicek et |
| | al., 1986; Celander and Haseltine, 1987; |
| | Thiesen et al., 1988; Celander et al., |
| | 1988; Chol et al., 1996; Reisman and |
| | Rotter, 1989 |
| Papilloma Virus | Campo et al., 1983; Lusky et al., 1983; |
| | Spandidos and Wilkie, 1983; Spalholz et |
| | al., 1985; Lusky and Botchan, 1986; Cripe |
| | et al., 1987; Gloss et al., 1987; Hirochika |
| | et al., 1987, Stephens and Hentschel, 1987 |
| Hepatitis B Virus | Bulla and Siddiqui, 1988; Jameel and |
| | Siddiqui, 1986; Shaul and Ben-Levy, |
| | 1987; Spandau and Lee, 1988 |
| Human Immunod- | Muesing et al., 1987; Hauber and Cullan, |
| eficiency Virus | 1988; Jakobovits et al., 1988; Feng and |
| | Holland, 1988; Takebe et al., 1988; |
| | Berkhout et al., 1989; Laspia et al., 1989; |
| | Sharp and Marciniak, 1989; Braddock |
| | et al., 1989 |
| Cytomegalovirus | Weber et al., 1984; Boshart et al., 1985; |
| | Foecking and Hofstetter, 1986 |
| Gibbon Ape | Holbrook et al., 1987; Quinn et al., 1989 |
| Leukemia Virus | |

The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation.

The term “promoter” will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between elements is flexible, so that promoter function is preserved when elements are inverted or moved relative to one another. In the tk promoter, the spacing between elements can be increased to 50 bp apart before activity begins to decline. Depending on the promoter, it appears that individual elements can function either co-operatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Aside from this operational distinction, enhancers and promoters are very similar entities.

Promoters and enhancers have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way.

In some embodiments, the promoter for use in the present invention is the cytomegalovirus (CMV) promoter. This promoter is commercially available from Invitrogen in the vector pcDNAIII, which is some for use in the present invention. Also contemplated as useful in the present invention are the dectin-1 and dectin-2 promoters. Below are a list of additional viral promoters, cellular promoters/enhancers and inducible promoters/enhancers that could be used in combination with the present invention. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

Another signal that may prove useful is a polyadenylation signal. Such signals may be obtained from the human growth hormone (hGH) gene, the bovine growth hormone (BGH) gene, or SV40.

The use of internal ribosome binding sites (IRES) elements are used to create multigene, or polycistronic, messages. IRES elements are able to bypass the ribosome scanning model of 5-methylatd cap-dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

In any event, it will be understood that promoters are DNA elements which when positioned functionally upstream of a gene leads to the expression of that gene. Most transgene constructs of the present invention are functionally positioned downstream of a promoter element.

2. Proteins, Peptides and Polypeptides

a. Biologically Functional Equivalents

The present invention is directed to enhancing an immune response by providing an effective amount of a MDA-7 polypeptide. In certain embodiments, the MDA-7 polypeptide is directly provided. In specific embodiments, the MDA-7 polypeptide is provided before therapy. In specific embodiments, the MDA-7 polypeptide is administered at the same time as administration of an immunogenic molecule, such as an antigen, for purposes of immune therapy. In other specific embodiments, the MDA-7 polypeptide is provided after therapy, and in some instances, after providing an immunogenic molecule for purposes of treating, diagnosing or prognosing induction of an immune response.

Additional embodiments of the invention encompass the use of a purified protein composition comprising MDA-7 protein, truncated versions of MDA-7, and peptides derived from MDA-7 amino acid sequence administered to cells or subjects for the inhibition of angiogenesis. Truncated molecules of MDA-7 include, for example, molecules beginning approximately at MDA-7 amino acid residues 46-49 and further N-terminal truncations. Specifically contemplated are molecules start at residue 46, 47, 48, 49, 50, 51, 52, 53, 54, 55, 56, 57, 58, 59, 60,61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, and 182, and terminate at residue 206. In additional embodiments, residues 1, 2, 3, 4, 5, 6, 7, 8, 9, 10, 11, 12, 13, 14, 15, 16, 17, 18, 19,20,21,22,23,24,25,26,27,28,29,30,31,32,33,34,35,36,37,38, 39, 40, 41, 42, 43, 44, 45, and 46 are included with other contiguous residues of MDA-7, as shown in SEQ ID NO:2.

As will be understood by those of skill in the art, modification and changes may be made in the structure of a MDA-7 polypeptide or peptide, an immunogenic molecule, or an immungene product and still produce a molecule having like or otherwise desirable characteristics. For example, certain amino acids may be substituted for other amino acids in a protein structure without appreciable loss of interactive binding capacity with structures such as, for example, antigen-binding regions of antibodies or binding sites on molecules such as Tat and RNA polymerase II. Since it is the interactive capacity and nature of a protein that defines that protein's biological functional activity, certain amino acid sequence substitutions can be made in a protein sequence (or, of course, its underlying DNA coding sequence) and nevertheless obtain a protein with like (agonistic) properties. It is thus contemplated by the inventors that various changes may be made in the sequence of HIV polypeptides or peptides (or underlying DNA) without appreciable loss of their biological utility or activity.

In terms of functional equivalents, the skilled artisan also understands it is also well understood by the skilled artisan that inherent in the definition of a biologically-functional equivalent protein or peptide, is the concept of a limit to the number of changes that may be made within a defined portion of a molecule that still result in a molecule with an acceptable level of equivalent biological activity. Biologically-functional equivalent peptides are thus defined herein as those peptides in which certain, not most or all, of the amino acids may be substituted. In particular, where small peptides are concerned, less amino acids may be changed. Of course, a plurality of distinct proteins/peptides with different substitutions may easily be made and used in accordance with the invention.

It is also well understood that where certain residues are shown to be particularly important to the biological or structural properties of a protein or peptide, e.g., residues in the active site of an enzyme, or in the RNA polymerase II binding region, such residues may not generally be exchanged. This is the case in the present invention, where residues shown to be necessary for inducing an immune response should not generally be changed, which is contemplated for both the MDA-7 polypeptide and the immunogene product.

Amino acid substitutions are generally based on the relative similarity of the amino acid sidechain substituents, for example, their hydrophobicity, hydrophilicity, charge, size, and the like. An analysis of the size, shape, and type of the amino acid side-chain substituents reveals that arginine, lysine, and histidine are all positively charged residues; that alanine, glycine, and serine are all a similar size; and that phenylalanine, tryptophan, and tyrosine all have a generally similar shape. Therefore, based upon these considerations, the following subsets are defined herein as biologically functional equivalents: arginine, lysine, and histidine; alanine, glycine, and serine; and phenylalanine, tryptophan, and tyrosine.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, some, those which are within ±1 are particularly preferred, some, and those within ±0.5 are even more particularly preferred some.

It is also understood in the art that the substitution of like amino acids can be made effectively on the basis of hydrophilicity, particularly where the biological functional equivalent protein or peptide thereby created is intended for use in immunological embodiments, as in the present case. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, some, those which are within ±1 are particularly preferred, some, and those within ±0.5 are even more particularly preferred.some.

While discussion has focused on functionally equivalent polypeptides arising from amino acid changes, it will be appreciated that these changes may be effected by alteration of the encoding DNA, taking into consideration also that the genetic code is degenerate and that two or more codons may encode the same amino acid. A table of amino acids and their codons is presented belowhereinabove for use in such embodiments, as well as for other uses, such as in the design of probes and primers and the like.

b. Synthetic Peptides

The compositions of the invention may include a peptide modified to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity.

Compositions for use in the present invention may also comprise peptides which include all L-amino acids, all D-amino acids, or a mixture thereof. The use of D-amino acids may confer additional resistance to proteases naturally found within the human body and are less immunogenic and can therefore be expected to have longer biological half lives.

The present invention also describes MDA-7 peptides and/or immunogens for use in various embodiments of the present invention. Specific peptides are assayed for their abilities to elicit an immune response. In specific embodiments that the peptides are relatively small in size, the peptides of the invention can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

The compositions of the invention may include a peptide modified to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity.

Compositions for use in the present invention may also comprise peptides which include all L-amino acids, all D-amino acids, or a mixture thereof. The use of D-amino acids may confer additional resistance to proteases naturally found within the human body and are less immunogenic and can therefore be expected to have longer biological half lives.

C. In Vitro Protein Production

Following transduction with a viral vector according to some embodiments of the present invention, primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

One embodiment of the foregoing involves the use of gene transfer to immortalize cells for the production and/or presentation of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Another embodiment of the present invention uses autologous B lymphocyte cell lines, which are transfected with a viral vector that expresses an immunogene product, and more specifically, an protein having immunogenic activity. Other examples of mammalian host cell lines include Vero and HeLa cells, other B- and T- cell lines, such as CEM, 721.221, H9, Jurkat, Raji, etc., as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection: for dhfr, which confers resistance to; gpt, which confers resistance to mycophenolic acid; neo, which confers resistance to the aminoglycoside G418; and hygro, which confers resistance to hygromycin.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent cells.

B. Enhancing an Immune Response

1. Double-Stranded RNA Activated Serine/Threonine Kinase (PKR)

The methods of the present invention are useful for enhancing an immune response. The methods exploit the role of the interferon induced, double stranded (ds) RNA-activated serine threonine protein kinase, PKR, in Ad-mda7 induced apoptosis. PKR is a 68 kDa serine/threonine kinase which is present predominantly in a latent form in the cytoplasm of mammalian cells (Jagus et al., 1999). Two dsRNA-binding domains reside in the amino terminus and interaction with dsRNA or other activators modifies the conformation of PKR allowing it to undergo autophosphorylation and activation (Zhang et al., 2001; Vattem et al., 2001). Once activated, PKR is able to phosphorylate a variety of substrate targets, the most well characterized being eIF2α which leads to inhibition of protein synthesis, growth suppression and apoptosis induction (Saelens et al., 2001; Sudhakar et al., 2000). Activation of PKR in HeLa, Cos 1, U937 and NIH3T3 tumor cells leads to apoptosis induction and cellular death (Jagus et al., 1999). Additionally, mouse embryo fibroblasts (MEFs) from PKR knock-out mice are resistant to apoptotic cell death in response to a variety of stimuli including dsRNA, TNF-α, and lipopolysaccharide (Der et al., 1997). In certain embodiments of the invention, MDA-7 and/or nucleic acids encoding MDA-7 may be used in combination with interferons to activate PKR in a cell. The activation of PKR by such a composition(s) may lead to an increase in activity of PKR. PKR activation in certain contexts may lead to apoptosis of a target cell in vitro or in vivo.

Upregulation of PKR leads to the induction of apoptosis in various cancer cell lines. Furthermore, in myclodysplasias, critical tumorigenic deletions of the IRF-1 gene on chromosome 5q (Beretta et al., 1996) appear associated with decreased PKR levels and immunohistochemical analyses of lung and colorectal cancers demonstrate an association with PKR expression and prolonged survival (Haines et al., 1992). PKR appears to mediate anti-tumorigenic activity through the activation of multiple transduction pathways culminating in growth inhibition and apoptosis induction. Activation of these pathways occurs after the latent, inactive homodimeric form is induced by activating signals to undergo conformational changes leading to auto-phosphorylation and activation (Vattem et al., 2001). Once activated, PKR is able to phosphorylate various substrate targets, which are important in growth control and apoptosis induction (Saelens et al., 2001; Sudhakar et al., 2000). Stimulation of the immune system has been linked to apoptosis (Albert et al., 1998; Chen et al., 2001; Saif-Muthama et al., 2000; Restifo et al., 2001). Further, artifical induction of apoptosis has been demonstrated to enhance the immunogenicity of a vaccine due to the stimulatory effect of dendritic cells that became activated by transfection of the apoptotic cells (Sasaki et al, 2001; Chattergoon et al., 2000).Several viral RNAs inhibit the activation of PKR (Kitajewski et al., Cell 45:195-200 (1986); Clarke et al., Nucleic Acids Res. 19:243-248 (1991); Ghadge et al., J. Virol. 68:4137-4151 (1994)), and others are efficient activators (Hovanessian, A. G., J. Interferon Res. 9:641-647 (1989)). The TAR sequence of HIV-1 mRNA transcript has been shown to both activate (Edery et al., Cell 56:303-312 (1989); SenGupta et al. Nucleic Acids Res. 17:969-978 (1989); Judware et al., J. Interferon Res. 13:153-160 (1993)) and prevent activation (Gunnery et al., Proc. Natl. Acad. Sci. USA 87:8687-8691 (1990)) of PKR at low concentrations.

Both human (Meurs et al., Cell 62:379-390 (1990)) and murine PKR (Feng et al., Proc. Natl. Acad. Sci. USA 89: 5447-5451 (1992); Baier et al, Nucleic Acids Res. 21:4830-4835 (1993)) have been cloned and sequenced and these two cDNAs share extensive nucleotide sequence identity (Feng et al., Proc. Natl. Acad. Sci. USA 89: 5447-5451 (1992)). Results from several studies have reported that the RNA-binding domain of PKR is localized to the N-terminal portion of the kinase (Feng et al., 1992; McCormack et al., 1992; Patel et al., 1994; Green et al., 1992; Patel et al., 1992). Although deletions of several short portions of PKR sequence rich in positively charged residues have been shown to diminish dsRNA-induced PRK activation. U.S. Pat. No. 6,326,466 describes a discrete PKR region or amino acid sequence motif which is both necessary and sufficient to bind to regulatory dsRNA (Feng et al., 1992). Further, PKR antagonists are described for treating diseases or conditions associated with premature or induced cell death, such as the T cell depletion due to HIV-1 infection.

In contrast, in certain embodiments of the present invention, a molecule that increases expression of PKR is contemplated. A method of administering the molecule is considered to increase MDA-7 levels to a host and enhance an immune respone in the host. Therefore, as an alternative embodiment of the present invention, there is a composition comprising a molecule exhibiting a biological activitiy that increases expression of PKR. In another alternative embodiment, the molecule is provided, either in combination with, before or after an immunogenic molecule, such as an antigen is provided. The molecule exhibiting a biological activity that increases expression of PKR may be an interferon.

Applicants have discovered that Ad-mda7 induces and activates the ds-RNA dependent protein kinase (PKR), which leads to phosphorylation of eIF-2α and the induction of apoptosis in lung cancer cells. Treatment with 2-aminopurine (2-AP), a serine/threonine kinase inhibitor, abrogates PKR activation, eIF2α phosphorylation and apoptosis induction by Ad-mda7. Additionally, PKR null but not wild-type fibroblasts are resistant to Ad-mda7 induced apoptosis. The data indicates that the activation of PKR is critical to Ad-mda7 mediated apoptosis. Further, PKR has been implicated as an important regulator of tumorigenesis, and activation by MDA-7 indicates an important role for MDA-7 in improving the current therapeutic, prognostic and diagnostic methods in the art. Additionally, PKR activation results in increased expression of many molecules involved in immune activation. Thus, activation of PKR by Ad-mda7 or other methods will be beneficial in the augmentation of immune responses directed against pathogenic agents.

2. Mitochondrial Permeability Transition

It has been stated earlier that stimulation of the immune system has been linked to apoptosis (Albert et al., 2001; Chen et al., 2001; Saif-Muthama et al., 2000; Restifo et al., 2001). Furthermore, it has been demonstrated through Phase I and II clinical trials in lung cancer patients that transferring adenovirus containing wild-type p53 (Ad-p53) into lung tumors induces apoptosis and cellular death. Unfortunately, some patients are resistant to Ad-p53 gene transfer due to molecular changes in the downstream mediators of apoptosis. Therefore, there is a need for other genes to be identified that enable the treatment of cancer patients who are resistant to wild-type p53 gene transfer. Mda-7 is a novel tumor suppressor gene that has been shown to induce apoptosis in a wide range of p53-sensitive and p53-resistant cancer cells, but it does not induce apoptosis in normal cells (Jiang et al., 1996; Su et al., 1998; Ekmekcioglu et al., 2001; Mhashilkar et al., 2001; Saeki et al., 2000).

It has been suggested that mitochondrial activation and cytochrome c release are the critical steps in cellular commitment to apoptosis. One class of pro-apoptotic stimuli (including p53, BAX, BAK and staurosporine) is dependent on changes in the mitochondrial membrane potential (MMP) in order to induce the opening of mitochondrial permeability transition (MPT)-dependent pores. These pores are multiprotein complexes that span the inner and outer mitochondrial membranes. Following changes in the MMP, they allow the release of cytochrome c and other apoptogenic factors into the cytosol from the intramitochondrial space. In this class of apoptotic agents, the release of cytochrome c through MPT-dependent pores can be blocked by cyclosporine A (CyA) and bonkregic acid thereby inhibiting the onset of apoptosis and cellular death. However, other agents that induce apoptosis such as Bid may not be dependent on changes in the MMP to induce cytochrome c release but instead are able to release cytochrome c through MPT-independent pores, which are not blocked by CyA or bonkregic acid.

It has been shown that MDA-7. induces cytochrome c release through MPT-independent pores that were not blocked by CyA. This unique mechanism of action was present in both p53-sensitive and p53-resistant cell lines demonstrating the molecular differences of action between Ad-mda-7 and Ad-p53. How Ad-mda-7 induces apoptosis through MPT-independent pores is not clear, but may be due in part to. activation of the extrinsic death receptor pathway as noted by increased levels of FasL. FasL can act through the extrinsic death receptor pathway by activating caspase 8 leading to Bid cleavage and mitochondrial activation. Kim et al. (2000) has also reported that Bid, unlike BAK or BAX, induces cytochrome c release through a pathway independent of MPT-dependent pores.

Pataer et al. (submitted 2002) has demonstrated that adenoviral mediated overexpression of MDA-7 leads to rapid apoptosis in p53-resistant and p53-sensitive lung cancer cells. Ad-mda-7's mitochondrial mechanism of action appears to work through MPT-independent pore release of cytochrome c with subsequent activation of executioner caspases and cellular cleavage. Upregulation of FasL, caspase 8 activation and Bid cleavage suggest that Ad-mda-7 may be acting preferentially through the extrinsic death receptor pathway with subsequent mitochondrial activation and MPT-independent cytochrome c release. Moreover, the use of Ad-mda-7 is a novel means of treating cancer patients resistant to Ad-p53 and other MPT-dependent cell death processes.

3. β-Catenin and PI3K Signaling Pathways

Signaling pathways controlled by both β-catenin and PI3K are involved in regulation of apoptotic and survival pathways as well as cell-cell adhesion, migration and metastasis. Genetic and epigenetic alterations in either. of these signaling pathways are known to be altered in a number of diverse tumor types, including those of the lung, breast and colon (Lebedeva et al., 2002; Novak et al., 1999). β-catenin, a key downstream effector of the Wnt pathway, binds to and activates transcription factors in the TCF/LEF family leading to transcription of TCF/LEF-responsive genes. β-catenin is involved in cell-cell adhesion, intracellular signaling and transcriptional regulation. Elevated levels of β-catenin have been found in many human tumors, notably colon and gastric carcinomas. Recently, elevated levels of β-catenin have been associated wtih poor prognosis in human adenocarcinoma of the breast. Furthermore, the putative β-catenin/TCF responsive genes include those that function in cell cycle progression and loss of cell differentiation, such as cyclin D1, matrilysin and c-myc, and these gene products are elevated in mammary tumors and cell lines expressing activated β-catenin (McCormick F, 1999). These observations indicate that the oncogenic Wnt pathway operates via β-catenin and its targets in the context of mammary hyperplasia and carcinoma (Michaelson et al., 2001; Smalley et al., 2001). In addition, the decreased expression of E-cadherin, APC and GSK-3β and the increased expression of β-catenin in breast cancer cells leads to an accumulation of β-catenin in the nucleus, thereby triggering the β-catenin-LEF/TCF signaling pathway (Yang et al., 2001). Molecules that are involved in relaying signals along the Wnt/β-catenin pathway are mutated or dysregulated in a variety of tumor types. For example, the majority of colon cancers have a mutation in the gene for adenomatous polyposis coli (APC) (Berrie C, 2001). APC binds to and promotes the degradation of β-catenin, so APC mutations lead to the accumulation of β-catenein. Colon cancers with wild-type APC have mutations in the gene for β-catenin such that β-catenin is resistant to APC-mediated degradation. Therefore, enhanced β-catenin activity is a common feature of most (>80%) colon cancers and also cancers of other tissues (Easwaran et al., 1999; Peifer et al., 2000).

In the Wnt signaling cascade, APC, axin, conductin and GSK-3β constitute the destruction complex which regulates the stability of β-catenin. In cells that do not receive Wnt signals, GSK-3β is believed to phosphorylate β-catenin, thus marking the later for proteosomal degradation. Wnt signaling inhibits GSK-3β activity. As a consequence, β-catenin would no longer be phosphorylated and can thus accumulate to form nuclear complexes with TCF/LEF factors and activate Wnt responsive genes, such as myc, cyclin D1, etc. (van Noort et al., 2002). Ad-mda-7 treatment results in increased expression of tumor suppressor genes such as APC, GSK-3β and E-cadherin, and decreased expression of proto-oncogenes involved in PI3K signaling. In Ad-mda-7 treated tumor cells, β-catenin is sequestered to the plasma membrane and blocked from translocating to the nucleus ultimately preventing transcriptional activation of growth promoting genes.

Expression and regulation of E-cadherin are very important in tumor progression and growth control. Ad-mda-7 mediated up-regulation of E-cadherin might play an important role in the mechanism to halt β-catenin transport to the nucleus. Correlation studies using human tumor specimens and functional experiments with cultured tumor cells and transgenic mouse models have indicated that the loss of E-cadherin is casually associated with the formation of epithelial cancers. Although the functional implication of such a “cadherin switch” remains to be elucidated, recent experimental results demonstrating an interaction of cadherins with tyrosine kinase receptors suggest that changes in cadherin expression may not only modulate tumor cell adhesion but may also affect signal transduction and, hence, the malignant phenotype (Parker et al., 2001).

The knowledge of the role of phospho-inositide 3-kinases (PI3Ks) in regulation of signal transduction, cytoskeletal rearrangements and membrane trafficking has expanded considerably. Evidence is emerging that members of the PI3K super-family and components of PI3K signaling play a role in the development of many human cancers. This complex pathway is known to be involved in the regulation of cell growth, differentiation, mobility, proliferation and survival and hence PI3K pathway components have become potential targets for the control of the growth and spread of cancer cells (Fry M J, 2001). Inhibition of the PI3K signaling pathway has been proposed as a potent method for inhibiting cancer cell proliferation (Katso et al., 2001; Cavallaro et al., 2002).

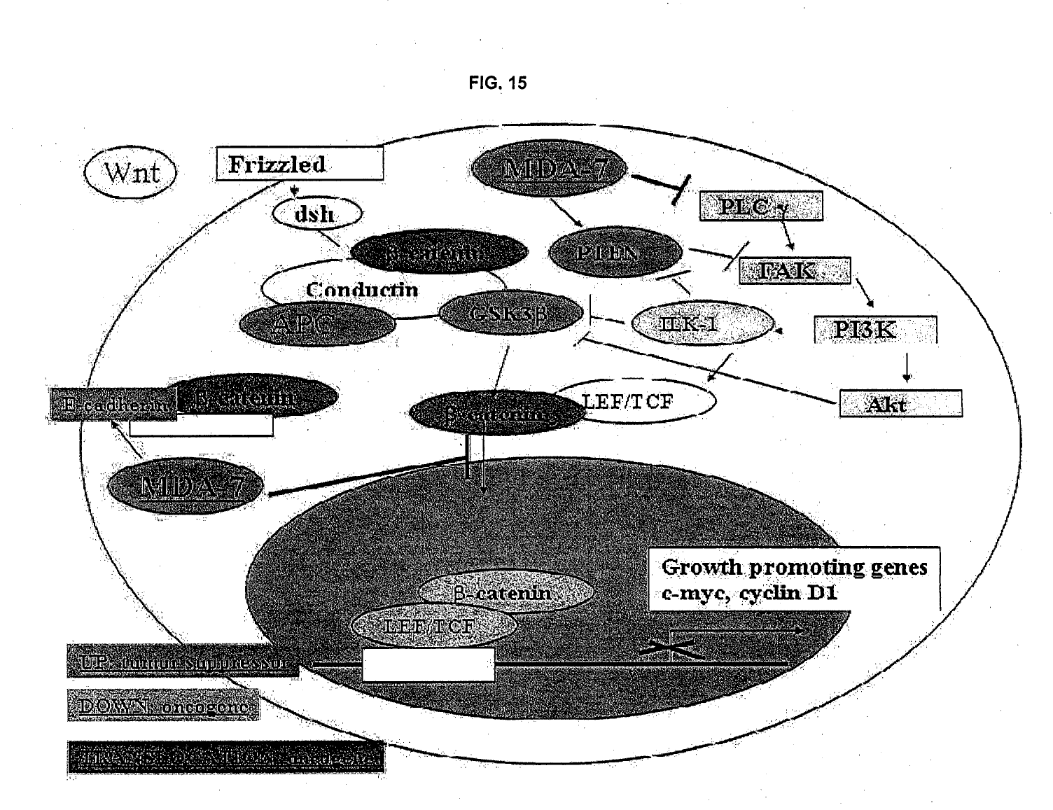

PI3K has fundamental significance in regulation of diverse cell functions such as growth, survival, and malignant transformation. PI3K itself possesses oncogenic activity as well as the ability to activate a number of other signaling proteins including oncoproteins. The anti-apoptotic effect of PI3K is realized by activation of proteins from other signaling pathway(s)-protein kinase B (Akt/PKB) and/or PKB-dependent enzymes (GSK-3β, ILK-1). PI3K plays a critical role in malignant transformation and can form complexes with some viral or cellular oncoproteins (src, ras, rac, T-antigen, etc.) whose transforming activities are realized only in the presence of PI3K. It has been shown that Ad-mda-7 is directly able to inhibit PI3K function and also suppress the functions of other proto-oncogenes which are regulated by PI3K (Mhashilkar et al., submitted 2002). Ad-mda-7, which encodes a novel tumor suppressor gene may up-regulate other tumor suppressors such as E-cadherin, GSK-3β, APC and PTEN. Importantly, Ad-mda-7 transduction in cancer cells is able to potently down-modulate the expression of oncoproteins such as PLC-γ, PI3K, Akt, FAK, and β-catenin (FIG. 15). During signal transduction from diverse membrane receptors using growth factor, integrins and other ligands, a complex cascade of molecular events has been described. Ligand-receptor engagement can activate a cascade of PLC-γ→FAK→PI3K→Akt, ultimately leading to de novo gene expression. Ad-mda-7 can down-regulate various members of this cascade. The tumor suppressor PTEN can block FAK, PI3K and Akt signaling. Therefore, the activity of Ad-mda-7 on these signaling molecules may be explained by their up-regulation by PTEN. However, Ad-mda-7 may also negatively regulate expression of PLC-γ, which is not regulated by PTEN. Thus, MDA-7 appears to function upstream of PLC-γ and PTEN. Ad-mda-7 triggers its anti-proliferative effects in breast and lung cancer cells by activating molecules from the β-catenin and PI3K pathways. It has been shown that oncogenic activation may lead to cross talk between molecules from the β-catenin and PI3K pathways. For example, β-catenin can be stabilized by the p85-α subunit of PI3K. In addition, cyclin D1, which can be activated by β-catenin stabilization in the nucleus, is regulated by the Wnt-1 and ILK signaling pathways and ILK induction of cyclin D1 involves the CREB signaling pathway in mammary epithelial cells (Woodfield et al., 2001; D'Amico et al., 2000).

It has been shown that Ad-mda-7 negatively regulates both the β-catenin and PI3K signaling pathways by increasing steady state levels of tumor suppressor proteins and decreasing expression of oncogenic proteins in breast and lung cancer cells. It is clear that there is considerable redundancy in the β-catenin and PI3K signaling pathways, however, Ad-mda-7 appears to coordinately regulate many of the members of these signaling pathways to produce antiproliferative, pro-apoptotic and anti-metastatic phenotypes. Ad-mda-7 infection causes redistribution of β-catenin from the nucleus to the plasma membrane, which modulates cell-cell adhesion and intracellular signaling, thus effectively inhibiting metastatic spread (Mhashilkar et al., submitted 2002).

4. G2 Cell Cycle Control

It has been shown that overexpression of MDA-7 using a replication defective adenovirus results in growth suppression and induction of apoptosis in a broad range of cancer cells, including melanoma, glioblastoma, osteosarcoma, and cancers of the breast, cervix, lung, colon, nasopharynx and prostate, but not in normal human epithelial, endothelial or fibroblast cells (Jiang et al., 1996; Su et al., 1998; Madiredi et al., 2000; Saeki et al., 2000; Mhashilkar et al., 2001). Furthermore, it has been reported that this MDA-7 mediated tumor suppression is induced through the activation of the caspase cascade and/or PKR, changes in the ratio of pro-apoptotic (BAX, BAK) to anti-apoptotic (BCL-2, BCL-XL) proteins, and/or an increase in cells in the G2/M cell cycle phase (Saeki et al., 2000; Lebedeva et al., 2002; Pataer et al., 2002).

MDA-7 is the ligand for two heterodimeric receptors, IL-22R1/IL-22R2 and IL-20R1/IL-20R2. The binding of MDA-7 to these receptors leads to the activation of the Jak-Stat pathways (Dumoutier et al., 2001; Wang et al., 2002; Kotenko et al., 2000). Jak1 and Tyk2, members of the Jak family of protein tyrosine kinases, associates with and is activated by the receptors for many cytokines including IL-10 (Aringer et al., 1999). IL-10 mediates Stat1 or Stat3 activation through the kinases Jak1 and Tyk2 (Kotenko et al., 2000).

The pro-inflammatory cytokine tumor necrosis factor-α (TNF-α) induces tumor suppression and apoptosis through the interaction between the caspase cascade, the JNK pathway, and IKK/NF-kB. Moreover, it induces BAX-BAK interactions and plays an important role in regulating immune responses (Baud et al., 2001, Wang et al., 1998; Sundararajan et al., 2001). TNF-α exerts its biological activity by binding to type 1 and type 2 receptors (TNF-R1 and TNF-R2) and activating multiple signaling pathways in many cell types (Tartaglia et al., 1992). The TNF-R1 signaling complex is composed of the trimerized receptor, TNF-R1-associated death domain protein (TRADD), FAS-associated death domain protein (FADD), TRAF2 and receptor interacting protein (RIP) (Locksley et al., 2001). FADD recruits and activates pro-caspase 8, initiating the apoptotic pathway in which caspase 3 and caspase 7 are two major effector caspases (Muzio et al., 1996; Cryns et al., 1998). Activated caspase 8 also cleaves Bid (BHS-interaction domain death agonist), which releases cytochrome c from the mitochondria to induce apoptosis (Li et al., 1998; Green et al., 1998). In contrast, TRAF2 and RIP are involved in the activation of c-Jun N-terminal kinase (JNK) and IKK resulting in activation of c-Jun and NF-kB, respectively (Ashkenazi et al., 1998). Although the effect of JNK/c-Jun activation on TNF-α-induced apoptosis is less clear, activation of IKK and NF-kB suppresses TNF-α-induced apoptosis in most cell types (Ahkenazi et al., 1998). The protection conveyed by the IKK/NF-kB pathway is overridden during TNF-α-induced apoptosis because IKK is proteolyzed by caspase 3-related caspases during TNF-α-induced apoptosis (Tang et al., 2001). These target proteins which make up the signaling pathway of TNF-α-induced apoptosis appear to be consistent with those of MDA-7-induced apoptosis due to activation of the caspase cascade, inhibition of NF-kB and activation of JNK. This suggests that TNF-R1, one of the TNF-A receptors, may be a critical part of MDA-7 ligands.

It has also been reported that MDA-7 induces G2/M cell cycle arrest through the inhibition of the Cdc25C pathway (Saeki et al., 2000; Lebedeva et al., 2002; Ekmekcioglu et al., 2001; Peng et al., 1997). Decrease of basal Chk1 and Chk2, which are activated by DNA damage, appear to be due to direct inhibition of Cdc25C by MDA-7 (Peng et al., 1997). Furthermore, p53 status might be related to the enhancement of G2 arrest by MDA-7 because p21 and p27, which are activated at G1 arrest were activated in LNCaP cells containing wild-type p53, but not in DU145 cells containing mutant p53. Additionally, the percentage of cells at G2 phase in LNCaP cells was significantly lower than in Du145 cells (Toyoshima et al., 1994). PKR might have also enhanced G2 arrest because PKR is activated in DU145 cells, but not in LNCaP cells, and it has been reported that activation of PKR induces G2/M arrest (Dagon et al., 2001; Zamanian-Daryoush et al., 1999). Moreover, Saito (submitted 2002) showed that cdc2, cyclin A and cyclin BI, at G2/M arrest induced by treatment with IFN-E and mezerein (MEZ) was consistent with cell cycle gene expression as previously reported (Tang et al., 2001). G2 arrest by Ad-mda-7 does not appear to be induced by DNA damage or by the inhibition of DNA replication because there were no aneuploid or polyploid chromosomes detected in DU145 and PC-3 cells containing mutant or deleted p53 (Tsuiki et al., 2001; Cross et al., 1995). These results suggest that MDA-7 may directly induce G2 arrest via inhibition of the Cdc25C pathway.

These effector functions of TNF-α seem to be similar to those of MDA-7. Therefore, Ad-MDA-7 may induce suppression of cell growth and apoptosis selectively in prostaste cancer cells through the activation of the caspase cascade, the Jak-Stat and JNK pathways, the inhibition of IKK/NF-kB pathways, and the induction of G2 phase cell cycle arrest through the inhibition of the Cdc25C pathway (Saito, submitted 2002).

5. Secreted MDA-7 is Antiangiogenic

The overexpression of MDA-7 protein in a wide variety of cancer cells inhibits their growth in vitro and in vivo. Recently, a secreted form of MDA-7 (sMDA-7) has been reported to be a potent inhibitor of angiogenesis. Ramesh et al. (submitted 2002) has shown in vitro that sMDA-7 inhibits endothelial cell differentiation (tube formation) as well as the migration of endothelial cells towards vascular endothelial growth factor and basic fibroblast growth factor. Furthermore, the anti-angiogenic activity of sMDA-7 against endothelial cells is typically mediated through the IL-22 receptor (IL-22r), as indicated by the activation of signal transducers and activators of transcription (STAT-3) following the addition of sMDA-7 protein. The co-administration of a blocking antibody to IL-22r with sMDA-7 results in the abrogation of tube formation inhibition. In vivo, sMDA-7 blocks neo-vascularization in a matrigel assay as evidenced by a reduction in vascularization and hemoglobin content. The inhibitory activity of sMDA-7 is twenty five-times more potent than recombinant endostatin at equal protein concentrations. In vivo mixing experiments of human lung tumor cells with 293 cells that stably express MDA-7 (1:1 ratio) demonstrated significant growth inhibition accompanied with reduced vascularization. Furthermore, systemic administration of sMDA-7 inhibited lung tumor growth in vivo in a mouse xenograft model. Tumor inhibition was due to the anti-angiogenic activity of sMDA-7 as shown by the decrease in tumor microvessel density as well as decreased hemoglobin content. This suggests that the tumor suppressive activity of the intracellular form of MDA-7 is mediated by direct tumor cell killing while the tumor suppressive activity of the secreted form of MDA-7 is due to inhibition of angiogenesis. These findings support the development of MDA-7-based therapeutics for the treatment of primary and distant metastases due to their ability to target both tumor cells and the tumor vasculature (Ramesh et al., submitted 2002).

Introduction of the mda-7 gene in a wide variety of cancer cells resulted in suppression of growth in vitro and in vivo with minimal toxicity observed in normal cells (Su et al., 1998; Saeki et al., 2000; Mhashilkar et al., 2001; Saeki et al., in press; Dumoutier et al., 2001). This anti-tumor activity has been attributed to the overexpression of MDA-7 protein. It has recently been demonstrated that the glycosylated form of MDA-7 is secreted in vitro (Madireddi et al., 2000; Jiang et al., 1996; Saeki et al., in press; Dumoutier et al., 2001). Although the secretion of MDA-7 and its binding to two distinct receptors has been reported, the functional significance of sMDA-7 protein in cancer has not been evaluated (Madireddi et al., 2000; Rich et al., 2001). It has been established in human PBMC that sMDA-7 functions as a pro-Th1 cytokine and causes the induction of IFN-γ, IL-6 and TNF-α (Caudel et al., in press). In a more recent study, the ability of Ad-mda-7 to inhibit endothelial cell differentiation as well as to decrease microvessel density in lung tumor xenografts following treatment with Ad-mda-7 has been confirmed (Saeki et al., 2000; Wang et al., 2002). Studies of anti-tumor activity and cytokine activity by MDA-7 are similar to those observed with other Th1-type cytokines such as IL-12 and IFN-γ (Ekmekcioglu et al., 2001; Ellerhorst et al., 2002; Huang et al., 2001).

Ramesh et al. (submitted 2002) has shown that sMDA-7 has potent anti-angiogenic activity both in vitro and in vivo, and that sMDA-7 can inhibit tumor growth in mice. Furthermore, sMDA-7 could not reverse the phenotype of differentiated endothelial cells but could block initiation of differentiation similarly to the effects of IFN-γ on endothelial cells (Lebedeva et al., 2002; Maheshwari et al., 1991). In addition to the inhibitory effects on tube formation, sMDA-7 blocked the migration of endothelial cells, an effect observed with other anti-angiogenic agents such as endostatin and maspin (Pataer et al., 2002; Madireddi et al., 2000; Zhang et al., 2000). Comparison of the inhibitory effect of sMDA-7 with that of recombinant human endostatin on tubular formation demonstrated sMDA-7 to be at least twenty-five times more potent than endostatin when tested at equal protein concentrations. It is speculated that sMDA-7 is more potent than endostatin because extremely low concentrations of sMDA-7 are required for anti-angiogenic activity and overexpression of sMDA-7 can directly inhibit tumor growth. Thus, the dual function of MDA-7 make it unique as a therapeutic agent (Ramesh et al., submitted 2002).

There is also evidence that the anti-angiogenic effect of sMDA-7 is receptor-mediated and involves the activation of signal transduction and activators of transcription −3 (STAT-3). Receptor-mediated anti-angiogenic activity has also been reported for IFN-γ and thrombospondin (Fickenscher et al., 2002; Jimenez et al., 2000). More recently, receptors for endostatin have been reported (Joki et al., 2001). Demonstration of PKR to be a critical mediator of Ad-mda-7 mediated apoptosis has been reported by Pataer et al. (2002). However, in the same study, PKR was shown not to be regulated in endothelial cells, thus eliminating the possibility that the anti-angiogenic activity of sMDA-7 is PKR-mediated (Pataer et al, 2002).

MDA-7 appears to act early in the differentiation program because it does not de-differentiate endothelial cells. Additionally, the mda-7 gene was identified as being up-regulated during differentiation of melanoma cells (Jiang et al., 1995). The loss of MDA-7 protein expression was significantly correlated with tumor invasiveness and MDA-7 protein was lost as tumors progressed, i.e. became less differentiated (Ellerhorst et al., 2002; Jiang et al., 1995). Thus, MDA-7 may play a role in regulating the differentiation process in both melanoma and endothelial cells. This indicates that mda-7 may be an effective therapeutic for treatment of primary and distant tumors.

6. MDA-7 Tumor Suppressor Activity is Mediated by Intracellular Protein

MDA-7 induces apoptosis in a diverse number of tumor types and releases a soluble MDA-7 protein product. Direct transfer of the supernatant from MDA-7 expressing cells containing soluble MDA-7 protein to native tumor cells was found to not induce bystander-mediated apoptosis. Similar results were obtained in co-culture experiments. Ad-mda7 (adenoviral vector harboring the mda-7 tumor suppressor gene) has been shown to have minimal toxicity on normal cells, while inducing rapid and high level apoptosis in a wide variety of cancer cell lines. Ad-mda7 transduced cancer cells expressed high levels of MDA-7 protein intracellularly. In addition, they secreted a soluble, glycosylated form of the protein that runs at a higher molecular weight than intracellular MDA-7. The soluble protein has been purified, and exhibits only limited cytotoxic effects on cancer cells. A study was planned to address the question of whether intracellular MDA-7 protein has enhanced killing activity if it is targeted to specific subcellular locations. Several plasmid constructs of mda-7 were created using vectors that target the expressed protein to various subcellular compartments. The mda-7 cDNA was engineered to delete the secretion signal sequence, and mda-7 expression vectors were constructed to direct expressed proteins to the cytoplasm, the nucleus, or the endoplasmic reticulum (ER). Additionally, a full-length mda-7 cDNA, including the secretion signal, was subcloned into the cytoplasmic backbone. The re-targeted vectors were evaluated for MDA-7 protein expression via transfection into lung tumor cells and all caused high levels of intracellular MDA-7 expression by Western blot analysis. Subcellular re-targeting of MDA-7 protein expression was confirmed via immunohistochemistry. Using flow cytometry and colony formation assays, the ability of re-targeted MDA-7 to kill cancer cells was investigated. The cytoplasmic and nuclear mda-7 constructs did not elicit cell death, whereas full-length (secreted) MDA-7 was cytotoxic. The ER-targeted mda-7 construct also elicited cell death in tumor cells. There are several conclusions that can be derived from this data. First, MDA-7 is a molecule with tumor suppressor activity as well as cytokine activity. Subcellular localization of MDA-7 affects tumor cell response, and intracellular MDA-7-induced apoptosis requires entry into the secretory pathway. Finally, it can be concluded that MDA-7 can elicit a signal from the ER compartment that results in apoptotic cell death and activation of cytoplasmic stress molecules. Additionally, it has been shown that MDA-7 protein targeted to the mitochondria causes an increase in cell death when compared to full-length MDA-7. Thus, targeting MDA-7 to the mitochondria further enhances its anti-tumor and anti-apoptotic effects.

7. Methods and Compositions in Prognosing a Candidate Patient

Therefore, the present invention provides a new method of enhancing an immune response. In certain embodiments, the invention is directed to methods and compositions useful in prognosing a candidate patient for immunotherapy. The candidate patient is administered or co-administered the MDA-7 polypeptide and an induced immune response is measured. One skilled in the art is aware of methods to measure an immune response, of which non-limiting examples are discussed in the sections below. The detection of an immune response indicates that the patient is a good candidate for immunotherapy, which refers to a patient that will benefit in any way from immunotherapy. In specific embodiments, the immunotherapy that is administered to the candidate patient is a composition of the present invention.

In other embodiments, the present invention includes a diagnostic or prognostic test that involves determining whether a subject can exhibit an immune response against an immunogenic molecule. The addition of MDA-7 may allow an immune response to be observed that would not be observed in its absence. In another embodiment, a diagnostic or prognostic test is employed to determine whether a subject exhibits an increased activity of a T-cell, a NK cell, or a macrophage. In another embodiment, the diagnostic or prognostic method is employed to determine whether a subject exhibits an increased cytokine concentration. In either case, if the subject does, the present invention includes eliciting an immune response using compositions described herein. In further embodiments, a subject who either exhibits or can exhibit an induced immune response is administered a treatment method to enhance the immune response.

In certain embodiments, the compositions and methods are directed to a relatively new addition to the family of cancer treatments: biological therapies, also known as immunotherapy, immune therapy, biotherapy or biological response modifier therapy. Immunotherapy exploits the body's natural immune system to either directly or indirectly fight cancer or to lessen the side effects that may be caused by some cancer treatments.

It is known in the art that the immune system is a complex network of cells and organs that work together to defend the body against attacks by foreign or non-self invaders. This network is one of the body's main defenses against disease. One mechanism used by the immune system to defend the body is to recognize a difference between a healthy cell and a foreign cell and then work to eliminate the foreign cell. Cancer develops when the integrity of the immune system is compromised in part or completely.

Cancer has become one of the leading causes of death in the Western world, second only behind heart disease. Current estimates project that one person in three in the U.S. will develop cancer, and that one person in five will die from cancer. Cancers can be viewed as altered cells that have lost the normal growth-regulating mechanisms. Genetic immunization, or vaccination, using naked DNA or using non-viral vectors has demonstrated considerable success in animal models of cancer and infectious disease. However, these studies have not correlated with results from human clinical trials, where, in general, only very limited immune induction/ augmentation has been observed using genetic immunization. The present invention describes a method for augmenting immune induction in humans by co-administering the mda-7 gene or MDA-7 protein to enhance the innate immune response, activating PKR and thereby enhancing immune responses against heterologous transgene or trans-protein products. Alternative embodiments of the invention include methods and compositions for the co-administration of a cytokine such as an interferon (e.g., IFN-α, IFN-β, and/or IFN-γ) with MDA-7 or a nucleic acid encoding MDA-7. The new methods of the present invention improves the efficacy of current immunotherapies.

The present invention contemplates employing any vaccine known in the art, and preferably those vaccines that suffer from low immune induction, and enhancing the immune response against the respective vaccine. Cancer vaccines are another form of immunotherapy. Vaccines for infectious diseases, such as measles, mumps, and tetanus, are effective because they expose the body's immune cells to weakened forms of antigens that are present on the surface of the infectious agent. This exposure causes the immune cells to produce more plasma cells, which make antibodies. T-cells that recognize the infectious agent also multiply and once activated, remember the exposure. Thus, the next time the agent enters the body, cells in the immune system are already prepared to respond and stop the infection.

Cancer vaccines help the patient's immune system recognize cancer cells. These vaccines may help the body reject tumors and prevent cancer from recurring. In contrast to vaccines against infectious disease, cancer vaccines are designed to be injected after the disease is diagnosed, rather than before. Cancer vaccines given when the tumor is small may be able to eradicate the cancer. For example, a cancer vaccine that is administered to a patient to prevent recurrence of skin cancer has been described and is currently undergoing clinical trials (MelanA/MART1 and gp100). Other cancer vaccines under investigation are Avicine®, an antigen-based therapy for treatment of advanced colorectal cancer, and an engineered fusion protein comprising a receptor molecule specific for malignant B-cells to treat and prevent recurrence of lymphoma. Other cancers serving as targets for cancer vaccines include cancers of the kidney, breast, ovary, and prostate.

Antibodies such as Herceptin and Rituxan are used in immunotherapy. Herceptin is used to treat metastatic breast cancer in patients with tumors that produce excess amounts of HER-2. Rituxan is used to treat B-cell non-Hodgkin's lymphoma recurrence or non-responsive to chemotherapy.

In the present invention, administration of an immunogenic molecule such as a receptor molecule specific for malignant B-cells induces an immune response against the receptor molecule in the patient. Further administration of a MDA-7 polypeptide enhances the immune response, thereby improving the efficacy of the immunotherapy and reducing the amount required for a therapeutic effect. In this case, the receptor molecule comprises a peptide derived from a tumor-specific or a tumor-associated epitope. By epitope, it is meant an antigenic determinant that comprises an antigen. An antigen employed in the present invention may have one or more epitopes provided at least one epitope is immunogenic and/or induces an immune response. The peptide that is administered may be operably linked to a carrier protein for delivery within the body. In other embodiments, the peptide may be operably linked to the MDA-7 polypeptide.

In cases involving a cancerous tumor, a combination treatment may involve administration of a cancer vaccine and of a nucleic acid molecule encoding MDA-7 polypeptide, which may occur before, after, or during the conventional cancer treatment, such as tumor resection, chemotherapy or radiotherapy. If the immune treatment occurs after tumor resection, the expression construct or vector encoding MDA-7 and/or the immunogenic molecule may be administered to the tumor bed.

In certain embodiments the nucleic acid is comprised within a viral vector or a non-viral vector. In other embodiments, the composition comprising the mda-7 is in a colloidal suspension, such as liposome, an emulsion or a proteinoid.

Though not adhering to a particular theory regarding the operability of these constructs, there is a notable amino acid homology of MDA-7 to IL-10 and across species in the D-helical region, located at the C-terminus, which is implicated in receptor binding. Thus, molecules preferably containing this 30-35 amino acid region are particularly some.

In certain embodiments, the present invention includes methods for enhancing an immune response comprising providing an effective amount of MDA-7 to enhance an immune response against a co-administered immunogenic molecule. The enhancement of an immune response is evidenced by an increase of cytokine expression or activity, proliferation of T cells or a population of T cells (for example, helper, cytotoxic, NK cells), proliferation of B cells or a population of B cells, cytotoxic T cell activity, or antibody production.

In certain embodiments of the invention, an antigen also is provided resulting in an immune response against the antigen and in such embodiments, the host receiving the antigen comprises an immune system. The antigen may be a tumor antigen, microbial antigen, viral antigen, or fungal antigen, or a combination thereof. In specific embodiments the antigen is a tumor antigen, such as PSA, CEA, MART, MAGE1, MAGE 3, gp100, BAGE, GAGE, TRP-1, TRP-2, AFP, tert, muc1, NY-ESO, bcr-ab1, or PMSA.

Additional embodiments of the invention include methods of enhancing or improving recovery or methods of reducing damage from traumatic treatment, which is a treatment that causes damage to normal cells. Such damage causes neutropenia, anemia, thrombocytopenia, and lymphopenia, for example. In certain embodiments, the traumatic treatment is chemotherapy and/or radiotherapy. It is contemplated that MDA-7 can be administered to a patient who will, is undergoing, or has undergone traumatic treatment. MDA-7 can be provided to a subject before, after or during treatment, preferably immune therapy.

In certain embodiments, the methods of enhancing an immune response comprise inducing the expression of an interferon or an interleukin. IL-6, interferon γ (IFNγ), tumor necrosis factor α (TNFα) by administering to a cell or patient an effective amount of MDA 7 polypeptide or a nucleic acid expressing the MDA-7 polypeptide, whereby induction of immune enhancing molecules, such as IL-6, IFNγ, or TNFα occurs. Alternatively, exogenous or recombinant interferons or interleukins may be provided (i.e., interferons or interleukins other than those provided by the cell or patient being treated).

Another object of the present invention is directed to a method of enhancing an immune response to an immunogenic molecule by providing the molecule and MDA-7, wherein the MDA-7 is provided to the subject by administering to the subject an expression construct comprising a nucleic acid sequence encoding at least 10, 20, 30, 40, 50, 60, 70, 80, 90, 100, 110, 120, 130, 140, 150, 160, 170, 180, 190, 200, or 206 contiguous amino acids of SEQ ID NO:2, wherein the nucleic acid sequence is under the transcriptional control of a promoter. A number of promoters are discussed herein and are contemplated for use with the invention, though the invention is in no way limited to those promoters. In specific embodiments, the expression construct is a viral vector. Viral vectors include an adenovirus vector, an adeno-associated virus vector, a herpesvirus vector, a retrovirus vector, a lentivirus vector, a vaccinia virus vector, or a polyoma vector.

A subject may be given MDA-7, the immunogenic molecule, or in certain embodiments an cytokine (e.g., an interferon) more than one time, such as two, three, four times or more. MDA-7, the immunogenic molecule and in certain embodiments a cytokine (e.g., interferon) may be given at the same time or at different times. Furthermore, it is contemplated that these compounds can be provided to a subject intravenously, directly, intraperitoneally, regionally, systemically, or orally.

Certain embodiments of the present invention provide methods of treating a tumor that includes decreasing a tumor size or decreasing a tumor growth rate comprising providing to a patient an immunogenic molecule, wherein the immunogenic molecule induces an immune response in the patient; and administering to the patient an effective amount of a MDA-7 polypeptide, wherein the MDA-7 enhances the induced immune response and decreases the tumor as compared to treatment with the immunogenic molecule. In such embodiments, the MDA-7 polypeptide may be considered an alternative adjuvant for therapy. It is contemplated that the MDA-7 polypeptide is administered in combination with other adjuvants known in the art as discussed previously. In various embodiments MDA-7 may be administered in combination with an interferon, such as IFN-α, IFN-β, or IFN-γ.

In yet another embodiment, the treatment of a wide variety of cancerous states is within the scope of the invention. For example, melanoma, non-small cell lung, small-cell lung, lung, hepatocarcinoma, retinoblastoma, astrocytoma, glioblastoma, leukemia, neuroblastoma, head, neck, breast, pancreatic, prostate, renal, bone, testicular, ovarian, mesothelioma, cervical, gastrointestinal, lymphoma, brain, colon or bladder. In general, the compositions and methods of the present invention are directed to treating any cancerous state that could benefit from an enhanced immune response.

In certain embodiments, an MDA-7 polypeptide or a nucleic acid encoding an MDA-7 polypeptide may be administered in combination with a cytokine or a nucleic acid encoding a cytokine. Cytokines may include, but are not limited to, interferon α (accession number E00175 and CAA23798, incorporated herein by reference), interferon β (accession number M28622 and AAA36040, incorporated herein by reference), or interferon γ (accession number X13274 and CAA31639, incorporated herein by reference)(Allen and Fantes, 1980; Lawn et al., 1981; and Diaz et al., 1993; each of which is incorporated herein by reference). Cytokines modulate cell growth, differentiation, and immune defenses in vertebrates. The Interferon (IFN) family is a unique class of cytokines that comprises secreted, multifunctional proteins. IFNs are components of the defenses of vertebrates against viral, bacterial, and parasitic infections, as well as certain tumors. They exert their various activities by inducing the synthesis of a large variety of proteins. There are direct and indirect indications that several of these proteins may have tumor-suppressor activities. The interferon-inducible proteins implicated include, but are not limited to: (i) a double-stranded RNA-activatable protein kinase that can phosphorylate and thereby inactivate the eukaryotic peptide chain initiation factor eIF-2; (ii) the interferon regulatory factors IRF-1 and IRF-2, which can modulate the expression of the interferons and of some interferon-inducible proteins; and (iii) RNase L, a latent endoribonuclease which can be activated by (2′-5′)oligoadenylates, the products of a family of enzymes which are also interferon-inducible. Compositions and methods of the invention may be used in combination with interferons or nucleic acids encoding interferons. Additional embodiments include compositions and methods for the activation of PKR. Activation of PKR in cell types such as cancer and other hyperproliferative cells typically induces apoptosis. Thus, methods and compositions that combine the administration of MDA-7 and INFs may be used as a therapeutic for enhancing an immune response and as an anti-cancer treatment. Exemplary methods and compositions of interferons are found in U.S. Pat. Nos. 6,379,701, 6,372,218, 6,350,589, 6,331,525, 6,250,469, 6,207,145, 6,204,022, and 6,177,074 each of which is incorporated herein by reference.

C. Gene Transfer

Compositions and methods of the invention are provided for administering the compositions of the invention to a patient.

1. Viral Transformation

a. Adenoviral Infection

One method for delivery of the recombinant DNA involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a recombinant gene construct that has been cloned therein.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5-tripartite leader (TPL) sequence which makes them some mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the some helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the some starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al, 1993), peripheral intravenous injections (Herz and Gerard, 1993) and stereotactic inoculation into the brain (Le Gal La Salle et al., 1993).

b. Retroviral Infection

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

c. AAV Infection

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al., 1986; Lebkowski et al., 1988; McLaughlin et al., 1988), which means it is applicable for use with the present invention. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

d. Other Viral Vectors

Other viral vectors may be employed as constructs in the present invention. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990).

A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been sugested that VEE may be an extremely useful vector for immunizations (Caley et al., 1997). It is contemplated in the present invention, that VEE virus may be useful in targeting dendritic cells.

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. (1991) recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In still further embodiments of the present invention, the nucleic acid encoding a MDA-7 to be delivered is housed within an infective virus that has been engineered to express a specific binding ligand. Alternatively, the nucleic acid encoding the MDA-7 polypeptide to be delivered is housed within an infective virus that has been engineered to express an immunogen. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

For example, to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Non-Viral Delivery

In addition to viral delivery of the nucleic acid encoding a MDA-7 protein, the following are additional methods of recombinant gene delivery to a given host cell and are thus considered in the present invention.

a. Lipid Mediated Transformation

In a further embodiment of the invention, the gene construct may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in lipid vehicle stability in the presence and absence of serum proteins. The interaction between lipid vehicles and serum proteins has a dramatic impact on the stability characteristics of lipid vehicles (Yang and Huang, 1997). Cationic lipids attract and bind negatively charged serum proteins. Lipid vehicles associated with serum proteins are either dissolved or taken up by macrophages leading to their removal from circulation. Current in vivo lipid delivery methods use subcutaneous, intradermal, intratumoral, or intracranial injection to avoid the toxicity and stability problems associated with cationic lipids in the circulation. The interaction of lipid vehicles and plasma proteins is responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

Recent advances in lipid formulations have improved the efficiency of gene transfer in vivo (Smyth-Templeton et al., 1997; WO 98/07408). A novel lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these lipid structures include a positive colloidal stabilization by cholesterol, two dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Lipid encapsulation has resulted in a lower toxicity and a longer serum half-life for such compounds (Gabizon et al., 1990). Numerous disease treatments are using lipid based gene transfer strategies to enhance conventional or establish novel therapies, in particular immune therapies.

In certain embodiments of the invention, the lipid vehicle may be complexed with a hemagglutinating virus (HVJ). This has been shown to facilitate fusion with the cell membrane and promote cell entry of lipid-encapsulated DNA (Kaneda et al., 1989). In other embodiments, the lipid vehicle may be complexed or employed in conjunction with nuclear non-histone chromosomal proteins (HMG-1) (Kato et al., 1991). In yet further embodiments, the lipid vehicle may be complexed or employed in conjunction with both HVJ and HMG-1.

3. Pharmaceutical Formulations and Delivery

In certain embodiments of the present invention, a method of enhancing an immune response by the delivery of an expression construct encoding a MDA-7 protein is contemplated. Alternatively, the method is directed to delivery of an expression construct encoding an immunogen. Alternatively, the expression construct comprises sequence encoding both the MDA-7 polypeptide and the immunogen. Examples of diseases and conditions involving an immune response include diseases that are prevented or treated with a vaccine. Including lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, breast cancer, bladder cancer and any other diseases or condition related to an immune response that may be treated by administering a MDA-7 polyprotein to enhance an induced immune response.

An effective amount of the pharmaceutical composition, generally, is defined as that amount sufficient to detectably and repeatedly to ameliorate, reduce, minimize or limit the extent of the disease or its symptoms. More rigorous definitions may apply, including elimination, eradication or cure of disease.

Preferably, patients will have adequate bone marrow function (defined as a peripheral absolute granulocyte count of >2,000/mm³ and a platelet count of 100,000/mm³), adequate liver function (bilirubin<1.5 mg/dl) and adequate renal function (creatinine<1.5 mg/dl).

a. Administration

In certain specific embodiments, it is desired to kill cells, inhibit cell growth, inhibit metastasis, decrease tumor or tissue size and otherwise reverse or reduce the malignant phenotype of tumor cells, using the methods and compositions of the present invention. The routes of administration will vary, naturally, with the location and nature of the lesion, and include, e.g., intradermal, parenteral, intravenous, intramuscular, intranasal, and oral administration and formulation.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles may advantageously be contacted by administering multiple injections to the tumor, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising MDA-7 and an immunogenic molecule or an MDA-7-encoding construct together with the immunogenic molecule. The perfusion may be continued post-resection, for example, by leaving a catheter implanted at the site of the surgery. Periodic post-surgical treatment also is envisioned.

An embodiment of the claimed invention transfers peptides or a combination of peptides into cells via perfusion. Continuous perfusion of an expression construct or a viral construct also is contemplated. The amount of construct or peptide delivered in continuous perfusion can be determined by the amount of uptake that is desirable. The present invention discloses an example of perfusion whereby a cell culture with an initial concentration of 106 cells/ml can first be labeled, washed, and then incubated with 100 μg of synthetic peptide for two hours.

Continuous administration also may be applied where appropriate, for example, where a tumor is excised and the tumor bed is treated to eliminate residual, microscopic disease. Delivery via syringe or catherization is some. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well, and often depend on tumor type, tumor location, disease progression, and health and age of the patient. Obviously, certain types of tumors will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) or viral particles for a viral construct. Unit doses range from 10³, 10⁴, 10⁵, 10⁶, 10⁷, 10⁸, 10⁹, 10¹⁰, 10¹¹, 10¹², 10¹³ pfu or viral particles (vp) and higher.

Protein may be administered to a patient in doses of or of at least 0.01. 0.05, 0.1, 0.2, 0.3, 0.4, 0.5, 0.6, 0.7, 0.8, 0.9, 1.0, 2.0, 3.0, 4.0, 5.0, 6.0, 7.0, 8.0. 9.0, 10, 15, 20, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000 or more ng/ml.

b. Injectable Compositions and Formulations

The some method for the delivery of an immunogenic molecule, an expression construct encoding a MDA-7 protein and/or an immunogen is via systemic administration. However, the pharmaceutical compositions disclosed herein may alternatively be administered parenterally, intravenously, intradermally, intramuscularly, or even intraperitoneally as described in U.S. Pat. No. 5,543,158; U.S. Pat. No. 5,641,515 and U.S. Pat. No. 5,399,363 (each specifically incorporated herein by reference in its entirety).

Injection of nucleic acid constructs may be delivered by syringe or any other method used for injection of a solution, as long as the expression construct can pass through the particular gauge of needle required for injection. A novel needeless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the some methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

c. Adjuvants

As is also well known in the art, the immunogenicity of an immunogenic molecule, immunogen or peptide composition can be enhanced by the use of non-specific stimulators of the immune response, known as adjuvants. Suitable adjuvants include all acceptable immunostimulatory compounds, such as cytokines, toxins, or synthetic compositions. In the present invention, the administering of an effective amount of a MDA-7 polypeptide enhances an immune response, thereby functioning as an adjuvant. Further, in other embodiments, a molecule that increases expression of PKR is considered to enhance an immune response and can be an acceptable immunostimulatory compound in the present invention.

However, other adjuvants may be used in addition to MDA-7 and they include IL-1, IL-2, 1L-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion. MHC antigens may even be used.

Exemplary, adjuvants include complete Freund's adjuvant (a non-specific stimulator of the immune response containing killed Mycobacterium tuberculosis), incomplete Freund's adjuvants and aluminum hydroxide adjuvant.

It is contemplated that in addition to MDA-7, other compounds with adjuvant activity may be included in certain aspects of the invention. Adjuvants, functions and mechanisms of delivery are well known in the art. Non-limiting examples of other adjuvants include Adjumer™ (i.e., PCPP salt; polyphosphazene); Adju-Phos (i.e., Aluminum phosphate gel); Algal Glucan (i.e., b-glucan; glucan); Algammulin (i.e., Gamma inulin/alum composite adjuvant); Alhydrogel (i.e., Aluminum hydroxide gel; alum); Antigen Formulation (i.e., SPT, AF); Avridine® (i.e., N,N-dioctadecyl-N′,N′-bis(2-hydroxyethyl) propanediamine; CP20,961); BAY R1005 (i.e., N-(2-Deoxy-2-L-leucylamino-b-D-glucopyranosyl)-N-octadecyldodecanoylamide hydroacetate); Calcitriol (i.e., 1a, 25-dihydroxyvitamin D3; 1,25-di(OH)2D3; 1,25-DHCC; 1a, 25-dihydroxycholecalciferol); Calcium Phosphate Gel (i.e., Calcium phosphate); Cholera holotoxin (CT) and Cholera toxin B subunit (CTB) (i.e., CT; CTB subunit; CTB); Cholera toxin A1-subunit-ProteinA D-fragment fusion protein (i.e., CTA1-DD gene fusion protein); CRL1005 (i.e., Block Copolymer P1205); Cytokine-containing Liposomes (i.e., Cytokine-containing Dehydration Rehydration Vesicles.); DDA (i.e., Dimethyl dioctadecylammonium bromide; dimethyldistearylammonium bromide (CAS Registry Number 3700-67-2)); DHEA (i.e., Dehydroepiandrosterone; androstenolone; prasterone); DMPC (i.e., Dimyristoyl phosphatidylcholine; 1,2-dimyristoyl-sn-3-phosphatidyl choline; (CAS Registry Number 18194-24-6)); DMPG (i.e., Dimyristoyl phosphatidylglycerol; sn-3-phosphatidyl glycerol-1, 2-dimyristoyl, sodium salt (CAS Registry Number 67232-80-8)); DOC/Alum Complex (i.e., Deoxycholic Acid Sodium Salt; DOC /Al(OH)3/mineral carrier complex); Freund's Complete Adjuvant (i.e., CIA; FCA); Freund's Incomplete Adjuvant (i.e., IFA; FIA); Gamma Inulin; Gerbu Adjuvant; GM-CSF (i.e., Granulocyte-macrophage colony stimulating factor; Sargramostim (yeast-derivedrh-GM-CSF)); GMDP (i.e., N-acetylglucosaminyl-(β1-4)-N-acetylmuramyl-L-alanyl-D-isoglutamine (CAS Registry Number 70280-03-4)); Imiquimod (i.e., 1-(2-methypropyl)-IH-imidazo[4,5-c]quinolin-4-amine; R-837; S26308); ImmTher™ (i.e., N-acetylglucosaminyl-N-acetyhnuramyl-Ala-D-isoGlu-L-Ala-glycerol dipalmitate; DTP-GDP); Immunoliposomes Containing Antibodies to Costimulatory Molecules (i.e., Immunoliposomes prepared from Dehydration-Rehydration Vesicles (DRVs)); Interferon-g (i.e., Actimmune® (rhIFN-gamma, Genentech, Inc.); immune interferon; IFN-g; gamma-interferon); Interleukin-1β(i.e., IL-10; IL-1; human Interleukin 1β mature polypeptide 1 17-259); Interleukin-2 (i.e., IL-2; T-cell growth factor; aldesleukin (des-alanyl-1, serine-125 human interleukin 2); Proleukin®; Teceleukin®); Interleukin-7 (i.e., IL-7); Interleukin-12 (i.e., IL-12; natural killer cell stimulatory factor (NKSF); cytotoxic lymphocyte maturation factor (CLMF)); ISCOM(s)™ (i.e., Immune stimulating complexes); Iscoprep 7.0.3.™^{;} Liposomes (i.e., Liposomes (L) containing protein or Th-cell and/or B-cell peptides, or microbes with or without co-entrapped interieukin-2, BisHOP or DOTMA; A, [L (Antigen)]); Loxoribine (i.e., 7-allyl-8-oxoguanosine); LT-OA or LT Oral Adjuvant (i.e., E. coli labile enterotoxin protoxin); MF59; MONTANIDE ISA 51 (i.e., Purified IFA; Incomplete Freund's adjuvant.); MONTANIDE ISA 720 (i.e., metabolizable oil adjuvant); MPL™ (i.e., 3-Q-desacyl-4′-monophosphoryl lipid A; 3D-MLA); MTP-PE (i.e., N-acetyl-L-alanyl-D-isoglutaminyl-L-alanine-2-(1,2-dipalmitoyl-sn-glycero-3-(hydroxy-phosphoryloxy)) ethylamide, mono sodium salt); MTP-PE Liposomes (i.e., MTP-PE Antigen presenting liposomes); Murametide (i.e., Nac-Mur-L-Ala-D-Gln-OCH3); Murapalmitine (i.e., Nac-Mur-L-Thr-D-isoGIn-sn-glycerol dipalmitoyl); D-Murapalmitine (i.e., Nac-Mur-D-Ala-D-isoGln-sn-glycerol dipalmitoyl); NAGO (i.e., Neuraminidase-galactose oxidase); Non-Ionic Surfactant Vesicles (i.e., NISV); Pleuran (i.e., b-glucan; glucan); PLGA, PGA, and PLA (i.e., Homo-and copolymers of lactic and glycolic acid; Lactide/glycolide polymers; poly-lactic-co-glycolide); Pluronic L121 (i.e., Poloxamer 401); PMMA (i.e., Polymethyl methacrylate); PODDS™ (i.e., Proteinoid microspheres); Poly rA:Poly rU (i.e., Poly-adenylic acid-poly-uridylic acid complex); Polysorbate 80 (i.e., Tween 80; Sorbitan mono-9-octadecenoate poly(oxy-1,2- ethanediyl) derivatives); Protein Cochleates; QS-21 (i.e., Stimulon™ QS-21 Adjuvant); Quil-A (i.e., Quil-A saponin, Quillaja saponin); Rehydragel HPA (i.e., High Protein Adsorbency Aluminum Hydroxide Gel; alum); Rehydragel LV (i.e., low viscosity alluminum hydroxide gel; alum); S-28463 (i.e., 4-Amino-otec,-dimethyl-2-ethoxymethyl-1H-imidazo[4,5-c]quinoline-1-ethanol); SAF-1 (i.e., SAF-m; Syntex Adjuvant Formulation); Sclavo peptide (i.e., IL-1b 163-171 peptide); Sendai Proteoliposomes, Sendai-containing Lipid Matrices (i.e., Sendai glycoprotein-containing vesicles; fusogenic proteoliposomes; FPLs); Span 85 (i.e., Arlacel 85, sorbitan trioleate); Specol; Squalane (i.e., Spinacane;Robane®;2,6,10,15,1 9,23-hexamethyltetracosane); Squalene (Spinacene; Supraene; 2,6,10,15,19, 23-hexamethyl-2,6,10,14,18,22 tetracosahexaene); Stearyl Tyrosine (i.e., Octadecyl tyrosine hydrochloride); Theramide™ (i.e., N-acetylglucosaminyl-N-acetylinuramyl-L-Ala-D-isoGlu-L-Ala-dipalmitoxy propylamide (DTP-DPP)); Threonyl-MDP (i.e., Termurtide™; [thr1]-MDP; N-acetyl muramyl-L-threonyl-D-isoglutamine); Ty Particles (i.e., Ty-VLPs, (Virus Like Particles)); Walter Reed Liposomes (i.e., Liposomes containing lipid A adsorbed to aluminum hydroxide, [L(Lipid A+Antigen)+Alum]).

In addition to adjuvants, it may be desirable to coadminister biologic response modifiers (BRM), which have been shown to upregulate T cell immunity or downregulate suppresser cell activity. Such BRMs include, but are not limited to, Cimetidine (CIM; 1200 mg/d) (Smith/Kline, Pa.); or low-dose Cyclophosphamide (CYP; 300 mg/m²) (Johnson/ Mead, N.J.) and cytokines such as γ-interferon, IL-2, or IL-12 or genes encoding proteins involved in immune helper functions, such as B-7.

d. Combination Treatments

In certain embodiments, the compositions of the present invention to increase the effectiveness of a vaccine by providing a MDA-7 polypeptide, or expression construct coding therefor. In some embodiments, the vaccine is a cancer vaccine. These compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

In one embodiment of the present invention, it is contemplated that mda-7 gene therapy is used in conjunction with immune therapy intervention, in addition to other pro-apoptotic or cell cycle regulating agents. Alternatively, the immune therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, for example gene therapy is “A” and the immunogenic molecule given as part of an immune therapy regime, such as an antigen, is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic expression constructs of the present invention to a patient will follow general protocols for the administration of such compounds, taking into account the toxicity, if any, of the vector. It is expected that the treatment cycles would be repeated as necessary. It also is contemplated that various standard therapies, as well as surgical intervention, may be applied in combination with the described therapy.

i. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

ii. Radiotherapy

Other factors that cause DNA damage and have been used extensively include what are commonly known as y-rays, X-rays, and/or the directed delivery of radioisotopes to tumor cells. Other forms of DNA damaging factors are also contemplated such as microwaves, proton beam irradiation (U.S. Pat. No. 5,760,395 and U.S. Pat. No. 4,870287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

In 1945, R. R. Wilson proposed the use of proton beams in the treatment of cancer. The advantage of protons in such treatment resides in the following physical characteristics (1) the radiation dose delivered by a proton penetrating tissue rises as the proton slows down, reaching a maximum near its stopping point (“Bragg peak”), and is zero beyond the stopping point, (2) protons in a monoenergetic beam have nearly the same range and therefore deliver a maximum dose at the same depth, and (3) protons being relatively heavy do not deviate much from a straight line as they come to rest.

To realize the full potential of the proton beam in the treatment of cancer and other diseases responsive to radiation treatment, it is necessary for the physician to known the exact location of the site to be treated and the characteristics of the tissue overlying the treatment site. It is only with advent of new imaging techniques such as computed tomography (CT scanning) and magnetic resonance imaging (MRI) that such information is now available with the required accuracy. Proton therapy for the treatment of cancer patients is now feasible.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

iii. Genes

In yet another embodiment, the immunogenic molecule is a provided as part of a gene therapy regime. Delivery of a vector encoding mda-7 in conjuction with a second vector encoding one of the following gene products will have a combined inducing effect on target tissues. Alternatively, a single vector encoding both genes may be used.

(a.) Antigens

In certain embodiments, the present invention is directed to improving immune therapy. An immune response against a tumor antigen can also be implemented with MDA-7. Tumor antigens include PSA, CEA, MART, MAGE1, MAGE3, gp100, BAGE, GAGE, TRP-1, TRP-2, PMSA, Mycobaterium tuberculosis soluble factor (Mtb), phenol soluble modulin (PSM), CMV-G, CMV-M, EBV capsid-EB nuclear antigen (EBNA), gp120, gp41, tat, rev, gag, toxa antigen, rubella antigen, mumps antigen, alpha-fetoprotein (AFP), adenocarcinoma antigen (ART-4), CAMEL, CAP-I, CASP-8, CDC27m, CDK4/m, CEA, CT, Cyp-B, DAM, ELF2M, ETV6-AMLI, ETS G250, GnT-V, HAGE, HER2/neu, HLA-A*0201-R1701, HPV-E7, HSP 70-2M, HST-2, hTERT, ICE, KIAA 0205, LAGE, LDLR/FUT, MC1R, MUCI, MUM-1, MUM-2, MUM-3, NA88-A, NY-ESO-I, p15, Pm1/RARalpha, PRAME, PSM, RAGE, RU1, RU2, SAGE, SART-1, SART-3, TEL/AML1, TPI/m, or WT1. Uses for inducing a response against tumor antigens are specifically contemplated and can be found in U.S. Pat. Nos. 5,552,293 and 6,132,980, which are specifically incorporated by reference.

(b.) Regulators of Programmed Cell Death

Apoptosis, or programmed cell death, is an essential process for normal embryonic development, maintaining homeostasis in adult tissues, and suppressing carcinogenesis (Kerr et al., 1972). The Bc1-2 family of proteins and ICE-like proteases have been demonstrated to be important regulators and effectors of apoptosis in other systems. The Bc1-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bc1-2 protein now is recognized to be a member of a family of related proteins, which can be categorized as death agonists or death antagonists.

Subsequent to its discovery, it was shown that Bc1-2 acts to suppress cell death triggered by a variety of stimuli. Also, it now is apparent that there is a family of Bc1-2 cell death regulatory proteins which share in common structural and sequence homologies. These different family members have been shown to either possess similar functions to Bc1-2 (e.g., Bc1XL, Bc1W, Mc1-1, A1, Bf1-1) or counteract Bc1-2 function and promote cell death (e.g., Bax, Bak, Bik, Bim, Bid, Bad, Harakiri).

(c.) Other agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the endothelial cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on endothelial cells. Increases intercellular signaling by elevating the number of GAP junctions would increase the anti-hyperproliferative effects on the neighboring endothelial cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is furhter contemplated that other agents that increase the sensitivity of a endothelial cell to apoptosis, such as the antibody c225, could be used in combination with the present invention to improve the treatment efficacy.

4. Identification of an Immunogenic Molecule

The present invention exploits Applicants' discovery of MDA-7 up-regulation of the interferon induced, ds-RNA dependent serine/threonine protein kinase (PKR). PKR appears to mediate anti-tumorigenic activity through the activation of multiple transduction pathways culminating in growth inhibition and apoptosis induction. Activation of these pathways occurs after the latent, inactive homodimeric form is induced by activating signals to undergo conformational changes leading to auto-phosphorylation and activation (Vattem et al., 2001). Once activated, PKR is able to phophorylate various substrate targets, which are important in growth control and apoptosis induction (Saelens et al., 2001; Sudharkar et al., 2000).

The activation of PKR is a critical event in Ad-mda7 apoptosis. The inhibition of PKR with the specific threonine/kinase inhibitor, 2 amino-purine (2-AP) led to almost complete reversal of Ad-mda7 apoptosis and abrogation of eIF-2α phosphorylation and protein synthesis inhibition. The inhibition of protein synthesis may be critical to the induction of apoptosis possibly because of regulation of one or more short-lived proteins involved in apoptosis inhibition. Alternatively, other pathways controlled by PKR may be important such as those involved in regulation of NF-κB, p53, MEK, IRF-1 or FADD (Jagus et al., 1999; Gil et al. 1999; Cuddihy et al., 1999; Balachandran et al., 1998).

Even though multiple pathways may be involved, PKR activation is critical for Ad-mda7 apoptosis since MEFs lacking PKR were unable to undergo apoptosis as opposed to MEFs with wild-type PKR. This inhibition of apoptosis appeared specific to mda-7 since transduction of MEFs lacking PKR with the pro-apoptotic Ad-Bak vector lead to unimpaired apoptosis. A model for these observations was synthesized in which MDA-7 and PKR are upstream of the pro-apoptotic Bak gene in the apoptosis cascade. In this model, MDA-7 induces PKR activation which leads to various cellular pathways that then induce caspase activation and apoptosis induction. Bak, being downstream of PKR, is not dependent on PKR activation to induce apoptosis. The data also indicated BID cleavage and caspase 8 activation, which is consistent with other work in the art that have demonstrated that PKR apoptosis is often mediated through activation of Fas, FADD, caspase-8 and BID (Balachandran et al., 1998).

Thus, adenoviral-mediated overexpression of MDA-7 led to the rapid induction and activation of PKR with subsequent phosphorylation of eIF-2α, other PKR target substrates and apoptosis induction. Specific inhibition of PKR by 2-AP in lung cancer cells abrogates Ad-mda7 induced PKR activation, PKR substrate target phosphorylation and apoptosis induction. As evidenced by PKR null fibroblasts, Ad-mda7 apoptosis is dependent on a functional PKR pathway. These results indicate a novel role for the multi-functional PKR gene as a critical mediator of Ad-mda7 apoptosis. Further, because PKR has been described herein as critical to MDA-7, induced apoptosis, and which has been suggested to induce an immune response, the present invention in certain embodiments contemplates inducing PKR expression to enhance an immune response, the data indicate that MDA-7 polypeptide is capable of enhancing an immune response.

In other embodiments, the methods of the present invention are directed to identifying immunogenic molecules. In particular, the present invention is useful in enhancing an immune response against a previously unidentified immunogenic molecule or a molecule possessing immunogencity at a level that is, for example, below the limit of detection of conventional immune detection methods.

The invention is further directed to methods of prognosing a candidate patient for immunotherapy. A diagnostic test according to the present invention can evaluate whether a patient is a candidate for long-term non-progression by assaying for an immune response against an immunogenic molecule, such as an antigen. Another diagnostic test encompassed by the present invention can evaluate whether a subject is a candidate for a treatment method that prevents the diseases and conditions involving an immune response.

In one embodiment, the present invention includes a diagnostic test that determines whether a subject can exhibit an immune response against an immunogenic molecule. In another embodiment, a diagnostic test is employed to determine whether a subject exhibits an increased activity of a T-cell, a NK cell, or a macrophage. In another embodiment, the diagnostic method is employed to determine whether a subject exhibits an increased cytokine concentration. In either case, if the subject does, the present invention includes eliciting an immune response using compositions described herein. In further embodiments, a subject who either exhibits or can exhibit an induced immune response is administered a treatment method to enhance the immune response.

D. Immune Stimulation

1. Inducing an Immune Response

Cytokines can promote an immune response to a compound. Because MDA-7 has cytokine activity, this effect can be utilized for therapeutic and preventative methods. It is contemplated that an immune response against any of the antigens described below would effect a therapeutic effect against a disease or condition associated with the antigen or effect a preventative therapy against that disease or condition.

In the present invention, MDA-7 enhances an immune response against an antigen associated with a disease or condition. In certain embodiments of the invention, antigens may be associated or derived from microbial, fungal, viral, or tumor agents. Examples of microbes from which antigens of the invention are drawn include, but are not limited to, the 83 or more distinct serotypes of pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcus faecalis, Enterococcus faecium, Staphylococci, such as Staphylococcus epidermidis, Staphylococcus aureus, particularly in the nasopharynx, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species and related organisms. The invention may also be useful against gram negative bacteria such as Klebsiella pneumoniae, Escherichia coli, Proteus, Serratia species, Acinetobacter, Yersinia pestis, Yershinia enterocolitica, Yersinia pseudotuberculosis, Francisella tularensis, Enterobacter species, Bacteriodes and Legionella species and the like. In addition, the invention may prove useful in controlling protozoan or macroscopic infections by organisms such as Cryptosporidium, Isospora belli, Toxoplasma gondii, Trichomonas vaginalis, Cyclospora species, for example, and for Chlamydia trachomatis and other Chlamydia infections such as Chlamydia psittaci, or Chlamydia pneumoniae, for example.

Bacterial antigens and/or virulence factors of pathogenic bacteria to which the present invention is drawn include, but are not limited to, Mycobacterium tuberculosis soluble factor (Mtb), phenol-soluble modulin (PSM) from Staphylococcus epidermidis, N. gonorrhea liposaccharide (LOS), Vibrio cholerae, Salmonella typhimurium, Shigella spp., Aeromonas hydrophilia, Pseudomonas aeruginosa, Clostridium botulinum, Bacillus anthroacis.

Examples of viruses against which viral antigens of the invention may be from include, but are not limited to, influenza A, B and C, parainfluenza, paramyxoviruses, Newcastle disease virus, respiratory syncytial virus, measles, mumps, adenoviruses, adenoassociated viruses, parvoviruses, Epstein-Barr virus, rhinoviruses, coxsackieviruses, echoviruses, reoviruses, rhabdoviruses, lymphocytic choriomeningitis, coronavirus, polioviruses, herpes simplex viruses, human immunodeficiency viruses, cytomegaloviruses (e.g., CMV-G and CMV-M antigens), papillomaviruses, virus B, varicella-zoster, poxyiruses, rubella, rabies, picomaviruses, rotaviruses and Kaposi associated herpes viruses, hepatitis A, B, C, D, E, F, G, and any other hepatitis viruses, West Nile virus, influenza viruses, paopvaviruses, retroviruses, dengue fever viruses, ebola viruses, and rubella viruses.

Examples of fungi against which antigens of the invention may be from include, but are not limited to, Pityrosporum orbiculare, Exophiala werneckii, by Piedraia horta, Trichosporon beigelii, Candida albicans, Sporothrix schenckii, Cladophialophora carrionii, Phialophora verrucosa, two species of Fonsecaea, Pseudallescheria boydii, Madurella mycetomatis, Madurella grisea, Exophiala jeanselmei, Acremonium falciforme, Exophiala jeanselmei, Phialophora richardsiae, Bipolaris spicifera, Wangiella dermatitidis, Histoplasma capsulatum, Coccidioides immitis, P. brasiliensis, Candida, Cryptococcus neoformans, Aspergillus fumigatus, Aspergillus flavus, Aspergillus niger, Pneumocystis carinii, Rhizopus, Rhizomucor, Absidia, Blastomyces dermititidis, Histoplasma capsulatum, Paracoccidiodes spp., and Basidiobolus.

Furthermore, it is contemplated that all or part of MDA-7 may be part of a fusion protein with another cytokine molecule and/or with an antigen against which an immune response is desired. This could be administered to a subject to induce or promote an immune response against the antigen.

The present invention includes methods for promoting an immune response in a subject comprising providing to the subject an effective amount of MDA-7 to promote an immune response. The promotion of an immune response is evidenced by an increase of cytokine expression or activity, proliferation of T cells or a population of T cells (for example, helper, cytotoxic, NK cells), proliferation of B cells or a population of B cells, cytotoxic T cell activity, or antibody production.

In certain embodiments of the invention, an antigen also is provided to the subject, resulting in an immune response against the antigen. The antigen may be a tumor antigen, microbial antigen, viral antigen, or fungal antigen, or a combination thereof. In specific embodiments the antigen is a tumor antigen, such as PSA, CEA, MART, MAGE1, MAGE 3, gp100, BAGE, GAGE, TRP-1, TRP-2, or PMSA.

Additional embodiments of the invention include methods of enhancing or improving recovery or methods of reducing damage from traumatic treatment, which is a treatment that causes damage to normal cells. Such damage causes neutropenia, anemia, thrombocytopenia, and lymphopenia, for example. In specific embodiments, the traumatic treatment is chemotherapy and/or radiotherapy. It is contemplated that immune therapy is enhanced by administering an effective amount of MDA-7 to a patient who will, is undergoing, or has undergone traumatic treatment. MDA-7 can be provided to a subject before, after or during treatment.

MDA-7 can also be administered to a patient in combination with a tumoricidal compound or a compound with a tumor cytostatic effect to enhance the ability of that compound to inhibit or kill tumor cells. Such compounds include tumor suppressors and compounds discussed below under the heading “Combination Therapy.” In specific embodiments, the tumoricidal compound is p53, Rb, WT, FHIT, p16, PTEN, APC, DCC, NF-1, NF-2, WT-1, MEN-I, MEN-II, zac1, p73, VHL, MMAC1, DBCCR-1, FCC, rsk-3, p27, or TRAIL.

An immune response against tumor antigens can also be implemented with MDA-7. Tumor antigens include PSA, CEA, MART, MAGE1, MAGE3, gp100, BAGE, GAGE, TRP-1, TRP-2, PMSA, Mycobaterium tuberculosis soluble factor (Mtb), phenol soluble modulin (PSM), CMV-G, CMV-M, EBV capsid-EB nuclear antigen (EBNA), gp120, gp41, tat, rev, gag, toxa antigen, rubella antigen, mumps antigen, alpha-fetoprotein (AFP), adenocarcinoma antigen (ART-4), CAMEL, CAP-I, CASP-8, CDC27m, CDK4/m, CEA, CT, Cyp-B, DAM, ELF2M, ETV6-AMLI, ETS G250, GnT-V, HAGE, HER2/neu, HLA-A*0201-R1701, HPV-E7, HSP 70-2M, HST-2, hTERT, ICE, KIAA 0205, LAGE, LDLR/FUT, MC1R, MUCI, MUM-1, MUM-2, MUM-3, NA88-A, NY-ESO-I, p15, Pm1/RARalpha, PRAME, PSM, RAGE, RU1, RU2, SAGE, SART-1, SART-3, TEL/AMLI, TPI/m, or WT1. Uses for inducing a response against tumor antigens are specifically contemplated and can be found in U.S. Pat. Nos. 5,552,293 and 6,132,980, which are specifically incorporated by reference.

A number of assays are well known to those of skill in the art regarding assaying for induction, promotion, or enhancement of an immune response, some of which are described in an example below and in the references incorporated by reference herein. One assay involves detecting an increase of expression of other cytokines, such as IL-6, TNF, IFN-alpha, IFN-beta, or IFN-gamma, GM-CSF, CSF, IL-1beta, IL-2, IL-4, IL-8, IL-10, or 1L-12. Other methods to detect an induced immune response involve increased activity of a T-cell, a NK cell, or a macrophage.

It is contemplated that any embodiment discussed with respect to MDA-7 and/or an immunogenic molecule such as an antigen, may be applied to methods of enhancing an immune response. More specifically, the embodiments discussed with respect to MDA-7 and enhancing an immune response against the respective immunogenic molecule, wherein the immunogenic molecule is previously identified or not previously identfied.

As a diagnostic method, the present invention contemplates assaying a T-cell response, which includes assaying cells from an autologous B-cell line (B-LCL), dendritic cells, or MHC matched cells. The term “autologous” is used to refer to cells derived from a subject from whom the effector cells are also derived. An autologous B-LCL can be prepared using peripheral blood mononuclear cells (PBMCs) from the subject who will be diagnosed or treated and transforming them. In a specific embodiment, an autologous B-LCL is made from the HIV-infected subject and used as a target cell in a T-cell response assay to predict long-term non-progression in the B-LCL donor.

Dendritic (DC) cells act as antigen presenting cells and play a key role in T-cell activation. They are unique among antigen presenting cells (APC) by virtue of their potent capacity to activate immunologically naive T cells (Steinman, 1991). DC express constitutively, or after maturation, several molecules that mediate physical interaction with and deliver activation signals to responding T cells. These include class I and class II MHC molecules, CD80 (B7-1) and CD86 (B7-2), CD40, CD11a/CD18 (LFA-1), and CD54 (ICAM-1) (Steinman, 1995; Steinman, 1991). DC can present antigen to both CD8+ and CD4+T lymphocytes. DC also secrete, upon stimulation, several T cell-stimulatory cytokines, including IL-1β, IL-6, IL-8, macrophage-inflammatory protein-1α (MIP-1α) and MIP-1γ (Mohamadzadeh, 1996; Ariizumi, 1995; Kitajima, 1995; Caux, 1994; Enk, 1992; Heufler, 1992; Matsue, 1992; Schreiber, 1992). Both of these properties, adhesion molecule expression and cytokine production, are shared by other APC (e.g., activated macrophages and B cells), which are substantially less competent in activating naive T cells.

In other embodiments, lymphocyte surface marker studies can be used to assay for the presence of such T-cell surface markers using various procedures that are known to one of ordinary skill in the art, including the use of immunofluorescence and flow cytometry. T-cell responses can be measured by a variety of protocols that are known to one of ordinary skill in the art. Some of these assays are described in fuller detail below.

a. ³[H]Thymidine Incorporation Assay

The proliferative responses of PBMCs from different samples can be determined by the standard ³[H]thymidine incorporation assay as described in published articles (Nehete, 1996; Nehete, 1995). The significance of T-cell proliferative responses to the individual E6 and E7 peptides (in terms of stimulation index [SI]) can be calculated as the fold increase of ³[H] thymidine incorporation by cells exposed to the peptide over that by the control to which no peptide was added. An SI value of at least 2.0, including at least about 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 or more, which are considered positive responses. Generally, an SI value is calculated by measuring the amount of radioactivity (cpm) in media from cells incubated with the peptide(s) and dividing by the amount of radioactivity in media from cells not incubated with peptide(s) (media alone).

b. Lysis Using ⁵¹[Cr]

Cell-mediated lympholysis (CML) can be used as an indication of T-cell response. Target cells can be labeled with radioactive chromium-51 (⁵¹[Cr]) prior to exposure to effector cells. The amount of 51 [Cr] released into the media is proportional to the level of cell-mediated lysis. In a specific embodiment of the present invention, autologous B-lymphocyte cell lines are cultured and then exposed to ⁵¹[Cr] sodium chromate for two hours before they are incubated with cells possessing cytotoxic activity.

c. γ-Interferon Production

Interferon gamma (γ-interferon), also called type II or immune interferon, is produced by T cells and NK cells. It is critical for the development of helper T cells. Because it is the primary macrophage-activating factor, it is a strong cytokine in cell-mediated immunity. γ-interferon increases the levels of MHC class I and MHC class II expression, which improves antigen presentation and other cognitive reactions. Furthermore, it amplifies the effects of TNF-α and raises expression levels of adhesion molecules on the surface of vascular endothelial cells, which leads to T cell adhesion and extravasation. Finally, as part of the claimed invention, γ-interferon is secreted by CTLs, enabling the level of γ-interferon to act as an indicator of CTL activity and thus of a CTL response. Determining γ-interferon levels is performed using standard assay methods.

d. Tetramer Assay

Tetramer assays are well known to those of skill in the art. See Altman, 1996.

e. Cytokine Production

Cytokines are proteins that play important roles in the regulation of immune responses as well as in the differentiation pathways of different cell types. They have a critical function in T cell regulation and development, and these include y-interferon, interleukin 1 (IL-1), IL-2, IL-4, IL-5, IL-6, IL-7, IL-10, IL-12, IL-13, IL-14, IL-15, lymphotoxin, MIF, TGF-β, TNF-α, and other chemotactic cytokines. Assays for cytokines are well known in the art.

The present invention also includes methods of determining whether a subject expresses or can express a molecule specific to indicating an immune response. Because the MDA-7 provides a means to enhance an immune response, a method of the present invention includes assaying for an immune system indicator such as expression of a protein, peptide or polypeptide that is differentially expressed by a cell comprising the immune system. There are numerous assays available to qualify and quantify expression levels of a molecule, and these can involve detecting DNA sequences that signify a particular haplotype or measuring protein or mRNA expression levels. These assays are well known by one of ordinary skill in the art. Some examples are provided below.

f. Serological Assay

The present invention includes the implementation of serological assays to evaluate the expression levels of immune system indicators. These assays take advantage of antigen-antibody interactions to quantify and qualify antigen levels. There are many types of assays that can be implemented, which one of ordinary skill in the art would know how to implement in the scope of the present invention.

g. ELISAs, Immunoassay and Immunohistological assay.

Immunoassays generally are binding assays. Certain some immunoassays are the various types of enzyme linked immunosorbent assays (ELISAs) and radioimmunoassays (RIA) known in the art. Immunohistochemical detection using tissue sections is also particularly useful. Immunoassays encompassed by the present invention include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo. Assays for the presence of an induced immune response may be performed directly on tissue samples. Methods for in vitro situ analysis are well known and involve assessing binding of antigen-specific antibodies to tissues, cells, or cell extracts. These are conventional techniques well within the grasp of those skilled in the art.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute some modes for its practice. However, those of skill in the art should, in light of the present disclosure, appreciate that many changes can be made in the specific embodiments which are disclosed and still obtain a like or similar result without departing from the spirit and scope of the invention.

### Example 1

### AD-MDA7 Induces PKR Expression and Apoptosis in Lung Cancer Cells

a. Cell Lines and Reagents

The human lung cancer cell lines A549 (wt p53), H1299 (p53 null) and H322J (mutant p53) were obtained from the American Type Culture Collection. PKR +/+ and PKR −/− mouse embryo fibroblast (MEF) cells were obtained from Dr. Glen Barber (University of Miami School of Medicine). MEF cells were maintained in Dulbecco's modified Eagle's medium (DMEM) containing 10% fetal bovine serum, 10 mM glutamine, 100 units/ml penicillin, 100 μg/ml streptomycin (Life Technologies, Inc., Grand Island, N.Y.) in a 5% CO₂ atmosphere at 37° C. 2-Aminopurine Nitrate Salt (2-AP) was obtained from Sigma Chemical Co. (St. Louis, Mo.).

b. Virus

Construction of the Ad-mda7, Ad-Bax, AdBak, Ad-p53 and Ad-Luc vectors have been previously reported (Pataer et al., 2000). The transduction efficiencies of adenoviral vectors in various cancer cell lines were determined by infecting cells with Ad-LacZ and then determining the titers needed to transduce at least 70% of the cells.

c. Flow Cytometry Analysis and XTT Assay

Apoptotic cells by propidium iodide staining and FACS analysis were measured. Cells were harvested, pelleted by centrifugation and resuspended in phosphate-buffered saline containing 50 μg/ml propidium iodide, 0.1% Triton X-100, and 0.1% sodium citrate. Samples were stored at 4° C. for 16 hours and vortexed prior to FACS analysis (Becton-Dickenson FACScan, Mountain View, Calif.; FL-3 channel). Cellular viability was assessed with the XTT assay by growing cells in 96-well plates at 100 μL volume/well. After treatment with Ad-mda7 or control vectors, cells were incubated with the tetrazolium salt XTT according to the Roche protocol (Roche Diagnostics, Mannheim, Germany). Viability was assessed spectrophotometrically on an enzyme-linked immunosorbent assay (ELISA) plate reader.

d. Immunoblot assay

Forty-eight hours after transfection, cell extracts were prepared for immunoblot assays as previously described (Pataer et al, 2000). The following antibodies were used: Bc1-2, Bak, Bax, PKR (K-17), eIF-2α, β-actin, p38 (A-12), phosphospecific p38 (D-8), stat1 (C-136), phosphospecific stat1 (A-2), stat3 (F-2), phosphospecific stat3 (B-7), anti-Tyk2 (C-20) and phosphospecific anti-Tyk (PY-99) (Santa Cruz Biotechnology, Santa Cruz, Calif.); Caspase 3, caspase-9, caspase-8 and Bid (PharMingen , San Diego, Calif.) and the phosphospecific PKR [pT451] and eIF-2a [pS51] (BioSource International, Camarillo, Calif.). The polyclonal and monoclonal antibodies to MDA-7 were obtained from Introgen Therapeutics, Inc. (Houston, Tex.).

e. Recombinant Adenovirus Production

Replication deficient human type 5 Adenovirus (Ad5) carrying the nucleic acid encoding extracellular human MDA-7 (or Luciferase gene) linked to an internal CMVIE promoter and followed by SV40 polyadenylation (pA) signal were constructed. A third control vector with just the CMV-pA construct also was constructed. The Ad-5 vectors harboring the gene cassettes were co-transfected with plasmid pJM17 (Graham and Prevec 1992) in 293 cells to rescue recombinant viruses Ad-mda7, AdLuc and AdCMVpA. Plaques were picked, virus stocks were grown and their genomes were confirmed as correct by PCR/restriction analysis and sequencing. Viruses were propagated in 293 cells and purified by HPLC.

f. Transduction and Cell Proliferation studies

Cancer or normal cell lines used in this study are infected with Ad-mda7 (with either AdCMVpA or AdLuc as controls) in increasing MOIs (viral particles/cell; 0, 100, 250, 500, 1000, 2500, 5000, 10000 vp/cell increasing concentrations). Cells were either plated at 500-2000 cells/well in 96-well format for Tritiated thymidine incoporation-Cell Proliferation Assay or plated at 10⁵-10⁶ cells/well in a 6 well plate for protein expression or Apoptosis assays or plated at 10⁴ cells/well for Alamar-blue assay.

For infection Ad-mda7 or AdLuc (or AdCMVpA) were used at increasing MOIs (based on viral particles/cell; MOI ranged from 0-10,000 viral particles/cell). For tritiated thymidne /apoptosis and protein expression and alomar assays, cells were analyzed 3 and 5 days post-infection.

g. Measurement of Cellular Protein Synthesis

Cells were treated with Ad-mda7 or Ad-Luc for 48 hrs. Where indicated, cells were treated with 1 mM 2-AP for 48 hrs. Measurement of protein synthesis was performed as previously described (14). After treatment, approximately 1×10⁶ cell equivalents were incubated with L-[U-¹⁴C] amino acid mixture (Amersham, Piscataway, N.J.) at 2 μCi/ml for 10 min at 37° C. The reaction was terminated by the addition of 20% (wt/vol) trichloroacetic acid and the radioactivity in the acid-precipitable fraction was measured in a scintillation counter.

h. Confocal Analysis

4×10⁵ cells/well were grown on chamber slides overnight and infected with Ad-mda7, Ad-Luc or PBS. 48 hrs later cells were washed with PBS and fixed overnight in 4% paraformaldehyde. Cells were then permeabilized for 20 min at 4° C. with 0.2% triton-X10O and blocked with 5% normal horse serum and 1% normal goat serum. Rabbit polyclonal anti-MDA7 and mouse monoclonal anti-PKR(B-10) were incubated overnight at 4° C. and developed with Rhodamine or FITC goat anti-rabbit IgG for 30-45 min at 37° C. Cells were then visualized under confocal microscopy.

i. Statistical Analysis

The data reported in the figures represent the mean of three or more independent experiments and the bars show the standard deviation (SD). ANOVA and two-tailed Student't test were used for statistical analysis of multiple groups and pair-wise comparison, respectively, with p<0.05 considered significant.

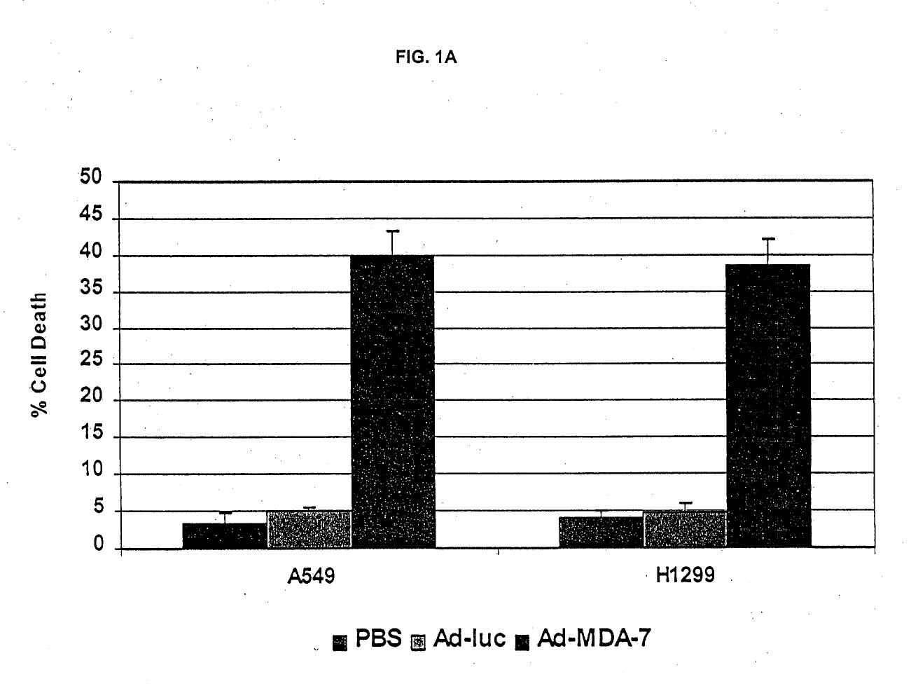

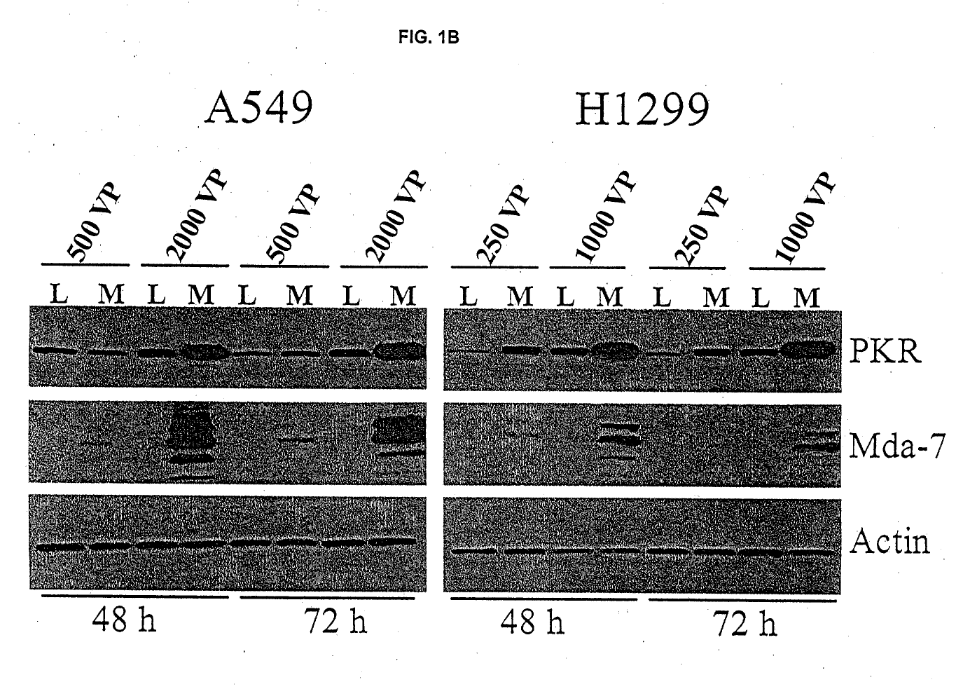

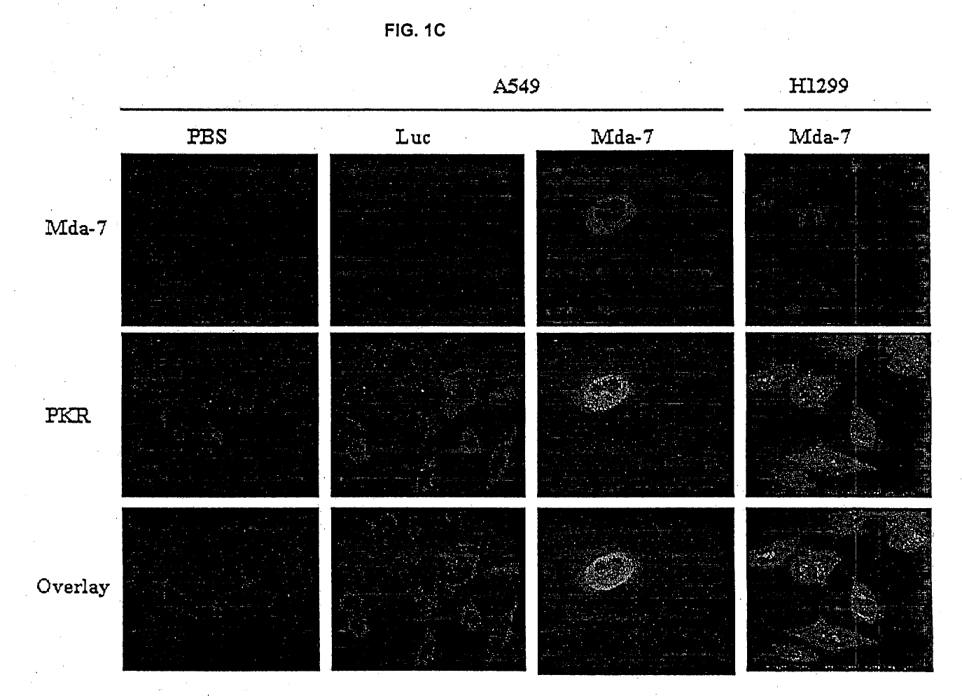

Flow cytometric analysis of apoptosis was performed on the A549 (wt p53), H1299 (null p53), and H322J (mutant p53) lung cancer cells 24 to 96 hrs following infection with Ad-mda7, Ad-Luc or PBS. Ad-mda7 resulted in a high percentage of apoptosis in all of three lung cancer cells (FIG. 1A). Inhibition of cellular viability by XTT assay after infection of Ad-mda7, Ad-Luc or PBS control were determined. Consistent with the FACS results, Ad-mda7 infected cells showed significant inhibition of cell growth 48 hours after transduction. Western blot expression and confocal microscopy demonstrated dose dependent increases in PKR (FIGS. 1B, 1C) following Ad-mda7 transduction but not after transduction with control vectors (Ad-Luc), PBS or other pro-apoptotic vectors such as Ad-Bax, Ad-p53 or Ad-Bak.

Therefore, up-regulation of the interferon induced, ds-RNA dependent serine/threonine protein kinase (PKR) was observed after treatment with Ad-mda7. Ad-mda7 transduction of lung cancer cells led to PKR induction in a p53 independent manner (FIGS. 1A and 1B). PKR upregulation was also observed in other types of cancer cells including colorectal and breast suggesting this relationship is not specific to lung cancer cells. Up-regulation of PKR also appears specific for Ad-mda7 becasuse little PKR change was observed following transduction of cancer cells with adenoviral vectors containing the luciferase reporter gene (FIGS. 1B and 1C), or other pro-apoptotic genes such as p53 (Ad-p53), Bax (Ad-Bax) or Bak (Ad-Bak). In addition, PKR induction does not appear due to non-specific caspase cleavage since blockage with caspase inhibitors did not abrogate PKR upregulation. Interestingly, the PKR functional status has been implicated as an important regulator of tumorigenesis (Jagus et al., 1999). Upregulation of PKR led to the induction of apoptosis in various cancer cell lines. Furthermore, in myelodysplasias, critical tumorigenic deletions of the IRF-1 gene on chromosome 5q appear associated with decreased PKR levels and immunohistochemical analyses of lung and colorectal cancers demonstrate an association with PKR expression and prolonged survival.

### Example 2

### AD-MDA7 Activates PKR, EIF2ALPHA and Other PKR Substrate Targets

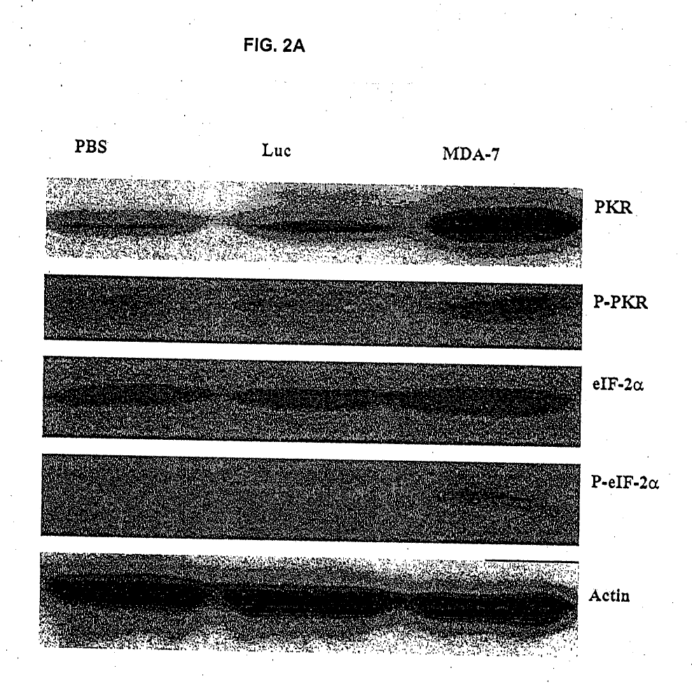

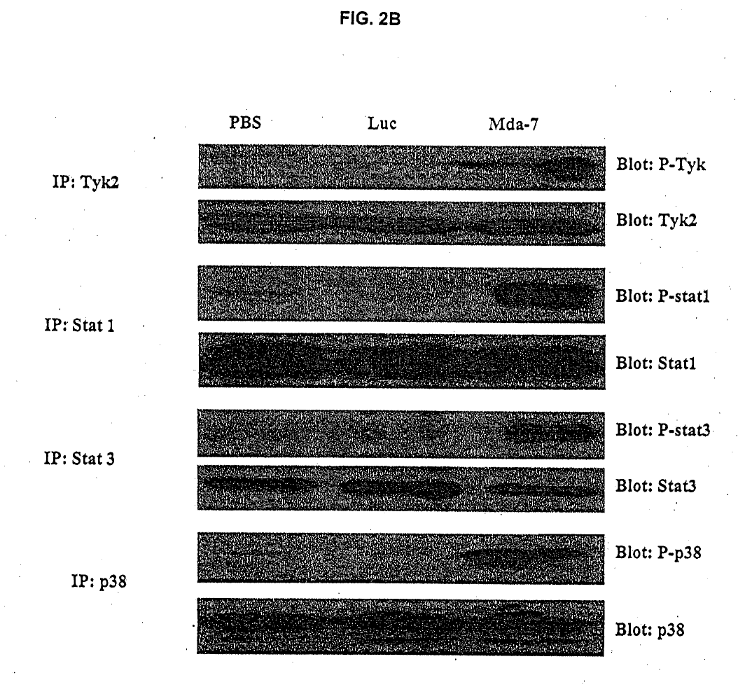

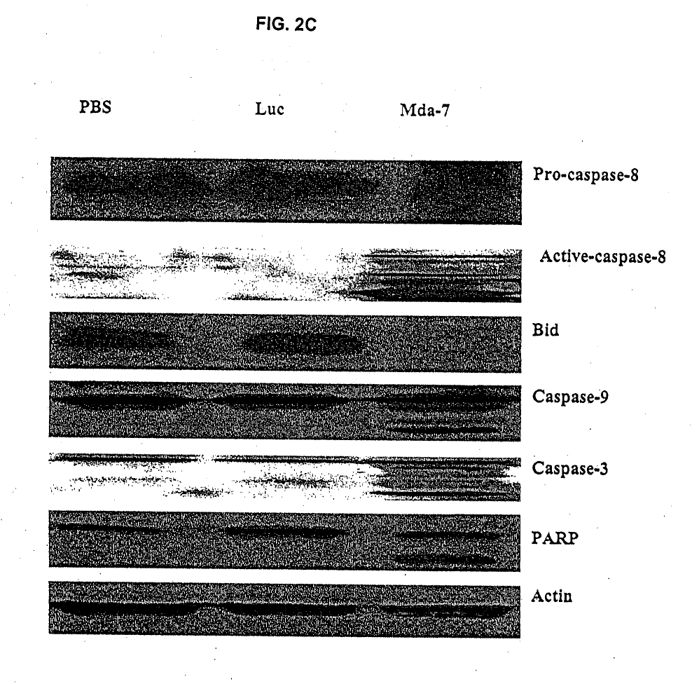

The activation of PKR in vitro by Ad-mda7 was observed utilizing the materials and methods described in Example 1. Ad-mda7 treated cells (A549) were assessed by immunoblot assay for the presence of phosphorylated PKR. Only Ad-mda7 treated cells demonstrated increased expression of PKR and its active phosphorylated form (FIG. 2A). Activation of the serine/threonine kinase was also demonstrated by phosphorylation of PKR's downstream targets: eIF-2α, Tyk2, Stat1, Stat3 and p38 (FIGS. 2A, 2B). Treatment with Ad-mda7 led to subsequent apoptosis induction with caspase 3, 8 and 9 activation, and BID and PARP cleavage (FIG. 2C). The activation of PKR appeared to be specific for Ad-mda7 and upstream of caspase activation since pretreatment with caspase inhibitors failed to block PKR phosphorylation.

PKR appears to mediate anti-tumorigenic activity through the activation of multiple transduction pathways culminating in growth inhibition and apoptosis induction. Activation of these pathways occurs after the latent, inactive homodimeric form is induced by activating signals to undergo conformational changes leading to auto-phosphorylation and activation (Vattem et al., 2001). Once activated, PKR is able to phophorylate various substrate targets, which are important in growth control and apoptosis induction (Saelens et al., 2000; Sudharkar et al., 2000). The immunopreciptation studies are consistent with this model showing PKR activation following Ad-mda7 transduction (FIG. 2A) leading to increases in phosphorylated (active) PKR and phosphorylated eIF-2α. The phosphorylation of several other PKR substrate targets, which may be associated with apoptosis induction and growth control including Stat1, Stat3, p38 and Tyk2 (Deb et al., 2001; Goh et al., 2000), were demonstrated.

The activation of PKR appears to be a critical event in Ad-mda7 apoptosis since inhibition of PKR with the specific threonine/kinase inhibitor, 2 amino-purine (2-AP) leads to almost complete reversal of Ad-mda7 apoptosis and abrogation of eIF-2α phosphorylation and protein synthesis inhibition. The inhibition of protein synthesis may be critical to the induction of apoptosis possibly because of regulation of one or more short-lived proteins involved in apoptosis inhibition. Alternatively, other pathways controlled by PKR may be important such as those involved in regulation of NF-κB, p53, MEK, IRF-1 or FADD (Jagus et al., 1999; Gil et al., 1999; Cuddihy et al., 1999; Balachandran et al., 1998).

### Example 3

### AD-MDA7 Apoptosis Induction Dependent on PKR Activation

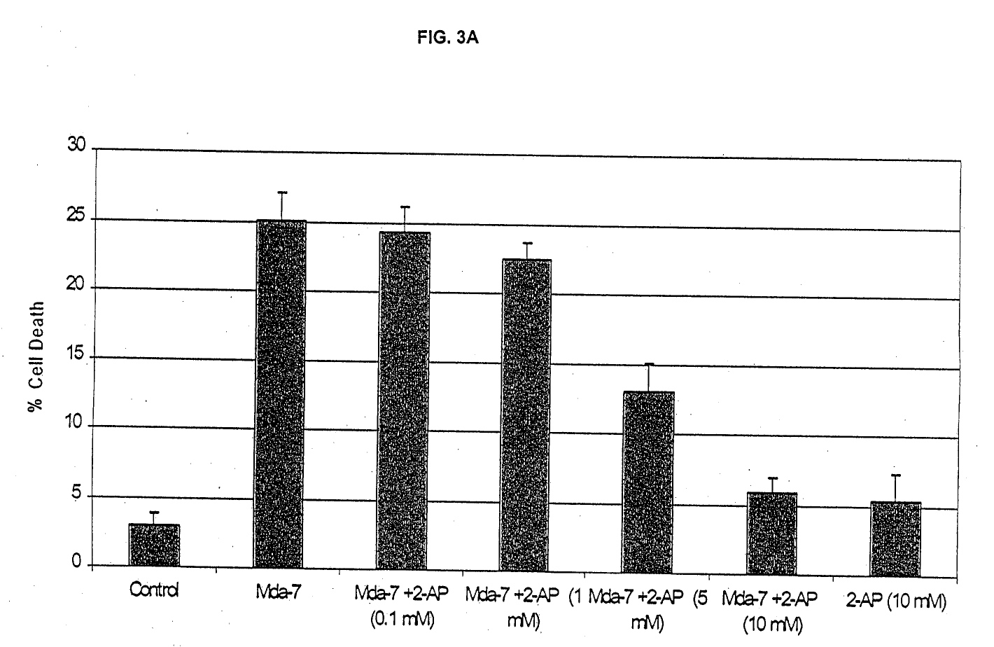

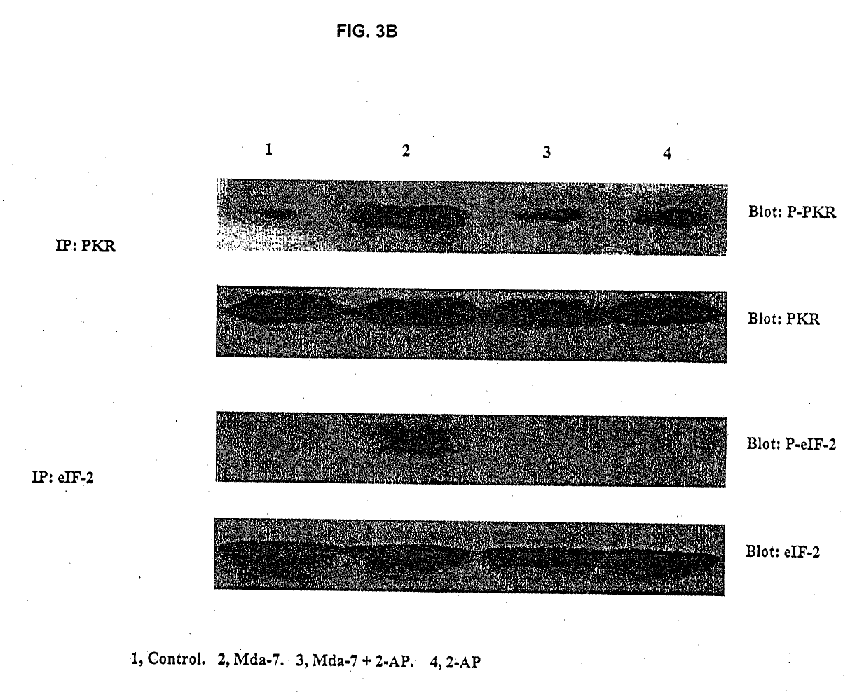

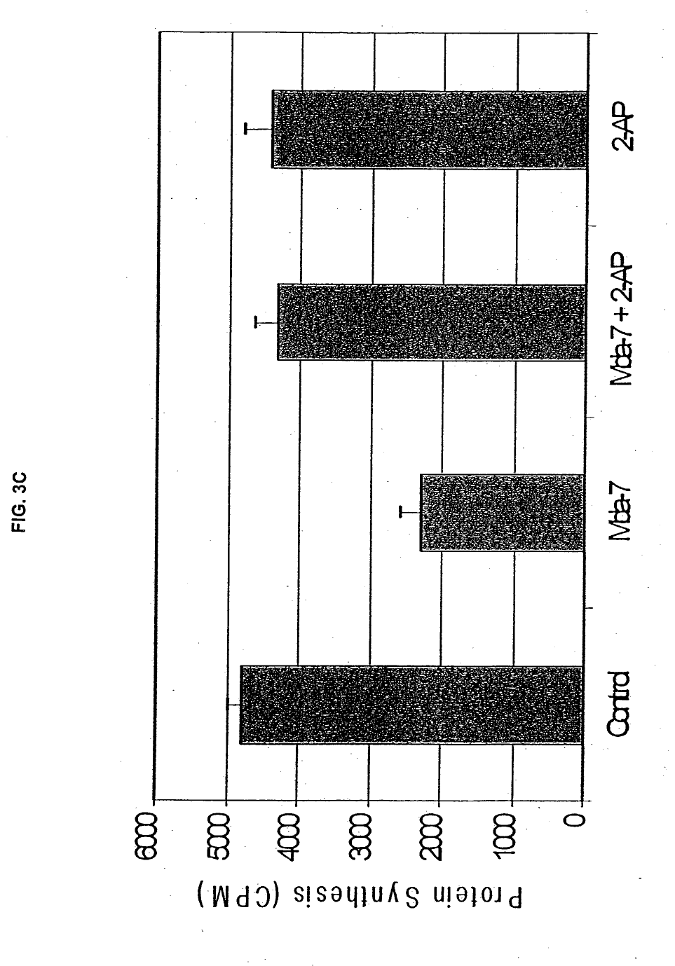

The effect of the specific serine/threonine kinase inhibitor 2 aminopurine (2-AP) was investigated to determine the relationship between Ad-mda7 apoptosis induction and PKR activation utilizing the materials and methods described in Example 1. A high concentration of 2-AP (10 mM) alone was found to have no effect on cell viability. However, in cells treated with Ad-mda7, 2-AP blocked apoptosis induction and cell killing in a dose dependent manner (FIG. 3A). Inhibition of apoptosis by 2-AP appeared specific to Ad-mda7 and was not seen following treatment with Ad-Bak, Ad-Bax, Ad-p53 or staurosporine. Immunoprecipitation studies demonstrated that 2-AP treatments attenuated PKR activation with inhibition of both PKR and eIF-2α phosphorylation (FIG. 3B). 2-AP's ability to block eIF-2α phosphorylation led to reversal of eIF-2α protein synthesis inhibition (FIG. 3C) and inhibition of apoptotic induction (FIG. 3A).

### Example 4

### AD-MDA7 Apoptosis Induction and PKR Activation in MEFS

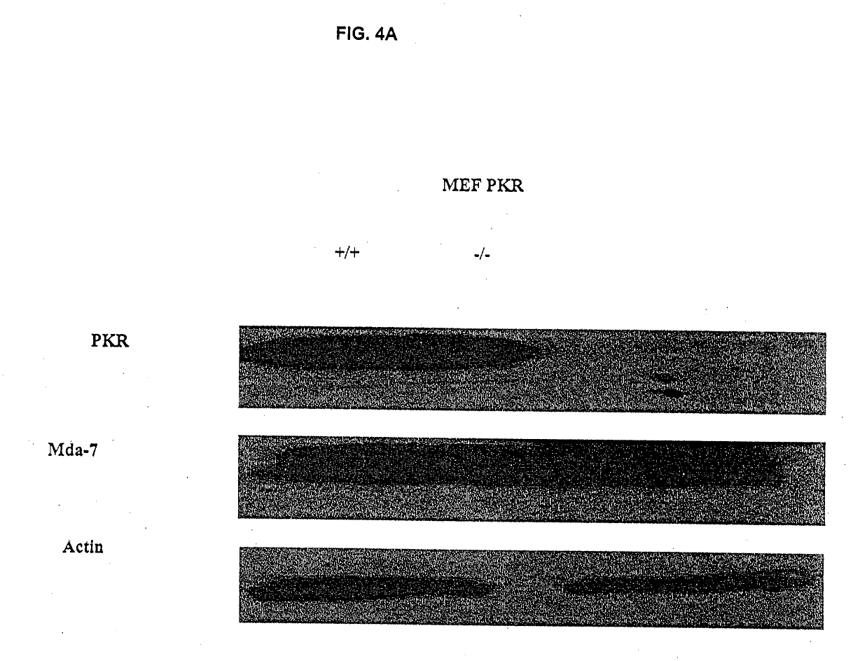

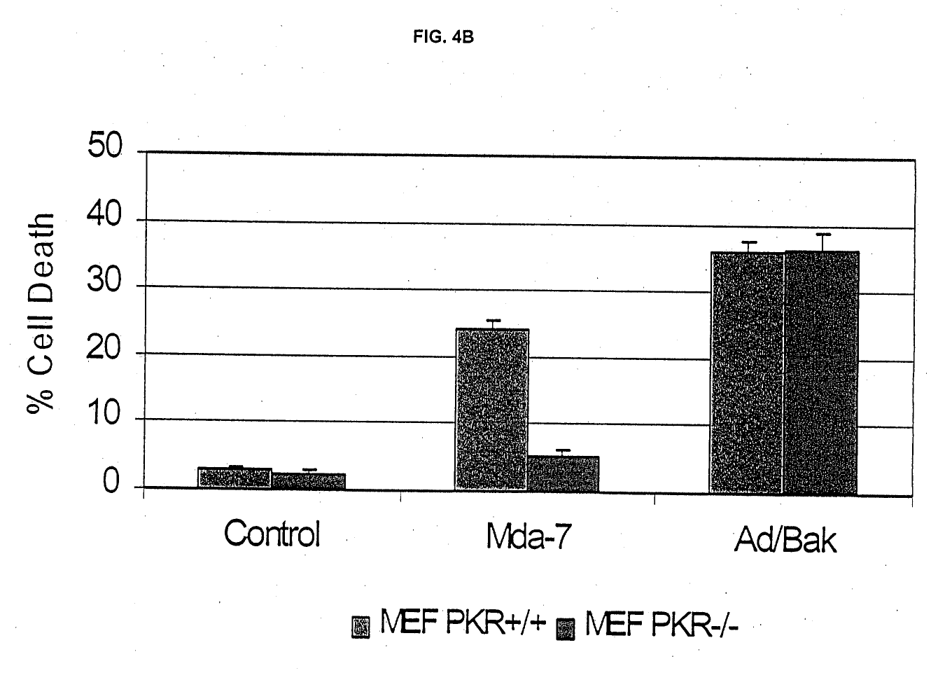

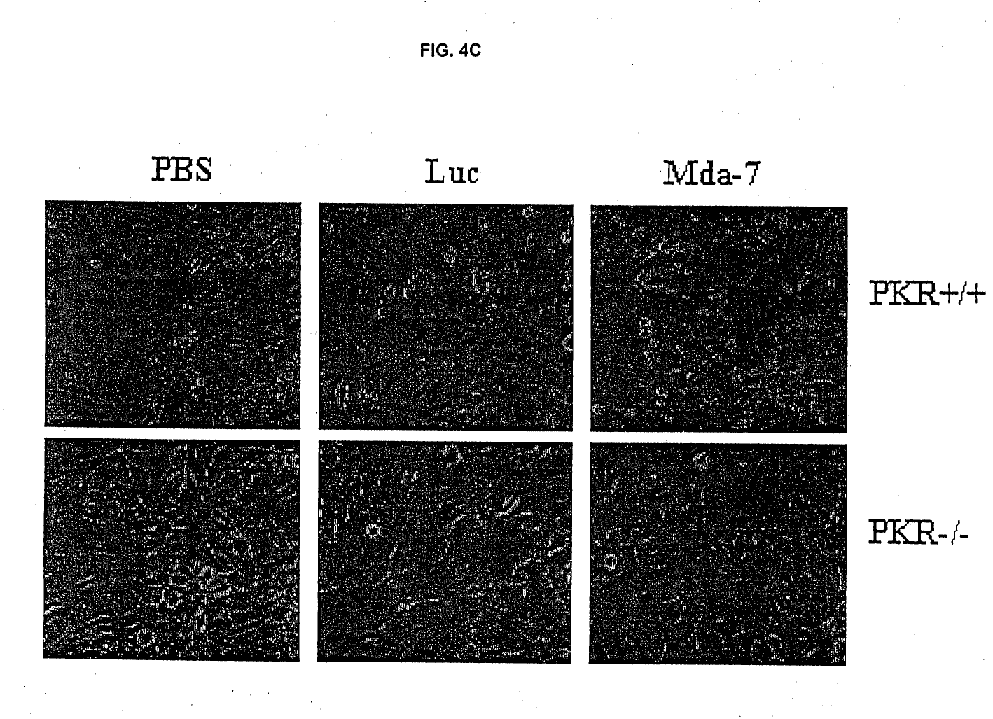

PKR activation and Ad-mda7 apoptotic activity was evaluated in MEFs obtained from PKR knock out mice using the materials and methods described in Example 1. Despite adequate transduction and expression of MDA-7 protein in both PKR null (−/−) and wild-type MEFs (FIG. 4A), only PKR wild-type MEFs underwent apoptosis induction following Ad-mda7 treatment (FIG. 4B) suggesting that Ad-mda7 induced cell killing was dependent on PKR. Unlike Ad-mda7, Ad-BAK apoptosis induction did not appear to be dependent on PKR genomic status (FIG. 4C) with apoptosis occurring in both PKR null and wild-type MEFs suggesting PKR activation was not necessary for activity of all pro-apoptotic genes.

Even though multiple pathways may be involved, PKR activation is critical for Ad-mda7 apoptosis because MEFs lacking PKR were unable to undergo apoptosis as opposed to MEFs with wild-type PKR. This inhibition of apoptosis appeared specific to mda-7 since transduction of MEFs lacking PKR with the pro-apoptotic Ad-BAK vector lead to unimpaired apoptosis. A model was synthesized based on these results in which MDA-7 and PKR are upstream of the pro-apoptotic BAK gene in the apoptosis cascade. In this model, MDA-7 induces PKR activation which leads to various cellular pathways that then induce caspase activation and apoptosis induction. BAK, being downstream of PKR, is not dependent on PKR activation to induce apoptosis. Interestingly, BID cleavage and caspase 8 activation was observed, which is consistent with previous work that have demonstrated that PKR apoptosis is often mediated through activation of Fas, FADD, caspase-8 and BID (Balachandran et al., 1998).

Adenoviral-mediated overexpression of MDA-7 led to the rapid induction and activation of PKR with subsequent phosphorylation of eIF-2α, other PKR target substrates and apoptosis induction. Specific inhibition of PKR by 2-AP in lung cancer cells abrogates Ad-mda7 induced PKR activation, PKR substrate target phosphorylation and apoptosis induction. As evidenced by PKR null fibroblasts, Ad-mda7 apoptosis is dependent on a functional PKR pathway.

### Example 5

### AD-MDA7 Induces PKR Expression and Apoptosis in Lung Cancer Cells

Materials and Methods

1. Cell Lines and Immunoblot Assay.

A549 and H1299 human lung cancer cell lines were obtained from the American Type Culture Collection (ATCC, Rockville, Md.). All cells were maintained in RPMI 1640 supplemented with 10% fetal bovine serum, 10 mM glutamine, 100 units/ml penicillin, 100 μg/ml streptomycin (Life Technologies, Inc., Grand Island, N.Y.) in a 5% CO₂ atmosphere at 37° C. The following antibodies were used: BAK, BAX, Bc1-2, Fas, FasL, FADD, TNFα, TNFR1, TRADD, β-actin, (Santa Cruz Biotechnology, Santa Cruz, Calif.), and cytochrome c (PharMingen, San Diego, Calif.).

2. Adenovirus Production and Transduction.

Construction of the Ad-mda-7, Ad-p53, Ad-LacZ, and Ad-Luc vectors have been reported previously (Pataer et al., 2000). The transduction efficiencies of adenoviral vectors in the cell lines were determined by infecting cells with Ad-LacZ. Subsequent experiments utilized viral titers needed to transduce at least 70% of the cells.

3. Apoptosis and Cellular Viability Assays.

Apoptotic cells were measured by propidium iodide staining and FACS analysis. Cells were harvested, pelleted by centrifugation and resuspended in phosphate-buffered saline (PBS) containing 50 μg/ml propidium iodide, 0.1% Triton X-100, and 0.1% sodium citrate. Samples were stored at 4° C. for 16 hours and vortexed prior to FACS analysis (Becton-Dickenson FACScan, Mountain View, Calif.; FL-3 channel). Cellular viability was assessed with the XTT assay by growing cells in 96-well plates in 100 μl volume/well. On day one, cells were transduced with Ad-mda-7, Ad-p53 or control vectors. On day two, the cells were incubated with the tetrazolium salt XTT for 4 to 24 hours according to the Roche protocol (Roche Diagnostics, Mannheim, Germany). Viability was assessed spectrophotometrically on an enzyme-linked immunosorbent assay (ELISA) plate reader.

4. Mitochondrial Membrane Potential Measurements.

The potential-sensitive fluorochrome tetramethylrhodamine, ethylester, perchlorate (TMRE) (Molecular Probes, Eugene, Oreg.) was used to measure ΔΨ_{m.} Cells were incubated with 25 nM TMRE for 30 minutes at 37° C. in the dark, washed in PBS, and analyzed by FACS on the FL-2 channel (FACScan: Becton Dickinson, Mountain View, Calif.).

5. Cytochrome c Release Measurements.

Release of cytochrome c from mitochondria was measured by immunoblotting. Cells were harvested by centrifugation and gently lysed for 5 minutes in ice-cold buffer containing 25 mM Tris and 5 mM MgCl₂, pH 7.4. Lysates were centrifuged for 5 min at 16,000 g, supernatants were mixed with 1×00 Laemmli's reducing SDS-PAGE sample buffer, and extracts from equal numbers of cells (10-20×10⁶) were resolved by 15% SDS-PAGE. Polypeptides were transferred to nitrocellulose membranes (0.2 μM, Schleicher & Scheull, Keene, N.H.), and cytochrome c was detected by immunoblotting with the monoclonal antibody clone 7H8.2C12 (Pharmingen, San Diego, Calif.).

6. Statistical Analysis.

The data reported in the figures represent the mean of three or more independent experiments and the bars show the standard deviation (SD). ANOVA and two-tailed Student's t test were used for statistical analysis of multiple groups and pair-wise comparison, respectively, with p<0.05 considered significant.

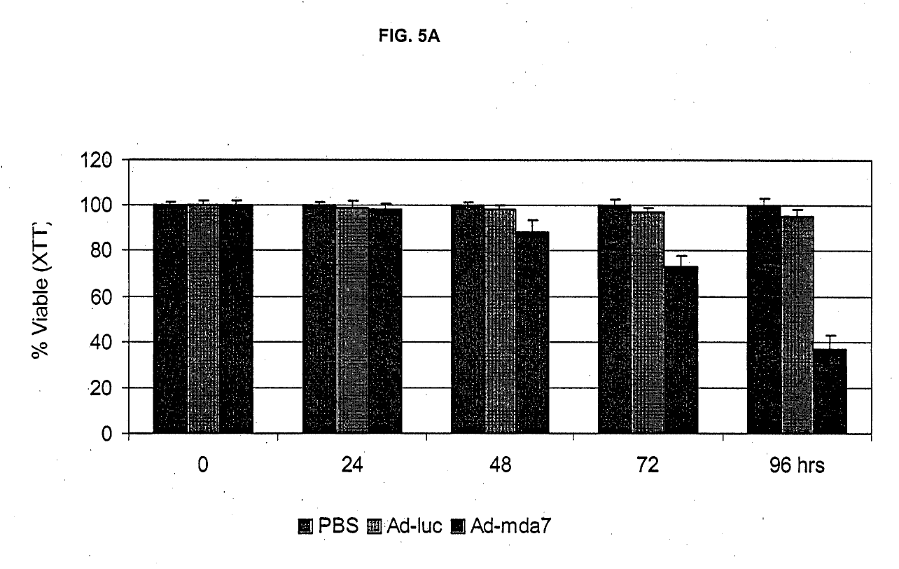

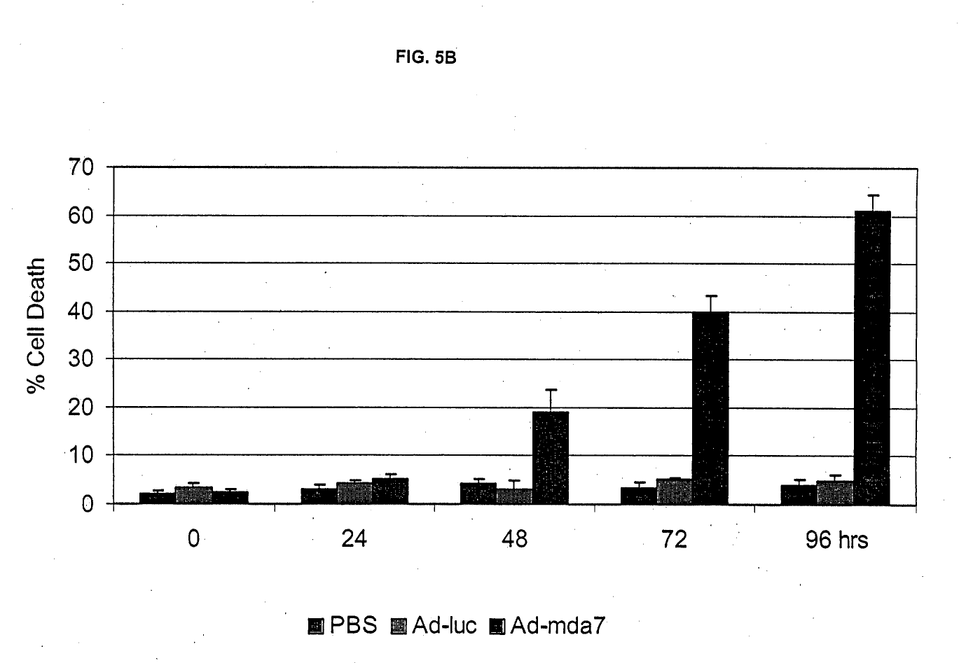

Cellular viability was assessed with the XTT assay. Transduction of both the p53-resistant (A549) and p53-sensitive (H1299) lung cancer cell lines resulted in marked decrease in cellular viability 48 to 72 hours after infection (FIG. 5A). Apoptosis induction was evaluated by flow cytometric analysis of subdiploid populations. Both A549 (p53-resistant) and H1299 (p53-sensitive) lung cancer cells demonstrated apoptosis induction 48 to 96 hrs following Ad-mda-7 transduction. No apoptosis was seen with Ad-Luc or PBS-treated cells (FIG. 5B). Transduction of both A549 and H1299 lung cancer cell lines with Ad-mda-7 resulted in cytochrome c release into the cytosol 48 hrs after treatment. This corresponded to the onset of apoptosis and suggest involvement of the mitochondria in the apoptotic cascade.

### Example 6

### AD-MDA-7 Effect on Mitochondrial Membrane Potential Changes and Apoptosis

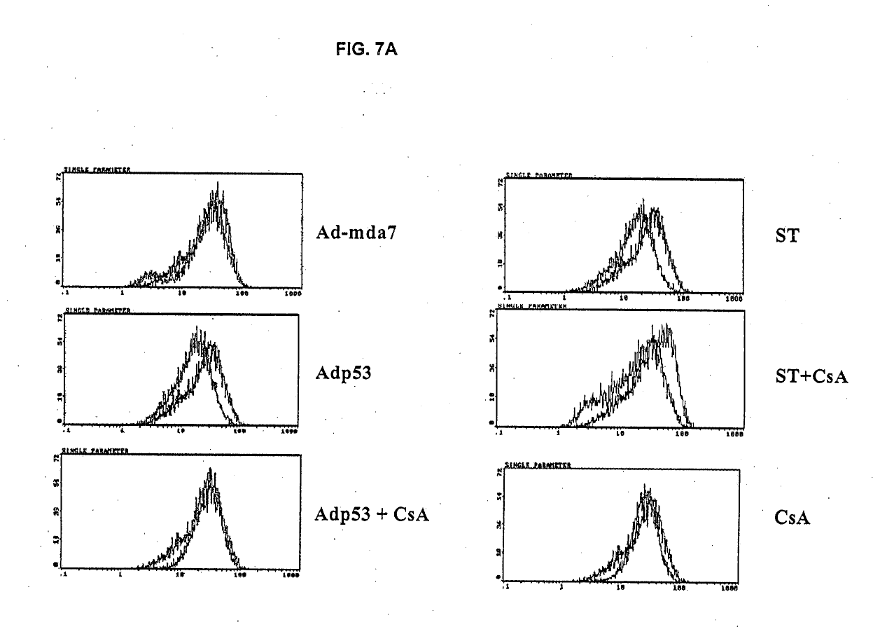

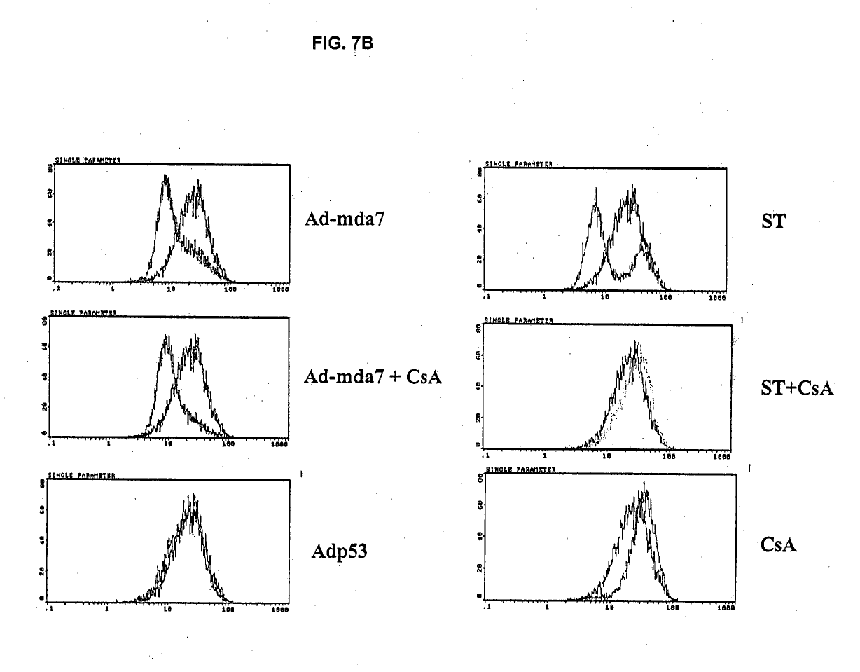

Disruption of the mitochondrial membrane potential (MMP) is a typical event in the development of apoptosis in MPT-dependent apoptotic agents. In H1299 cells, both Ad-p53 and staurosporine are able to induce MMP changes and the induction of apoptosis through MPT-dependent pores using the materials and methods as described in Example 5. Both the MMP changes and apoptosis are inhibited by CsA which selectively blocks MPT-dependent pores (FIGS. 6A, 7A). Ad-mda-7, however, does not induce MMP changes but is still able to induce apoptosis which is not inhibited by CsA. In A549 cells, Ad-p53 is unable to induce apoptosis or MMP changes (FIGS. 6B, 7B). Staurosporine, however, induces MMP changes and apoptosis both of which are blocked by CsA because of inhibition of MMP-dependent pores. Interestingly, Ad-mda-7 induces MMP changes but these changes are not reversed by CsA. Additionally, CsA is unable to inhibit Ad-mda-7 induced apoptosis. Together, these data suggest that Ad-mda-7-induced apoptosis and cytochrome c release occurs through MMP-independent pores which are not blocked by CsA.

### Example 7: Cyclosporine A Does not Prevent the Loss of the Mitochondrial Membrane Potential

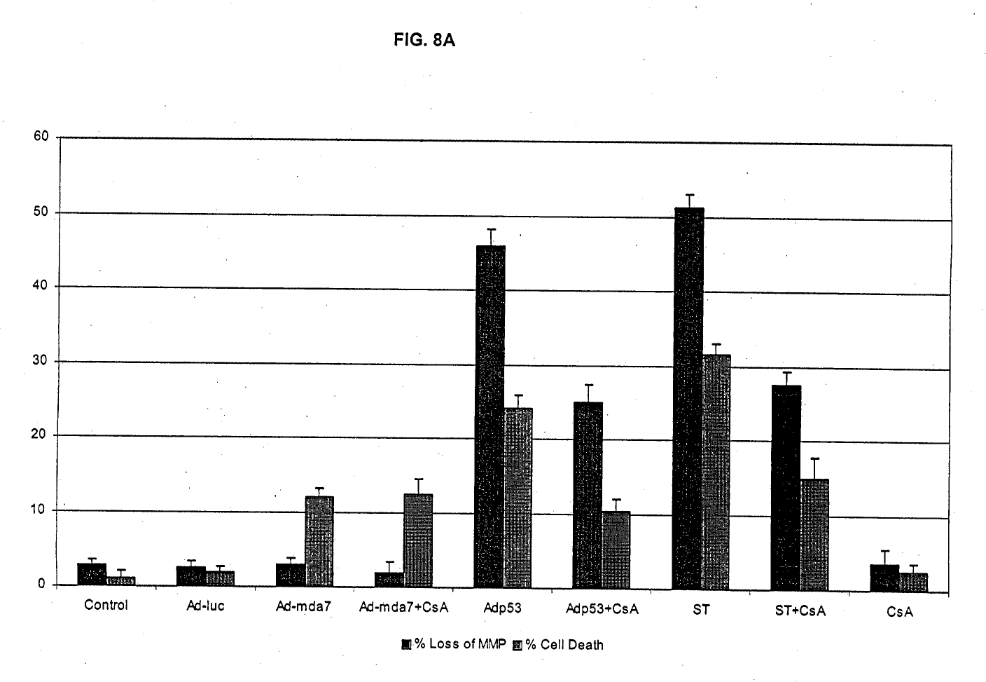

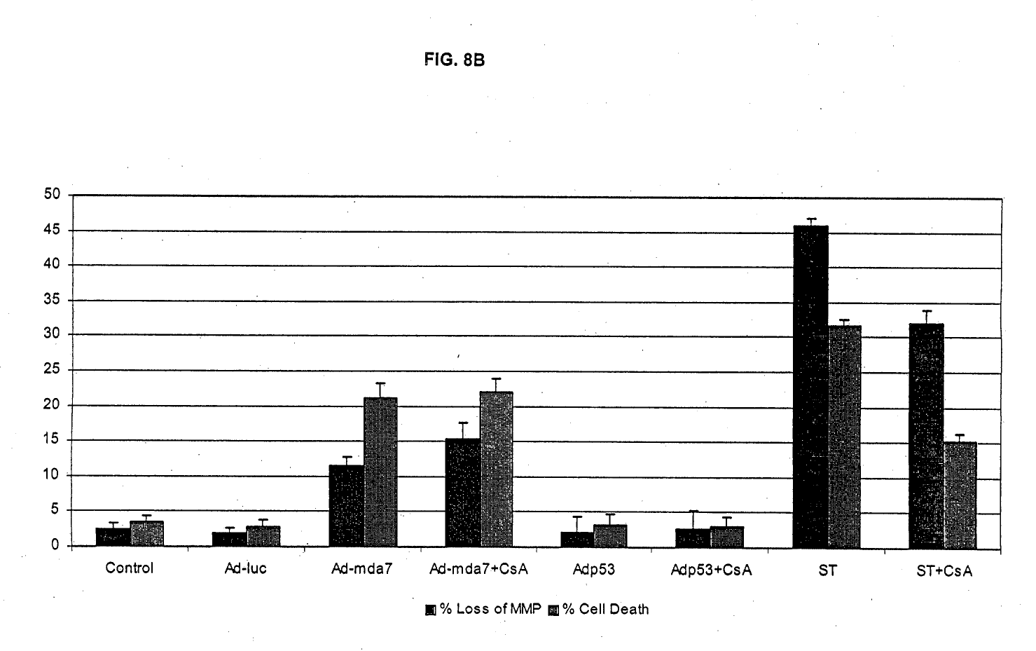

To determine the effect of Cyclosporine A on mitochondrial membrane potential, H1299 cells (FIG. 8A) and A549 cells (FIG. 8B) were treated with Ad-mda-7, Ad-p53 and Staurosporine (1 μM) as described above. Where indicated, the cells were pre-treated with Cyclosporine A at a concentration of 10 μM. The cells were then lysed and the MMP was determined with TMRE. It was found that Cyclosporine A does not affect changes in MMP.

### Example 8

### AD-MDA-7 Up-Regulates the Extrinsic Pathway

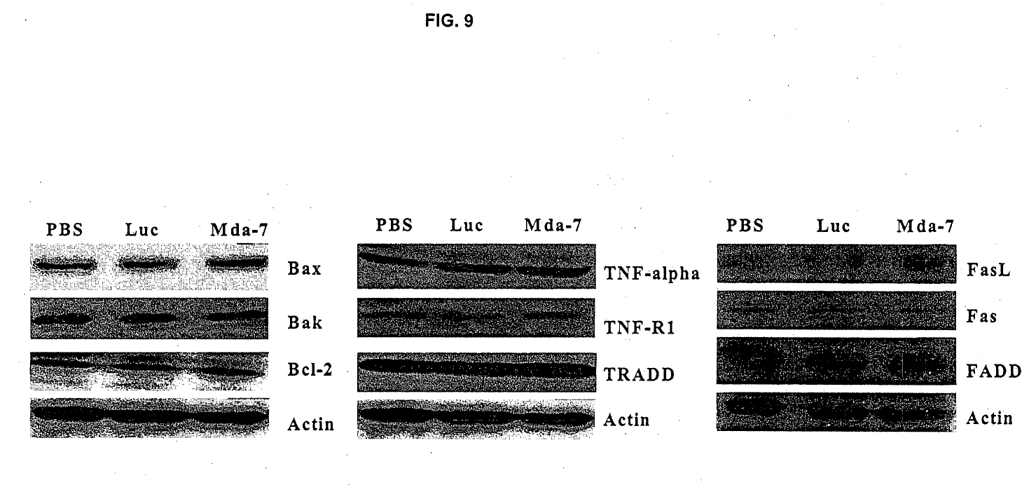

To determine if Ad-mda-7 activated the mitochondria through the Bc1-2 family of genes or the death receptor pathway, Ad-mda-7-treated cells (A549) were assessed by immunoblot assay for changes in BAK, BAX, Bc1-2, TNF-α, TNF-R1, TRADD, FasL, Fas and FADD expression (FIG. 9), as described above. No differences in Bc1-2 family members were seen but a significant up-regulation of FasL was noted. Additionally, previous studies have demonstrated the activation of caspase 8 and cleavage of BID consistent with activation of the extrinsic pathway possibly through Ad-mda-7 up-regulation of FasL.

### Example 9

### Effects on Mitochondrial Membrane Potential

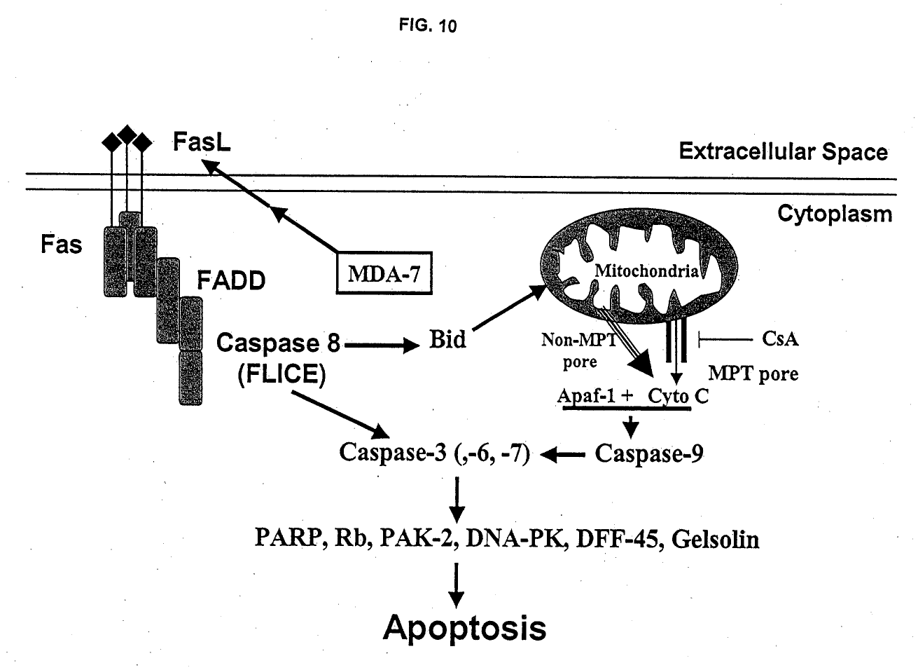

FIG. 10 represents a schematic demonstrating the effects of several pro-apoptotic genes that induce MMP changes (i.e. BAX, BAK, and p53), which open MMP-dependent pores and allow the release of cytochrome c and the formation of apoptosome with APAF-1 and caspase 8. This apoptosome activates the executioner phase of apoptosis with caspase-3, -6 and -7 ultimately cleaving a variety of cellular substrates. These pro-apoptotic agents can be inhibited by CsA or bonkregic acid which block the MMP-dependent pores preventing MMP changes and cytochrome c release.

### Example 10

### AD-MDA-7 Regulates β-Catenin Pathway in Breast and Lung Cancer Cells

Materials and Methods

1. Cell Lines

Breast cancer lines MDA-MB-453, T47D, MCF-7, SkBr3 and lung cancer lines H1299 and A549 were obtained from the American Type Culture Collection (ATCC, Rockville, Md.). The cells were grown in DMEM medium (GIBCO/BRL, Life Technologies, Grand Island, N.Y.) and 10% fetal bovine serum. Human Umbilical Vein Endothelial Cells (HUVECs) were obtained from Clonetics Inc. (San Diego, Calif.). Anti-APC rabbit-polyclonal, anti-GSK-3β monoclonal, anti-PLC-γ monoclonal, anti-FAK mAb, anti-pAKT, anti-ILK-1, anti-PTEN and secondary antibodies such as anti-mouse-FITC/rhodamine and anti-rabbit FITC/rhodamine were purchased from Santa-Cruz Biotechnology; anti-β-catenin mAb, anti-β-catenin-mAb-FITC, anti-E-cadherin-mAb were from Transduction Labs. Anti-MDA-7-polyclonal and monoclonal antibodies were made as previously described (Mhashilkar et al., 2001).

2. Recombinant Adenovirus Production and Transduction.

Replication-deficient human type 5 Adenovirus (Ad5) carrying the mda-7 gene was produced as described earlier (Su et al., 1998). Ad-p53 and Ad-Luc (Luciferase) have been described previously (Mhashilkar et al., 2001; Saeki et al., 2000). Cell lines were infected with Ad-mda-7 (with Ad-Luc as control) at various MOIs as described previously (Mhashilkar et al., 2001). Note that the vp/pfu ratio for these viral preparations was 25.

3. Microarray Analysis.

MICROMAX Human cDNA System I-Direct kit (NEN Life Science Products, Inc.) containing 2400 human cDNA general screening microarrays and MICROMAX Direct System: Human Oncogenes and Tumor Suppressors (NEN Life Science Products, Inc.) containing 280 human cDNAs associated with cancer were used. mRNA was isolated from H1299 cells treated with Ad-mda-7 or Ad-Luc (1000 vp/cell for 24 hr) and analyzed according to manufacturer's instructions.

4. Western Blot Analysis.

Cell lysates (10⁵-10⁶ cells were suspended in 500 μL of Laemmli buffer with 5% 2-β-mercaptoethanol (2βME)) were analyzed by SDS polyacrylamide gel electrophoresis and Western blot analysis using the Super-Signal substrate for Horseradish Peroxidase (Pierce Inc.) as described (Mhashilkar et al., 2001).

5. β-Catenin-Induced Luciferase Assay.

The TOPFLASH kit (Clontech, Palo Alto, Calif.) utilizes a plasmid which has the TCF/LEF promoter which drives the expression of Luciferase. The binding of β-catenin to TCF/LEF and its translocation to the nucleus induces Luciferase activity. Cancer cells were transfected with TCF promoter plasmid (1 μg/well using Lipofectamine). The next day, the cells were transduced with Ad-mda-7 or Ad-GFP at 1000 vp/cell. After 48 hours, cells were washed and lysed with the Reporter lysis kit and analyzed for Luciferase activity.

6. Trypan Blue Assay.

Cell viability was analyzed by a trypan blue exclusion assay. Adenovirus-vector treated cancer cells were trypsinized and a small aliquot was suspended in a 1:1 volume with 0. 1% trypan blue. Total cell numbers and cell viability counts were assessed using a hemocytometer by light microscopy.

7. FACS Analysis and Annexin V Assay.

Regulation of surface markers, such as E-cadherin, and apoptosis was determined by FACS analysis using antibodies and an Annexin V assay kit as described earlier (Mhashilkar et al., 2001).

8. Cell-Cell Adhesion Assay.

Cells were trypsinized 24 hours after vector treatment and a single cell suspension was made using constant mixing (>98% single cells at t=0). Cells were placed in eppendorf tubes in PBS/1% FBS at 10⁶ cells/ml at room temperature. An aliquot of cells was analyzed every two hours to monitor the percent of single viable cells remaining in the suspension (i.e., cell clumps were not counted).

9. Immunofluorescence Assay.

Cells growing in chamber slides (Nunc) were treated with vectors and 48 hours later were analyzed for MDA-7 protein expression and/or modulation of different proteins from β-catenin and PI3K pathways. Cells were fixed with an Ethanol:Acetic Acid mixture (9.5:0.5) and then treated with primary antibody for 1 hour at 4° C. The cells were washed extensively with PBS and treated with secondary antibody. The slides were analyzed using a Nikon Fluorescent microscope and photographed using a Nikon Digital camera (DXM1200 System).

10. Cell Migration Assay.

Tumor cells (A549) were seeded at 5×10⁵ cells/well in six-well tissue culture plates. The following day, cells were infected with Ad-mda-7 or Ad-Luc at MOI of 3000 viral particles (vp)/cell for 4 hours. Following infection, cells were replenished with complete medium. Twenty-four hours after infection, cells were harvested and used for a migration assay. Briefly, cell sedimentation manifolds (Creative Scientific Methods, Mesa, Ariz.) were placed on Teflon-coated slides. The cell sedimentation manifold was removed, and fresh RPMI-1640 containing 10% FBS was added. The circular area occupied by attached cells in each well was imaged using a Nikon digital camera attached to an inverted microscope

11. Statistical Analysis.

The statistical significance of the experimental results was calculated using Student's t-test.

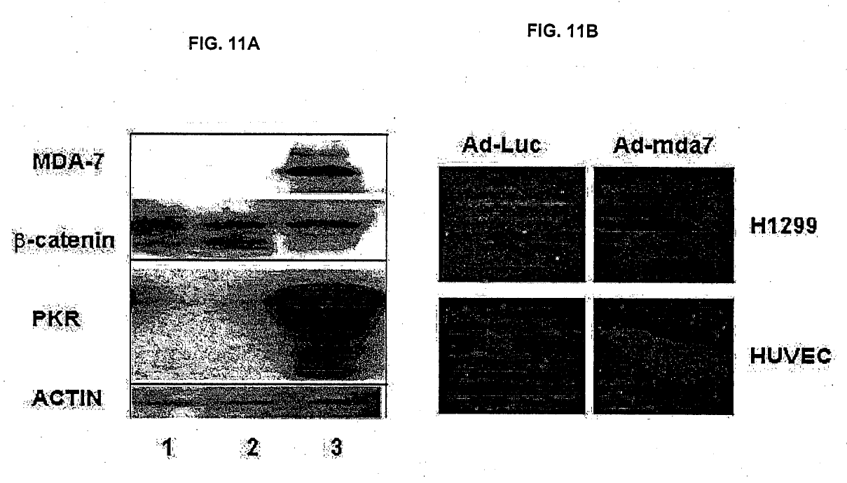

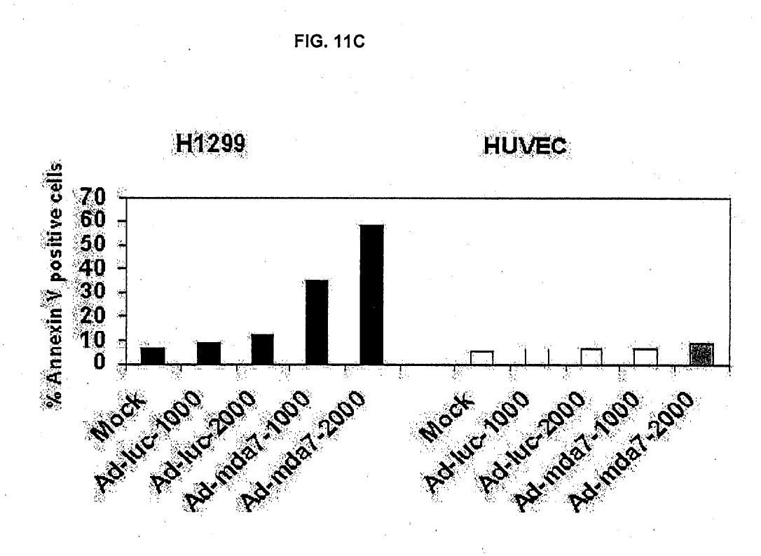

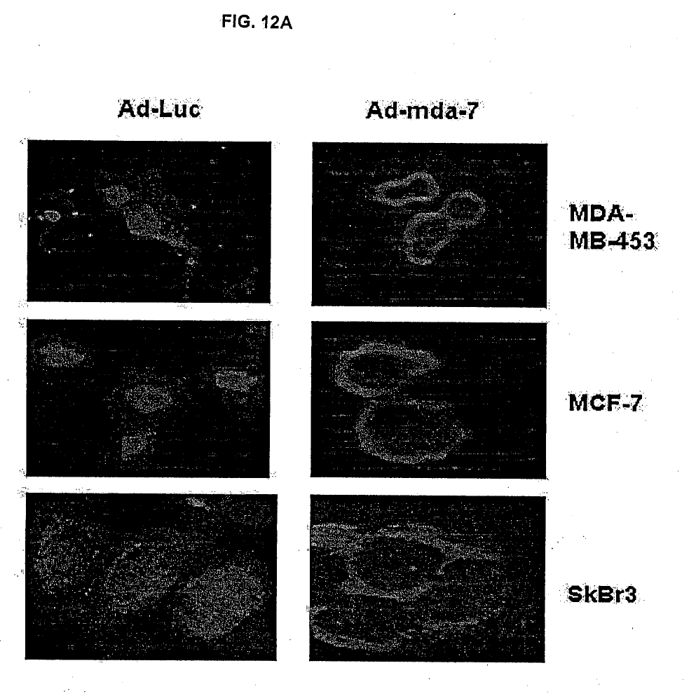

Ad-mda-7 transduced breast (MDA-MB-453) cancer cells showed elevated levels of MDA-7 protein, but only a modest decrease in the steady state levels of β-catenin protein compared to untreated or Ad-Luc-treated cells (FIG. 11A). Similar results were obtained in other breast and lung cancer cell lines. Immunofluorescence studies demonstrated cytoplasmic MDA-7 staining only in Ad-mda-7-transduced tumor cells (H1299) and normal cells (HUVEC), with typical punctate cytoplasmic staining observed (FIG. 11B). Ad-mda-7 induced apoptosis in H1299 tumor cells but not in normal HUVEC cells (FIG. 11C.). Ad-mda-7 also strongly induced apoptosis in all lung (n=5) and breast (n=6) tumor cell lines tested (Jiang et al., 1996; Mhashilkar et al, 2001; Saeki et al., 2000; Pataer et al, 2002).

### Example 11

### Subcellular Localization and Distribution of β-Catenin After Ad-Mda-7 Treatment

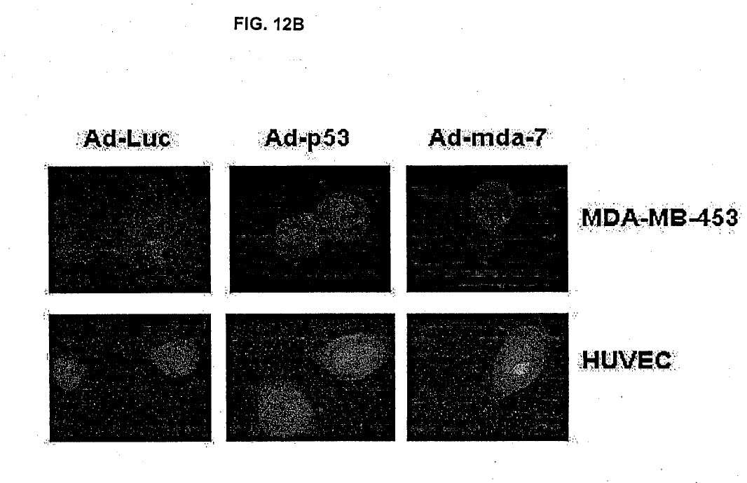

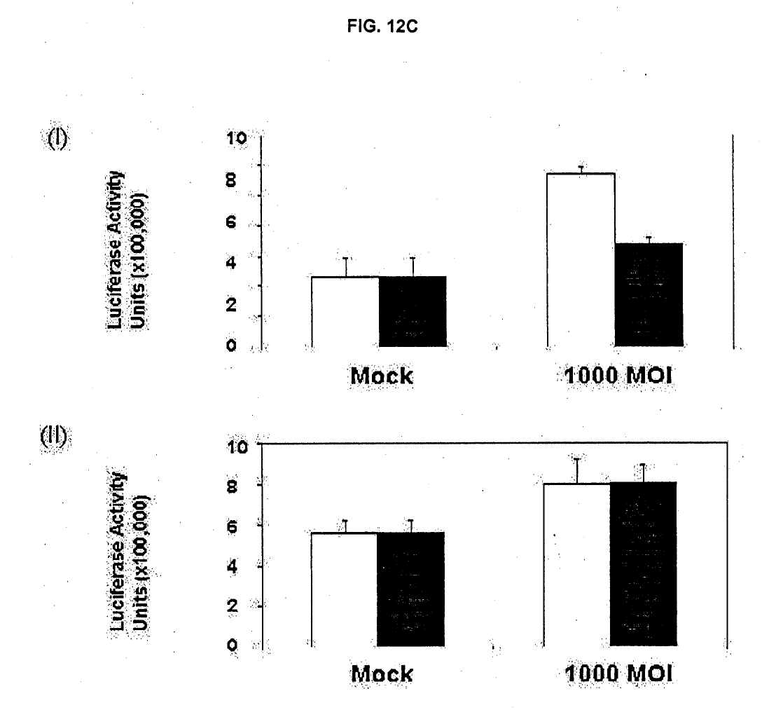

The subcellular localization and distribution of β-catenin after Ad-mda-7 treatment was evaluated. Untreated or Ad-Luc-treated breast cancer cells (MDA-MB-453, MCF-7 and SkBr3) exhibited the typical cytoplasmic and nuclear β-catenin staining observed in most tumor cell lines (FIG. 12A). However, Ad-mda-7-transduced cells typically demonstrated a loss of nuclear staining and β-catenin was found to be localized to the plasma membrane in MDA-7 expressing cells. This redistribution of cytoplasmic/nuclear β-catenin to the plasma membrane was also observed in NSCLC cells (H1299). Note that the doses of vector used and timing of these experiments were selected to minimize apoptosis induction; these cells were 75% viable by trypan blue exclusion assay. To determine whether this -catenin redistribution was a function of cells undergoing early stages of apoptosis, the breast and lung tumor cells were treated with Ad-p53, a potent inducer of apoptosis in these cell lines. In MDA-MB-453 cells, the subcellular distribution of β-catenin was identical in Ad-p53, Ad-Luc and untreated controls whereas β-catenin redistribution was only observed after Ad-mda-7 treatment (FIG. 12B), suggesting that the β-catenin redistribution was not simply a result of an apoptotic signal. Similar results were seen in H1299 lung tumor cells.

It has previously been shown that Ad-mda-7 exhibits tumor-selectivity for apoptosis induction. Thus, normal human endothelial cells (HUVECs) were analyzed for β-catenin redistribution in response to Ad-mda-7. HUVECs treated with Ad-mda-7, Ad-p53 or Ad-Luc all showed a similar pattern of nuclear/cytoplasmic diffuse β-catenin staining as observed in untreated cells (FIG. 12B). Therefore, the exclusion of β-catenin from the nucleus appears to be an activity specific to MDA-7 overexpression and appears to manifest only in tumor cells.

The consequences of redistributing β-catenin were evaluated by analyzing the functional activity of β-catenin-mediated transactivation using the TopFlash/FopFlash system. This system uses the TCF/LEF promoter to drive expression of Luciferase. In H1299 NSCLC cells, Ad-mda-7 treatment significantly inhibited luciferase activity of the TopFlash-based assay compared to the control Ad-GFP construct (p=0.001). However, in the FopFlash-based assay (where the plasmid has a mutated TCF/LEF promoter, and is therefore β-catenin-independent), there was no effect on luciferase (FIG. 12C). A similar inhibition of β-catenin-mediated luciferase activity was demonstrated in MDA-MB-453 cells.

### Example 12

### AD-MDA-7 Up-Regulates E-Cadherin, Inhibits Cell Migration And Promotes Cell-Cell Adhesion

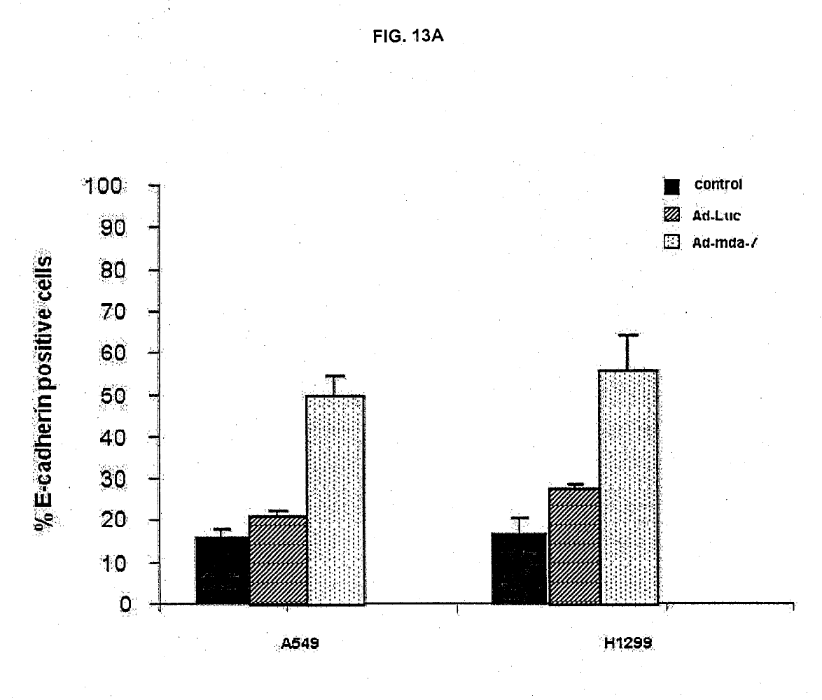

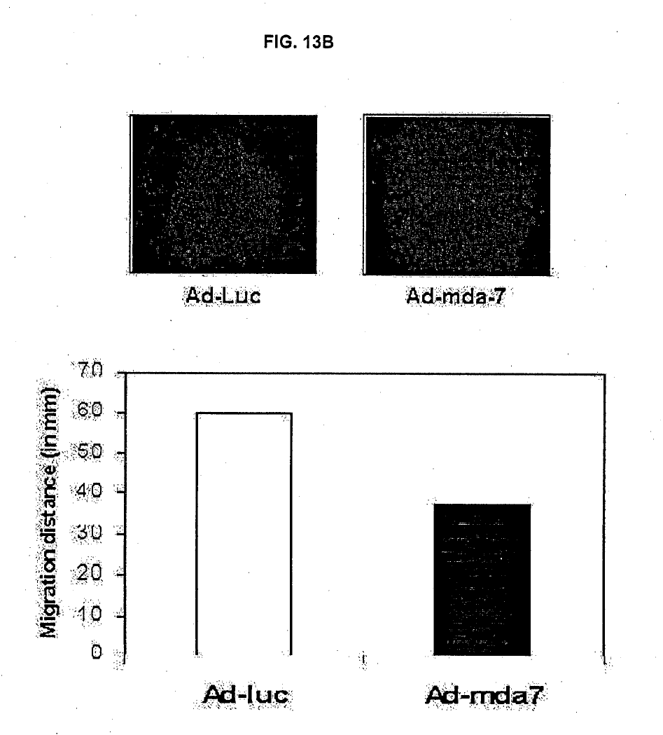

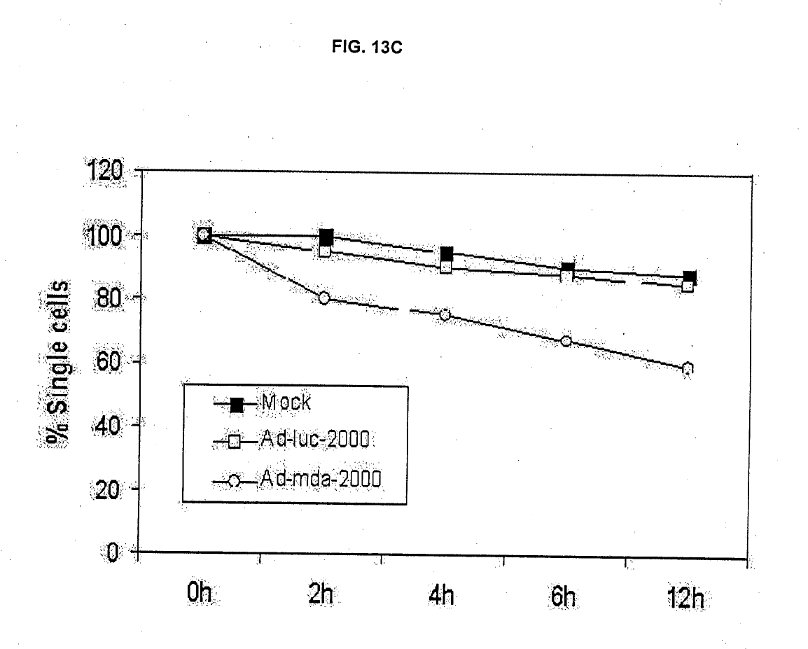

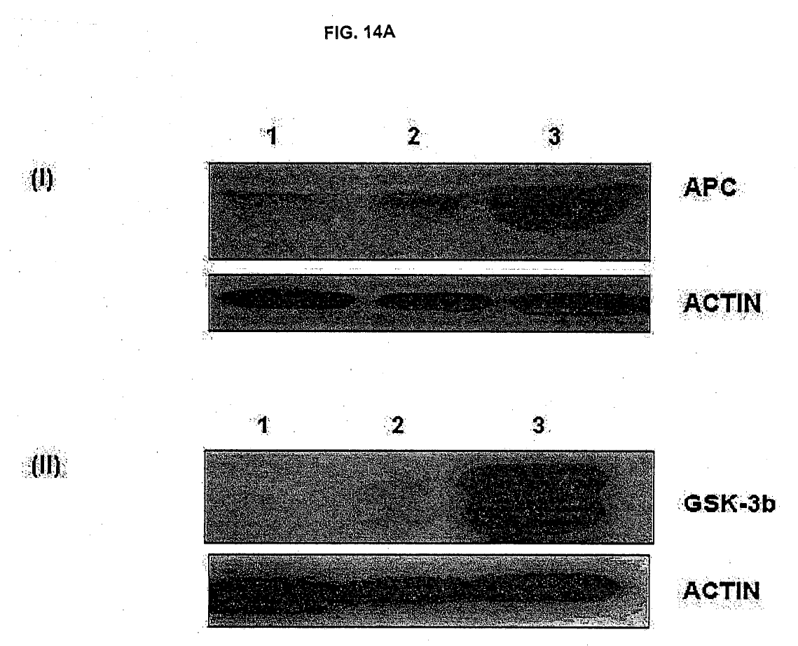

E-cadherin represents a family of membrane receptors that mediate calcium-dependent homophilic cell-cell adhesion. Disruption in expression or function of cadherins can result in uncontrolled cell migration and proliferation during tumor development (Ivanov et al., 2001). In both breast and lung cancer cells a significant increase (p=0.001) in E-cadherin levels after Ad-mda-7 transduction was found, as seen by surface staining using anti-E-cadherin monoclonal antibody and flow cytometry (FIG. 13A) using the materials and methods described in Example 10. The functional consequences of the up-regulation of E-cadherin were further evaluated by using assays to monitor cell migration and cell-cell adhesion. Cells were treated with Ad-mda-7 or control Ad-Luc and evaluated in a monolayer cell migration assay. Ad-mda-7 significantly decreased cell migration compared to Ad-Luc treated cells (FIG. 13B). Homotypic cell-cell adhesion was monitored in dispersed single cells as a function of time. Ad-mda-7 treated cells aggregated and exhibited homotypic adhesion at a significantly higher rate than Ad-Luc or mock treated cells. Thus the percentage of single cells was reduced in Ad-mda-7 treated cells (FIG. 13C).

### Example 13

### AD-MDA-7 Modulates APC, GSK-3B, PLC-Γ and Other Proto-Oncogenes from PI3K Pathway

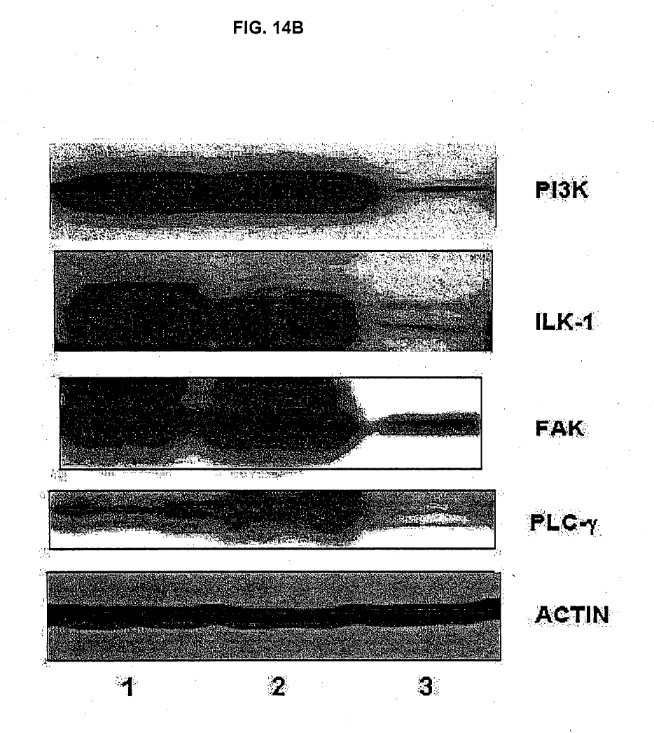

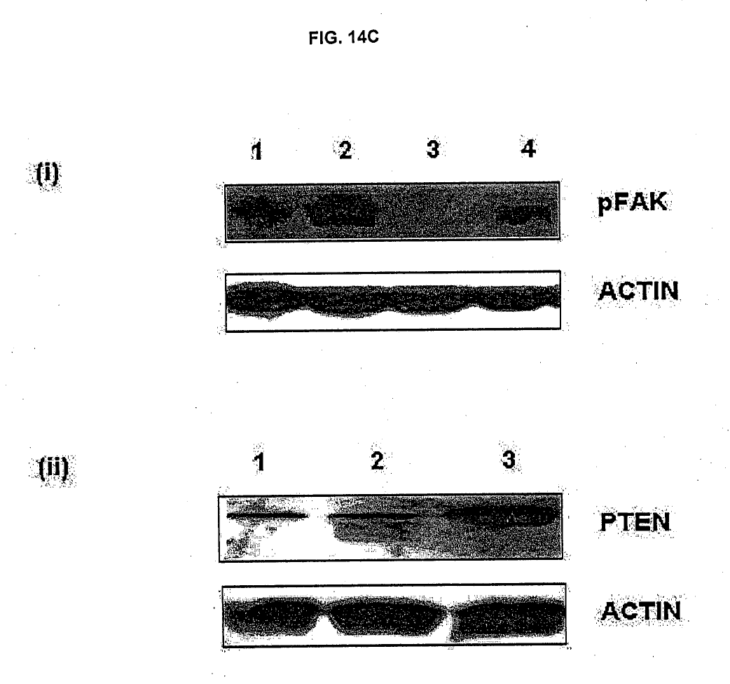

The APC and glycogen synthase kinase-3β (GSK-3p) molecules serve as negative regulators of β-catenin. Ad-mda-7 transduction of both MDA-MB-453 cells (FIG. 14A) and H1299 cells -demonstrated up-regulation of the tumor suppressor proteins, APC and GSK-3β. Ad-mda-7 transduced cells were evaluated for expression of various proto-oncogenes from the PI3K pathway. Expression of proto-oncogenes such as PI3K, FAK, ILK-1 and PLC-y were strongly inhibited by Ad-mda-7 but were unaffected by Ad-Luc in H1299 cells (FIG. 14B). H1299 lung cancer cells were evaluated for regulation of pFAK (FIG. 14C(i)). Ad-mda-7 strongly downregulated pFAK and PI3K expression in both H1299 and A549 NSCLC lines. The PI3K inhibitor LY294002 was used as a positive control. Ad-mda-7 was more potent at inhibiting pFAK than LY294002 in H1299 cells (compare lanes 3 and 4 of FIG. 14C(i)). Ad-mda-7 treatment of breast and lung cell lines also decreased expression of Akt and pAkt. Ad-mda-7 strongly up-regulated expression of the tumor suppressor, PTEN, a pivotal negative regulator of the PI3K signaling pathway (FIG. 14C (ii)).

### Example 14

### MDA-7 Expression Following AD-MDA-7 Infection in Prostate Cancer and Epithelial Cells

Material and Methods

1. Cell Lines and Cell Culture.

The human prostate cancer cell lines, DU145, LNCaP, and PC-3 were obtained from American Type Culture Collection (Manassas, Va.). The normal prostate epithelial cell line, PrEC was obtained from Clonetics (San Diego, Calif.), DU145, LNCaP, and PC-3 cells were grown in RPMI 1640 medium with 10% fetal bovine serum, antibiotics and L-glutamine (GIBCO/BRL). PrEC cells were incubated in PrEBM medium with supplements according to supplier's instructions.

2. Construction of Recombinant Adenoviral Vector.

The replication-deficient human type 5 adenoviral (Ad5) vectors carrying the mda-7 gene was constructed briefly as follows. The mda-7 gene was linked to an internal CMV-IE promoter and followed by an SV40 polyadenylation tail [poly(A)]. Ad-Luc (Luciferase), was used as a control vector. Briefly, Ad5 vectors harboring the gene cassettes were co-transfected with plasmid pJM17 in HEK 293 cells to obtain recombinant Ad-mda-7, or Ad-Luc viruses. Plaques were picked, virus stocks were grown, and their genomes were confirmed as correct by PCR/Restriction analysis and DNA sequencing. Viruses were propagated in 293 cells and purified by chromatography.

3. Gene Transduction

Preliminary experiments using an Ad vector encoding green fluorescent protein (Ad-GFP) showed that an adenovirus dose delivered at a multiplicity of infection (MOI) of 3000 can infect more than 93.4% of DU145 and PC-3 cells, 76.2% of LNCaP cells, and 82% of PrEC cells. Therefore, an MOI of 3000 for Ad-mda-7 and Ad-Luc was used in all subsequent experiments. Cells were plated in a 10-cm dish at 5-10×10⁵ cells/dish for protein expression or FACS analysis of the cell cycle. Cells were exposed to Ad vectors 24 h after plating.

4. Cell Proliferation Assay

All of the cell lines were plated in six well tissue culture plates at a density of 1×10⁵ cells/well. Tumor cells were then infected with Ad-mda-7 or Ad-Luc or treated with PBS as a mock control. Cells in each treatment group were plated in triplicate and cultured for 5 days. At designated time points, cells were then harvested via trypsinization and stained with 0.4% trypan blue (GIBCO BRL, Grand Island, N.Y., USA) to reveal dead cells. Viable cells were counted using a hemocytometer.

5. Apoptotic Staining

Cells were seeded in six well tissue culture dishes at a density of 1×10⁵ cells per well and infected with Ad-mda-7 or Ad-Luc. 72 h post-infection, cells were analyzed for apoptosis using Hoechst 33258 staining (Sigma Chemical Co., St. Louis, Mo., USA). Apoptotic cells were determined via apoptotic body and/or chromosome condensation.

6. Cell Cycle Analysis

Cells were seeded in 10 cm culture dishes (5-10×10⁵ cells/dish) and infected with Ad-mda-7, Ad-Luc, or treated with PBS. At specific times post-treatment, cells were harvested via trypsinization, washed once with ice-cold PBS, fixed with 70% ethanol and stored at −20° C. Cells were then washed twice with ice-cold PBS and treated with RNase (30 min at 37° C., 500 units/ml; Sigma Chemical Co.), and DNA was stained with PI (50 μg/ml; Boehringer Mannheim, Indianapolis, Ind., USA). The cell cycle phase and apoptotic rate (cells at sub-G0/G1 phase) were analyzed using a FACScan (EPICS XL-MCL; Beckman Coulter, Inc., Fullerton, Calif., USA).

7. Mitotic Index.

Cells were harvested at 72 h after infection with Ad-mda-7 and fixed in a manner to be used for cell cycle analysis. Following fixation, the cells were stained with propidium iodide, treated with RNase and then analyzed by fluorescence microscopy. For each sample, at least 500 cells were randomly counted by fluorescence microscopy, and mitotic cells were visually identified by their lack of a nuclear membrane and by evidence of chromosome condensation.

8. Immunoblot Analysis

Cells were infected with Ad-mda-7, Ad-Luc, or treated with PBS. Cells were incubated for an indicated number of hours at 37° C. and then collected to prepare whole cell lysates. For this preparation, cells were lysed with lx SDS sample buffer (62.5 mM Tris-HCL, 2% SDS, 10% glycerol, 50 mM dithiothereitol, 4 mM urea, 0.01% bromophenol blue) containing protease inhibitors (Roche, Germany). Protein samples were heated in a water bath at 95° C. for 5 min. After centrifugation at 12,000 rpm for 10 min, the protein samples were collected from the supernatant, and the protein was quantified using the BCA reaction (Pierce Chemical Co., Rockford, Ill., USA). Equal amounts of protein (50 μg) were separated via 10% SDS-PAGE (BioRad Laboratories, Hercules, Calif., USA) and electrotransferred onto nitrocellulose membranes (Hybond-ECL; Amersham Biosciences, Inc., Piscataway, N.J., USA). The blots were blocked with 5% milk in PBS-Tween 20 (0.1%) at room temperature for 1 h and probed with a primary antibody at the appropriate dilution at 4° C. overnight. The blots were then washed twice in PBS-Tween 20 for 15 min and then incubated with a horseradish peroxidase-conjugated secondary antibody at dilution of 1:2000 (Amersham Biosciences, Inc.) in 5% milk/PBS-Tween 20 at room temperature for 1 h. After being washed twice in PBS-Tween 20 for 15 min, the proteins were visualized on enhanced chemiluminescence (ECL) film (Hyperfilm; Amersham Biosciences, Inc.) using ECL Western Blot Detection Reagent (Amersham Biosciences, Inc.). In addition, for analysis of phosphoproteins, Tris-buffered saline was used instead of PBS throughout. The blots were reprobed with antibodies against β-actin (Sigma Chemical Co.) where indicated, to ensure equal loading and transferal of proteins. Recombinant MDA-7 protein was used to generate rabbit polyclonal that was further purified by affinity chromatography. This antibody was used at dilutions of 1:5000 (from stock of 1 mg/ml); caspase 9 (1:500, rabbit polyclonal), caspase 3 (1:500, rabbit polyclonal), PARP (1:250, mouse monoclonal), (Pharmingen, San Diego, DA, USA); β-actin (1:5000, mouse monoclonal) (Sigma Chemical Co.); phospho-JNK (1:1000, rabbit polyclonal); NFkB (1:500, rabbit polyclonal), phospho-STAT3 (1:500, mouse monoclonal), (Santa Cruz Biotechnology); PKR, phospho-Tyk2, phospho-STAT1, Cdc25C (1:1000, rabbit polyclonal), (Cell Signaling Technology, Inc.); cyclin BI (1:200, mouse monoclonal; Lab Vision Corp., Fremont, Calif., USA); phospho-Jak1 (1:500, goat polyclonal), p27Kip1 (1:500, rabbit polyclonal), Chk1 (1:1000, mouse monoclonal), Cdc2 (1:500, mouse monoclonal), (Santa Cruz Biotechnology); Chk2 (1:1500, rabbit polyclonal), (Novus Biologicals, Littleton, Co, USA); p21WAF1 (Oncogene Research Products, Boston, Mass.); cyclin A (1:2000, mouse monoclonal), (Sigma Chemical Co.); cyclin E (1:1000, mouse monoclonal), (BD Transduction Laboratories).

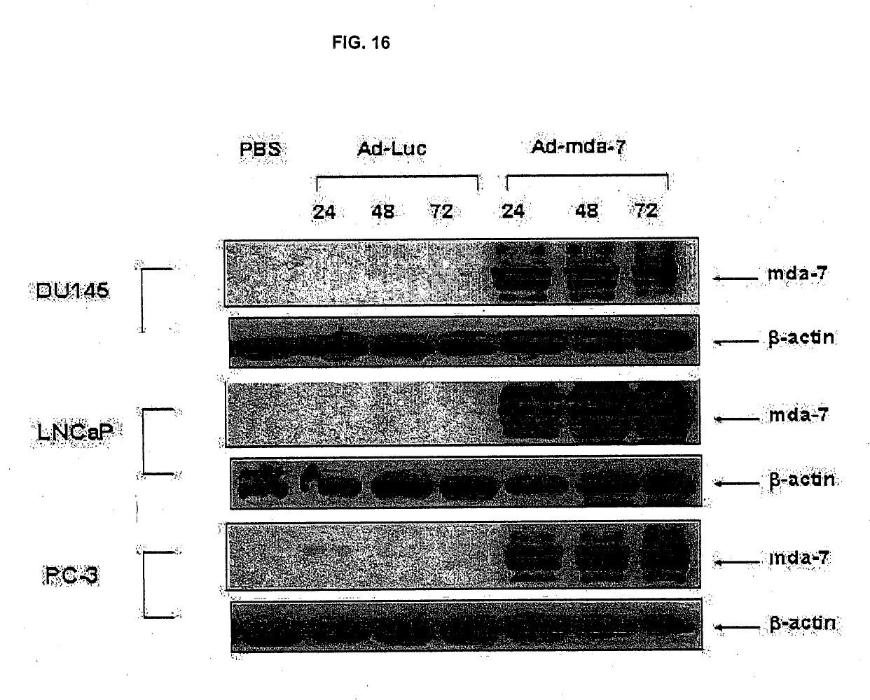

To detect MDA-7 expression in cells, DU145, LNCaP, and PC-3 were grown in six well tissue culture plates (1×10⁵ cells/well) and infected with an adenoviral vector encoding MDA-7 (Ad-mda-7) or an adenoviral vector encoding luciferase (Ad-Luc). Mock-infected cells using phosphate-buffered saline (PBS) served as negative controls. 24 h, 48 h and 72 h post-transfection, total cell lysates from all cells were fractionated by using sodium dodecyl sulfate-polyacrylamide gel electrophoresis (SDS-PAGE) and evaluated for protein expression of over-expressed Ad-mda-7 (FIG. 16). Mda-7 was successfully induced and expressed in all cells from 24 h to 72 h after infection. Similar results were found with PrEC cells.

### Example 15

### Inhibition of Cell Proliferation in Prostate Cancer Cells due to Overexpression of MDA-7

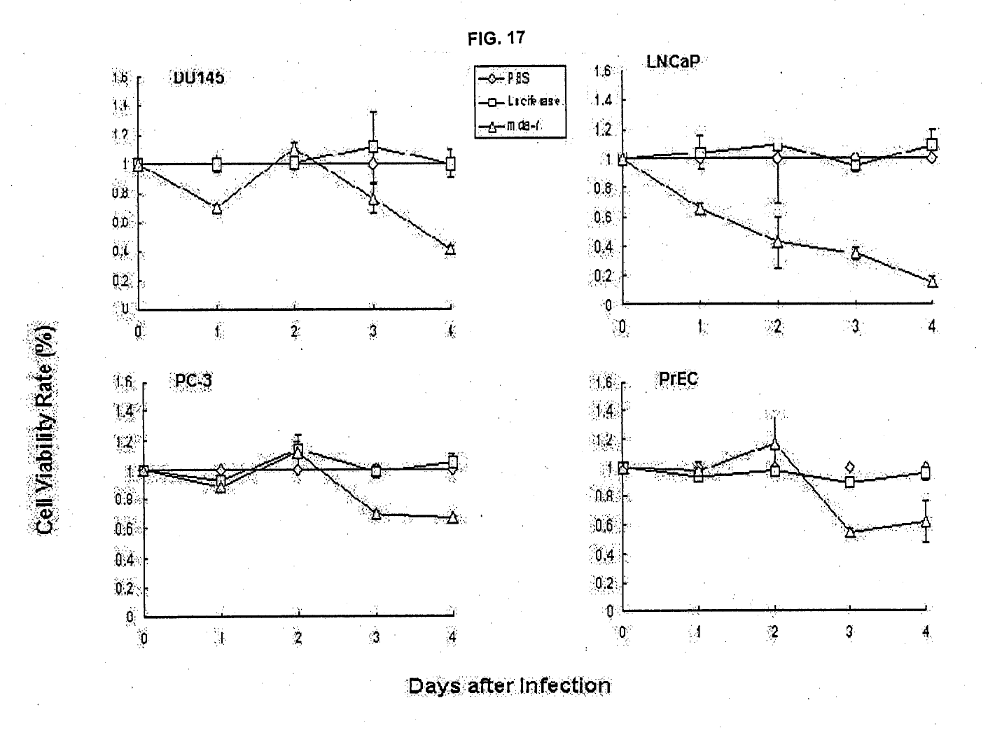

DU145, LNCaP, PC-3 and PrEC cells were grown in six well tissue culture plates (1×10⁵ cells/well) and infected as described in Example 13. Viable cells were counted daily 1-5 days post-infection. Significant inhibition of cell proliferation (p<0.01) was observed on day 4 in DU145 and LNCaP cells infected with Ad-mda-7, when compared with that in control cells infected with Ad-Luc or cells treated with PBS. Significant suppression of cell proliferation was not observed in PC-3 and PrEC cells infected with Ad-Luc or Ad-mda-7 (FIG. 17).

### Example 16

### Induction of Apoptosis in Prostate Cancer Cells due to Overexpression of MDA-7

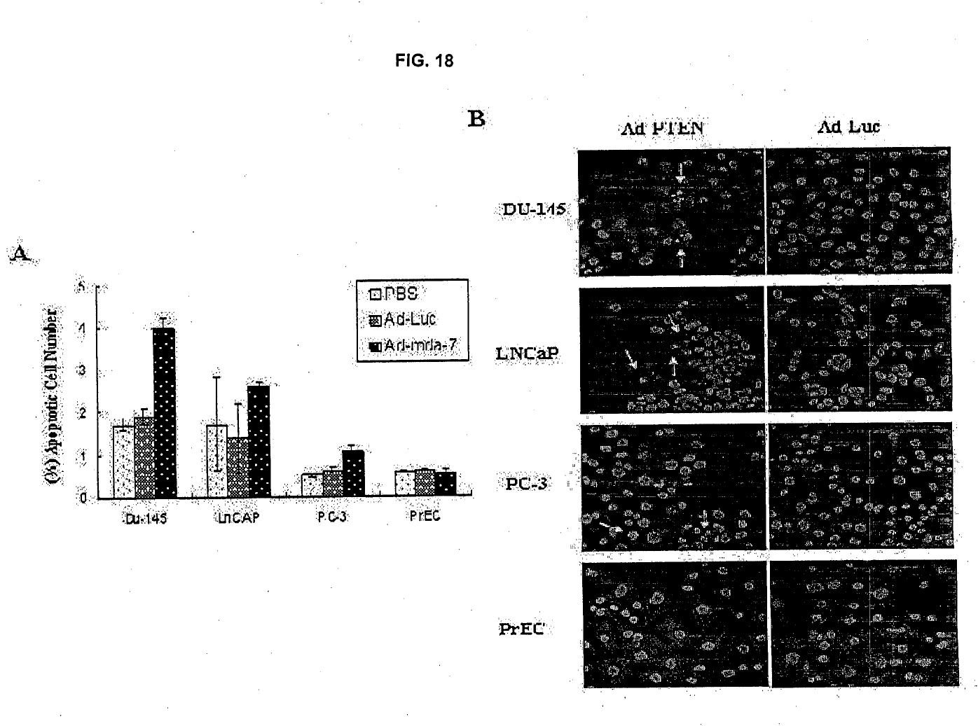

After infection with Ad-mda-7, DU145, LNCaP, PC-3 and PrEC cells were analyzed for apoptotic changes using a FACScan and Hoechst 33258 staining. 72 h after Ad-mda-7 infection, an increase in the number of cells in sub-G0/G1 phase, which is an indicator of apoptotic changes, was observed in the three cancer cell lines tested using fluorescence-activated cell sorter (FACS) analysis. However, no changes were observed in cells infected with Ad-Luc or in cells treated with PBS. In contrast, normal cells infected with either Ad-mda-7 or Ad-Luc did not demonstrate a significant change in the number of cells in sub-G0/G1 phase (FIG. 18A). To confirm these results, Hoechst 33258 staining was performed 72 h after infection. Cancer cells underwent apoptosis following Ad-mda-7 infection, but normal cells did not. No changes were observed in any of the cells infected with Ad-Luc (FIG. 18B).

### Example 17

### Induction of G2 Cell Cycle Arrest due to overexpression of MDA-7

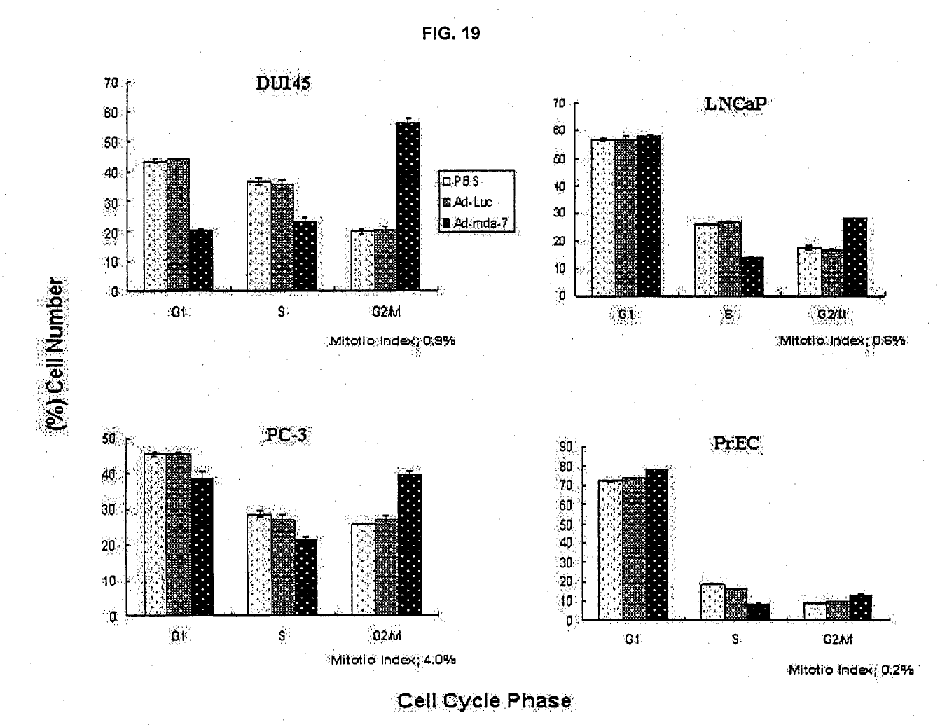

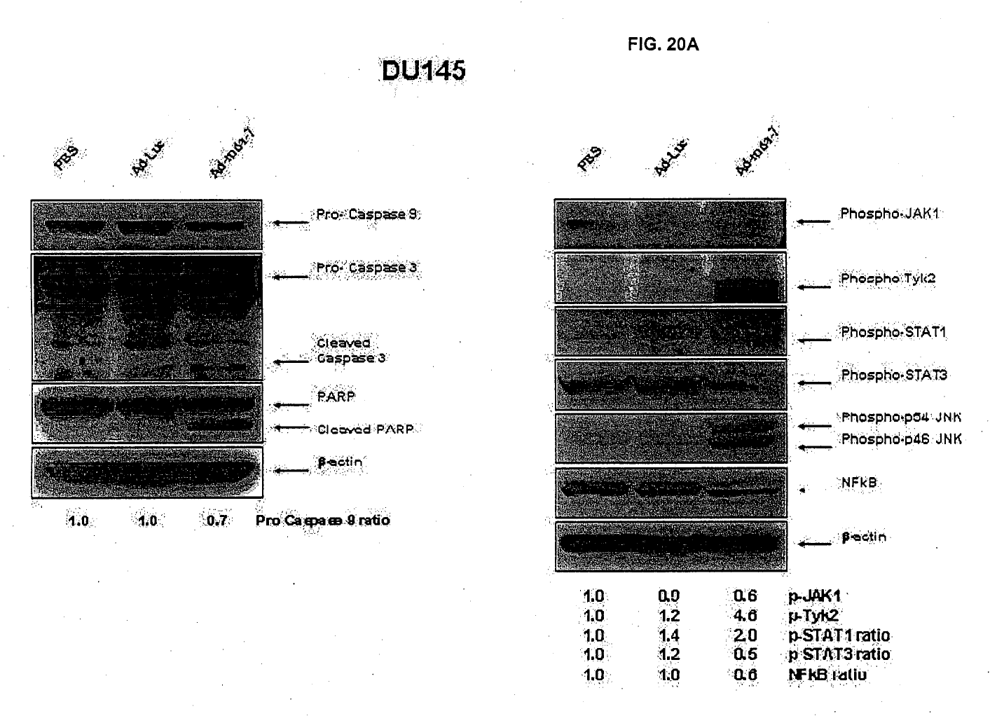

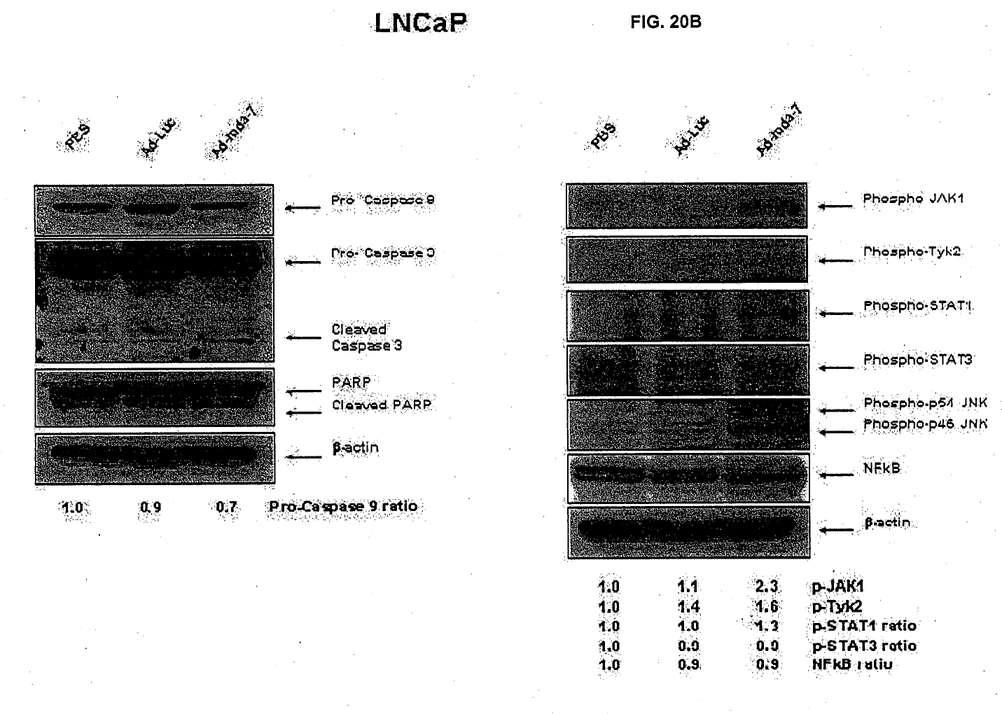

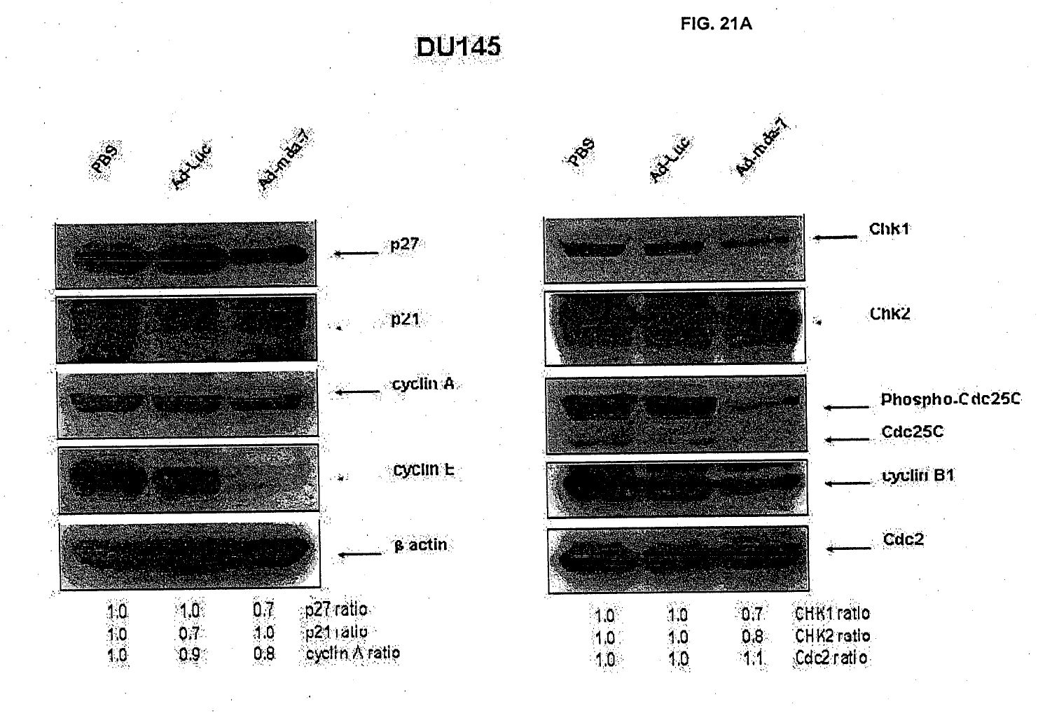

To determine whether MDA-7 is capable of inducing G2/M cell cycle arrest in prostate cancer cells as reported in previous studies of human lung, breast and melanoma cancer cell lines (Saeki et al., 2000), cell cycle phases were analyzed using a FACScan as described in Example 13. Cell cycle analysis using propidium iodide (PI) staining indicated an increase in the percentage of DU145, LNCaP, PC-3 in the G2/M population 72 h after infection with Ad-mda-7 as compared with cancer cells infected with Ad-Luc or those treated with PBS (FIG. 19). However, G2/M-phase inhibition in PrEC cells was obviously weak when compared with that in the cancer cells. This suggests that MDA-7 may have selectively affected tumor cells. To determine whether MDA-7 induces G2- or M-phase arrest, the mitotic indices of DU145, LNCaP, PC-3, and PrEc cells were measured (FIG. 19). These results show that Ad-mda-7 induces G2-phase, but not M-phase, arrest.

### Example 18

### Inhibitory Function of MDA-7 in Prostate Cancer Cells

To determine whether apoptosis was induced by Ad-mda-7, the activation of caspases in DU145 and LNCaP cells were evaluated as described in Example 13. This analysis demonstrated activation of a caspase cascade including cleavage of caspase 9, caspase 3, and poly (ADP-ribose) polymerase (PARP) at 72 h post-Ad-mda-7 infection in both DU145 and LNCaP cells (FIG. 20, A and B). These results suggest that MDA-7 induced apoptosis is via a caspase cascade in prostate cancer cells.

Furthermore, the signaling function of MDA-7 in DU145 and LNCaP cells was analyzed using Western blotting to investigate how MDA-7 induces growth suppression and apoptosis (FIG. 20, A and B). Phosphorylation or inhibition was observed of the downstream signaling targets: Stat1, Stat3, JNK, and NFkB. MDA-7 induced an increase of phosphorylated Jak1 in LNCaP cells, a significant increase of phosphorylated Tyk2 in DU145 cells, and a slight decrease of phosphorylated Jak1 in DU145 cells. Phosphorylation of Stat1 was increased in both cell lines but phosphorylation of Stat3 was decreased or unchanged due to Ad-mda-7. Ad-mda-7 clearly induced phosphorylation of JNK in both cell lines and decreased NFkB in DU145. These results demonstrate that Ad-mda-7 can modulate intracellular signaling pathways in prostate cancer cells.

### Example 19

### G2 Cell Cycle Arrest Associated with Downregulation of CDC25C

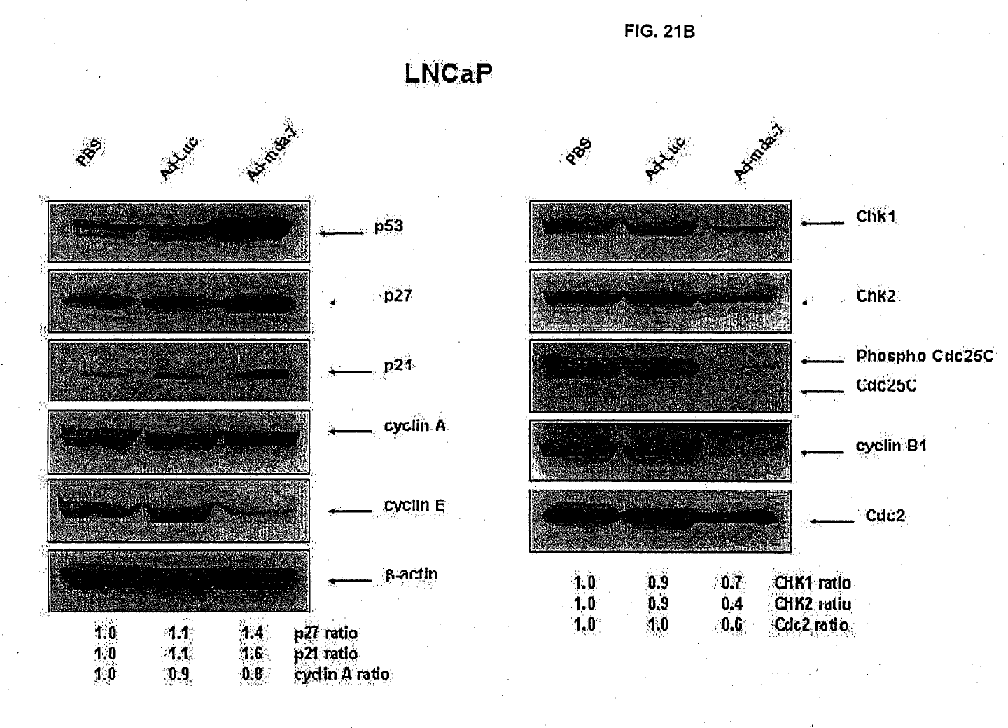

To investigate the mechanism through which Ad-mda-7 significantly induces G2 arrest in prostate cancer cells, proteins related to the G1/S and G2/M cell cycle checkpoint were evaluated by Western blot analysis as described in Example 13. In this analysis, DU145 and LNCaP cells were exposed to PBS or infected with Ad-mda-7 or Ad-Luc. Total cell lysates were prepared 72 h after exposure or infection, FACS analysis was performed and protein concentrations were resolved using SDS-PAGE. Both cell lines treated with Ad-mda-7 demonstrated reduced expression of both phosphorylated and nonphosphorylated Cdc25C and decreased expression of Chk1, Chk2, and cyclin B1 compared with control cells (cells that were not treated or treated with Ad-Luc) (FIG. 21, A and B). However, Ad-mda-7 significantly decreased Cdc2 in LNCaP cells but not in DU145 cells. Further, cyclinA and cycline, which are related to the G1/S cell cycle checkpoint and S phase were reduced by the addition of Ad-mda-7 in both cell lines. p27 and p21 related with G1/S and/or G2/M cell cycle checkpoint were increased in LNCaP cells but not in DU145 cells. This suggested that Ad-mda-7 may increase p27 and p21 through the enhancement of p53 (FIG. 21B). These results indicate that MDA-7 induces G2 cell cycle arrest associated with downregulation of Cdc25C and are consistent with FACS analysis of the cell cycle as described.

### Example 20

### Secreted Mda-7 Inhibits Endothelial Cell Differentiation and Migration

Materials and Methods

1. Cell Culture.

The human non-small cell lung cancer (NSCLC) cell line A549 (adenocarcinoma) and human embryonic kidney cells (293) obtained from the American Type Culture Collection (ATCC; Rockville, Md.) were grown in Hams/F12 medium (A549) and DMEM (293) supplemented with 10% fetal bovine serum (GIBCO-BRL, Grand Island, N.Y.). The human umbilical vein endothelial cells (HUVEC) and human dermal microvascular endothelial cells (HMVEC) purchased from Clonetics (Walkerville, Md.) were grown in endothelial cell basal medium with 5% fetal bovine serum and additional reagents supplied as a “bullet kit” by the manufacturer. Endothelial cells were used at passage 3-9 in the present study.

2. Production and Purification of Secreted MDA-7 Protein.

Production of MDA-7 protein was achieved by transfecting 293 cells with a eukaryotic expression vector carrying the full-length mda-7 cDNA. Following transfection, cells were selected in hygromycin (0.4 μg/ml) for 14 days. The stable cell line (293-mda-7) was tested for soluble MDA-7 protein production by Western blot analysis and by ELISA. Cells plated at 1×10⁶ cells/well (293-mda-7) produced approximately 30-50 ng/ml of sMDA-7 in 24 h as determined by ELISA. To purify the MDA-7 protein in large scale, 293-mda-7 cells were grown to 90% confluency in 150 mm tissue culture plates. The tissue culture supernatant was collected and pooled for protein purification by affinity chromatography as described previously (Blumberg et al., 2001). The size and purity of the sMDA-7 protein was determined by silver stain gel and by Western blot analyses. 3. Endothelial Cell Assays.

Endothelial cell differentiation (tube formation) assays were done using an in vitro angiogenesis assay kit (Chemicon, Temecula, Calif.). Briefly, HUVEC and HMVEC cells were grown to 80% confluency, collected, resuspended in growth medium and plated at a concentration of 2×10⁴ cells/well in a 96-well plate coated with matrigel (Chemicon, Temecula, Calif.). To the wells, varying concentrations of MDA-7 protein were added and incubated for 24 h at 37° C. Wells that were not treated with MDA-7 protein served as a negative control. The ability of sMDA-7 to inhibit tube formation was determined and quantitated by counting the number of tubes under bright field microscopy. All samples were tested in duplicate. For comparative studies using MDA-7 and endostatin (Calbiochem), cells were plated as described above and exposed to varying concentration of the proteins. For neutralization assay experiments HUVEC cells grown in 6-well plates were pretreated with IL-22R neutralizing antibody (5 ng/ml) for 24 h prior to performing the tube assay. All other experimental procedures were the same as described above.

4. Endothelial Cell Migration Assay.

Migration assays were performed using HUVEC cells. Cells were starved overnight in basal medium containing 0.5% fetal bovine serum (FBS), collected and re-suspended in the same medium and seeded at a concentration of 1×10⁵ cells/well in the upper surface of a 24-well transwell insert with a filter size of 8 μm (Millipore, Cambridge, Mass.). The insert was placed in a 6-well plate that contained medium plus VEGF (100 ng/ml) or VEGF plus MDA-7 (10 ng/ml). The plates containing the transwell were incubated at 37° C. overnight to allow migration. The following day, the wells were disassembled, membranes fixed in crystal violet and the number of cells that had migrated to the lower wells were counted under high power magnification (×40).

5. Western Blot Analysis.

To determine the regulation of pSTAT-3 expression following the addition of sMDA-7, Western blot analysis was performed as described previously (Wang et al., 2002). Briefly, cells were harvested by trypsinization and resuspended in lysis buffer (62.5 mM Tris-HCl, 2% SDS, 10% glycerol, 4 M urea). Protein samples (50 μg) were each diluted into a 20 μl solution of lysis buffer and 5% 2-mercaptoethanol (Bio-Rad Laboratories, Hercules, Calif.) and heated in a water bath at 95° C. for 5 min. Protein extracts were then separated by 10% SDS-PAGE in a vertical-slab gel electrophoresis cell (Bio-Rad). The separated proteins were transferred from gel to nitrocellulose membrane (Hybond-ECL; Amersham Pharmacia Biotech, Buckinghamshire, England) and then blocked in a blocking solution (5% dry milk and 0.3% Tween 20 in PBS) for 1 hour. Membranes were incubated with the primary antibodies against pSTAT-3 (1:1000), and β-actin (1:10000). The membranes were then incubated with horseradish peroxidase-labeled secondary antibodies (Amersham). Finally, the proteins were visualized on enhanced chemiluminescence film (Hyperfilm, Amersham) by application of Amersham's Enhanced Chemiluminescence Western blotting detection system.

6. In Vivo Angiogenesis Assay using the Matrigel Plus Assay

To determine the anti-angiogenic activity of sMDA-7, an in vivo angiogenesis assay was performed. Briefly, sMDA-7 and bFGF were mixed with 500 μl of matrigel (Beckton and Dickenson) on ice and injected subcutaneously into athymic nude mice. Animals receiving matrigel containing only bFGF (60 ng) served as a positive control while animals receiving matrigel containing no growth factor served as a negative control. Each group consisted of 5 animals. Experiments were terminated on day 10 and the matrigel was harvested, photographed and subjected to hemoglobin analysis as described previously (Caudel et al., 2002).

7. In Vivo Experiments.

Prior to the start of the experiments, parental 293 cells and 293-mda-7 cells were tested for their ability to form tumors by injecting 10⁶ cells subcutaneously in nude mice which were observed for a period of 1 month. No tumor formation was observed at this cell concentration. For in vivo mixing experiments, human lung tumor cells (A549) were grown to 90% confluency, collected and resuspended in sterile PBS at a concentration of 5×10⁶ cells/ml. These tumor cell suspensions were then mixed with equal number (5×10⁶ cells/ml) of parental 293 cells or 293-mda-7 cells, gently vortexed, and injected subcutaneously into athymic BALB/c female nude mice (10⁶ cells/animal). Each group consisted of 8 animals. Tumor growth was monitored and measured as described previously (Maheshwari et al., 1991). At the end of experimentation, animals were euthanized by CO₂ inhalation and tumors harvested for further analyses.,

To evaluate the effect of sMDA-7 on distant tumors, subcutaneous tumors were established by injecting A549 tumor cells (5×10⁶ cells) into the lower right flank of nude mice. When the tumors were 50-60 mm³ in size, animals were divided into two groups (n=10/group). One group received matrigel containing parental 293 cells (1×10⁶), and the other group received 293-mda-7 cells (1×10⁶). Matrigel containing the cells were injected subcutaneously into the upper right flank of the tumor-bearing mice. The tumors were measured as described and the effects of sMDA-7 on tumor growth were monitored. At the end of the experiment, animals were euthanized and tumors harvested for further analyses.

8. Immunohistochemical Analysis.

Tissues were stained for CD31 and TUNEL as described previously (Wang et al., 2002). Negative controls included tissue sections stained without primary antibody or stained with an isotypic antibody. Tissue sections were analyzed, quantitated and results interpreted in a blind fashion.

9. Statistical Analysis.

Student's t-test was used to calculate the statistical significance of the experimental results.

### Example 21

### Secreted MDA-7 (SMDA-7) Inhibits Endothelial Cell Differentiation

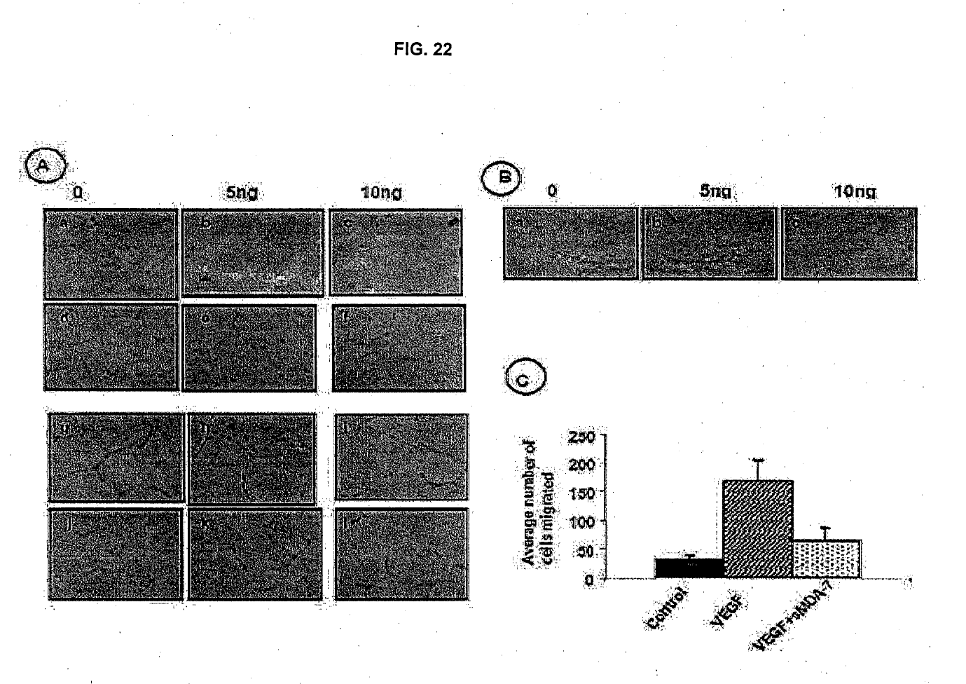

Secreted MDA-7 (sMDA-7) inhibits endothelial cell differentiation. The ability of sMDA-7 to inhibit endothelial cell differentiation was evaluated in HUVEC cells and HMVEC cells (FIG. 22A). The addition of sMDA-7 protein resulted in a significant inhibition of endothelial tube formation. The inhibitory effect was dose-dependent with complete abrogation of tubular formation occurring at a concentration of 10 ng/ml (FIG. 22A). In contrast, cells that were untreated demonstrated no inhibition of tube formation (FIG. 22A). Deletion experiments determined that the observed inhibition of tube formation by endothelial cells was due to sMDA-7 protein and not due to unrelated proteins in the preparation. Immuno-depletion of sMDA-7 prior to addition onto HUVEC cells resulted in a lack of inhibition of endothelial cell tube formation (FIG. 22B). Furthermore, the inhibitory effect by sMDA-7 was 25-times more potent than recombinant human endostatin against HUVEC cells and HMVEC cells (FIG. 22A) when used at the same protein concentrations. Thus the inhibitory activity of sMDA-7 on endothelial cell tube formation suggests that sMDA-7 possesses potent anti-angiogenic activity.

To determine if sMDA-7 could inhibit endothelial cell migration, migration assays were performed in the presence of vascular endothelial growth factor (VEGF). SMDA-7 (10 ng/ml) blocked endothelial cell migration in response to the angiogenic inducer whereas no inhibitory effect was observed in the controls (FIG. 22C). Inhibition was observed to be dose-dependent with complete inhibition occurring at 50 ng. A similar inhibitory activity was observed using basic fibroblast growth factor (bFGF) as an inducer.

Assays were performed to determine whether inhibition of tube formation by sMDA-7 was mediated via IFN-γ, and IP-10 production. Tissue culture supernatant from sMDA-7 treated HUVEC cells were collected at various time points and analyzed for IFN-γ and IP-10 by ELISA. IP-10 but not IFN-γ was induced by sMDA-7. However, the amount of IP010 produced (15-32 pg/ml) was not significant and cannot be responsible for the significant inhibitory effects observed with sMDA-7.

### Example 22

### SMDA-7 Activates STAT-3 Expression In Endothelial Cells

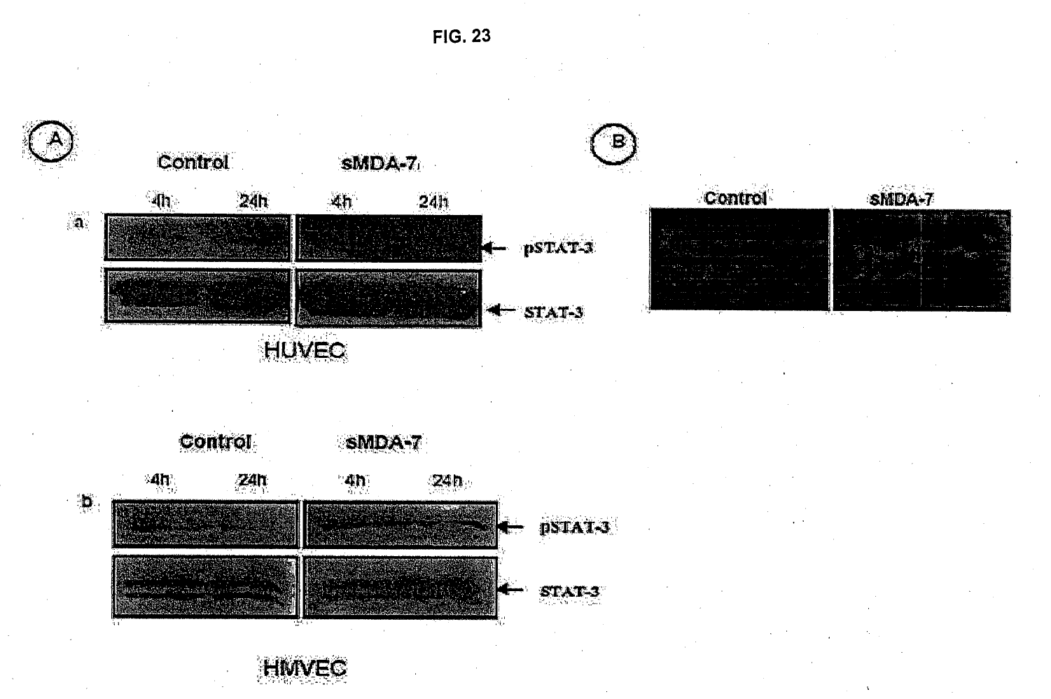

The signaling mechanism that may be triggered by sMDA-7 upon its addition to endothelial cells was also evaluated using the materials and methods as described in Example 1. STAT-3 activation in HUVEC cells and HMVEC cells (FIG. 23A) was analyzed by Western blot. The addition of sMDA-7 to endothelial cells resulted in a significant increase in the phosphorylated form of STAT-3 (pSTAT-3) protein expression as early as 4 h. Increase in pSTAT-3 expression was observed to be time-dependent with maximal expression occurring at 24 h after treatment (FIG. 23A). Additional evidence for pSTAT-3 activation is demonstrated by the increased nuclear localization of pSTAT-3 protein in HUVEC cells after treatment with sMDA-7 while no changes were observed in untreated control cells (FIG. 23B).

### Example 23

### Inhibition of Endothelial Cell Differentiation into Capillary-Like Structures (Tube Formation) by MDA-7 is Through STAT-3 Activation and is Partially Restored by Blocking IL-22R1.

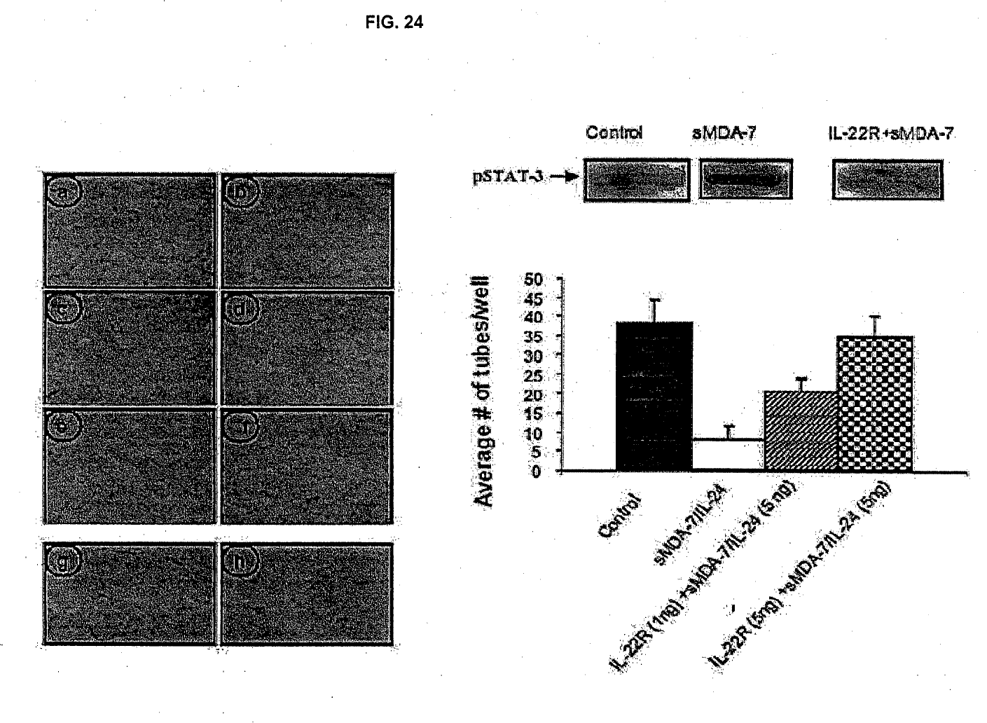

Inhibitory effects of sMDA-7 on endothelial cell differentiation are receptor-mediated. Experiments were performed using the materials and methods described in Example 19 to determine whether the inhibitory effect of sMDA-7 on endothelial cells is receptor-mediated by using a blocking antibody against the interleukin-22 receptor1 (IL-22R1) in the presence or absence of sMDA-7 (FIG. 24). Treatment of HUVEC cells with sMDA-7 (5 ng) resulted in complete inhibition of tube formation compared to untreated control cells (FIG. 24). However, pre-treatment of HUVEC cells with IL-22R1 blocking antibody resulted in the complete abrogation of the inhibitory effects of MDA-7 on tube formation (FIG. 24). The abrogation by the IL-22R1 antibody was observed to be dose-dependent. Addition of 1 ng of blocking antibody (1:5 ratio) resulted only in a partial restoration of tubes (<60%, FIG. 24) while addition of 5 ng of the blocking antibody (1:1 ratio) resulted in complete restoration (>90%, FIG. 24) of tubular formation by HUVEC cells. Addition of neutralizing antibody alone had no significant effect on the ability of HUVEC to forms tubes (FIG. 24).

To further determine the specificity of the IL-22R1 antibody, additional experiments were performed using a neutralizing antibody against the IL-10 receptor (IL-10R). In the presence of IL-10R neutralizing antibody, sMDA-7 was able to inhibit tubular formation by HUVEC cells compared to untreated control (FIG. 24). These results indicate that sMDA-7 mediates its specific inhibitory effects on endothelial cells using the IL-22R1. Semi-quantitation analysis demonstrated that sMDA-7 significantly inhibited the number of tubes formed (p=0.001) in cells treated with sMDA-7/IL-24 and anti-1L-22R1 antibody compared to untreated control cells (FIG. 24).

Further evidence that the inhibitory effect of sMDA-7 is receptor-mediated is demonstrated by the activation of pSTAT-3 protein expression in HUVEC cells. A significant increase in the phosphorylated form of STAT-3 (pSTAT-3) protein expression was observed after the addition of sMDA-7 protein in HUVEC cells (FIG. 24). However, no increase in sMDA-7-mediated pSTAT-3 expression was observed in the presence of IL-22R1 antibody (FIG. 24). These results indicate that the sMDA-7 activity against endothelial cells is L-22R1 mediated.

### Example 24

### Matrigel Encapsulated SMDA-7 Protein Inhibits Angiogenesis In Vivo

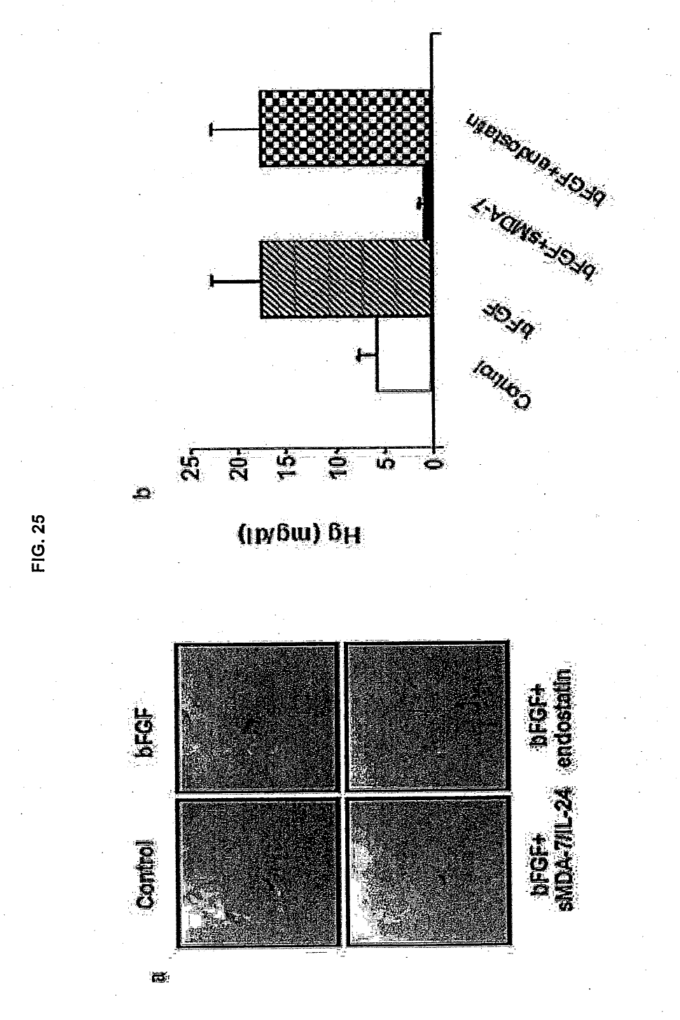

To determine if sMDA-7 could inhibit angiogenesis in vivo, a matrigel assay was utilized as described in Example 19. SMDA-7 protein (10 ng) encapsulated into matrigel containing bFGF was implanted subcutaneously in nude mice. Examination of the matrigel 10 days after implantation demonstrated inhibition of bFGF-induced angiogenesis by sMDA-7 when compared to controls (FIG. 25A). Determination of the hemoglobin content in the matrigel demonstrated a very significant (p=0.0001) inhibitory effect by sMDA-7 on bFGF-induced angiogenesis compared to controls that contained no bFGF, contained only bFGF, or contained recombinant human endostatin (FIG. 25B). Surprisingly the reduction in hemoglobin content by sMDA-7 was much stronger than that observed in the control that had no bFGF.

### Example 25

### In Vivo Mixing of 293-MDA7 Cells Secreting MDA-7 with Tumor Cells Inhibits Subcutaneous Human Lung Tumor Xenografts

The ability of sMDA-7 to inhibit tumor growth was tested by in vivo mixing experiments as described in Example 19. A549 tumor cells were mixed with either parental 293 cells or with 293-mda-7 cells (1:1 ratio) and injected subcutaneously in the lower right flank of mice. Tumor measurements were initiated when tumors were palpable. A significant inhibition (p=0.001) of tumor growth was observed in animals that received a mixture of A549 and 293-mda-7 cells compared to animals that received a mixture of A549 and parental 293 cells (FIG. 26A). Injection of 293 or 293-mda-7 cells alone do not form tumors in nude mice.

Animals were euthanized on day 22 post-implantation and tumors were harvested and further evaluated. Western blot analysis demonstrated MDA-7 protein expression in tumors that contained a mixture of A549 tumor cells and 293-mda-7 cells compared to controls that contained a mixture of A549 tumor cells and 293 cells (FIG. 26B). Histopathological -examination of tumor tissue did not reveal any significant difference in the tumor cell proliferative index or in tumor cell infiltration between control and experimental animals (FIG. 26C). However, reduced vascularization as indicated by CD31 staining was observed in tumors that contained 293-mda-7 cells compared to control tumors that contained 293 cells (FIG. 26C). Reduction in tumor vascularization correlated with decreased hemoglobin content in tumors harvested from animals that received a mixture of A549 and 293-mda-7 cells compared to animals that received a mixture of A549 and parental 293 cells (FIG. 26D). TUNEL staining of tumor tissues from experimental animals demonstrated endothelial cells and tumor cells surrounding the endothelial cell undergoing apoptotic cell death as indicated by the brown staining (FIG. 26C). In contrast, no TUNEL positive staining was observed in control tumor tissues.

### Example 26

### Matrigel Encapsulated 293 Cells Secreting MDA-7 Inhibit Subcutaneous Human Lung Tumor Xenografts

Mice were inoculated subcutaneously with A549 tumor cells in the lower right flank as described in Example 19. When the tumors had reached a size of 50 mm³, 293 cells producing sMDA-7 (293-mda-7) or parental 293 cells (control) were encapsulated in matrigel and implanted subcutaneously at a site distant (upper right flank) from the tumor and the tumor growth was monitored. The growth of A549 lung tumor xenografts was significantly inhibited (p=0.001) by 293 cells secreting MDA-7 when compared to the control group (FIG. 27A). In comparison to the control, the growth of the tumors was suppressed by 50% after implantation of the encapsulated 293 cells producing MDA-7 protein. To confirm that the inhibitory effect was due to sMDA-7, serum samples from animals were tested for MDA-7 protein by Western blot analysis and ELISA. Intense banding of sMDA-7 at the expected 40 Kd size was observed in the serum of animals implanted with 293 cells producing MDA-7 by Western blot analysis (FIG. 27A). However, faint bands were also observed in the control that may be due to some cross-reactivity with serum proteins. The serum levels of sMDA-7 detected by ELISA was approximately 50 ng/ml.

At the end of experimentation, tumors and injected matrigel containing 293-mda-7 cells were harvested and stained. Immunohistochemical analysis of the matrigel from animals receiving 293-mda-7 cells using a monoclonal anti-MDA-7 antibody demonstrated MDA-7 protein expression as evidenced by the brown staining (FIG. 27B). In contrast, MDA-7 was not detected in the matrigel from animals receiving parental 293 cells (FIG. 27B). Additionally, tumors treated with 293-mda-7 had less vascularization than tumors treated with parental 293 cells, as evidenced by CD31 positive staining (FIG. 27B). Histopathological analysis of tumor tissues demonstrated no differences between animals receiving 293 cells and 293-mda-7 cells (FIG. 27B).

### Example 27

### AD-MDA7 Induces Growth Arrest and Apoptosis Selectively in Tumor Cells via Intracellular MDA-7 Protein

In order to determine the effect of subcellular localization of MDA-7 on protein function, adenoviral vectors were constructed to direct targeting of MDA-7 protein. Targeted vectors, purchased from Invitrogen, were developed that direct subcloned MDA-7 proteins to the cytoplasm, mitochondria, or endoplasmic reticulum (ER). Each vector adds a C-terminal myc tag to expressed proteins. The vector directing proteins to the cytoplasm contains a standard expression vector backbone, while the vectors directing proteins to the mitochondria and ER, in addition to having backbones identical to the cytoplasmic vector, contain signal sequences appropriate to those compartments. The mitochondrial targeting vector has an N-terminal mitochondrial targeting signal, while the ER targeting vector has an N-terminal ER signal peptide sequence and a C-terminal ER retention sequence. Mda-7 was subcloned into these vectors by using PCR to delete both the stop codon and the first 48 amino acids, constituting the secretion signal, from full-length mda-7 cDNA. PCR was also used to provide restriction sites compatible with the Invitrogen targeting vectors, and in frame with the C-terminal myc tag contained in the vectors. The forward PCR primer used (with SalI site) was ttttttGTCGACatggcccagggccaagaattcc (SEQ ID NO:3). The reverse PCR primer used (with NotI site) was ttttttGCGGCCGCgagcttgtagaatttctgc (SEQ ID NO:4). These plasmids have been demonstrated to direct MDA-7 protein successfully to the appropriate subcellular compartment.

The adenoviral constructs were made by removing the mda-7 gene and accompanying signal sequences from these targeting plasmids using the restriction endonucleases Pm1I and XbaI and subcloning them into the adenoviral shuttle vector using standard methods.

The MDA-7 protein was originally described as a nuclear protein (Jiang et al). Analysis of the predicted primary sequence indicates that the MDA-7 protein contains a prototypic signal sequence, which is likely to be responsible for directing secretion of the protein. The translated protein product demonstrates a strongly hydrophobic region at the N-terminus (FIG. 28). The MDA-7 protein is predicted to be cleaved at amino acid 48, resulting in the remaining protein product of amino acids 49-206 being secreted from the cell. To facilitate analysis of the secreted MDA-7 protein product, a stable cell line expressing mda-7 has been constructed using HEK 293 cells. Supernatants from these cells show a strongly immunoreactive MDA-7 band at approximately 40 kD (FIG. 28). The major secreted MDA-7 protein band has been sequenced and it was verified that amino acid 49 is the first amino acid of the extracellular protein. Treatment of cells with Ad-mda7 causes secretion of the MDA-7 protein both from tumor and normal cells. The kinetics of MDA-7 released from tumor cells are slightly delayed compared to kinetics of intracellular MDA-7 expression after Ad-mda7 treatment. However, the kinetics of release are slightly different between tumor and normal cells. Tumor cells tend to secrete MDA-7 protein into the media within 24 hours post-transduction, whereas normal cells display somewhat slower kinetics of release. The absolute levels of MDA-7 protein released from tumor and normal cells, however, are comparable.

The presence of Ad-mda7 is known to activate stress genes (FIG. 29A). H1299 non-small cell lung cancer cells were treated with Ad-mda7 or Ad-Luc (at 500 vp/cell or 1000 vp/cell) and evaluated for expression of stress-related proteins using Western blot analysis. FIG. 29A shows that Ad-mda7 causes a significant increase in the stress proteins BiP/GRP78, GADD34, PP2A and Caspase 7. These stress protesins are implicated in activation of the mammalian stress response known as Unfolded Protein Response (UPR). To confirm this result, additional members of the UPR pathway were analyzed, including: caspases 7, 12 and XBP-1. As shown in FIG. 29B, the additional UPR-associated proteins are upregulated due to Ad-mda7 transduction, suggesting that UPR was the mechanism by which MDA-7 was killing cancer cells. The expression of PERK, another protein characteristic of UPR activation, was evaluated. No detectable levels of PERK were identified in H1299 cells. Previous studies have indicated that PERK is only expressed in secretory cells, such as beta-islet cells. Thus, lack of PERK expression in NSCLC cells is not surprising.

Ad-mda7 was also shown to disrupt calcium flux and mitochondrial stability by immunohistochemistry (FIG. 30). Analytical studies were carried out on Ad-mda7 or Ad-Luc transduced H1299 cancer cells. Calcium flux and mitochondrial integrity were analyzed via confocal microscopy. As demonstrated in FIG. 30, the presence of Ad-mda7 causes an increase in intramitochondrial calcium levels resulting in mitochondrial instability. This instability in conjunction with an increase in several stress-related proteins may account for an increase in apoptosis in the presence of Ad-mda7.

### Example 28

### MDA-7 is Heavily Glycosylated

The higher molecular weight of the secreted MDA-7 protein, as seen in FIG. 28, is indicative of glycosylation, and is consistent with the presence of three predicted N-glycosylation sites in the MDA-7 sequence. Secreted MDA-7 protein stably expressed via 293-mda7 cells was treated with different glycosidases including glycopeptidase F (glycoF), sialidase, and endoglycosidase H (EndoH). As seen in FIG. 31B, digestion of the secreted protein with glycoF, which cleaves the sugar-protein bond, causes substantial reduction in the molecular weight of MDA-7. The same is seen to a lesser extent after the digestion with sialidase and EndoH. This is further confirmed after various combinations of deglycosidases were added (FIG. 31B). Furthermore, the experiment indicates that the secreted MDA-7 protein has variable glycosylation as multiple bands are observed in the untreated sample whereas the de-glycosylated material is much less complex.

### Example 29

### Secreted MDA-7 does not Cause Substantial Cell Death

Since glycosylated proteins generally acquire sugars during sorting through the ER and Golgi apparatus, the effect of glycosylation and secretion inhibitors on MDA-7 intracellular processing and subsequent secretion was evaluated. Tunicamycin inhibits the addition of sugars to proteins within the ER, while brefeldin A inhibits vesicle transport of proteins from the ER to the Golgi. Both disrupt the secretion of proteins. The cytotoxicity caused by Ad-mda7 in the presence of these glycosylation and secretion inhibitors was evaluated. Tunicamycin and brefeldin A levels were titrated until inhibition of secretion of MDA-7 protein could be detected. As shown in FIGS. 26A and 26B, treatment of H1299 cells with 2 μg/ml tunicamycin completely blocks secretion of MDA-7 protein, whereas less than 1 μg/ml of brefeldin A is required for this effect. Note that blocking secretion using either drug results in substantial intracellular accumulation of MDA-7 protein. When assayed for cytotoxicity using a trypan blue viability assay, however, glycosylation inhibitors did not abrogate cell killing by Ad-mda7. However, the combination of Ad-mda7 and 2 μg/ml tunicamycin caused enhanced cell death, even though 2 μg/ml tunicamycin did not induce death. The enhanced killing may be due to the tunicamycin sensitizing cells to apoptotic death. Note that the Ad-Luc/tunicamycin control sample exhibited comparable cytotoxicity to the Ad-Luc and tunicamycin treated samples. Therefore, secreted MDA-7 protein is unable to induce killing in cancer cells and is not required for Ad-mda7 mediated apoptosis and eventual killing of cancer cells (FIG. 32B).

This result suggested that it was the intracellular and not the secreted form of MDA-7 that was primarily responsible for eliciting cell death. This hypothesis was further tested by adding secreted MDA-7 to tumor cells and monitoring cell death. Initial studies adding supernatant from Ad-mda7 transduced cells did indicate cell death; however, careful analysis demonstrated that cell death was caused by residual Ad-mda7 in the culture supernatant. When culture supernatants were treated to minimize Ad-mda7 contamination, negligible cell death was induced by MDA-7 containing supernatants. When supernatant from 293 cells was applied to tumor cell cultures, only low levels of cell death were seen (approximately 10-15% above background levels) confirming that the secreted MDA-7, at least at the concentrations released by these cells, was not sufficient to elicit substantial cell death in tumor cells. Co-administration of anti-MDA7 antibodies inhibited death by MDA-7 whereas control IgG had not effect. When anti-MDA-7 was added to Ad-mda7 infected cultures, only a minor effect on cell killing was observed, indicating that the primary killing activity from Ad-mda7 treated cultures was due to intracellular protein.

### Example 30

### Cell Mixing Bystander Anayses

It has previously been shown that Ad-mda7 had a potential bystander effect in the H460 cell line (Mhashilkar et al., 2001). In that study, H460 cells were transduced with Ad-mda7 and immunostained with anti-MDA-7 antibody in conjunction with Annexin V staining. By confocal analysis, it was observed that some cells were Annexin V positive but were negative for MDA-7 expression. The frequency of Annexin V positive/MDA-7 negative cells was low and was not observed in multiple cells lines. Thus, to further evaluate the contribution of secreted MDA-7 in inducing apoptosis in neighboring cells, a series of bystander experiments were performed. Ad-mda7 transduced cells were mixed with native cells that had been previously labled with Ad-GFP and the cultures were scored for apoptosis in the GFP positive cells. The levels of apoptotic GFP positive cells were low. These studies confirmed that the secreted MDA-7 was not responsible for eliciting the high level of apoptosis observed in Ad-mda7 treated tumor cell cultures, and further demonstrated that cell-cell contact did not enhance bystander effect.

### Example 31

### Subcellular Targeting of MDA-7

If secretion of MDA-7 is not required for Ad-mda7 mediated apoptosis, then how does MDA-7 expression result in cell death? This question was addressed by re-targeting the MDA-7 protein to various sub-cellular compartments and evaluated as to how this affects MDA-7 mediated cytotoxicity. Also addressed was the question of whether MDA-7 being released into the cytosol or nucleus during supra-physiological expression in Ad-mda7 infected cells was responsible for inducing death. In order to investigate the effects of subcellular localization of MDA-7 protien on cell viablilty, expression vectors designed to target MDA-7 expression to different subcellular compartments were constructed. In constructing these vectors, the secretion signal sequence in the mda-7 cDNA was first deleted. As shown in FIG. 33A, the nuclear targeting vector contains three nuclear localization signals, the ER targeting vector contains an ER signal sequence and retention signal, and the cytoplasmic targeting vector contains no targeting signals, allowing the default expression of proteins in the cytoplasm. The full-length plasmid uses the cytoplasmic targeting vector backbone but contains full-length mda-7 cDNA. All proteins expressed by these plasmids also contain a myc tag at the C-terminus. FIG. 33B shows that each vector successfully promotes the expression of MDA-7 protein within the cell, while only the full-length mda-7 cDNA, including the N-terminal secretion signal sequence, permits secretion of MDA-7 protein into the media.

Western analysis was used to look at the expression of MDA-7 protein in lysates and supernatants of transfected H1299 cells using different constructs of mda-7 as described in FIG. 33A. Addition of the myc tag did not adversely affect MDA-7 protein stability as a control full-length mda-7 expression plasmid (without a myc tag) expressed comparable levels of MDA-7 protein. The myc tag did not appear to interfere with protein processing or secretion as the full-length myc-tagged protein showed two bands similar to the full length MDA-7 protein expressed by fill-length plasmid or Ad-mda7, except that these bands are larger due to the myc tag (FIG. 33B). The myc-tagged MDA-7 appeared to be secreted and glycosylated similarly to native MDA-7 from full-length MDA-7 and Ad-mda-7 treated cells.

### Example 32

### Subcellular Localization of MDA-7 Affects Cytotoxicity

The vectors were transiently transfected into H1299 cells, and the subsequent targeted MDA-7 protein expression determined by immunohistochemistry. As shown in FIG. 34, each vector successfully targets MDA-7 protein to the intended subcellular compartment. The MDA-7 protein expressed from the full-length plasmid can be seen in secretory granules within the cell, consistent with the results observed after Ad-mda7 transduction. The precise subcellular localization of the targeted plasmids was confimed by comparison with the expression patterns of molecules known to reside in these compartments. For example, full-length MDA-7 was shown to co-localize in secretory vesicles. Nuclear targeted MDA-7 co-localized with Hoescht staining and ER-targeted MDA-7 co-stained with BiP.

The anti-tumor effects were analyzed to determine the effect the targeted MDA-7 protein has on cell viability. This was achieved by colony formation assays. As can be seen in FIG. 35, neither nuclear nor cytoplasmic mda-7 expression constructs had an effect on the formation of stable transfectant colonies. Full-length, secreted MDA-7 protein and MDA-7 protein targeted to the ER, however, cause a reduction in colony formation, indicating the lethality of MDA-7 in these environments.

Further analysis to determine the cytotoxic effect of Ad-mda7 on cells is shown in FIG. 36. H1299 cells were transfected with mda-7 targeting plasmids and evaluated in the live/dead assay. MDA-7 protein targeted to the ER inhibits cancer cell proliferation as seen by an increased number of dead cells (red, Ethidium bromide staining). Mock, cytoplasmic and nuclearly targeted MDA-7 show minimal cell killing. Additionally, Hoescht staining was used as a screen to evaluate cytotoxic effects of re-targeted MDA-7 expression (FIG. 37). It was found that nuclear or cytoplasmic MDA-7 expression had no effect on nuclear morphology. Cells containing secreted or ER-localized MDA-7 protein, however, have disrupted nuclear morphology indicative of apoptosis.

### Example 33

### Mitochondrial-Targeted MDA-7 Subcellular Localization

PC3 prostate tumor cells were transduced with plasmids encoding GFP control, full-length MDA-7 or mitochondrially targeted MDA-7 and evaluated in a colony formation assay. Full-length MDA-7 resulted in a 35% decrease in colony formation compared to the control, whereas mitochondrially targeted MDA-7 further reduced colony formation and viability of PC3 cells. Thus, targeting MDA-7 to the mitochondria further enhances its anti-tumor and pro-apoptotic effects (FIG. 38).

### Example 34

### Suppression of Adenovirus-Mediated MDA-7 Activation of NF-KB Induces a Synergistic Therapeutic Effect in Human Lung Cancer Cells

Studies were conducted to determine whether transgenic MDA-7 expression results in NF-κB activation, and to evaluate the role of NF-κB in protecting tumor cells from MDA-7 induced apoptosis. Adenovirus-medicated mda-7 (Ad-mda7) gene transfer in two NSCLC cell lines (H1299 and A549) resulted in NF-κB activation as demonstrated by electromobility shift assay (EMSA) (FIG. 39A). Marked activation of NF-κB was observed between 20-36 hours in cells treated with Ad-mda7 but not in control cells treated with PBS, or cells treated with Ad-luc (vector expressing luciferase) (FIG. 39A). Furthermore, activation of NF-κB occurred in a dose-dependent manner, with increasing concentrations of Ad-mda7 resulting in increased NF-κB activation. Coinciding with NF-κB activation was the degradation of an inhibitor of NF-κB (I-κBalpha). Furthermore, transfection of H1299 cells with an adenoviral vector overexpressing dominant negative mutant I-KB (Ad-mIkB) significantly inhibited Ad-mda7 induced transcriptional and DNA binding activity of NF-κB resulting in increased tumor cell apoptosis, when compared to control cells that were treated with Ad-luc (FIG. 39B).

Additionally, inhibition of MDA-7 mediated NF-κB activation by Sulindac (FIG. 40), a non-steroidal anti-inflammatory drug, resulted in a synergistic therapeutic effect. Sulindac, but not indomethacin, inhibited the activation of the NF-κB pathway. Sulindac inhibited TNF-mediated NF-κB activation in a dose-dependent manner (FIG. 41). In addition, Ad-mda7 synergizes with Sulindac to induce apoptosis in H1299 cells (FIG. 42). There was also a marked increase in the sub-GI population by combination treatment of Sulindac and Ad-mda7 (FIG. 43). These results suggest that MDA-7 expression in lung cancer cells induces NF-κB, and its inhibition using Ad-mIkB or Sulindac, enhances the therapeutic effect (FIG. 44).

### Example 35

### AD-MDA-7 Activates the Immune System in Patients with Advanced Cancer

In an ongoing Phase I dose-escalating clinical trial, mda-7 was administered via intratumoral injection to patients with advanced carcinoma using a non-replicating adenoviral construct (Ad-mda7). Patients had histologically confirmed carcinoma with at least one lesion that was accessible for needle injection that was surgically resectable, a Karnofsky performance status of ≧70%, and acceptable hemotologic, renal and hepatic function. Patients with active CNS metastases, chronic immunosuppressive use, or prior participation in a therapy requiring the administration of adenovirus were excluded from participation. Patients with surgically resectable advanced cancers received single intratumoral injections of 2×10¹⁰ to 2×10¹² viral particles (vp) (FIG. 45). To date, eight cohorts (18 patients) have completed enrollment.

To characterize the effects of intratumoral mda-7 treatment, systemic immune responses to Ad-mda7 were analyzed via serum cytokines and lymphocyte subsets. A majority of patients exhibited transient increases in systemic cytokines (L-6, 14/18 patients tested; IL-10, 15/18; γIFN, 8/18; TNFα, 10/18) (FIG. 46, FIG. 47). Some high dose patients also exhibited increased intratumoral expression of 1L-6, γIFN and IL-10 cytokine mRNAs. Further, CD3+ CD8+ T cells were increased by 30±13% at day 15 following mda-7 treatment (FIG. 48, FIG. 49). These findings suggest that MDA-7 increases systemic T_{H}1 cytokine production and mobilizes CD8+ T cells. FIG. 46 show that after Ad-mda7 injection, circulating 1L-6, IFN-gamma, IL-10 and TNF-alpha substantially increase and then fall to baseline levels by day 30. Cytokine increases correlate with increases in CD8+ cells and inversion of CD4/CD8 ratios. Thus, the results suggest immune activation by Ad-mda7 and is consistent with the pro-TH1 activity of rhMDA-7 in culture.

### Example 36

### Secreted Mda-7 Inhibits Endothelial Cell Differentiation and Migration

Materials and Methods

1. Cell Culture.

The human non-small cell lung cancer (NSCLC) cell line A549 (adenocarcinoma) and human embryonic kidney cells (293) obtained from the American Type Culture Collection (ATCC; Rockville, Md.) were grown in Hams/F12 medium (A549) and DMEM (293) supplemented with 10% fetal bovine serum (GIBCO-BRL, Grand Island, N.Y.). The human umbilical vein endothelial cells (HUVEC) and human dermal microvascular endothelial cells (HMVEC) purchased from Clonetics (Walkerville, Md.) were grown in endothelial cell basal medium with 5% fetal bovine serum and additional reagents supplied as a “bullet kit” by the manufacturer. Endothelial cells were used at passage 3-9 in the present study.

2. Production and Purification of Secreted MDA-7 Protein.

Production of MDA-7 protein was achieved by transfecting 293 cells with a eukaryotic expression vector carrying the full-length mda-7 cDNA. Following transfection, cells were selected in hygromycin (0.4 μg/ml) for 14 days. The stable cell line (293-mda-7) was tested for soluble MDA-7 protein production by Western blot analysis and by ELISA. Cells plated at 1×10⁶ cells/well (293-mda-7) produced approximately 30-50 ng/ml of sMDA-7 in 24 h as determined by ELISA. To purify the MDA-7 protein in large scale, 293-mda-7 cells were grown to 90% confluency in 150 mm tissue culture plates. The tissue culture supernatant was collected and pooled for protein purification by affinity chromatography as described previously (Blumberg et al., 2001). The size and purity of the sMDA-7 protein was determined by silver stain gel and by Western blot analyses.

3. Endothelial Cell Assays.

Endothelial cell differentiation (tube formation) assays were done using an in vitro angiogenesis assay kit (Chemicon, Temecula, Calif.). Briefly, HUVEC and HMVEC cells were grown to 80% confluency, collected, resuspended in growth medium and plated at a concentration of 2×10⁴ cells/well in a 96-well plate coated with matrigel (Chemicon, Temecula, Calif.). To the wells, varying concentrations of MDA-7 protein were added and incubated for 24 h at 37° C. Wells that were not treated with MDA-7 protein served as a negative control. The ability of sMDA-7 to inhibit tube formation was determined and quantitated by counting the number of tubes under bright field microscopy. All samples were tested in duplicate. For comparative studies using MDA-7 and endostatin (Calbiochem), cells were plated as described above and exposed to varying concentration of the proteins. For neutralization assay experiments HUVEC cells grown in 6-well plates were pretreated with IL-22R neutralizing antibody (5 ng/ml) for 24 h prior to performing the tube assay. All other experimental procedures were the same as described above.

4. Endothelial Cell Migration Assay.

Migration assays were performed using HUVEC cells. Cells were starved overnight in basal medium containing 0.5% fetal bovine serum (FBS), collected and re-suspended in the same medium and seeded at a concentration of 1×10⁵ cells/well in the upper surface of a 24-well transwell insert with a filter size of 8 μm (Millipore, Cambridge, Mass.). The insert was placed in a 6-well plate that contained medium plus VEGF (100 ng/ml) or VEGF plus MDA-7 (10 ng/ml). The plates containing the transwell were incubated at 37° C. overnight to allow migration. The following day, the wells were disassembled, membranes fixed in crystal violet and the number of cells that had migrated to the lower wells were counted under high power magnification (×40).

5. Determination of IP-10 and IFN-γ Production

HUVEC was seeded in six-well plates (1×10⁵/well) and treated with sMDA-7 (10 ng/ml). Cell culture supernatant was collected at 6 h, 24 h, and 48 h after treatment, centrifuged at 1200 rpm, and analyzed for IP-10 and IFN-γ protein production using commercially available ELISA kits. Assays were performed as recommended by the manufacturer (R&D Systems, Minneapolis, Minn.). Cells treated with recombinant IFN-γ served as positive controls for IP-10 while cells treated with Ad-mda7 (3000 vp/cell) served as positive control for IFN-γ. Untreated cells served as negative controls in these experiments. Samples were analyzed in quadruplicate and data represented as the average value for each concentration of sMDA-7 tested.

6. Western Blot Analysis.

To determine the regulation of pSTAT-3 expression following the addition of sMDA-7, Western blot analysis was performed as described previously (Wang et al., 2002). Briefly, cells were harvested by trypsinization and resuspended in lysis buffer (62.5 mM Tris-HCl, 2% SDS, 10% glycerol, 4 M urea). Protein samples (50 μg) were each diluted into a 20 μl solution of lysis buffer and 5% 2-mercaptoethanol (Bio-Rad Laboratories, Hercules, Calif.) and heated in a water bath at 95° C. for 5 min. Protein extracts were then separated by 10% SDS-PAGE in a vertical-slab gel electrophoresis cell (Bio-Rad). The separated proteins were transferred from gel to nitrocellulose membrane (Hybond-ECL; Amersham Pharmacia Biotech, Buckinghamshire, England) and then blocked in a blocking solution (5% dry milk and 0.3% Tween 20 in PBS) for 1 hour. Membranes were incubated with the primary antibodies against pSTAT-3 (1:1000), and β-actin (1:10000). The membranes were then incubated with horseradish peroxidase-labeled secondary antibodies (Amersham). Finally, the proteins were visualized on enhanced chemiluminescence film (Hyperfilm, Amersham) by application of Amersham's Enhanced Chemiluminescence Western blotting detection system.

7. Immunofluorescence Assay

Activation of STAT-3 was also determined by immunofluorescence assay. HUVEC seeded in two-well chamber slides (1×10⁴ cells/well) was treated with sMDA-7 (10 ng/ml) for 4 h, washed in PBS, fixed in cold acetic acid and stained for phosphorylated STAT-3 (pSTAT-3) using rabbit anti-human pSTAT-3 antibody (1:1000, Cell Signaling Technology, Beverly, Mass.) and rhodamine labeled anti-rabbit secondary antibody (1:5000; Molecular Probes, Eugene, Oreg.). Slides were mounted using anti-fade mounting reagent (Vector Laboratories). Pictures were taken 1-2 h after staining using a fluorescence microscope.

8. In Vivo Angiogenesis Assay using the Matrigel Plus Assay

To determine the anti-angiogenic activity of sMDA-7, an in vivo angiogenesis assay was performed. Briefly, sMDA-7 and bFGF were mixed with 500 μl of matrigel (Beckton and Dickenson) on ice and injected subcutaneously into athymic nude mice. Animals receiving matrigel containing only bFGF (60 ng) served as a positive control while animals receiving matrigel containing no growth factor served as a negative control. Each group consisted of 5 animals. Experiments were terminated on day 10 and the matrigel was harvested, photographed and subjected to hemoglobin analysis as described previously (Caudel et al., 2002).

9. In Vivo Experiments.

Prior to the start of the experiments, parental 293 cells and 293-mda-7 cells were tested for their ability to form tumors by injecting 106 cells subcutaneously in nude mice which were observed for a period of 1 month. No tumor formation was observed at this cell concentration. For in vivo mixing experiments, human lung tumor cells (A549) were grown to 90% confluency, collected and resuspended in sterile PBS at a concentration of 5×10⁶ cells/ml. These tumor cell suspensions were then mixed with equal number (5×10⁶ cells/ml) of parental 293 cells or 293-mda-7 cells, gently vortexed, and injected subcutaneously into athymic BALB/c female nude mice (106 cells/animal). Each group consisted of 8 animals. Tumor growth was monitored and measured as described previously (Maheshwari et al., 1991). At the end of experimentation, animals were euthanized by CO₂ inhalation and tumors harvested for further analyses.

To evaluate the effect of sMDA-7 on distant tumors, subcutaneous tumors were established by injecting A549 tumor cells (5×10⁶ cells) into the lower right flank of nude mice. When the tumors were 50-60 mm in size, animals were divided into two groups (n=10/group). One group received matrigel containing parental 293 cells (1×10⁶), and the other group received 293-mda-7 cells (1×10⁶). Matrigel containing the cells were injected subcutaneously into the upper right flank of the tumor-bearing mice. The tumors were measured as described and the effects of sMDA-7 on tumor growth were monitored. At the end of the experiment, animals were euthanized and tumors harvested for further analyses.

10. Immunohistochemical Analysis.

Tissues were stained for CD31 and TUNEL as described previously (Wang et al., 2002). Negative controls included tissue sections stained without primary antibody or stained with an isotypic antibody. Tissue sections were analyzed, quantitated and results interpreted in a blind fashion.

11. Statistical Analysis.

Student's t-test was used to calculate the statistical significance of the experimental results.

### Example 37

### Secreted MDA-7 (SMDA-7) Inhibits Endothelial Cell Differentiation

Secreted MDA-7 (sMDA-7) inhibits endothelial cell differentiation. The ability of sMDA-7 to inhibit endothelial cell differentiation was evaluated in HUVEC cells and HMVEC cells (FIG. 22A). The addition of sMDA-7 protein resulted in a significant inhibition of endothelial tube formation. The inhibitory effect was dose-dependent with complete abrogation of tubular formation occurring at a concentration of 10 ng/ml (FIG. 22A). In contrast, cells that were untreated demonstrated no inhibition of tube formation (FIG. 22A). Deletion experiments determined that the observed inhibition of tube formation by endothelial cells was due to sMDA-7 protein and not due to unrelated proteins in the preparation. Immuno-depletion of sMDA-7 prior to addition onto HUVEC cells resulted in a lack of inhibition of endothelial cell tube formation (FIG. 22B). Furthermore, the inhibitory effect by sMDA-7 was 25-times more potent than recombinant human endostatin against HUVEC cells and HMVEC cells (FIG. 22A) when used at the same protein concentrations. Thus the inhibitory activity of sMDA-7 on endothelial cell tube formation suggests that sMDA-7 possesses potent anti-angiogenic activity.

To determine if sMDA-7 could inhibit endothelial cell migration, migration assays were performed in the presence of vascular endothelial growth factor (VEGF). SMDA-7 (10 ng/ml) blocked endothelial cell migration in response to the angiogenic inducer whereas no inhibitory effect was observed in the controls (FIG. 22C). Inhibition was observed to be dose-dependent with complete inhibition occurring at 50 ng. A similar inhibitory activity was observed using basic fibroblast growth factor (bFGF) as an inducer.

Assays were performed to determine whether inhibition of tube formation by sMDA-7 was mediated via IFN-γ, and IP-10 production. Tissue culture supernatant from sMDA-7 treated HUVEC cells were collected at various time points and analyzed for IFN-γ and IP-10 by ELISA. IP-10 but not IFN-γ was induced by sMDA-7. However, the amount of IP010 produced (15-32 pg/ml) was not significant and cannot be responsible for the significant inhibitory effects observed with sMDA-7.

### Example 38

### SMDA-7 Activates STAT-3 Expression in Endothelial Cells

The signaling mechanism that may be triggered by sMDA-7 upon its addition to endothelial cells was also evaluated using the materials and methods as described in Example 1. STAT-3 activation in HUVEC cells and HMVEC cells (FIG. 23A) was analyzed by Western blot. The addition of sMDA-7 to endothelial cells resulted in a significant increase in the phosphorylated form of STAT-3 (pSTAT-3) protein expression as early as 4 h. Increase in pSTAT-3 expression was observed to be time-dependent with maximal expression occurring at 24 h after treatment (FIG. 23A). Additional evidence for pSTAT-3 activation is demonstrated by the increased nuclear localization of pSTAT-3 protein in HUVEC cells after treatment with sMDA-7 while no changes were observed in untreated control cells (FIG. 23B).

### Example 39

### Inhibition of Endothelial Cell Differentiation into Capillary-Like Structures (Tube Formation) by MDA-7 is Through STAT-3 Activation and is Partially Restored by Blocking IL-22R1.

Inhibitory effects of sMDA-7 on endothelial cell differentiation are receptor-mediated. Experiments were performed using the materials and methods described in Example 19 to determine whether the inhibitory effect of sMDA-7 on endothelial cells is receptor-mediated by using a blocking antibody against the interleukin-22 receptor1 (IL-22R1) in the presence or absence of sMDA-7 (FIG. 24). Treatment of HUVEC cells with sMDA-7 (5 ng) resulted in complete inhibition of tube formation compared to untreated control cells (FIG. 24). However, pre-treatment of HUVEC cells with IL-22R1 blocking antibody resulted in the complete abrogation of the inhibitory effects of MDA-7 on tube formation (FIG. 24). The abrogation by the IL-22R1 antibody was observed to be dose-dependent. Addition of 1 ng of blocking antibody (1:5 ratio) resulted only in a partial restoration of tubes (<60%, FIG. 24) while addition of 5 ng of the blocking antibody (1:1 ratio) resulted in complete restoration (>90%, FIG. 24) of tubular formation by HUVEC cells. Addition of neutralizing antibody alone had no significant effect on the ability of HUVEC to forms tubes (FIG. 24).

To further determine the specificity of the IL-22R1 antibody, additional experiments were performed using a neutralizing antibody against the IL-10 receptor (IL-10R). In the presence of IL-10R neutralizing antibody, sMDA-7 was able to inhibit tubular formation by HUVEC cells compared to untreated control (FIG. 24). These results indicate that sMDA-7 mediates its specific inhibitory effects on endothelial cells using the IL-22R1. Semi-quantitation analysis demonstrated that sMDA-7 significantly inhibited the number of tubes formed (p=0.001) in cells treated with sMDA-7/IL-24 and anti-IL-22R1 antibody compared to untreated control cells (FIG. 24).

Further evidence that the inhibitory effect of sMDA-7 is receptor-mediated is demonstrated by the activation of pSTAT-3 protein expression in HUVEC cells. A significant increase in the phosphorylated form of STAT-3 (pSTAT-3) protein expression was observed after the addition of sMDA-7 protein in HUVEC cells (FIG. 24). However, no increase in sMDA-7-mediated pSTAT-3 expression was observed in the presence of IL-22R1 antibody (FIG. 24). These results indicate that the sMDA-7 activity against endothelial cells is IL-22R1 mediated.

### Example 40

### Matrigel Encapsulated SMDA-7 Protein Inhibits Angiogenesis In Vivo

To determine if sMDA-7 could inhibit angiogenesis in vivo, a matrigel assay was utilized as described in Example 19. SMDA-7 protein (10 ng) encapsulated into matrigel containing bFGF was implanted subcutaneously in nude mice. Examination of the matrigel 10 days after implantation demonstrated inhibition of bFGF-induced angiogenesis by sMDA-7 when compared to controls (FIG. 25A). Determination of the hemoglobin content in the matrigel demonstrated a very significant (p=0.0001) inhibitory effect by sMDA-7 on bFGF-induced angiogenesis compared to controls that contained no bFGF, contained only bFGF, or contained recombinant human endostatin (FIG. 25B). Surprisingly the reduction in hemoglobin content by sMDA-7 was much stronger than that observed in the control that had no bFGF.

### Example 40

### In Vivo Mixing of 293-MDA7 Cells Secreting MDA-7 with Tumor Cells Inhibits Subcutaneous Human Lung Tumor Xenografts

The ability of sMDA-7 to inhibit tumor growth was tested by in vivo mixing experiments as described in Example 19. A549 tumor cells were mixed with either parental 293 cells or with 293-mda-7 cells (1 :1 ratio) and injected subcutaneously in the lower right flank of mice. Tumor measurements were initiated when tumors were palpable. A significant inhibition (p=0.001) of tumor growth was observed in animals that received a mixture of A549 and 293-mda-7 cells compared to animals that received a mixture of A549 and parental 293 cells (FIG. 26A). Injection of 293 or 293-mda-7 cells alone do not form tumors in nude mice.

Animals were euthanized on day 22 post-implantation and tumors were harvested and further evaluated. Western blot analysis demonstrated MDA-7 protein expression in tumors that contained a mixture of A549 tumor cells and 293-mda-7 cells compared to controls that contained a mixture of A549 tumor cells and 293 cells (FIG. 26B). Histopathological examination of tumor tissue did not reveal any significant difference in the tumor cell proliferative index or in tumor cell infiltration between control and experimental animals (FIG. 26C). However, reduced vascularization as indicated by CD31 staining was observed in tumors that contained 293-mda-7 cells compared to control tumors that contained 293 cells (FIG. 26C). Reduction in tumor vascularization correlated with decreased hemoglobin content in tumors harvested from animals that received a mixture of A549 and 293-mda-7 cells compared to animals that received a mixture of A549 and parental 293 cells (FIG. 26D). TUNEL staining of tumor tissues from experimental animals demonstrated endothelial cells and tumor cells surrounding the endothelial cell undergoing apoptotic cell death as indicated by the brown staining (FIG. 26C). In contrast, no TUNEL positive staining was observed in control tumor tissues.

### Example 41

### Matrigel Encapsulated 293 Cells Secreting MDA-7 Inhibit Subcutaneous Human Lung Tumor Xenografts

Mice were inoculated subcutaneously with A549 tumor cells in the lower right flank as described in Example 19. When the tumors had reached a size of 50 mm³, 293 cells producing sMDA-7 (293-mda-7) or parental 293 cells (control) were encapsulated in matrigel and implanted subcutaneously at a site distant (upper right flank) from the tumor and the tumor growth was monitored. The growth of A549 lung tumor xenografts was significantly inhibited (p=0.001) by 293 cells secreting MDA-7 when compared to the control group (FIG. 27A). In comparison to the control, the growth of the tumors was suppressed by 50% after implantation of the encapsulated 293 cells producing MDA-7 protein. To confirm that the inhibitory effect was due to sMDA-7, serum samples from animals were tested for MDA-7 protein by Western blot analysis and ELISA. Intense banding of sMDA-7 at the expected 40 Kd size was observed in the serum of animals implanted with 293 cells producing MDA-7 by Western blot analysis (FIG. 27A). However, faint bands were also observed in the control that may be due to some cross-reactivity with serum proteins. The serum levels of sMDA-7 detected by ELISA was approximately 50 ng/ml.

At the end of experimentation, tumors and injected matrigel containing 293-mda-7 cells were harvested and stained. Immunohistochemical analysis of the matrigel from animals receiving 293-mda-7 cells using a monoclonal anti-MDA-7 antibody demonstrated MDA-7 protein expression as evidenced by the brown staining (FIG. 27B). In contrast, MDA-7 was not detected in the matrigel from animals receiving parental 293 cells (FIG. 27B). Additionally, tumors treated with 293-mda-7 had less vascularization than tumors treated with parental 293 cells, as evidenced by CD31 positive staining (FIG. 27B). Histopathological analysis of tumor tissues demonstrated no differences between animals receiving 293 cells and 293-mda-7 cells (FIG. 27B).

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,367,110

U.S. Pat. No. 4,452,901

U.S. Pat. No. 4,554,101

U.S. Pat. No. 4,797,368

U.S. Pat. No. 5,028,592

U.S. Pat. No. 5,139,941

U.S. Pat. No. 5,221,605

U.S. Pat. No. 5,238,808

U.S. Pat. No. 5,310,687

U.S. Pat. No. 5,399,363

U.S. Pat. No. 5,466,468

U.S. Pat. No. 5,543,158

U.S. Pat. No. 5,552,293

U.S. Pat. No. 5,641,515

U.S. Pat. No. 5,739,169

U.S. Pat. No. 5,801,005

U.S. Pat. No. 5,824,311

U.S. Pat. No. 5,830,880

U.S. Pat. No. 5,846,225

U.S. Pat. No. 5,846,233

U.S. Pat. No. 5,846,945

U.S. Pat. No. 6,132,980

U.S. Pat. No. 6,177,074

U.S. Pat. No. 6,204,022

U.S. Pat. No. 6,207,145

U.S. Pat. No. 6,250,469

U.S. Pat. No. 6,326,466

U.S. Pat. No. 6,331,525

U.S. Pat. No. 6,350,589

U.S. Pat. No. 6,372,218

U.S. Pat. No. 6,379,701

WO 0026368

WO 0005356

WO 9828425

WO 9807408

Allen, G.; Fantes, K. H., Nature 287: 408-411, 1980.

Aksentijevich et al., Hum Gene Ther 7(9): 1111-22, 1996.

Albert et al., 1998.

Angel et al., Cell 49(6): 729-39, 1987.

Angel et al., Mol Cell Biol 7(6): 2256-66, 1987.

Ariizumi, 1995.

Aringer et al., Life Sci 64: 2173-86, 1999.

Ashkenazi et al., Science 281: 1305-8, 1998.

Atchison et al., Cell 46(2): 253-62, 1986.

Atchinson and Perry, 1986.

Austin-Edward et al., 1998.

Ausubel, ed., Current protocols in molecular biology, New York, John Wiley & Sons, 1996. Baichwal, Vectors for gene transfer derived from animal DNA viruses: Transient and stable expression of transferred genes. Gene Transfer. R. Kucherlapate. New York, Plenum Press: 117-148, 1986.

Baier et al., Nucleic Acids Res. 21:4830-4835, 1993.

Bakhshi et al., Cell 41(3): 899-906, 1985.

Balachandran, S., Kim, C. N., Yeh, W-C., Mak, T. W., Bhalla, K., and Barber, G. N. Activation of the dsRNA-dependent protein kinase, PKR, induces apoptosis through FADD-mediated death signaling. EMBO J., 17:6888-6902, 1998.

Banerji et al., Cell 27(2 Pt 1): 299-308, 1981.

Banerji et al., Cell 33(3): 729-40, 1983.

Barany and Merrifield, 1979.

Beretta et al., 1996.

Berkhout et al., J Virol 63(12): 5501-4, 1989.

Berrie C P, Expert Opin Investig Drugs 10(6): 1085-98, 2001.

Blanar et al., Embo J 8(4): 1139-44, 1989.

Blumberg et al., Cell 104: 9-19, 2001.

Bodine et al, Embo J 6(10): 2997-3004, 1987.

Boshart et al., Cell 41(2): 521-30, 1985.

Bosze et al., Embo J 5(7): 1615-23, 1986.

Braddock et al., Cell 58(2): 269-79, 1989.

Bukowski et al., Clin Cancer Res 4(10): 2337-47, 1998.

Bulla et al., J Virol 62(4): 1437-41, 1988.

Caley et al., J Virol 71(4): 3031-8, 1997.

Campbell et al., Mol Cell Biol 8(5): 1993-2004, 1988.

Camper et al., Biotechnology 16: 81-7, 1991.

Campo et al., Nature 303(5912): 77-80, 1983.

Caudel et al., J Immunol, in press 2002.

Caux, 1994.

Cavallaro et al., Cancer Lett 176(2): 123-8, 2002.

Celander et al., J Virol 61(2): 269-75, 1987.

Celander, D., B. L. Hsu, et al., J Virol 62(4): 1314-22, 1988.

Chang et al., Mol Cell Biol 9(5): 2153-62, 1989.

Chang, Hepatology 14: 134A, 1991.

Chattergoon et al., 2000.

Chatterjee et al., Proc Natl Acad Sci USA 86(23): 9114-8, 1989.

Chen et al., 2001.

Chol et al., Eur J Biochem 239(3): 579-87, 1996.

Christodoulides et al., 1998.

Clark et al., Hum Gene Ther 6(10): 1329-41, 1995.

Cleary et al., Cell 47(1): 19-28, 1986.

Cleary et al., Proc Natl Acad Sci USA 82(21): 7439-43, 1985.

Clake et al., Nucleic Acids Res. 19:243-248 (1991). Coffin, Retroviridae and their replication. Fields Virology. Fields. New York, Raven Press: 1437-1500, 1990.

Cohen et al., J Cell Physiol Suppl Suppl 5: 75-81, 1987.

Costa et al., Mol Cell Biol 8(1): 81-90, 1988.

Couch, Am. Rev. Resp. Dis. 88: 394-403, 1963.

Coupar et al., Gene 68(1): 1-10, 1988.

Cripe et al., Embo J 6(12): 3745-53, 1987.

Cross et al., Science 267: 1353-6, 1995.

Cryns et al., Genes Dev 12: 1551-70, 1998.

Cuddihy, A. R., Li, S., Tam, N. W. N., Wong, A. H-T., Taya, Y., Abraham, N., Bell, J.C., and Koromilas, A. E. Double-stranded-RNA-activated protein kinase PKR enhances transcriptional activation by tumor suppre4ssor p53. Mol. Cell. Biol., 19: 2475-2484, 1999.

Culotta et al., Mol Cell Biol 9(3): 1376-80, 1989.

Dagon et al., Oncogene 20: 8045-56, 2001.

D'Amico et al., J Biol Chem 275(42): 32649-57, 2000.

Dandolo et al., J Virol 47(1): 55-64, 1983.

Davidson et al., J Immunother 21(5): 389-98, 1998.

Davis et al., J Virol 70(6): 3781-7, 1996.

Deb, A., Haque, S. J., Mogensen, T. R., Silverman, R. H., and Williams, B. R. G. RNA-dependent protein kinase PKR is required or activation of NF-κB by IFN-y in a STAT1-independent pathway. J. Immunol, 166: 6170-6180, 2001.

Der, S. D., Yang, Y. L., Weissman, C., and Williams, B. R. A dsRNA-activated protein kinase-dependent pathway mediating stress-induced apoptosis. Proc. Natl. Acad. Sci., 94: 3279-3283, 1997.

Deschamps et al., Science 230(4730): 1174-7, 1985.

Dumoutier et al., J Immunol 167: 3545-49, 2001.

Easwaran et al., J Biol Chem 274(23): 16641-5, 1999.

Edbrooke et al., Mol Cell Biol 9(5): 1908-16, 1989.

Edery et al., Cell 56:303-312, 1989.

Edlund et al., Science 230(4728): 912-6, 1985.

Ekmekcioglu et al., Intl. J. Cancer. 94(1), 54-59, 2001.

Ellerhorst et al., J Clin Oncol 20: 1069-74, 2002.

Enk, 1992.

el-Kareh et al., Crit Rev Biomed Eng 25(6): 503-71, 1997.

Erlandsson, Cancer Genet Cytogenet 104(1): 1-18, 1998.

Felgner et al., Proc Natl Acad Sci USA 84(21): 7413-7, 1987.

Feng et al., Nature 334(6178): 165-7, 1988.

Feng G. et al., Indentification of Double-Stranded RNA-Binding Domains in the Interferon-Induced Double-Standed RNA-Activated p68 Kinase, PNAS USA, vol. 89, No. 12, Jun. 15, 1992, pp 5447-5451.

Fickenscher et al., Trends Immunol 23: 89-96, 2002.

Firak et al., Mol Cell Biol 6(11): 3667-76.

Flotte et al., Am J Respir Cell Mol Biol 7(3): 349-56, 1992.

Flotte et al., Proc Natl Acad Sci USA 90(22): 10613-7, 1993.

Flotte, et al., Gene Ther 2(1): 29-37, 1995.

Foecking et al., Gene 45(1): 101-5, 1986.

Fraley et al., Proc Natl Acad Sci USA 76(7): 3348-52, 1979.

Freshner, 1992.

Friedmann, Science 244(4910): 1275-81, 1989.

Fry M J, Breast Cancer Res 3(5): 304-12, 2001.

Fujita et al., Cell 49(3): 357-67, 1987.

Gabizon et al., Cancer Res 50(19): 6371-8, 1990.

Gertig and Hunter, 1997.

Ghadge et al., J. Virol. 68:4137-4151 (1994).

Ghosh et al., Targeted Diagn Ther 4: 87-103, 1991.

Ghosh-Choudhury et al., Embo J 6(6): 1733-9, 1987.

Gil, J., Alcami, J., and Esteban, M. Induction of apoptosis by double-stranded-RNA-dependent protein kinase (PKR) involves the α subunit of eukaryotic translation initiation factor 2 and NF-κB. Mol. Cell. Biol., 19: 4653-4663, 1999.

Gillies et al., Cell 33(3): 717-28, 1983.

Gloss et al., Embo J 6(12): 3735-43, 1987.

Godbout et al., 1988.

Goh, K. C., deVeer, M. J., and Williams, B. R. G. The protein kinase PKR is required for p38 MAPK activation and the innate immune response to bacterial endotoxin. EMBO J., 19: 4292-4297, 2000.

Gomez-Foix et al., J Biol Chem 267(35): 25129-34, 1992.

Goodbourn et al., Cell 45(4): 601-10, 1986.

Goodbourn and Maniatis, 1985.

Graham et al., Biotechnology 20: 363-90, 1992.

Graham et al., J Gen Virol 36(1): 59-74, 1977.

Graham et al., Manipulation of adenovirus vector. Methods in molecular biology: Gene transfer and expression protocol. Murray. Clifton, N.J., Humana Press. 7: 109-128, 1991.

Green et al., Science 281: 1309-12, 1998.

Green et al., 1992.

Greene et al., Adv Exp Med Biol 254: 55-60, 1989.

Grosschedl et al., Cell 41(3): 885-97, 1985.

Grunhaus et al., Seminars in Virology 3: 237-252, 1992.

Gunnery et al., Proc. Natl. Acad. Sci. USA 87:8687-8691, 1990.

Haines, G. K., Ghadge, G. D., Thimmapaya, G., and Radosevich, J. A. Expression of PKR (p68) recognized by the monoclonal antibody TJ4C4 in human lung neoplasms. Virchows Arch. B. Cell Pathol. 62: 151-158, 1992.

Hanibuchi et al., Int J Cancer 78(4): 480-5, 1998.

Haslinger et al., Proc Natl Acad Sci USA 82(24): 8572-6, 1985.

Hauber et al., J Virol 62(3): 673-9, 1988.

Hellstrand et al., Acta Oncol 37(4): 347-53, 1998.

Hen et al., Nature 321(6067): 249-51, 1986.

Hensel et al., Lymphokine Res 8(3): 347-51, 1989.

Hermonat et al., Proc Natl Acad Sci USA 81(20): 6466-70, 1984.

Herr et al., Cell 45(3): 461-70, 1986.

Herz et al., Proc Natl Acad Sci USA 90(7): 2812-6, 1993.

Hesdorffer et al., DNA Cell Biol 9(10): 717-23, 1990.

Heufler, 1992.

Hirochika et al., J Virol 61(8): 2599-606, 1987.

Hirsch et al., Mol Cell Biol 10(5): 1959-68, 1990.

Ho et al., Cancer 83(9): 1894-907, 1998.

Holbrook et al., Virology 159(1): 178-82, 1987.

Hovanessian, A. G., J. Interferon Res. 9:641-647, 1989.

Horlick et al., Mol Cell Biol 9(6): 2396-413, 1989.

Horwich et al, J Virol 64(2): 642-50, 1990.

Huang et al., Oncogene 20: 7051-63, 2001.

Huang et al., Cell 27(2 Pt 1): 245-55, 1981.

Hug et al., Mol Cell Biol 8(8): 3065-79, 1988.

Hui et al, Infect Immun 66(11): 5329-36, 1998.

Hwang et al., Mol Cell Biol 10(2): 585-92, 1990.

Icely et al., TIK, a novel serine/threonine kinase is recognized by antibodies directed against phosphotyrosine, The Journal of Biological Chemistry, vol. 266 (24), pp. 16073-16077, 1991.

Imagawa et al., Cell 51(2): 251-60, 1987.

Imbra, et al., Nature 323(6088): 555-8, 1986.

Imler et al., Mol Cell Biol 7(7): 2558-67, 1987.

Imperiale et al., Mol Cell Biol 4(5): 875-82, 1984.

Ivanov et al., Biochem (Mosc) 66: 1, 2001.

Jagus, R., Joshi, B., and Barber, G. N. PKR, apoptosis and cancer. Int. J. Biochem, Cell Biol., 31: 123-138, 1999.

Jakobovits et al., Mol Cell Biol 8(6): 2555-61, 1988.

Jameel et al., Mol Cell Biol 6(2): 710-5, 1986.

Jaynes et al., Mol Cell Biol 8(1): 62-70, 1988.

Jiang et al., Proc Natl Acad Sci USA 93(17): 9160-5, 1996.

Jiang, H., Lin, J. J., Su, Z-A., Goldstein, N. I., and Fisher P. B. Subtraction hybridization identifies a novel melanoma differentiation associated gene, mda-7, modulated during human melanoma differentiation, growth and progression. Oncogene, 11: 2477-2486, 1995.

Jimenez et al., Nat Med 6: 41-8, 2001.

Johnson et al., Mol Cell Biol 9(8): 3393-9, 1989.

Joki et al., Nat Biotech 19: 29-34, 2001.

Jones et al., Cell 13(1): 181-8, 1978.

Judware R. et al., 1991.

Judware R. et al., Inhibition of the dsRNA-Dependent Protein Kinase By A Peptide Derived From the Human Immunodeficiency Virus Type 1 Tat Protein, Journal of Interferon Research, vol. 13, No. 2, April 1993, pp 153-160.

Kadesch et al., Mol Cell Biol 6(7): 2593-601, 1986.

Kaneda et al., Science 243(4889): 375-8, 1989.

Kaplitt et al., Nat Genet 8(2): 148-54, 1994.

Karin et al., Mol Cell Biol 7(2): 606-13, 1987.

Karlsson et al., Embo J 5(9): 2377-85, 1986.

Katinka et al., Cell 20(2): 393-9, 1980.

Kato et al., J Biol Chem 266(6): 3361-4, 1991.

Katso et al., Annu Rev Call Dev Biol 17: 615-75, 2001.

Kawamoto et al., Mol Cell Biol 8(1): 267-72, 1988.

Kerr et al., Br J Cancer 26(4): 239-57, 1972.

Kiledjian et al., Mol Cell Biol 8(1): 145-52, 1988.

Kim et al., J Biol Chem 275(50): 39474-81, 2000.

Kitajewski et al., Cell 45:195-200 (1986).

Kitajima, 1995.

Klamut et al., Mol Cell Biol 10(1): 193-205, 1990.

Koch, et al., Mol Cell Biol 9(1): 303-11, 1989.

Kolmel et al., J Neurooncol 38(2-3): 121-5, 1998.

Kotin et al., Proc Natl Acad Sci USA 87(6): 2211-5, 1990.

Kriegler et al., Blood 63(6): 1348-52, 1984.

Kriegler et al., Cell 38(2): 483-91, 1984.

Kriegler et al., Mol Cell Biol 3(3): 325-39, 1983.

Kromilas et al., 1992.

Kuhen et al., Mechanism of interferon action: Sequence of the human interferon-inducible RNA-dependent protein kinase (PKR) deduced from genomic clones, Gene 178, pp. 191-193, 1996.

Kuhl et al., Cell 50(7): 1057-69, 1987.

Kumar et al., 1994.

Kunz et al., Nucleic Acids Res 17(3): 1121-38, 1989.

Larsen et al., J Biol Chem 261(31): 14373-6, 1986.

Laspia et al., Cell 59(2): 283-92, 1989.

Latimer et al., Mol Cell Biol 10(2): 760-9, 1990.

Laughlin et al., J Virol 60(2): 515-24. 1986.

Lawn et al., The human interferon gene family. Sixth Int. Cong. Hum. Genet., Jerusalem 55 only, 1981.

Le Gal La Salle et al., Science 259(5097): 988-90, 1993.

Lebedeva et al., Oncogene 21(5): 708-18, 2002.

Lebkowski et al., Mol Cell Biol 8(10): 3988-96, 1988.

Lee et al., A Testis Cytoplasmic RNA Binding Protein that has the properties of a translation repressor, Molecular and Cellular Biology, vol. 16(6), pp. 3023-3034, 1996.

Lee et al., Nature 294(5838): 228-32, 1981.

Lee et al., Nucleic Acids Res 12(10): 4191-206, 1984.

Leonardo et al., 1989.

Levrero et al., Gene 101(2): 195-202, 1991.

Li et al., Cell 94: 491-501, 1998.

Lin et al., Mol Cell Biol 10(2): 850-3, 1990.

Liu et al., J Biol Chem 270(42): 24864-70, 1995.

Locksley et al., Cell 104: 487-501, 2001.

Luo et al., 1994.

Luria et al., Embo J 6(11): 3307-12, 1987.

Lusky et al., Mol Cell Biol 3(6): 1108-22, 1983.

Lusky et al., Proc Natl Acad Sci USA 83(11): 3609-13, 1986.

Macejak and Samow, 1991.

Madireddi et al., Cancer Gene Therapy: Past Achievements and Future Challenges: 239-261, 2000.

Madireddi et al., J Cell Physiol 184: 36-46, 2000.

Madireddi et al., Adv Exp Med Biol 465: 239-61, 2000.

Magi-Galluzzi et al., Anal Quant Cytol Histol 20(5): 343-50, 1998.

Maheshwari et al., J Cell Physiol 146: 164-9, 1991.

Majors et al., Proc Natl Acad Sci USA 80(19): 5866-70, 1983.

Mangray et al., Front Biosci 3: D1 148-60, 1998.

Mann et al., Cell 33(1): 153-9, 1983.

Maran et al., 1994.

Markowitz et al., J Virol 62(4): 1120-4, 1988.

Matsue, 1992.

Mayer et al., Cancer Metastasis Rev 17(2): 211-8, 1998.

McCarty et al., J Virol 65(6): 2936-45, 1991.

McCormack et al., 1992.

McCormkick F, Trends in Cell Biol 12:53-86, 1999.

McLaughlin et al., J Virol 62(6): 1963-73, 1988.

McNeall et al., Gene 76(1): 81-8, 1989.

Merrifield, 1986.

Meurs et al., Cell 62:379-390, 1990.

Meurs et al., 1993.

Meurs et al., Molecular cloning and characterization of the human double stranded RNA activated protein kinase induced by interferon, Cell, v9olume 62, pp. 379-390, July 1997.

Mhashilkar et al., Mol Med 7(4): 271-82, 2001.

Mhashilkar et al., MDA-7 negatively regulates the β-catenin and PI3K signaling pathway in breast and lung tumor cells, submitted for publication 2002.

Michaelson et al., Oncogene 20(37): 5093-9, 2001.

Miksicek et al., Cell 46(2): 283-90, 1986.

Mohamadzadeh, 1996.

Mordacq et al., Genes Dev 3(6): 760-9, 1989.

Moreau et al., Nucleic Acids Res 9(22): 6047-68, 1981.

Mougin et al., Ann Biol Clin (Paris) 56(1): 21-8, 1998.

Muesing et al., Cell 48(4): 691-701, 1987.

Mumby et al., Cell Regul 2(8): 589-98, 1991.

Muzio et al., Cell 85: 817-27, 1996.

Muzyczka, Curr Top Microbiol Immunol 158: 97-129, 1992.

Natoli et al., Biochem Pharmacol 56(8): 915-20, 1998.

Neuberger et al., Nucleic Acids Res 16(14B): 6713-24, 1988.

Ng et al., Nucleic Acids Res 17(2): 601-15, 1989.

Nicolas et al., Retroviral vectors. Vectors: A survey of molecular cloning vectors and their uses.

Rodriguez and Denhardt, Stoneham: 494-513, 1988.

Nicolau et al., Biochim Biophys Acta 721(2): 185-90, 1982.

Nicolau et al., Methods Enzymol 149: 157-76, 1987.

Novak et al., Cell Mol Life Sci 56(5-6): 523-37, 1999.

Ohara, Gan To Kagaku Ryoho 25(6): 823-8, 1998.

Ohi et al., Gene 89(2): 279-82, 1990.

Ondek et al., Embo J 6(4): 1017-25, 1987.

Ornitz et al., Mol Cell Biol 7(10): 3466-72, 1987.

Palmiter et al., Cell 29(2): 701-10, 1982.

Parker et al., Br J Cancer 85(12): 1958-63, 2001.

Paskind et al., Virology 67(1): 242-8, 1975.

Pataer, A., Fang, F., Yu, R., Kagawa, S., Hunt, K. K., McDonnell, T. J., Roth, J. A., and Swisher, S. G. Adenoviral Bak overexpression mediates caspase-dependent tumor killing. 60: 788-792, 2000.

Pataer et al., Cancer Res 62: 2239-43, 2002.

Pataer et al., Adenoviral MDA-7 induces apoptosis in lung cancer cells through mitochondrial permeability transition (MPT) independent cytochrome c release, submitted for publication 2002.

Patel R. and Sen G., Characterization of the Interactions Between Double-Stranded RNA and the Double-Stranded RNA Binding Domain of the Interferon Induced Protein Kinase, Cellular and Molecular Biology Research, vol. 40, No. 7/8, 1994.

Patel R. and Sen G., Identification of the Double-Stranded RNA-Binding Domain of the Human Interferon-Inducible Protein Kinase, The Journal of Biological Chemistry, vol. 267, No. 11, Apr. 15, 1992, pp 7671-76.

Pech et al., Mol Cell Biol 9(2): 396-405, 1989.

Peifer et al., Science 14: 1837-51, 2000.

Pelletier et al., Nature 334(6180): 320-5, 1988.

Peng et al., Science 277: 1501-5, 1997.

Perez-Stable et al., Mol Cell Biol 10(3): 1116-25, 1990.

Petryshyn et al., 1988

Petryshyn et al., 1984.

Philip et al., J Biol Chem 268(22): 16087-90, 1993.

Picard et al., Embo J 4(11): 2831-8, 1985.

Pietras et al., Oncogene 17(17): 2235-49, 1998.

Pinkert et al., Genes Dev 1(3): 268-76, 1987.

Ponta et al., Proc Natl Acad Sci USA 82(4): 1020-4, 1985.

Qin et al., 1998.

Queen and Baltimore, 1983.

Quinn et al, Mol Cell Biol 9(11): 4713-21, 1989.

Racher, Biotechnology Techniques 9: 169-174, 1995.

Ragot et al., Nature 361(6413): 647-50, 1993.

Redondo et al., Science 247(4947): 1225-9, 1990.

Reisman et al., Oncogene 4(8): 945-53, 1989.

Renan, Radiother Oncol 19(3): 197-218, 1990.

Resendez et al., Mol Cell Biol 8(10): 4579-84, 1988.

Restifo et al., 2001.

Rice et al., Discovery and in Vitro Development of AIDS Antiviral Biopharmaceuticals, Advances in Pharmacology, vol. 33, pp. 389-438, 1995.

Rich et al., Curr Biol 11: R531-4, 2001.

Rich et al., Hum Gene Ther 4(4): 461-76, 1993.

Ridgeway, Mammalian expression vectors. Vectros: A survey of molecular cloning vectors and their uses. Rodriguez and Denhardt, Stoneham: 467-492, 1988.

Rippe et al., Mol Cell Biol 9(5): 2224-7, 1989.

Rittling et al., Nucleic Acids Res 17(4): 1619-33, 1989.

Rosenfeld et al., Cell 68(1): 143-55, 1992.

Rosenfeld et al., Science 252(5004): 431-4, 1991.

Roux et al, Proc Natl Acad Sci USA 86(23): 9079-83, 1989.

Saito Y, submitted 2002.

Saeki et al., Oncogene, in press.

Saeki et al., Gene Ther 7(23):2051-7, 2000.

Saelens et al., 2000.

Saelens, X., Kalai, M., and Vandenabeele, P. Translation inhibition in apoptosis: caspase-dependent PKR activation and eIF2-α phosphorylation. J. Biol. Chem., 276: 41620-41628, 2001.

Saif-Muthama et al., 2000.

Sambrook et al., In: Molecular Cloning: A Laboratory Manual, 2nd Ed., Cold Spring Harbor Press, Cold Spring Harbor, N.Y., 1989.

Samulski et al., Embo J 10(12): 3941-50, 1991.

Samulski et al., J Virol 63(9): 3822-8, 1989.

Sasaki et al., 2001.

Schaefer et al., J Immunol. 166, 5859, 2001.

Schaffner et al., J Mol Biol 201(1): 81-90, 1988.

Schreiber, 1992.

Searle et al., Mol Cell Biol 5(6): 1480-9, 1985.

SenGupta et al, Nucleic Acids Res. 17:960-978, 1989.

Sharp et al., Cell 59(2): 229-30, 1989.

Shaul et al., Embo J 6(7): 1913-20, 1987.

Shelling et al., Gene Ther 1(3): 165-9, 1994.

Sherman et al., Proc Natl Acad Sci USA 86(17): 6739-43, 1989.

Si.e.,i et al., Proc Natl Acad Sci USA 85(24): 9456-60, 1988.

Si.e.,i et al., 1986.

Sleigh et al., Embo J 4(13B): 3831-7, 1985.

Smalley et al., Mammary Gland Biol Neoplasia 6(1): 37-52, 2001.

Smyth-Templeton et al., 1997.

Solodin et al., Biochemistry 34(41): 13537-44, 1995.

Solyanik et al., Cell Prolif 28(5): 263-78, 1995.

Soo et al., J. Cell. Biochem. 74, 1, 1999.

Spalholz et al., Cell 42(1): 183-91, 1985.

Spandau et al., J Virol 62(2): 427-34, 1988.

Spandidos et al., Embo J 2(7): 1193-9, 1983.

Steinman et al., Annu Rev Immunol 9: 271-96, 1991.

Steinman, 1995.

Stephens et al., Biochem J 248(1): 1-11, 1987.

Stewart and Young, 1984.

Stokke et al., Cell Prolif 30(5): 197-218, 1997.

Stratford-Perricaudet et al., Gene transfer into animals: the promise of adenovirus. Human Gene Transfer. Cohen-Haguenauer and Boiron. France, John Libbey Eurotext: 51-61, 1991.

Stratford-Perricaudet et al., Hum Gene Ther 1(3): 241-56, 1990.

Stuart et al, Nature 317(6040): 828-31, 1985.

Su, A-A., Madireddi, M. T., Lin, J. J., Young, C. S. H., Kitada, S., Reed, J. C., Goldstein, N. I., and Fisher, P. B. The cancer growth suppressor gene mda-7 selectively induces apoptosis in human breast cancer cells and inhibits tumor growth in nude mice. Proc. Natl. Acad. Sci. USA., 95: 14400-14405, 1998.

Sudhakar, A., Ramachandran, R., Ghosh, S., Hasnain, S. E., Kaufman, R. J., and Ramaiah, K. V. A. Phosphorylation of serine 51 in initiation factor 2α (eIF2α) promotes complex formation between eIF2α(P) and causes inhibition in the guanine nucleotide exchange activity of eIF2B. Biochem., 39: 12929-12938, 2000.

Sullivan et al., Mol Cell Biol 7(9): 3315-9, 1987.

Sundararajan et al., J Biol Chem 276: 45120-7, 2001.

Swartzendruber et al., J Cell Physiol 85(2 Pt 1): 179-87, 1975.

Takebe et al., Mol Cell Biol 8(1): 466-72, 1988.

Tam et al., 1983.

Tang et al., Mol Cell 8: 1005-16, 2001.

Tartaglia et al., Immunol Today 13: 151-3, 1992.

Tavernier et al., Nature 301(5901): 634-6, 1983.

Taylor et al., Mol Cell Biol 10(1): 165-75, 1990.

Taylor et al., Mol Cell Biol 10(1): 176-83, 1990.

Taylor, et al., J Biol Chem 264(27): 16160-4, 1989.

Temin, Retrovirus vectors for gene transfer: Efficient integration into and expression of exogenous DNA in vertebrate cell genome. Gene Transfer. Kucherlapati. New York, Plenum Press: 149-188, 1986.

Thierry et al., Proc Natl Acad Sci USA 92(21): 9742-6, 1995.

Thiesen et al., J Virol 62(2): 614-8, 1988.

Top et al., J Infect Dis 124(2): 155-60, 1971.

Toyoshima et al., Cell 78: 67-74, 1994.

Tratschin et al., Mol Cell Biol 4(10): 2072-81, 1984.

Tratschin et al., Mol Cell Biol 5(11): 3251-60, 1985.

Treisman et al., Cell 42(3): 889-902, 1985.

Tronche et al., Mol Biol Med 7(2): 173-85, 1990.

Tronche et al., Mol Cell Biol 9(11): 4759-66, 1989.

Trudel et al., Genes Dev 1(9): 954-61, 1987.

Tsujimoto and Croce, 1986.

Tsujimoto et al., Science 228(4706): 1440-3, 1985.

Tsukamoto et al., Nat Genet 9(3): 243-8, 1995.

Tsuiki et al., Oncogene 20: 420-9, 2001.

Tyndall et al., Nucleic Acids Res 9(23): 6231-50, 1981.

Van Noort et al., J Biol Chem (accepted for 2002 publication).

Vasseur et al., Proc Natl Acad Sci USA 77(2): 1068-72, 1980.

Vattem, K. M., Staschke, K. A., and Wek, R. C. Mechanism of activation of the double-stranded-RNA-dependent protein kinase, PKR: role of dimerization and cellular localization in the stimulation of PKR phosphorylation of eukaryotic initiation factor-2 (eIF2). Eur. J. Biochem., 268: 3674-3684, 2001.

Walsh et al., J Clin Invest 94(4): 1440-8, 1994.

Wang et al., J Biol Chem 277: 7341-7, 2002.

Wang et al., J Biol Chem 281: 1680-83, 1998.

Wang et al., Cell 47(2): 241-7, 1986.

Weber et al., Cell 36(4): 983-92, 1984.

Wei et al., Gene Ther 1(4): 261-8, 1994.

Winoto et al., Embo J 8(3): 729-33, 1989.

Woodfield et al., Biochem J 360(Pt 2): 335-44, 2001.

Wong et al., Gene 10(2): 87-94, 1980.

Yamamoto et al., Cold Spring Harb Symp Quant Biol 47 Pt 2: 977-84, 1983.

Yang et al., Int J Oncol 18(3): 541-8, 2001.

Yang et al., J Virol 68(8): 4847-56, 1994.

Yang et al., Proc Natl Acad Sci USA 87(24): 9568-72, 1990.

Yoder et al., Blood 82(Suppl.): 347A, 1994.

Yutzey et al., Mol Cell Biol 9(4): 1397-405, 1989.

Zamanian-Daryoush et al., Oncogene 18: 315-26, 1999.

Zhang et al., J Biol. Chem. 275: 24436-43, 2000.

Zhang, F., Romano, P. R., Nagamura-Inoue, T., Tian, B., Dever, T. E., Mathews, M. B., Ozato, K., and Hinnebusch, A. G. Binding of double-stranded RNA to protein kinase PKR is required for dimerization and promotes critical autophosphorylation events in the activation loop. J. Biol. Chem., 276: 26946-24958, 2001.

Zhou et al., Exp Hematol 21(7): 928-33, 1993.

Zhou et al., J Exp Med 179(6): 1867-75, 1994.

Zhu et al., Science Jul. 9, 1993;261(5118):209-11.

What is claimed is:

1. An immunogenic composition comprising: (a) an immunogenic molecule or a nucleic acid encoding an immunogenic molecule; and (b) a recombinant MDA-7 polypeptide or an isolated nucleic acid expressing the MDA-7 polypeptide.

2. The composition of claim 1, wherein the composition is in a pharmaceutically acceptable diluent.

3. The composition of claim 1, wherein the immunogenic molecule is an antigen.

4. The composition of claim 3, wherein the antigen is a tumor antigen.

5. The composition of claim 4, wherein the tumor antigen is PSA, CEA, MAGE1, MAGE3, gp100, AFP, her2, tert, muc1, NY-ESO, bcr-ab1, trp1, trp2, MART, BAGE, GAGE, or PMSA.

6. The composition of claim 3, wherein the antigen is a microbial, viral or fungal antigen.

7. The composition of claim 1, wherein the immunogenic molecule is at least one polypeptide.

8. The composition of claim 1, wherein the immunogenic molecule is a T-cell activation molecule.

9. The composition of claim 1, wherein the MDA-7 polypeptide comprises amino acids from 49 to 206 of SEQ ID NO:2.

10. The composition of claim 14, wherein the MDA-7 polypeptide comprises the sequence of SEQ ID NO:2.

11. The composition of claim 1 comprising a recombinant MDA-7 polypeptide.

12. The composition of claim 1, wherein the nucleic acid is an expression vector.

13. The composition of claim 12, wherein the expression vector is a viral vector.

14. The composition of claim 13, wherein the viral vector is an adenoviral vector, a retroviral vector, a vaccinia viral vector, an adeno-associated viral vector, a polyoma viral vector, an alphaviral vector, a rhabdoviral vector or a herpes viral vector.

15. The composition of claim 1, wherein the MDA-7 polypeptide comprises a secretory signal.

16. The composition of claim 1, wherein the composition further comprises a colloidal carrier.

17. The immunogenic composition of claim 1, further comprising a cytokine or an isolated nucleic acid encoding the cytokine.

18. A method of promoting an immune response in a patient comprising administering to the patient an effective amount of a MDA-7 polypeptide or a nucleic acid encoding the MDA-7 polypeptide, wherein the MDA-7 polypeptide promotes the immune response in the patient.

19. The method of claim 18, further comprising providing to a patient an immunogenic molecule or a nucleic acid encoding the immunogenic molecule.

20. The method of claim 19, wherein the immune response is against the immunogenic molecule.

21. The method of claim 19, wherein the immunogenic molecule is an antigen.

22. The method of claim 21, wherein the antigen is a tumor antigen.

23. The method of claim 22, wherein the tumor antigen is PSA, CEA, MAGE1, MAGE3, gp100, AFP, her2, tert, muc1, NY-ESO, bcr-ab1, trp1, trp2, MART, BAGE, GAGE, or PMSA.

24. The method of claim 22, wherein i) the immunogenic molecule and ii) the MDA-7 polypeptide or the nucleic acid encoding the MDA-7 are administered before chemotherapy, radiotherapy or surgery.

25. The method of claim 22, wherein the immunogenic molecule and the MDA-7 polypeptide are administered during chemotherapy, radiotherapy or surgery.

26. The method of claim 22, wherein the immunogenic molecule and the MDA-7 are administered to the patient after chemotherapy, radiotherapy or surgery.

27. The method of claim 21, wherein the antigen is a microbial, viral, or fungal antigen.

28. The method of claim 19, wherein the immunogenic molecule comprises at least one polypeptide.

29. The method of claim 18, further comprising identifying a patient in need of promoting an immune response.

30. The method of claim 18, wherein the MDA-7 is provided to the patient by administering a vector comprising an isolated nucleic acid sequence encoding the MDA-7 polypeptide.

31. The method of claim 30, wherein the vector is an expression vector.

32. The method of claim 31, wherein the expression vector is a viral vector.

33. The method of claim 32, wherein the viral vector is an adenoviral vector, a retroviral vector, a vaccinia viral vector, an adeno-associated viral vector, a polyoma viral vector, an alphaviral vector, a rhabdoviral vector or a herpes viral vector.

34. The method of claim 30, wherein the vector further comprises the nucleic acid sequence encoding the immunogenic molecule.

35. The method of claim 18, further comprising detecting the immune response.

36. The method of claim 35, wherein the immune response comprises increasing activity of a T-cell, a NK cell, a macrophage, or a dendritic cell.

37. The method of claim 35, wherein the immune response comprises increasing a cytokine concentration in the patient or inducing maturation of a dendritic cell.

38. The method of claim 37, wherein the cytokine is an interferon or an interleukin.

39. The method of claim 38, wherein the interferon is IFN-α, IFN-β, or IFN-γ.

40. The method of claim 38, wherein the interleukin is IL-1β, IL-2, IL-4, IL-6, IL-8, IL-10, or IL-12.

41. The method of claim 18, wherein the MDA-7 polypeptide comprises amino acids from 49 to 206 of SEQ ID NO:2.

42. The method of claim 18, wherein the MDA-7 polypeptide comprises a sequence of SEQ ID NO:2.

43. The method of claim 18, wherein the MDA-7 polypeptide further comprises a secretory signal.

44. The method of claim 43, wherein the secretory signal is further defined as a positively charged N-terminal region in combination with a hydrophobic core.

45. The method of claim 18, wherein the MDA-7 polypeptide is administered systemically to the patient by continuous infusion or by intravenous injection.

46. The method of claim 18, wherein the MDA-7 polypeptide is administered as a direct injection to an immuno-compromised site.

47. The method of claim 18, wherein the immunogenic molecule is Mycobaterium tuberculosis soluble factor (Mtb), phenol soluble modulin (PSM), CMV-G, CMV-M, EBV capsid-EB nuclear antigen (EBNA), gp120, gp41, tat, rev, gag, toxa antigen, rubella antigen, mumps antigen, alpha-fetoprotein (AFP), adenocarcinoma antigen (ART-4), BAGE, CAMEL, CAP-I, CASP-8, CDC27m, CDK4/m, CEA, CT, Cyp-B, DAM, ELF2M, ETV6-AMLI, ETS G250, GnT-V, HAGE, HER2/neu, HLA-A*0201-R1701, HPV-E7, HSP 70-2M, HST-2, hTERT, ICE, KIAA 0205, LAGE, LDLR/FUT, MAGE, MART, MC1R, MUCI, MUM-1, MUM-2, MUM-3, NA88-A, NY-ESO-I, p15, Pm1/RARalpha, PRAME, PSA, PSM, RAGE, RU1, RU2, SAGE, SART-1, SART-3, TEL/AML1, TPI/m, TRP-1, TRP-2, or WT1.

48. The method of claim 48, wherein the nucleic acid sequence encoding the immunogen further comprises an expression vector.

49. A method of treating cancer in a patient comprising providing to the patient a tumor antigen; and administering an effective amount of a MDA-7 polypeptide, wherein the MDA-7 polypeptide provides the patient with a therapeutic benefit.

50. The method of claim 49, wherein the tumor antigen is PSA, CEA, MAGE1, MAGE3, gp100, AFP, her2, tert, muc1, NY-ESO, bcr-ab1, trp1, trp2, MART, BAGE, GAGE, or PMSA.

51. A method of treating a tumor in a patient comprising (a) providing to the patient an immunogenic molecule to induce an immune response against the immunogenic molecule; and (b) administering to the patient an effective amount of a MDA-7 polypeptide, wherein the MDA-7 enhances the induced immune response and decreases the tumor as compared to treatment with the immunogenic molecule alone.

52. The method of claim 51, wherein the immunogenic molecule is a tumor antigen.

53. The method of claim 52, wherein the tumor antigen is PSA, CEA, MAGE1, MAGE3, gp100, AFP, her2, tert, muc1, NY-ESO, bcr-ab1, trp1, trp2, MART, BAGE, GAGE, or PMSA.

54. The method of claim 51, wherein the decrease is a decrease in tumor size or tumor growth rate.

55. A therapeutic composition comprising a recombinant MDA-7 polypeptide or an isolated nucleic acid encoding the MDA-7 polypeptide and at least one cytokine or an isolated nucleic acid encoding the cytokine.

56. The composition of claim 55, wherein the cytokine is further defined as an interferon α, interferon β, or interferon γ.

57. The composition of claim 56, wherein the cytokine is further defined as interferon γ.

58. The composition of claim 55, wherein an amino acid sequence of the MDA-7 polypeptide is that set forth in SEQ ID NO:2.

59. The composition of claim 55, wherein an amino acid sequence of the MDA-7 polypeptide comprises amino acids 49 to 206 of SEQ ID NO:2.

60. The composition of claim 55, wherein a nucleotide sequence of the nucleic acid encoding an MDA-7 polypeptide is the nucleic acid sequence set forth in SEQ I) NO: 1.

61. A method of enhancing an immune response against an immunogen comprising (a) providing to a patient a polypeptide having an amino acid sequence of the immunogen; and (b) administering to the patient an effective amount of a first composition comprising an MDA-7 polypeptide or a nucleic acid encoding an MDA-7 polypeptide and a second composition comprising an interferon or a nucleic acid encoding the interferon.

62. The method of claim 61, wherein the interferon is interferon α, interferon β, or interferon γ.

63. The method of claim 62, wherein the interferon is interferon γ.

64. The method of claim 61, wherein the first and second compositions are administered in the same pharmaceutical preparation.

65. The method of claim 61, wherein the first and second compositions are administered in different pharmaceutical preparations.

66. A method of inducing anti-angiogenesis of a tumor in a patient comprising administering to IL-22 receptor-positive cells in the patient an effective amount of an MDA-7 polypeptide to bind the IL-22 receptor and induce anti-angiogenesis of the tumor.

67. The method of claim 66, wherein the 1L-22 receptor-positive cells are endothelial cells.

68. The method of claim 67, wherein the endothelial cells are not adjacent to the tumor.

69. The method of claim 66, wherein the MDA-7 polypeptide is secreted MDA-7 and is glycosylated.

70. A method of inducing cell death in a cell, comprising obtaining an MDA-7 targeting construct, wherein the MDA-7 targeting construct includes a nucleic acid encoding an MDA-7 polypeptide and a targeting sequence under the control of a promoter, and contacting the cell with an amount of the MDA-7 targeting construct that is effective to deliver the MDA-7 targeting construct to the cell, wherein cell death of the cell is induced.

71. The method of claim 70, wherein the targeting construct comprises DNA encoding MDA-7, wherein said DNA does not encode a functional MDA-7 signal peptide.

72. The method of claim 70, wherein the targeting construct comprises DNA encoding MDA-7 and a nuclear localization signal peptide.

73. The method of claim 70, wherein the targeting construct comprises DNA encoding MDA-7 and an endoplasmic reticulum signal peptide.

74. The method of claim 70, wherein the targeting construct comprises DNA encoding MDA-7 and a mitochondrial signal peptide.

75. A method for inducing cell death in a tumor cell comprising administering to the cell i) an MDA-7 polypeptide or a nucleic acid encoding the MDA-7 polypeptide and ii) an inhibitor of NF-κB, COX-2, Hsp9O, or a protein kinase.

76. A method for inducing cell death in a tumor cell comprising administering to the cell i) an MDA-7 polypeptide or a nucleic acid encoding the MDA-7 polypeptide and ii) an anti-inflammatory agent.
